(19) United States
(12) Patent Application Publication
(10) Pub. No.: US 2025/0250299 A1
(43) Pub. Date: Aug. 7, 2025

(54) MACROCYCLIC IMMUNOMODULATORS

(71) Applicant: Bristol-Myers Squibb Company, Princeton, NJ (US)

(72) Inventors: Michael A. POSS, Lawrenceville, NJ (US); Martin Patrick ALLEN, Flemington, NJ (US); Jennifer X. QIAO, Princeton, NJ (US); Claude A. QUESNELLE, Skillman, NJ (US); Tammy C. WANG, Lawrenceville, NJ (US); Tao WANG, Glastonbury, CT (US); Yunhui ZHANG, Princeton, NJ (US); Zhongxing ZHANG, Madison, CT (US)

(21) Appl. No.: 18/852,751

(22) PCT Filed: Mar. 28, 2023
(86) PCT No.: PCT/US2023/065057

§ 371 Date: Sep. 30, 2024

Related U.S. Application Data
(60) Provisional application No. 63/324,433, filed on Mar. 28, 2022.

(51) Int. Cl. C07K 7/52; A61K 38/00

(52) U.S. Cl. CPC C07K 7/52; A61K 38/00

(57) ABSTRACT

In accordance with the present disclosure, macrocyclic compounds have been discovered that bind to PD-1 and are capable of inhibiting the interaction of PD-1 with PD-L1. These macrocyclic compounds exhibit in vitro immunomodulatory efficacy thus making them therapeutic candidates for the treatment of various diseases including cancer and infectious diseases.

### CROSS-REFERENCE TO RELATED APPLICATIONS

This application claims the priority benefit of U.S. Provisional Application No. 63/324,433, filed Mar. 28, 2022, which is incorporated herein by reference in its entirety.

### FIELD

The present disclosure provides macrocyclic compounds that bind to PD-1 and are capable of inhibiting the interaction of PD-1 with PD-L1. These macrocyclic compounds exhibit in vitro immunomodulatory efficacy thus making them therapeutic candidates for the treatment of various diseases including cancer.

### BACKGROUND

Human cancers harbor numerous genetic and epigenetic alterations, generating neoantigens potentially recognizable by the immune system (Sjoblom et al., 2006). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells (Agata et al., supra; Okazaki et al., Curr. Opin. Immunol., 14:779-782 (2002); Bennett et al., J. Immunol., 170:711-718 (2003)).

The PD-1 protein is a 55 kDa type I transmembrane protein that is part of the Ig gene superfamily (Agata et al., Int. Immunol., 8:765-772 (1996)). PD-1 contains a membrane proximal immunoreceptor tyrosine inhibitory motif (ITIM) and a membrane distal tyrosine-based switch motif (ITSM) (Thomas, M. L., J. Exp. Med., 181:1953-1956 (1995); Vivier, E. et al., Immunol. Today, 18:286-291 (1997)). Although structurally similar to CTLA-4, PD-1 lacks the MYPPY motif that is critical for CD80 CD86 (B7-2) binding. Two ligands for PD-1 have been identified, PD-L1 (B7-H1) and PD-L2 (b7-DC). Activation of T cells expressing PD-1 has been shown to be downregulated upon interaction with cells expressing PD-L1 or PD-L2 (Freeman et al., J. Exp. Med, 192:1027-1034 (2000); Latchman et al., Nat. Immunol., 2:261-268 (2001); Carter et al., Eur. J. Immunol., 32:634-643 (2002)). Both PD-L1 and PD-L2 are B7 protein family members that bind to PD-1, but do not bind to other CD28 family members. The PD-L1 ligand is abundant in a variety of human cancers (Dong et al., Nat. Med, 8:787-789 (2002)). The interaction between PD-1 and PD-L1 results in a decrease in tumor infiltrating lymphocytes, a decrease in T-cell receptor mediated proliferation, and immune evasion by the cancerous cells (Dong et al., J. Mol. Med, 81:281-287 (2003); Blank et al., Cancer Immunol. Immunother., 54:307-314 (2005); Konishi et al., Clin. Cancer Res., 10:5094-5100 (2004)). Immune suppression can be reversed by inhibiting the local interaction of PD-1 with PD-L1, and the effect is additive when the interaction of PD-1 with PD-L2 is blocked as well (Iwai et al., Proc. Natl. Acad Sci. USA, 99:12293-12297 (2002); Brown et al., J Immunol., 170:1257-1266 (2003)).

When PD-1 expressing T cells contact cells expressing its ligands, functional activities in response to antigenic stimuli, including proliferation, cytokine secretion, and cytotoxicity, are reduced. PD-1/PD-L1 or PD-L2 interactions down regulate immune responses during resolution of an infection or tumor, or during the development of self tolerance (Keir, M. E. et al., Annu. Rev. Immunol., 26:Epub (2008)). Chronic antigen stimulation, such as that which occurs during tumor disease or chronic infections, results in T cells that express elevated levels of PD-1 and are dysfunctional with respect to activity towards the chronic antigen (reviewed in Kim et al., Curr. Opin. Imm. (2010)). This is termed “T cell exhaustion”. B cells also display PD-1/PD-ligand suppression and “exhaustion”.

In addition to enhancing immunologic responses to chronic antigens, blockade of the PD-1/PD-L1 pathway has also been shown to enhance responses to vaccination, including therapeutic vaccination in the context of chronic infection (Ha, S. J. et al., “Enhancing therapeutic vaccination by blocking PD-1-mediated inhibitory signals during chronic infection”, J. Exp. Med, 205(3):543-555 (2008); Finnefrock, A. C. et al., “PD-1 blockade in rhesus macaques: impact on chronic infection and prophylactic vaccination”, J. Immunol., 182(2):980-987 (2009); Song, M.-Y. et al., “Enhancement of vaccine-induced primary and memory CD8+t-cell responses by soluble PD-1”, J. Immunother., 34(3):297-306 (2011).

The PD-1 pathway is a key inhibitory molecule in T cell exhaustion that arises from chronic antigen stimulation during tumor disease. Accordingly, agents that block the interaction of PD-1 with PD-L1 are desired.

### SUMMARY

The present disclosure provides macrocyclic compounds which inhibit the PD-1/PD-L1 protein/protein interaction, and are thus useful for the amelioration of various diseases, including cancer.

In one aspect, the present disclosure provides a compound of formula (I):

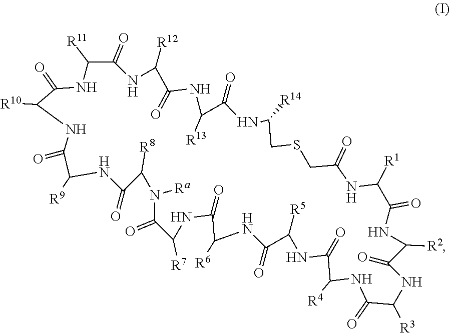

or a pharmaceutically acceptable salt thereof, wherein:

R¹ is selected from C₁-C₆alkyl, aminoC₁-C₆alkyl, arylC₁-C₆alkyl, heteroarylC₁-C₆alkyl, hydroxyC₁-C₆alkyl, —X—R³¹, —(CH₂)_{z}—O—(CH₂)_{z}-triazolyl-X—R³⁵, and NH₂C(X″)NHC₁-C₆alkyl, wherein X″ is O or NH, and wherein the aryl part of the arylC₁-C₆alkyl and the heteroaryl part of the heteroarylC₁-C₆alkyl are optionally substituted with one or two groups independently selected from carboxy, carboxyC₁-C₆alkoxy, —X—R³¹, and —O—(CH₂)_{z}-triazolyl-X-R³⁵;

R² is selected from arylC₁-C₆alkyl, and heteroarylC₁-C₆alkyl, wherein the aryl part of the arylC₁-C₆alkyl and the heteroaryl part of the heteroarylC₁-C₆alkyl are optionally substituted with one or two groups independently selected from carboxy, carboxyC₁-C₆alkoxy, -X—R³¹, and —O—(CH₂)_{z}-triazolyl-X—R³⁵;

R³ is carboxyC₁-C₃alkyl;

R⁴ is selected from arylC₁-C₆alkyl and heteroarylC₁-C₆alkyl, wherein the aryl part of the arylC₁-C₆alkyl and the heteroaryl part of the heteroarylC₁-C₆alkyl are optionally substituted with one or two C₁-C₆alkyl groups;

R⁵ is selected from C₁-C₆alkyl, arylC₁-C₆alkyl, —X—R³¹, and —(CH₂)_{z}—O—(CH₂)_{z}-triazolyl-X—R³⁵, wherein the aryl part of the arylC₁-C₆alkyl is optionally substituted with one or two groups independently selected from carboxy, carboxyC₁-C₆alkoxy, hydroxy, —X—R³¹, and —O—(CH₂)_{z}-triazolyl-X—R³⁵;

R⁶ is aryl-arylC₁-C₃alkyl;

R⁷ is selected from C₁-C₆alkyl, arylC₁-C₆alkyl, —X—R³¹, and —(CH₂)_{z}—O—(CH₂)_{z}-triazolyl-X—R³⁵, wherein the aryl part of the arylC₁-C₆alkyl is optionally substituted with one or two groups independently selected carboxy, carboxyC₁-C₆alkoxy, —X—R³¹, and —O—(CH₂)_{z}-triazolyl-X—R³⁵;

R⁸ is selected from C₁-C₆alkyl, aminoC₁-C₆alkyl, —X—R³¹, and —O—(CH₂)_{z}—triazolyl-X—R³⁵;

R⁹ is C₁-C₆alkyl;

R¹⁰ is selected from amidoC₁-C₆alkyl, aminoC₁-C₆alkyl, —X—R³¹, and —(CH₂)_{z}—O—(CH₂)_{z}-triazolyl-X—R³⁵;

R¹¹ is (C₃-C₈cycloalkyl)C₁-C₆alkyl;

R¹² is selected from C₁-C₆alkyl;

R¹³ is selected from arylC₁-C₆alkyl, carboxyC₁-C₆alkyl, hydroxyC₁-C₆alkyl, —X—R³¹, and —(CH₂)_{z}—O—(CH₂)_{z}-triazolyl-X—R³⁵, wherein the aryl part of the arylC₁-C₆alkyl is optionally substituted with one or two groups independently selected carboxy, carboxyC₁-C₆alkoxy, —X—R³¹, and —O—(CH₂)_{z}-triazolyl-X—R³⁵;

R¹⁴ is —C(O)NH₂ or —C(O)NHCHR¹⁵C(O)NHR⁵⁰, wherein:

R¹⁵ is selected from hydrogen, C₁-C₆alkyl, and aminoC₁-C₆alkyl; and

R⁵¹ is selected from hydrogen and NH₂C(O)CH₂(OCH₂CH₂)₂—; and

R³¹ is —CO₂H, —C(O)NR^{w}R^{x}, —CH₃, alexa-5-SDP, and biotin;

each z is independently 1, 2, 3, 4, 5, or 6; and

R³⁵ is selected from —CO₂H, —C(O)NR^{w}R^{x}, CH₃, biotin, 2-fluoropyridine, —C(O)—(CH₂)₂—C(O)-vitamin E, and —C(O)-vitamin E; wherein R^{x} and R^{W} are independently selected from hydrogen and C₁-C₆alkyl;

X is a chain of between 1 and 172 atoms wherein the atoms are selected from carbon and oxygen and wherein the chain may contain one, two, three, or four groups selected from —NHC(O)NH—, and —C(O)NH— embedded therein; and wherein the chain is optionally substituted with one to six groups independently selected from —CO₂H, —C(O)NH₂, —CH₂C(O)NH₂, and —(CH₂)CO₂H;

provided that at least one of R¹, R², R⁵, R⁷, R⁸, R¹⁰, and R¹³ is, or is substituted with, a group selected from —X—R³¹, —(CH₂)_{z}—O—(CH₂)_{z}-triazolyl-X—R³⁵, and —O—(CH₂)_{z}-triazolyl-X—R³⁵.

In some aspects, vitamin E refers to

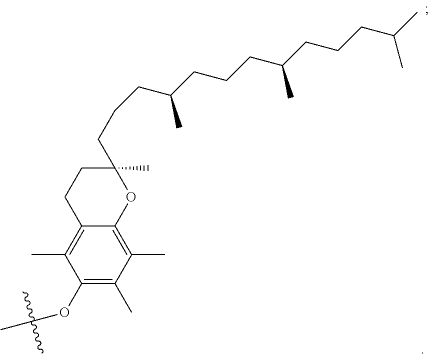 [SMILES: *Oc1c(C)c(C)c2c(c1C)CC[C@@](C)(CCC[C@H](C)CCC[C@H](C)CCCC(C)C)O2 ; "."]

where [symbol] denotes the point of attachment to the carbonyl group.

In some aspects, alexa-5-SDP refers to

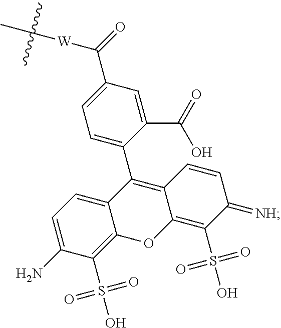

wherein W is O or NH and [symbol] denotes the point of attachment to the parent molecular moiety.

In some aspects, biotin refers to

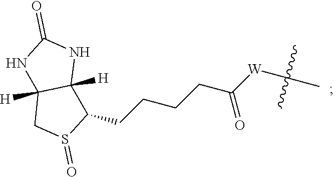

wherein W is O or NH and [symbol] denotes the point of attachment to the parent molecular moiety.

In some aspects, R³¹ is —CO₂H. In some aspects, R³¹ is —NH₂.

In some aspects, R³⁵ is —CO₂H. In some aspects, R³⁵ is —NH₂.

In some aspects, the present disclosure provides a compound of formula (I), or a pharmaceutically acceptable salt thereof, wherein:
R¹ is selected from C₁-C₆alkyl, aminoC₁-C₆alkyl, arylC₁-C₆alkyl, heteroarylC₁-C₆alkyl, hydroxyC₁-C₆alkyl, HO₂C(CH₂)₁₀C(O)NH(CH₂)_{z}— and NH₂C(X″)NHC₁-C₆alkyl, wherein z is 1, 2, 3, or 4 and X″ is O or NH, and wherein the aryl part of the arylC₁-C₆alkyl and the heteroaryl part of the heteroarylC₁-C₆alkyl are optionally substituted with one or two groups independently selected from carboxy, carboxyC₁-C₆alkoxy, and HO₂C(CH₂)₁₆C(O)NHCH(CO₂H)(CH₂)₂C(O)NH(CH₂CH₂O)₁₁(CH₂)₂triazolylCH₂O—;
R² is selected from arylC₁-C₆alkyl, and heteroarylC₁-C₆alkyl, wherein the aryl part of the arylC₁-C₆alkyl and the heteroaryl part of the heteroarylC₁-C₆alkyl are optionally substituted with one or two groups independently selected from carboxy, carboxyC₁-C₆alkoxy, HO₂C(CH₂)₁₀C(O)NH(CH₂)_{z}—, and HO₂C(CH₂)₁₆C(O)NHCH(CO₂H)(CH₂)₂C(O)NH(CH₂CH₂O)₁₁(CH₂)₂triazolylCH₂O—, wherein z is 1, 2, 3, or 4;
R⁵ is selected from C₁-C₆alkyl, arylC₁-C₆alkyl, and HO₂C(CH₂)₁₆C(O)NHCH(CO₂H)(CH₂)₂C(O)NH(CH₂CH₂O)₁₁(CH₂)₂triazolylCH₂OCH₂—, wherein the aryl part of the arylC₁-C₆alkyl is optionally substituted with one or two groups independently selected from carboxy, carboxyC₁-C₆alkoxy, hydroxy, and HO₂C(CH₂)₁₆C(O)NHCH(CO₂H)(CH₂)₂C(O)NH(CH₂CH₂O)₁₁(CH₂)₂triazolylCH₂O—;
R⁷ is selected from C₁-C₆alkyl, arylC₁-C₆alkyl, HO₂C(CH₂)₁₀C(O)NH(CH₂)_{z}—, HO₂C(CH₂)₁₆C(O)NHCH(CO₂H)(CH₂)₂C(O)NH(CH₂CH₂O)₁₁(CH₂)₂triazolylCH₂OCH₂—, wherein z is 1, 2, 3, or 4, and wherein the aryl part of the arylC₁-C₆alkyl is optionally substituted with one or two groups independently selected carboxy, carboxyC₁-C₆alkoxy, and HO₂C(CH₂)₁₆C(O)NHCH(CO₂H)(CH₂)₂C(O)NH(CH₂CH₂O)₁₁(CH₂)₂triazolylCH₂O—;
R⁸ is selected from C₁-C₆alkyl, aminoC₁-C₆alkyl, HO₂C(CH₂)₁₀C(O)NH(CH₂)_{z}—, HO₂C(CH₂)₁₆C(O)NHCH(CO₂H)(CH₂)₂C(O)NH(CH₂CH₂O)₁₁(CH₂)₂triazolylCH₂OCH₂—, wherein z is 1, 2, 3, or 4;
R¹⁰ is selected from amidoC₁-C₆alkyl, aminoC₁-C₆alkyl, and HO₂C(CH₂)₁₀C(O)NH(CH₂)_{z}—, wherein z is 1, 2, 3, or 4;
R¹³ is selected from arylC₁-C₆alkyl, carboxyC₁-C₆alkyl, hydroxyC₁-C₆alkyl, HO₂C(CH₂)₁₀C(O)NH(CH₂)_{z}—, wherein z is 1, 2,3 or 4, and HO₂C(CH₂)₁₆C(O)NHCH(CO₂H)(CH₂)₂C(O)NH(CH₂CH₂O)₁₁(CH₂)₂triazolylCH₂OCH₂—; provided that at least one of R¹, R², R⁵, R⁷, R⁸, R¹⁰, and R¹³ is, or is substituted with, a group selected from
HO₂C(CH₂)₁₆C(O)NHCH(CO₂H)(CH₂)₂C(O)NH(CH₂CH₂O)₁₁(CH₂)₂triazolylCH₂O—, HO₂C(CH₂)₁₀C(O)NH(CH₂)_{z}, and
HO₂C(CH₂)₁₆C(O)NHCH(CO₂H)(CH₂)₂C(O)NH(CH₂CH₂O)ln(CH₂)₂triazolylCH₂OCH₂—.

In some aspects, at least one of R¹, R⁷, R⁸, R¹⁰, and R¹³ is HO₂C(CH₂)₁₀C(O)NH(CH₂)_{z}—, wherein z is 1, 2, 3, or 4.

In some aspects, at least one of R¹, R², R⁵, and R⁷ is arylC₁-C₆alkyl or heteroarylC₁-C₆alkyl wherein the aryl part of the arylC₁-C₆alkyl and the heteroaryl part of the heteroarylC₁-C₆alkyl are substituted with HO₂C(CH₂)₁₆C(O)NHCH(CO₂H)(CH₂)₂C(O)NH(CH₂CH₂O)₁₁(CH₂)₂triazolylCH₂O— or HO₂C(CH₂)₁₀C(O)NH(CH₂)_{z}—.

In some aspects, at least one of R⁵, R⁷, R⁸, and R¹³ is HO₂C(CH₂)₁₆C(O)NHCH(CO₂H)(CH₂)₂C(O)NH(CH₂CH₂O)₁₁(CH₂)₂triazolylCH₂OCH₂—.

In some aspects, R² is arylC₁-C₆alkyl, wherein the aryl part of the arylC₁-C₆alkyl is optionally substituted with one or two groups independently selected from carboxy, carboxyC₁-C₆alkoxy, HO₂C(CH₂)₁₀C(O)NHCH₂—, and HO₂C(CH₂)₁₆C(O)NHCH(CO₂H)(CH₂)₂C(O)NH(CH₂CH₂O)₁₁(CH₂)₂triazolylCH₂O—.

In some aspects, R⁴ is arylC₁-C₆alkyl, wherein the aryl part of the arylC₁-C₆alkyl is optionally substituted with one or two C₁-C₆alkyl groups.

In some aspects, R⁵ is arylC₁-C₆alkyl, wherein the aryl part of the arylC₁-C₆alkyl is optionally substituted with one or two groups independently selected from carboxy, carboxyC₁-C₆alkoxy, hydroxy, and HO₂C(CH₂)₁₆C(O)NHCH(CO₂H)(CH₂)₂C(O)NH(CH₂CH₂O)₁₁(CH₂)₂triazolylCH₂O—.

In some aspects, R⁷ is arylC₁-C₆alkyl, wherein the aryl part of the arylC₁-C₆alkyl is optionally substituted with one or two groups independently selected carboxy, carboxyC₁-C₆alkoxy, and HO₂C(CH₂)₁₆C(O)NHCH(CO₂H)(CH₂)₂C(O)NH(CH₂CH₂O)₁₁(CH₂)₂triazolylCH₂O—.

In some aspects, R⁸ is aminoC₁-C₆alkyl.

In some aspects, R¹⁰ is aminoC₁-C₆alkyl.

In some aspects, the present disclosure provides a pharmaceutical composition comprising a compound of any of the above aspects, or a pharmaceutically acceptable salt thereof.

In some aspects, the present disclosure provides a method of enhancing, stimulating, and/or increasing an immune response in a subject in need thereof, wherein the method comprises administering to the subject a therapeutically effective amount of a compound of any of the above aspects, or a pharmaceutically acceptable salt thereof.

In some aspects, the present disclosure provides a method of blocking the interaction of PD-1 with PD-L1 in a subject, wherein the method comprises administering to the subject a therapeutically effective amount of a compound of any of the above aspects, or a pharmaceutically acceptable salt thereof.

### DETAILED DESCRIPTION

Unless otherwise indicated, any atom with unsatisfied valences is assumed to have hydrogen atoms sufficient to satisfy the valences.

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

As used herein, the phrase “or a pharmaceutically acceptable salt thereof” refers to at least one compound, or at least one salt of the compound, or a combination thereof. For example, “a compound of Formula (I) or a pharmaceutically acceptable salt thereof” includes, but is not limited to, a compound of Formula (I), two compounds of Formula (I), a pharmaceutically acceptable salt of a compound of Formula (I), a compound of Formula (I) and one or more pharmaceutically acceptable salts of the compound of Formula (I), and two or more pharmaceutically acceptable salts of a compound of Formula (I).

The term “C₁-C₆alkoxy”, as used herein, refers to a C₁-C₆alkyl group attached to the parent molecular moiety through an oxygen atom.

The term “C₁-C₆alkyl”, as used herein, refers to a group derived from a straight or branched chain saturated hydrocarbon containing from one to six carbon atoms.

The term “amido,” as used herein, refers to —C(O)NH₂.

The term “amidoC₁-C₆alkyl,” as used herein, refers to an amido group attached to the parent molecular moiety through a C₁-C₆alkyl group.

The term “amino,” as used herein, refers to —NH₂.

The term “aminoC₁-C₆alkyl,” as used herein, refers to an amino group attached to the parent molecular moiety through a C₁-C₆alkyl group.

The term “aryl,” as used herein, refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The aryl groups of the present disclosure can be attached to the parent molecular moiety through any substitutable carbon atom in the group. Representative examples of aryl groups include, but are not limited to, indanyl, indenyl, naphthyl, phenyl, and tetrahydronaphthyl.

The term “arylC₁-C₆alkyl,” as used herein, refers to an aryl group attached to the parent molecular moiety through a C₁-C₆alkyl group.

The term “carboxy”, as used herein, refers to —CO₂H.

The term “carboxyC₁-C₆alkoxy,” as used herein, refers to a carboxyC₁-C₆alkyl group attached to the parent molecular moiety through an oxygen atom.

The term “carboxyC₁-C₆alkyl”, as used herein, refers to a carboxy group attached to the parent molecular moiety through a C₁-C₆alkyl group.

The term “C₃-C₈cycloalkyl”, as used herein, refers to a saturated monocyclic or bicyclic hydrocarbon ring system having three to eight carbon atoms and zero heteroatoms. The bicyclic rings can be fused, spirocyclic, or bridged. Representative examples of cycloalkyl groups include, but are not limited to, cyclopropyl, cyclopentyl, octahydropentalene, and bicyclo[3.1.1]heptyl.

The term “(C₃-C₈cycloalkyl)C₁-C₆alkyl”, as used herein, refers to a C₃-C₈cycloalkyl group attached to the parent molecular moiety through a C₁-C₆alkyl group.

The terms “halo” and “halogen”, as used herein, refer to F, Cl, Br, or I.

The term “heteroaryl,” as used herein, refers to an aromatic five- or six-membered ring where at least one atom is selected from N, O, and S, and the remaining atoms are carbon. The term “heteroaryl” also includes bicyclic systems where a heteroaryl ring is fused to a four- to six-membered aromatic or non-aromatic ring containing zero, one, or two additional heteroatoms selected from N, O, and S; and tricyclic systems where a bicyclic system is fused to a four- to six-membered aromatic or non-aromatic ring containing zero, one, or two additional heteroatoms selected from N, O, and S. The heteroaryl groups are attached to the parent molecular moiety through any substitutable carbon or nitrogen atom in the group. Representative examples of heteroaryl groups include, but are not limited to, alloxazine, benzo[1,2-d:4,5-d′]bisthiazole, benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, purine, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl.

The term “heteroarylC₁-C₆alkyl,” as used herein, refers to a heteroaryl group attached to the parent molecular moiety through a C₁-C₆alkyl group.

The term “hydroxy,” as used herein, refers to —OH.

The term “hydroxyC₁-C₆alkyl,” as used herein, refers to a hydroxy group attached to the parent molecular moiety through a C₁-C₆alkyl group.

The term “immune response” refers to the action of, for example, lymphocytes, antigen presenting cells, phagocytic cells, granulocytes, and soluble macromolecules that results in selective damage to, destruction of, or elimination from the human body of invading pathogens, cells or tissues infected with pathogens, cancerous cells, or, in cases of autoimmunity or pathological inflammation, normal human cells or tissues.

The terms “Programmed Death Ligand 1”, “Programmed Cell Death Ligand 1”, “PD-L1”, “PDL1”, “hPD-L1”, “hPD-L1”, and “B7-H1” are used interchangeably, and include variants, isoforms, species homologs of human PD-L1, and analogs having at least one common epitope with PD-L1. The complete PD-L1 sequence can be found under GENBANK® Accession No. NP_054862.

The terms “Programmed Death 1”, “Programmed Cell Death 1”, “Protein PD-1”, “PD-1”, “PD1”, “hPD-1” and “hPD-1” are used interchangeably, and include variants, isoforms, species homologs of human PD-1, and analogs having at least one common epitope with PD-1. The complete PD-1 sequence can be found under GENBANK® Accession No. U64863.

The term “treating” refers to i) inhibiting the disease, disorder, or condition, i.e., arresting its development; and/or ii) relieving the disease, disorder, or condition, i.e., causing regression of the disease, disorder, and/or condition and/or symptoms associated with the disease, disorder, and/or condition.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds can have a variety of potential uses, for example as standards and reagents in determining biological activity. In the case of stable isotopes, such compounds can have the potential to favorably modify biological, pharmacological, or pharmacokinetic properties.

An additional aspect of the subject matter described herein is the use of the disclosed compounds as radiolabeled ligands for development of ligand binding assays or for monitoring of in vivo adsorption, metabolism, distribution, receptor binding or occupancy, or compound disposition. For example, a macrocyclic compound described herein can be prepared using a radioactive isotope and the resulting radiolabeled compound can be used to develop a binding assay or for metabolism studies. Alternatively, and for the same purpose, a macrocyclic compound described herein can be converted to a radiolabeled form by catalytic tritiation using methods known to those skilled in the art.

The macrocyclic compounds of the present disclosure can also be used as PET imaging agents by adding a radioactive tracer using methods known to those skilled in the art.

Those of ordinary skill in the art are aware that an amino acid includes a compound represented by the general structure:

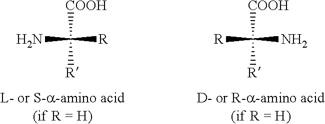

where R and R′ are as discussed herein. Unless otherwise indicated, the term “amino acid” as employed herein, alone or as part of another group, includes, without limitation, an amino group and a carboxyl group linked to the same carbon, referred to as “a” carbon, where R and/or R′ can be a natural or an un-natural side chain, including hydrogen. The absolute “S” configuration at the “α” carbon is commonly referred to as the “L” or “natural” configuration. In the case where both the “R″ and the “R″”(prime) substituents equal hydrogen, the amino acid is glycine and is not chiral.

Where not specifically designated, the amino acids described herein can be D- or L-stereochemistry and can be substituted as described elsewhere in the disclosure. It should be understood that when stereochemistry is not specified, the present disclosure encompasses all stereochemical isomeric forms, or mixtures thereof, which possess the ability to inhibit the interaction between PD-1 and PD-L1. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds of the present disclosure can exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

Certain compounds of the present disclosure can exist as tautomers, which are compounds produced by the phenomenon where a proton of a molecule shifts to a different atom within that molecule. The term “tautomer” also refers to one of two or more structural isomers that exist in equilibrium and are readily converted from one isomer to another. All tautomers of the compounds described herein are included within the present disclosure.

The pharmaceutical compounds of the disclosure can include one or more pharmaceutically acceptable salts. A “pharmaceutically acceptable salt” refers to a salt that retains the desired biological activity of the parent compound and does not impart any undesired toxicological effects (see e.g., Berge, S. M. et al., J. Pharm. Sci., 66:1-19 (1977)). The salts can be obtained during the final isolation and purification of the compounds described herein, or separately be reacting a free base function of the compound with a suitable acid or by reacting an acidic group of the compound with a suitable base. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Administration of a therapeutic agent described herein includes, without limitation, administration of a therapeutically effective amount of therapeutic agent. The term “therapeutically effective amount” as used herein refers, without limitation, to an amount of a therapeutic agent to treat a condition treatable by administration of a composition comprising the PD-1/PD-L1 binding inhibitors described herein. That amount is the amount sufficient to exhibit a detectable therapeutic or ameliorative effect. The effect can include, for example and without limitation, treatment of the conditions listed herein. The precise effective amount for a subject will depend upon the subject's size and health, the nature and extent of the condition being treated, recommendations of the treating physician, and therapeutics or combination of therapeutics selected for administration.

For administration of the macrocyclic peptides described herein, the dosage ranges from about 0.0001 to 100 mg/kg, and more usually 0.01 to 40 mg/kg, of the host body weight. For example dosages can be 0.3 mg/kg body weight, 1 mg/kg body weight, 3 mg/kg body weight, 5 mg/kg body weight, 10 mg/kg body weight, 20 mg/kg body weight, 30 mg/kg body weight, 40 mg/kg body weight, or within the range of 10-40 mg/kg. An exemplary treatment regime entails administration once per day, bi-weekly, tri-weekly, weekly, once every two weeks, once every three weeks, once every four weeks, once a month, once every 3 months or once every three to 6 months. Preferred dosage regimens for a macrocyclic peptide of the disclosure include 1 mg/kg body weight or 3 mg/kg body weight via intravenous administration, with the macrocyclic peptide being given using one of the following dosing schedules: (i) every four weeks for six dosages, then every three months; (ii) every three weeks; (iii) 3 mg/kg body weight once followed by 1 mg/kg body weight every three weeks.

In another aspect, the disclosure pertains to methods of inhibiting growth of tumor cells in a subject using the macrocyclic compounds of the present disclosure. In certain embodiments, the compounds of the present disclosure are capable of binding to PD-1, disrupting the interaction between PD-1 and PD-L1, competing with the binding of PD-1 with certain anti-PD-1 monoclonal antibodies that are known to block the interaction with PD-L1, and enhancing CMV-specific T cell IFNγ secretion. As a result, the compounds of the present disclosure can be useful for modifying an immune response, treating diseases such as cancer, stimulating a protective autoimmune response, or to stimulate antigen-specific immune responses (e.g., by co-administration of PD-L1 blocking compounds with an antigen of interest). For example, the compounds of the present disclosure can be used to treat cancers selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and hematological malignancies.

Compounds of the present disclosure can also be used in treating infectious diseases, such as those caused by a virus. Examples of such viruses include, but are not limited to, HIV, Hepatitis A, Hepatitis B, Hepatitis C, herpes viruses, and influenza.

Compounds of the present disclosure can also be used in treating septic shock.

### Pharmaceutical Compositions

In another aspect, the present disclosure provides a composition, e.g., a pharmaceutical composition, containing one or a combination of the compounds described within the present disclosure, formulated together with a pharmaceutically acceptable carrier. Pharmaceutical compositions of the disclosure also can be administered in combination therapy, i.e., combined with other agents. For example, the combination therapy can include a macrocyclic compound combined with at least one other anti-inflammatory or immunosuppressant agent. Examples of therapeutic agents that can be used in combination therapy are described in greater detail below in the section on uses of the compounds of the disclosure.

As used herein, “pharmaceutically acceptable carrier” includes any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active compound can be coated in a material to protect the compound from the action of acids and other natural conditions that can inactivate the compound.

A pharmaceutical composition of the disclosure also can include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present disclosure can be administered via one or more routes of administration using one or more of a variety of methods known in the art. As will be appreciated by the skilled artisan, the route and/or mode of administration will vary depending upon the desired results. In some embodiments, the routes of administration for macrocyclic compounds of the disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

Examples of suitable aqueous and non-aqueous carriers that can be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be desirable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Alternatively, the compounds of the disclosure can be administered via a non-parenteral route, such as a topical, epidermal or mucosal route of administration, for example, intranasally, orally, vaginally, rectally, sublingually or topically.

Any pharmaceutical composition contemplated herein can, for example, be delivered orally via any acceptable and suitable oral preparation. Exemplary oral preparations include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

A tablet can, for example, be prepared by admixing at least one compound of Formula (I) and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials include, but are not limited to, hydroxypropyl-methylcellulose and hydroxypropyl-cellulose. Exemplary time delay materials include, but are not limited to, ethyl cellulose and cellulose acetate butyrate.

Hard gelatin capsules can, for example, be prepared by mixing at least one compound of Formula (I) and/or at least one salt thereof with at least one inert solid diluent, such as, for example, calcium carbonate; calcium phosphate; and kaolin.

Soft gelatin capsules can, for example, be prepared by mixing at least one compound of Formula (I) and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I) and/or at least one pharmaceutically acceptable salt thereof with at least one excipient suitable for the manufacture of an aqueous suspension, including, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example, heptadecathylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) and/or at least one pharmaceutically acceptable salt thereof in either a vegetable oil, such as, for example, arachis oil, sesame oil, and coconut oil; or in mineral oil, such as, for example, liquid paraffin. An oily suspension can also contain at least one thickening agent, such as, for example, beeswax, hard paraffin, and cetyl alcohol. In order to provide a palatable oily suspension, at least one of the sweetening agents already described herein above, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I) and/or at least one pharmaceutically acceptable salt thereof with at least one dispersing and/or wetting agent, at least one suspending agent, and/or at least one preservative. Suitable dispersing agents, wetting agents, and suspending agents are already described above. Exemplary preservatives include, but are not limited to, for example, antioxidants, e.g., ascorbic acid. In addition, dispersible powders and granules can also contain at least one excipient, including, but not limited to, for example, sweetening agents, flavoring agents, and coloring agents.

An emulsion of at least one compound of Formula (I) and/or at least one pharmaceutically acceptable salt thereof can, for example, be prepared as an oil-in-water emulsion. The oily phase of the emulsions comprising the compounds of Formula (I) can be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase can comprise merely an emulsifier, it can comprise a mixture of at least one emulsifier with a fat or an oil or with both a fat and an oil. Suitable emulsifying agents include, but are not limited to, for example, naturally-occurring phosphatides, e.g., soy bean lecithin, esters or partial esters derived from fatty acids and hexitol anhydrides, such as, for example sorbitan monoleate, and condensation products of partial esters with ethylene oxide, such as, for example, polyoxyethylene sorbitan monooleate. In some embodiments, a hydrophilic emulsifier is included together with a lipophilic emulsifier which acts as a stabilizer. It is also sometimes desirable to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present disclosure include Tween 60, Span 80, cetostearyl alcohol, myristyl alcohol, glyceryl monostearate, sodium lauryl sulfate, glyceral disterate alone or with a wax, or other materials well known in the art.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Robinson, J. R., ed., Sustained and Controlled Release Drug Delivery Systems, Marcel Dekker, Inc., New York (1978).

Therapeutic compositions can be administered with medical devices known in the art. For example, in one embodiment, a therapeutic composition of the disclosure can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medication through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the compounds of the disclosure can be formulated to ensure proper distribution in vivo. For example, the blood-brain barrier (BBB) excludes many highly hydrophilic compounds. To ensure that therapeutic compounds of the disclosure cross the BBB (if desired), they can be formulated, for example, in liposomes. For methods of manufacturing liposomes, see, e.g., U.S. Pat. Nos. 4,522,811, 5,374,548, and 5,399,331. The liposomes can comprise one or more moieties which are selectively transported into specific cells or organs, thus enhance targeted drug delivery (see, e.g., Ranade, V. V., J. Clin. Pharmacol., 29:685 (1989)). Exemplary targeting moieties include folate or biotin (see, e.g., U.S. Pat. No. 5,416,016 to Low et al.); mannosides (Umezawa et al., Biochem. Biophys. Res. Commun., 153:1038 (1988)); macrocyclic compounds (Bloeman, P. G. et al., FEBSLett., 357:140 (1995); Owais, M. et al., Antimicrob. Agents Chemother., 39:180 (1995)); surfactant protein A receptor (Briscoe et al., Am. J. Physiol., 1233:134 (1995)); p¹²⁰ (Schreier et al., J. Biol. Chem., 269:9090 (1994)); see also Keinanen, K. et al., FEBSLett., 346:123 (1994); Killion, J. J. et al., Immunomethods 4:273 (1994).

In certain embodiments, the compounds of the present disclosure can be administered parenterally, i.e., by injection, including, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and/or infusion.

In some embodiments, the compounds of the present disclosure can be administered orally, i.e, via a gelatin capsule, tablet, hard or soft capsule, or a liquid capsule. The compounds can be made by methods known in the art including those described below and including variations within the skill of the art. Some reagents and intermediates are known in the art. Other reagents and intermediates can be made by methods known in the art using readily available materials. Any variables (e.g. numbered “R″ substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

### Examples

The following Examples are included to demonstrate various aspects of the present disclosure. It should be appreciated by those of skill in the art that the techniques disclosed in the Examples that follow represent techniques discovered by the inventors to function well in the practice of the disclosure, and thus can be considered to constitute preferred modes for its practice. However, those of skill in the art should, in light of the present disclosure, appreciate that many changes can be made in the specific Compounds which are disclosed and still obtain a like or similar result without departing from the spirit and scope of the disclosure.

The compounds can be made by methods known in the art including those described below and including variations within the skill of the art. Some reagents and intermediates are known in the art. Other reagents and intermediates can be made by methods known in the art using readily available materials. Any variables (e.g. numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

Abbreviations used in the schemes generally follow conventions used in the art. Chemical abbreviations used in the specification and Compounds are defined as follows: Ph=phenyl; Bn=benzyl; i-Bu=iso-butyl; i-Pr=iso-propyl; Me=methyl; Et=ethyl; Pr=n-propyl; Bu=n-butyl; t-Bu=tert-butyl; Trt=trityl; TMS=trimethylsilyl; TIS=triisopropylsilane; Et20=diethyl ether; HOAc or AcOH=acetic acid; MeCN or AcCN=acetonitrile; DMF=N,N-dimethylformamide; EtOAc=ethyl acetate; THF=tetrahydrofuran; TFA=trifluoroacetic acid; TFE=α,α,α-trifluoroethanol; Et₂NH=diethylamine; NMN=N-methylmorpholine; NMP=N-methylpyrrolidone; DCM=dichloromethane; TEA=trimethylamine; min.=minute(s); h or hr=hour(s); L=liter; mL or mL=milliliter; μL=microliter; g=gram(s); mg=milligram(s); mol=mole(s); mmol=millimole(s); meq=milliequivalent; rt or RT=room temperature; sat or sat′d=saturated; aq.=aqueous; mp=melting point; BOP reagent=benzotriazol-1-yloxy-tris-dimethylamino-phosphonium hexafluorophosphate (Castro's reagent); PyBOP reagent=benzotriazol-1-yloxy-tripyrrolidino phosphonium hexafluorophosphate; HBTU=2-(1H-Benzotriazol-1-yl)-1,1,3,3-tetramethyluronim hexafluorophosphate; HATU=O-(7-Azabenzotriazol-1-yl)-1,1,3,3-tetramethyluronim hexafluorophosphate; HCTU=2-(6-Chloro-1-H-benzotriazol-1-yl)-1,1,3,3-tetramethyluronium hexafluorophosphate; T3P=2,4,6-tripropyl-1,3,5,2,4,6-trioxatriphosphorinane-2,4,6-trioxide; DMAP=4-(dimethylamino)pyridine; DIEA=diisopropylethylamine; Fmoc or FMOC=fluorenylmethyloxycarbonyl; Boc or BOC=tert-butyloxycarbonyl; HOBT or HOBT9H20=1-hydroxybenzotriazole hydrate; C₁-HOBt=6-Chloro-benzotriazole; HOAT=1-hydroxy-7-azabenzotriazole; HPLC=high performance liquid chromatography; LC/MS=high performance liquid chromatography/mass spectrometry; MS or Mass Spec=mass spectrometry; NMR=nuclear magnetic resonance; Sc or SC or SQ=subcutaneous; and IP or ip=intraperitoneal.

### Example 1: General Synthetic Procedures and Analytical Methods

The macrocyclic compounds of the present disclosure can be produced by methods known in the art, such as they can be synthesized chemically, recombinantly in a cell free system, recombinantly within a cell or can be isolated from a biological source. Chemical synthesis of a macrocyclic compound of the present disclosure can be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. A preferred method to synthesize the macrocyclic compounds and analogs thereof described herein is chemical synthesis using various solid-phase techniques such as those described in Chan, W. C. et al, eds., Fmoc SolidPhase Synthesis, Oxford University Press, Oxford (2000); Barany, G. et al, The Peptides: Analysis, Synthesis, Biology, Vol. 2: “Special Methods in Peptide Synthesis, Part A”, pp. 3-284, Gross, E. et al, eds., Academic Press, New York (1980); in Atherton, E., Sheppard, R. C. SolidPhase Peptide Synthesis: A PracticalApproach, IRL Press, Oxford, England (1989); and in Stewart, J. M. Young, J. D. Solid-Phase Peptide Synthesis, 2nd Edition, Pierce Chemical Co., Rockford, IL (1984). The preferred strategy is based on the (9-fluorenylmethyloxycarbonyl) group (Fmoc) for temporary protection of the α-amino group, in combination with the tert-butyl group (tBu) for temporary protection of the amino acid side chains (see for example Atherton, E. et al, “The Fluorenylmethoxycarbonyl Amino Protecting Group”, in The Peptides: Analysis, Synthesis, Biology, Vol. 9: “Special Methods in Peptide Synthesis, Part C”, pp. 1-38, Undenfriend, S. et al, eds., Academic Press, San Diego (1987).

The compounds can be synthesized in a stepwise manner on an insoluble polymer support (also referred to as “resin”) starting from the C-terminus of the peptide. A synthesis is begun by appending the C-terminal amino acid of the peptide to the resin through formation of an amide or ester linkage. This allows the eventual release of the resulting peptide as a C-terminal amide or carboxylic acid, respectively.

The C-terminal amino acid and all other amino acids used in the synthesis are required to have their α-amino groups and side chain functionalities (if present) differentially protected such that the α-amino protecting group may be selectively removed during the synthesis. The coupling of an amino acid is performed by activation of its carboxyl group as an active ester and reaction thereof with the unblocked α-amino group of the N-terminal amino acid appended to the resin. The sequence of α-amino group deprotection and coupling is repeated until the entire peptide sequence is assembled. The peptide is then released from the resin with concomitant deprotection of the side chain functionalities, usually in the presence of appropriate scavengers to limit side reactions. The resulting peptide is finally purified by reverse phase HPLC.

The synthesis of the peptidyl-resins required as precursors to the final peptides utilizes commercially available cross-linked polystyrene polymer resins (Novabiochem, San Diego, CA; Applied Biosystems, Foster City, CA). Preferred solid supports are: 4-(2′,4′-dimethoxyphenyl-Fmoc-aminomethyl)-phenoxyacetyl-p-methyl benzhydrylamine resin (Rink amide MBHA resin); 9-Fmoc-amino-xanthen-3-yloxy-Merrifield resin (Sieber amide resin); 4-(9-Fmoc)aminomethyl-3,5-dimethoxyphenoxy)valerylaminomethyl-Merrifield resin (PAL resin), for C-terminal carboxamides. Coupling of first and subsequent amino acids can be accomplished using HOBt, 6-C₁-HOBt or HOAt active esters produced from DIC/HOBt, HBTU/HOBt, BOP, PyBOP, or from DIC/6-C₁-HOBt, HCTU, DIC/HOAt or HATU, respectively. Preferred solid supports are: 2-chlorotrityl chloride resin and 9-Fmoc-amino-xanthen-3-yloxy-Merrifield resin (Sieber amide resin) for protected peptide fragments. Loading of the first amino acid onto the 2-chlorotrityl chloride resin is best achieved by reacting the Fmoc-protected amino acid with the resin in dichloromethane and DIEA. If necessary, a small amount of DMF may be added to solubilize the amino acid.

The syntheses of the compound analogs described herein can be carried out by using a single or multi-channel peptide synthesizer, such as an CEM Liberty Microwave synthesizer, or a Protein Technologies, Inc. Prelude (6 channels) or Symphony (12 channels) or Symphony X (24 channels) synthesizer.

Useful Fmoc amino acids derivatives are shown in Table 1.

TABLE 1
Compounds of Orthogonally Protected Amino Acids used in Solid Phase Synthesis

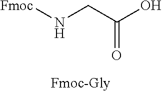

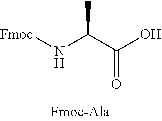

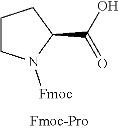

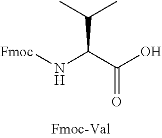

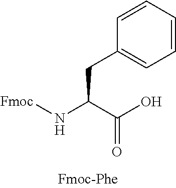

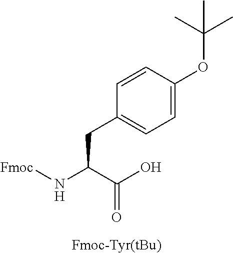

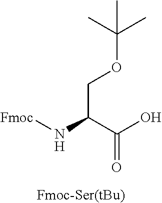

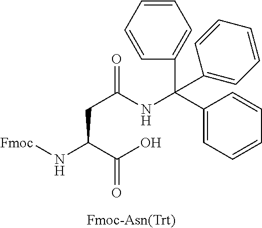

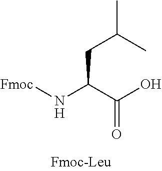

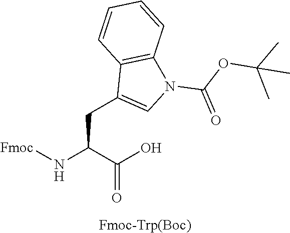

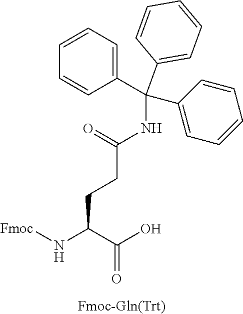

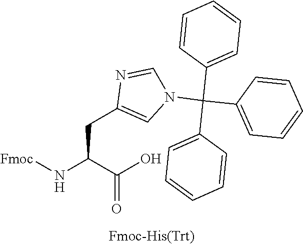

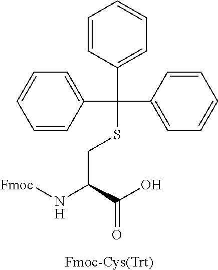

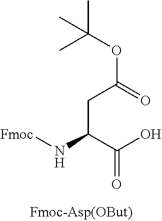

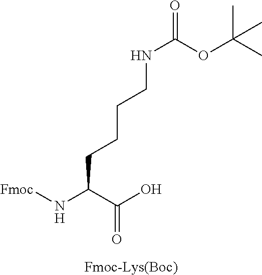

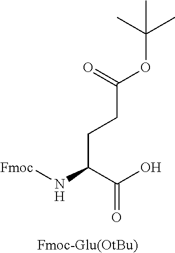

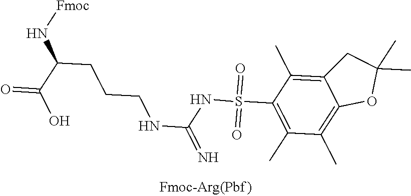

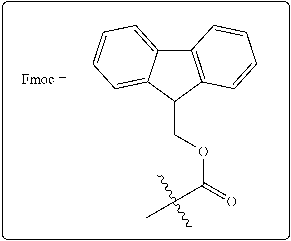 [SMILES: *C(=O)OCC1c2ccccc2-c2ccccc21]

The peptidyl-resin precursors for their respective compounds may be cleaved and deprotected using any standard procedure (see, for Compound, King, D. S. et al, Int. J Peptide Protein Res., 36:255-266 (1990)). A desired method is the use of TFA in the presence of TIS as scavenger and DTT or TCEP as the disulfide reducing agent. Typically, the peptidyl-resin is stirred in TFA/TIS/DTT (95:5:1 to 97:3:1), v:v:w; 1-3 mL/100 mg of peptidyl resin) for 1.5-3 hrs at room temperature. The spent resin is then filtered off and the TFA solution was cooled and Et20 solution was added. The precipitates were collected by centrifuging and decanting the ether layer (3×). The resulting crude peptide is either redissolved directly into DMF or DMSO or CH₃CN/H₂O for purification by preparative HPLC or used directly in the next step.

Compounds with the desired purity can be obtained by purification using preparative HPLC on a Waters Model 4000 or a Shimadzu Model LC-8A liquid chromatography. The solution of crude compound is injected into a YMC S5 ODS (20×100 mm) column and eluted with a linear gradient of MeCN in water, both buffered with 0.100 TFA, using a flow rate of 14-20 mL/min with effluent monitoring by UV absorbance at 217 or 220 nm. The structures of the purified compounds can be confirmed by electro-spray MS analysis.

List of unnatural amino acids referred to herein is provided in Table 2.

TABLE 2
Compounds of Orthogonally Protected Amino Acids used in Solid Phase Synthesis

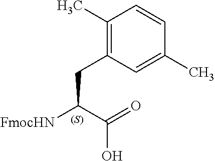

Fmoc-Phe(2,5-di-Me)—OH

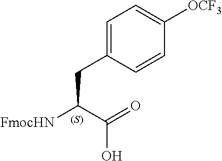

Fmoc-Phe(4-OCF₃)—OH

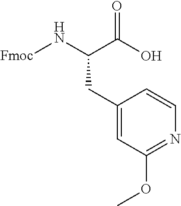

Fmoc-4-Pyr(2-OMe)—OH

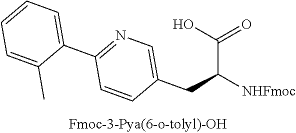

(S)-2-((((9H-fluoren-9-
yl)methoxy)carbonyl)amino)-3-(6-
(o-tolyl)pyridin-3-yl)propanoic acid

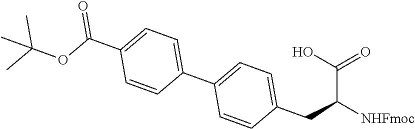

Fmoc-Bip(4′-COOtBu)-OH

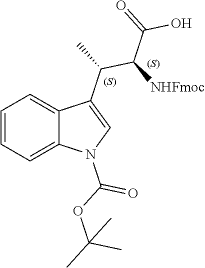

Fmoc-(S)-β-Me-Trp(Boc)-OH

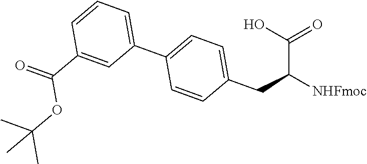

Fmoc-Bip(3′-COOtBu)-OH

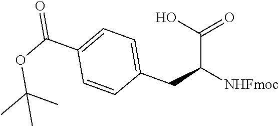

Fmoc-Phe(4-COOtBu)

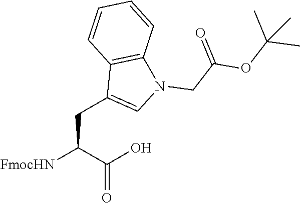

Fmoc-Trp(CH₂COOtBu)-OH

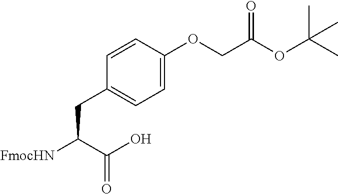

Fmoc-Tyr(CH₂COOtBu)-OH

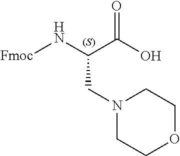

Fmoc-Ala(β-morpholin-4-yl)-OH
(2S)-2-({[(9H-fluoren-9-
yl)methoxy]carbonyl}amino)-3-
(morpholin-4-yl)propanoic acid

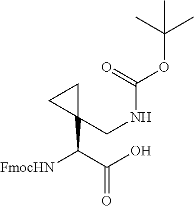

(S)-2-Fmoc-amino-2-(1-Boc-
aminomethyl-cyclopropyl)acetic acid

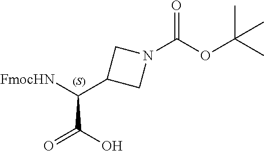

(S)-2-Fmoc-amino-2-(1-Boc-azetidin-3-
yl)acetic acid

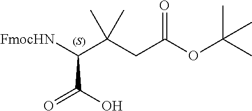

Fmoc-β,β-diMe-Glu(tBu)-OH

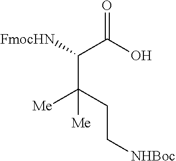

Fmoc-β,β-diMe-Orn(Boc)-OH

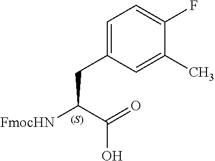

Fmoc-Phe(3-Me—4-F)—OH

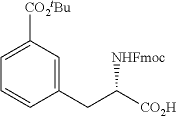

Fmoc-Phe(3-COOtBu)-OH

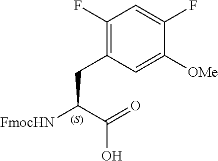

Fmoc-Phe(2,4-di-F—5-OMe)—OH

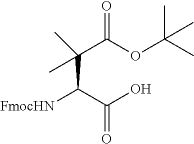

Fmoc-β,β-DiMe-Asp(tBu)-OH

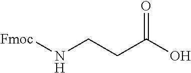

Fmoc-β-Ala-OH

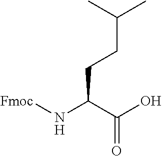

Fmoc-HomoLeu-OH

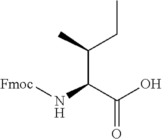

Fmoc-Ile-OH

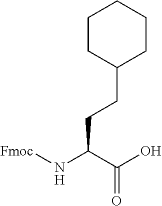

Fmoc-HomoCha-OH

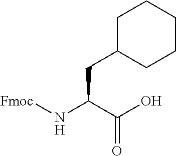

Fmoc-Cha-OH

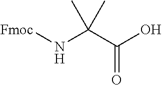

(Fmoc-Aib-OH)

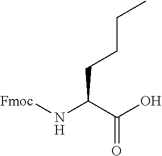

Fmoc-Nle-OH

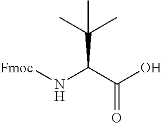

Fmoc-tBu-Gly-OH
(Fmoc-Tle-OH)

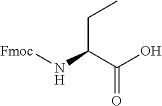

Fmoc-Abu-OH

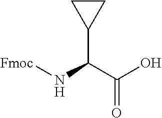

Fmoc-Cyclopropyl-Gly-OH

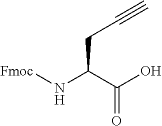

(Fmoc-Pra-OH)

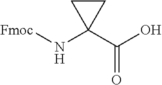

1-(Fmoc-
amino)cyclopropane
carboxylic acid

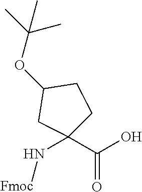

Fmoc-1-amino-3-OtBu-
cyclopentane-1-carboxylic acid

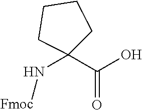

Fmoc-1-aminocyclopentane-
1-carboxylic acid

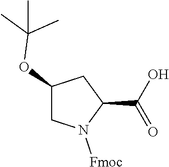

Fmoc-cis-Hyp(tBu)-OH

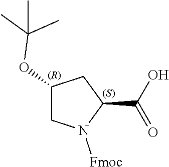

Fmoc-Hyp(tBu)-OH

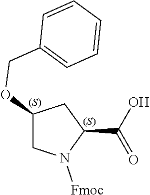

Fmoc-cis-Hyp(Bn)-OH

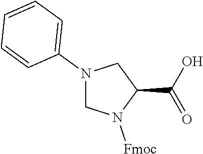

Fmoc-NPh-AzaPro-OH

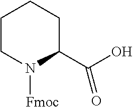

Fmoc-Pip-OH

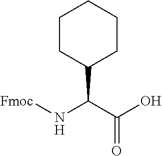

Fmoc-Chg-OH

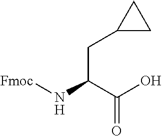

Fmoc-Ala(β-cyclopropyl)-OH
Fmoc-Cyclopropyl-Ala-OH

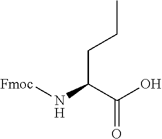

Fmoc-Nva-OH

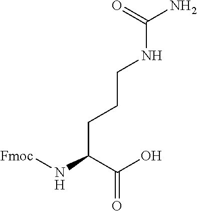

Fmoc-Cit-OH

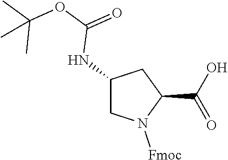

Fmoc-trans-4-NHBoc-Pro-OH

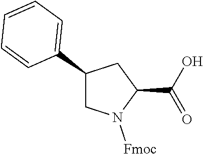

Fmoc-cis-4-Ph-Pro-OH

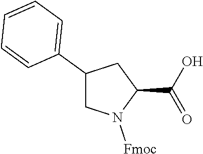

Fmoc-4-Ph-Pro-OH

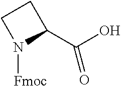

Fmoc-azetidine-2-carboxylic acid
Fmoc-Azt-OH

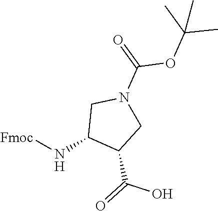

Fmoc-isoPro(Boc)-OH

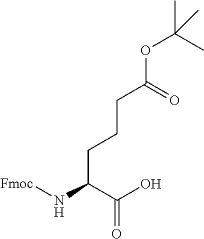

Fmoc-homoGlu(OtBu)-OH

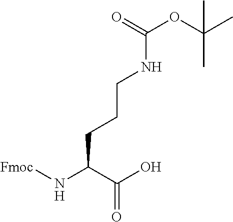

Fmoc-Orn(Boc)-OH

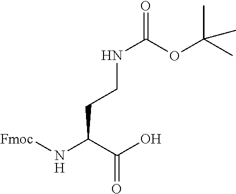

Fmoc-Dab(Boc)-OH

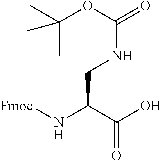

Fmoc-Dap(Boc)-OH

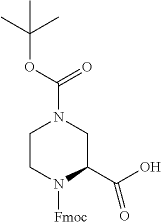

1-Fmoc-4-(tert-butoxycarbonyl)
piperazine-2-carboxylic acid
1-Fmoc-4-Boc-piperazine-OH

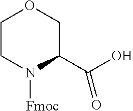

4-N-Fmoc-morpholinecarboxylic
acid

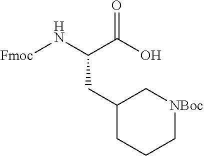

Fmoc-Ala(β-1-Boc-piperidin-3-yl)-OH

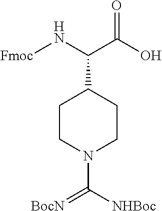

(2S)-2-{1-[(1Z)-{[(tert-
butoxy)carbonyl]amino}({[(tert-
butoxy)carbonyl]imino})methyl]
piperidin-4-yl}-2-
({[(9H-fluoren-9-yl)methoxy]
carbonyl}amino)acetic acid

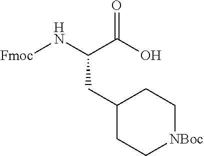

Fmoc-Ala(β-1-Boc-piperidin-4-yl)-OH

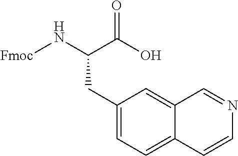

Fmoc-Ala(β-isoquinolin-7-yl)-OH

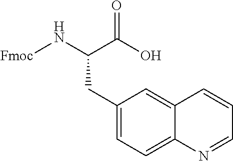

Fmoc-Ala(β-quinolin-6-yl)-OH

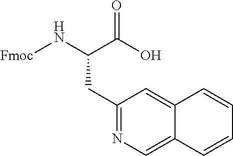

Fmoc-Ala(β-isoquinolin-3-yl)-OH

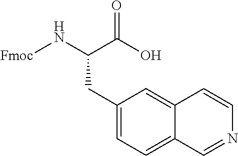

Fmoc-Ala(β-
isoquinolin-6-yl)-OH

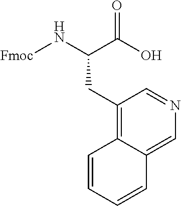

Fmoc-Ala(β-isoquinolin-4-yl)-OH

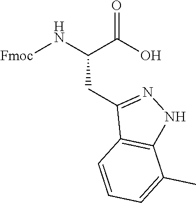

W(1,2-N 2,7-Me)

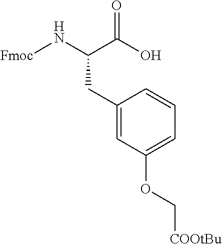

Fmoc-Phe(3-OCH₂COOtBu)-OH

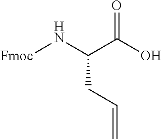

Fmoc-AllylGly-OH

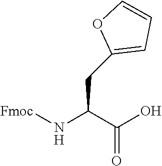

Fmoc-2-furanyl-Ala-OH

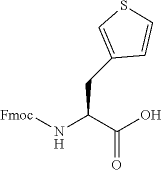

Fmoc-3-thienyl-Ala-OH

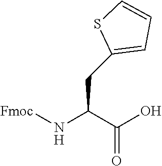

Fmoc-2-thienyl-Ala-OH

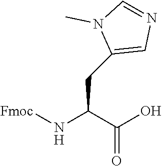

Fmoc-His(3-Me)—OH

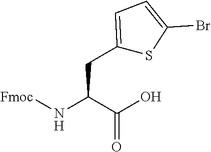

Fmoc-2-thienyl-5-Br-Ala-OH

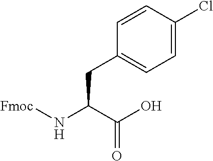

Fmoc-Phe(4-Cl)—OH

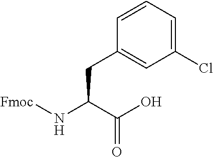

Fmoc-Phe(3-Cl)—OH

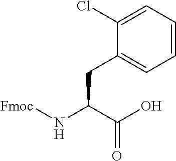

Fmoc-Phe(2-Cl)—OH

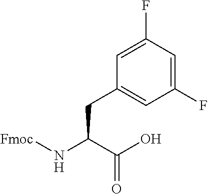

Fmoc-Phe(3,5-di-F)—OH

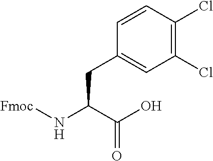

Fmoc-Phe(3,4-di-Cl)—OH

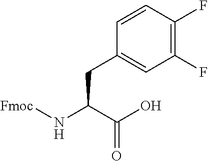

Fmoc-Phe(3,4-diF)—OH

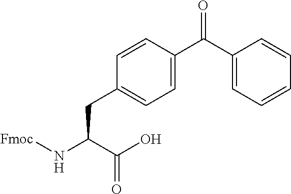

Fmoc-Bpa-OH

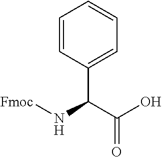

Fmoc-PhenylGly-OH
(Fmoc-Phg-OH)

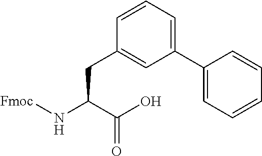

Fmoc-3-Ph-Phe-OH

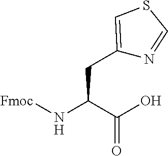

Fmoc-4-Thiazolylalanine
Fmoc-Ala(4-Thiazol-3-yl)

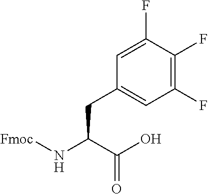

Fmoc-Phe(3,4,5-tri-F)—OH

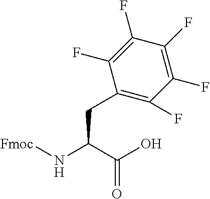

Fmoc-Phe(2,3,4,5,6-penta-F)—OH

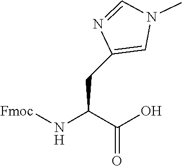

Fmoc-His(1-Me)—OH

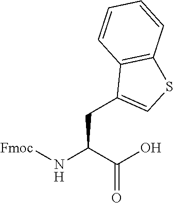

Fmoc-Bzt-OH

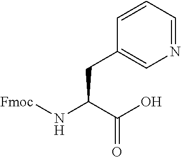

Fmoc-3-Py-Ala-OH
(Fmoc-3-Pya-OH)

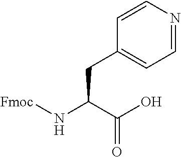

Fmoc-4-Py-Ala-OH
(Fmoc-4-Pya-OH)

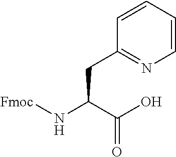

Fmoc-2-Py-Ala-OH
(Fmoc-2-Pya-OH)

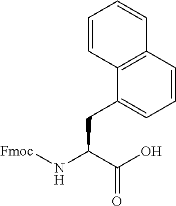

Fmoc-1-Nal-OH

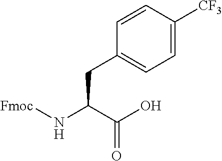

Fmoc-Phe(4-CF₃)—OH

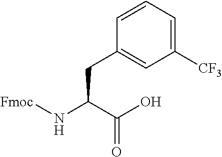

Fmoc-Phe(3-CF₃)—OH

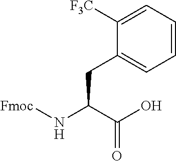

Fmoc-Phe(2-CF₃)—OH

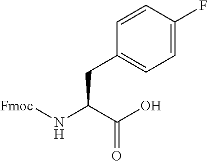

Fmoc-Phe(4-F)—OH

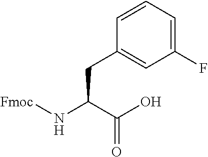

Fmoc-Phe(3-F)—OH

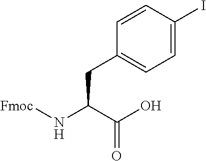

Fmoc-Phe(4-I)—OH

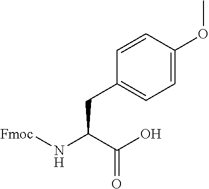

Fmoc-Tyr(Me)—OH
(Fmoc-Phe(4-OMe)—OH)

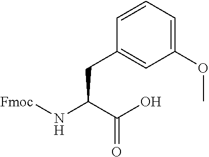

Fmoc-Phe(-3-OMe)—OH

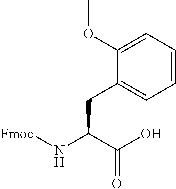

Fmoc-Phe(2-OMe)—OH

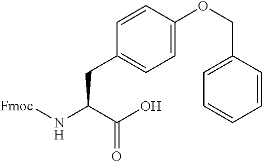

Fmoc-Tyr(Bn)-OH

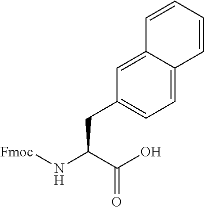

Fmoc-2-Nal-OH

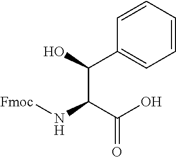

Fmoc-β-hydroxy-Phe-OH

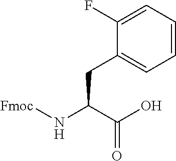

Fmoc-Phe(2-F)—OH

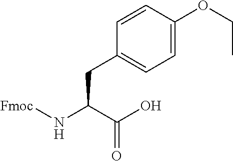

Fmoc-Tyr(Et)-OH

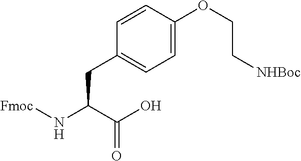

Fmoc-Tyr(CH₂CH₂NHBoc)-OH

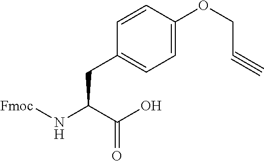

Fmoc-Tyr(propargyl)-OH

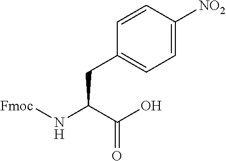

Fmoc-Phe(4-NO₂)—OH

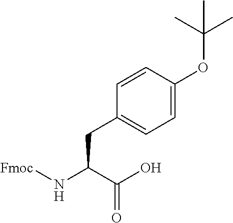

Fmoc-Tyr(tBu)-OH

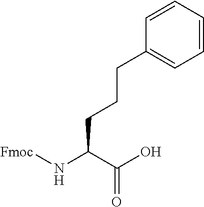

Fmoc-homohomoPhe-OH

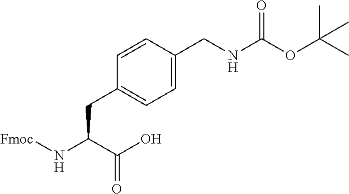

Fmoc-Phe(4-aminomethyl(Boc))-OH

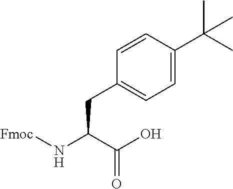

Fmoc-Phe(4-tBu)-OH

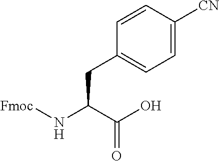

Fmoc-Phe(4-CN)—OH

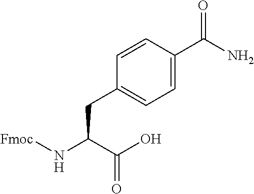

Fmoc-Phe(4-CONH₂)—OH

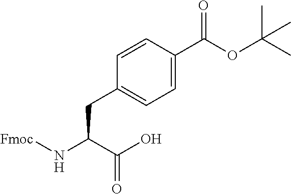

Fmoc-Phe(4-COOtBu)-OH

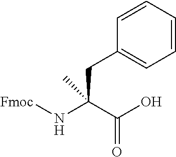

Fmoc-α-Me-Phe-OH

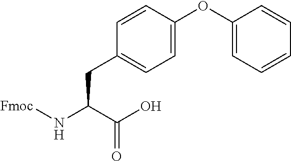

Fmoc-Phe(OPh)-OH
(Fmoc-Tyr(Ph)-OH)

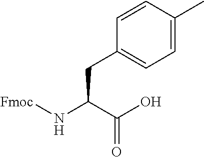

Fmoc-Phe(4-Me)—OH

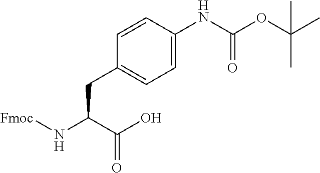

Fmoc-Phe(4-NHBoc)-OH

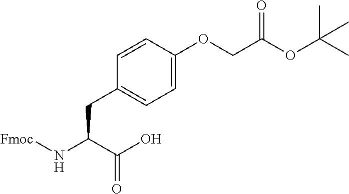

Fmoc-Tyr(CH₂COOtBu)-OH

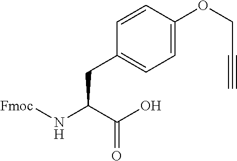

Fmoc-Tyr(propargyl)-OH

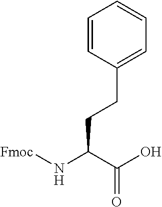

Fmoc-HomoPhe-OH

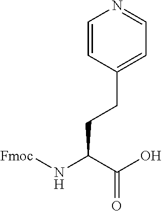

Fmoc-Homo-4-Py-Ala-OH

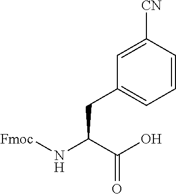

Fmoc-Phe(3-CN)—OH

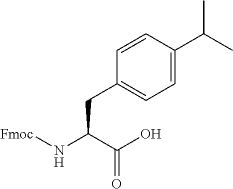

Fmoc-4-Phe(iPr)-OH

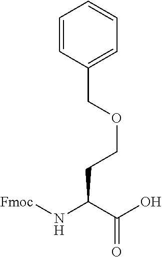

Fmoc-Homo-Ser(Bn)-OH

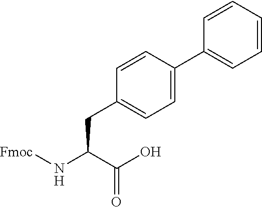

Fmoc-Bip-OH

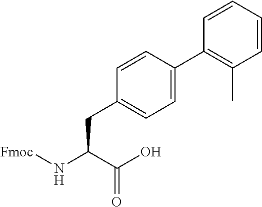

Fmoc-Bip(2′-Me)—OH

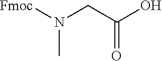

Fmoc-N—Me-Gly-OH
(Fmoc-mGly-OH)

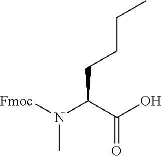

Fmoc-N—Me-Nle-OH
(Fmoc-mNle-OH)

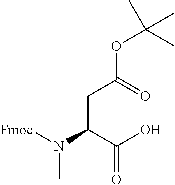

Fmoc-N—Me-Asp(OtBu)-OH
(Fmoc-mAsp(OtBu)-OH)

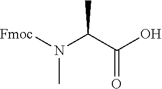

Fmoc-N—Me-Ala-OH
(Fmoc-mAla-OH)

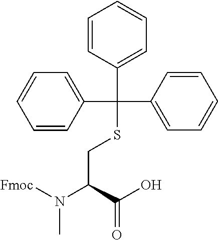

Fmoc-N—Me-Cys(Trt)-OH
(Fmoc-mCys(Trt)-OH)

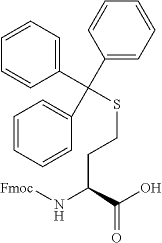

Fmoc-HomoCys(Trt)-OH

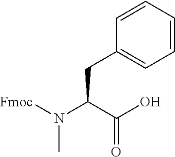

Fmoc-N—Me-Phe-OH
(Fmoc-mPhe-OH)

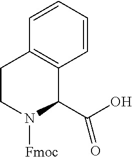

Fmoc-Iso-Tic-OH

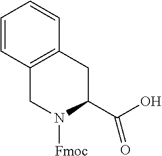

Fmoc-Tic-OH

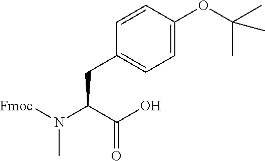

Fmoc-N—Me-Tyr(OtBu)-OH
(Fmoc-mTyr(OtBu)-OH)

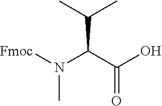

Fmoc-N—Me-Val-OH
(Fmoc-mVal-OH)

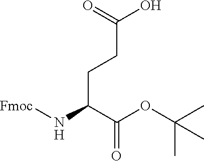

Fmoc-Glu-OtBu

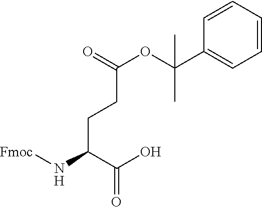

Fmoc-Glu(2-phenylisopropyloxy)-OH

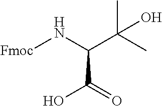

Fmoc-S-hydroxyvaline
Fmoc-V(β-OH)—OH

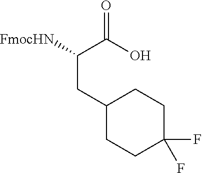

Fmoc-Ala(β-Cha(4,4-di-F))—OH

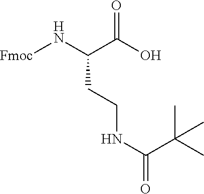

Fmoc-Dab(COtBu)-OH

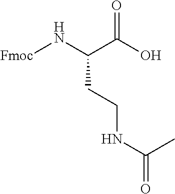

Fmoc-Dab(Ac)-OH

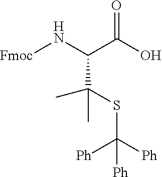

Fmoc-ß,ß-dimethyl-Cys(Trt)-OH
Fmoc-Pen(Trt)-OH

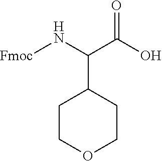

2-NHFmoc-2-(oxan-4-yl)acetic
acid

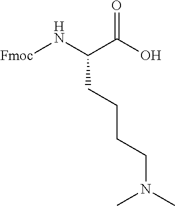

Fmoc-Lys(N,N-di-Me)—OH

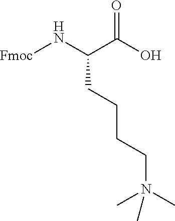

FMOC-Lys(trimethyl)-OH

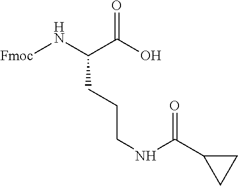

Fmoc-Orn(CO-cyclopropyl)-OH

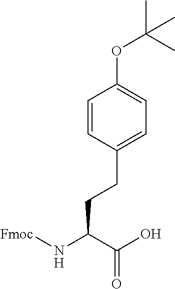

Fmoc-HomoTyr(OtBu)-OH

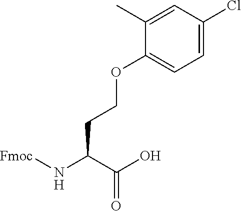

Fmoc-4-Cl—2-Me-phenoxyhomoSer-OH

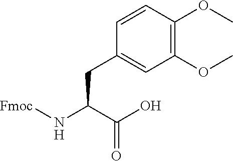

Fmoc-Phe(3,4-di-OMe)—OH

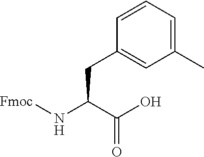

Fmoc-Phe(3-Me)—OH

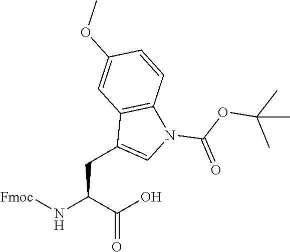

Fmoc-5-MeO-Trp(Boc)-OH

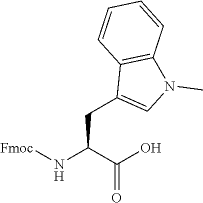

Fmoc-Nme-Trp-OH

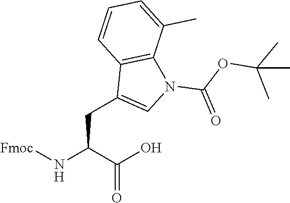

Fmoc-7-Me-Trp(Boc)-OH

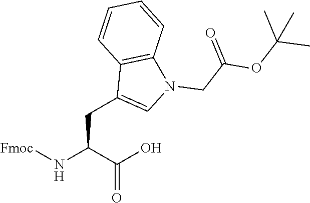

Fmoc-N-AcOH-Trp-OH

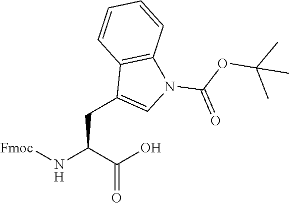

Fmoc-Trp(Boc)-OH

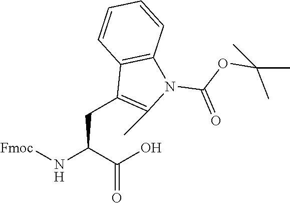

Fmoc-2-Me-Trp(Boc)-OH

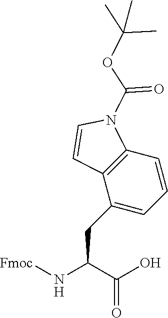

Fmoc-Iso-Trp(Boc)-OH

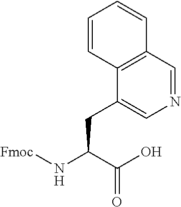

Fmoc-Ala-(β-isoquinolin-4-yl)-OH

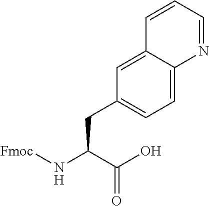

Fmoc-Ala-(β-quinolin-6-yl)-OH

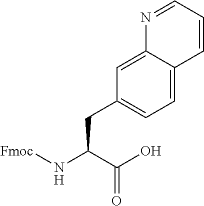

Fmoc-Ala-(β-quinolin-7-yl)-OH

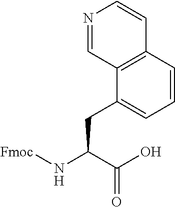

Fmoc-Ala-(β-isoquinolin-8-yl)-OH

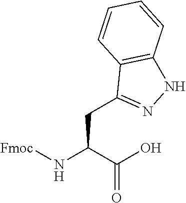

Fmoc-Ala(β-indazol-3-yl)-OH

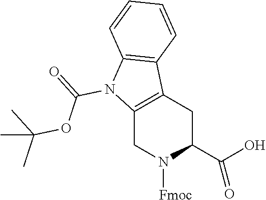

Fmoc-Trp(Boc)Tic-OH

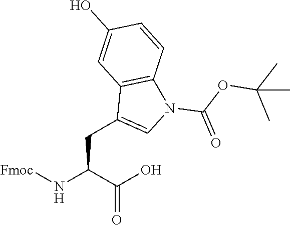

Fmoc-5-OH-Trp(Boc)-OH

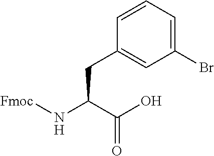

Fmoc-Phe(3-Br)—OH

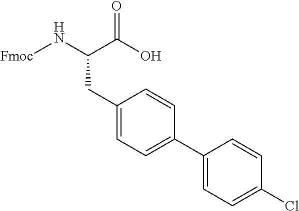

Fmoc-Bip(4′-Cl)—OH

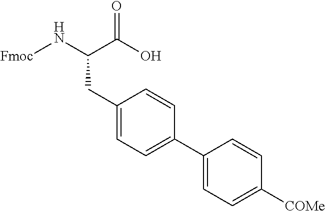

Fmoc-Bip(4′-COMe)—OH

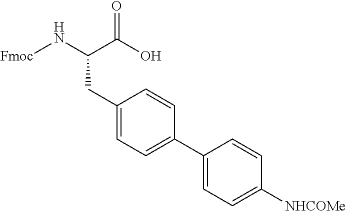

Fmoc-Bip(4′-NHCOMe)—OH

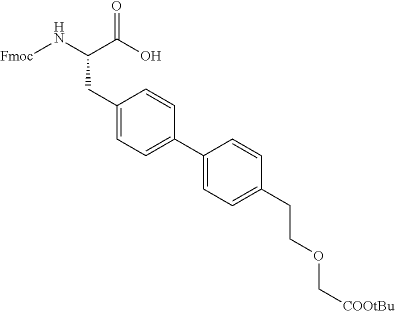

Fmoc-Bip(4′-
CH₂CH₂OCH₂COOtBu)-OH

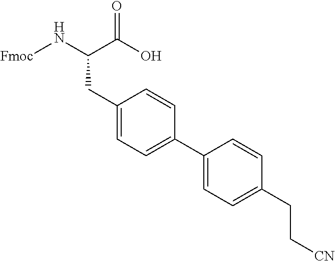

Fmoc-Bip(4′-CH₂CH₂CN)—OH

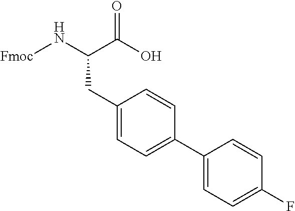

Fmoc-Bip(4′-F)—OH

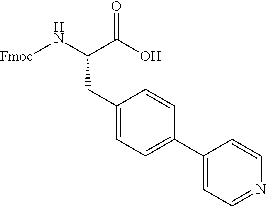

Fmoc-Bip(4′-Pyr)-OH

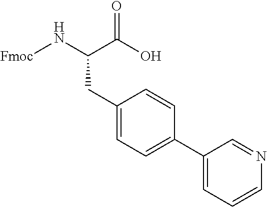

Fmoc-Bip(3′-Pyr)-OH

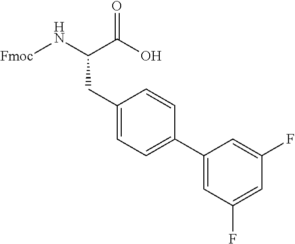

Fmoc-Bip(3′,5′-F)—OH

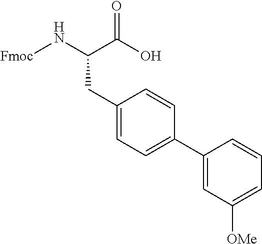

Fmoc-Bip(3′-OMe)—OH

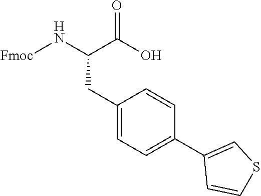

Fmoc-Bip(3′-Thio)-OH

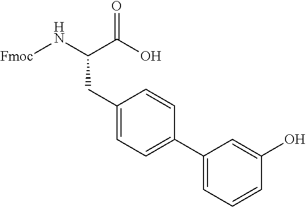

Fmoc-Bip(3′-OH)—OH

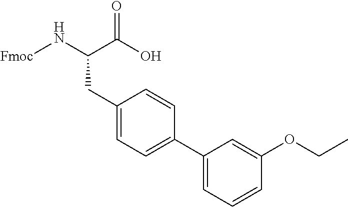

Fmoc-Bip(3′-OEt)-OH

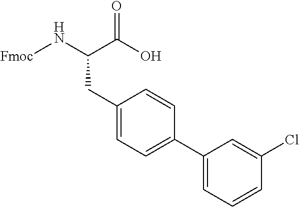

Fmoc-Bip(3′-Cl)—OH

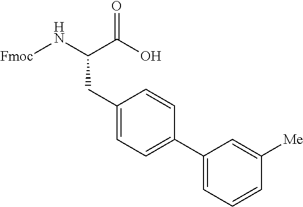

Fmoc-Bip(3′-Me)—OH

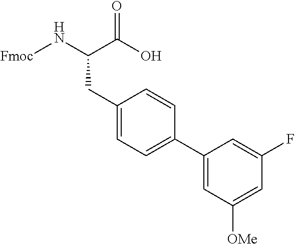

Fmoc-Bip(3′-F,5′-OMe)—OH

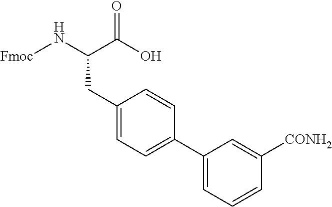

Fmoc-Bip(3′-CONH₂)—OH

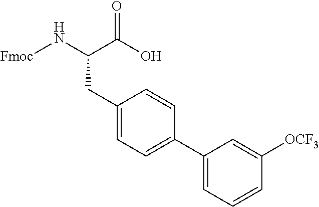

Fmoc-Bip(3′-OCF₃)—OH

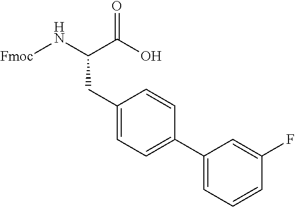

Fmoc-Bip(3′-F)—OH

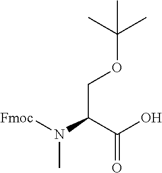

Fmoc-N—Me-Ser(tBu)-OH
(Fmoc-mSer(tBu)-OH)

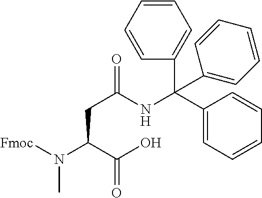

Fmoc-N—Me-Asn(Trt)-OH
(Fmoc-mAsn(Trt)-OH)

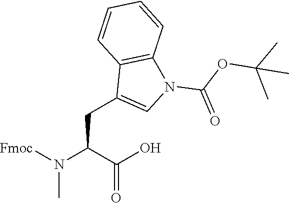

Fmoc-N-MeTrp(Boc)-OH
(Fmoc-mTrp(Boc)-OH)

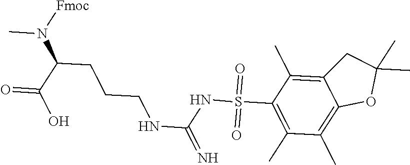

Fmoc-N—Me-Arg(Pbf)-OH
(Fmoc-mArf(Pbf)-OH)

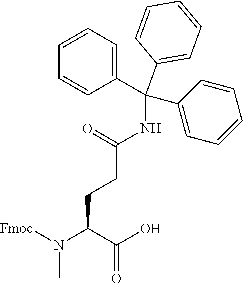

Fmoc-N—Me-Gln(Trt)

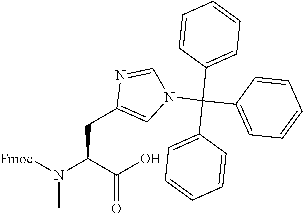

Fmoc-N—Me-His(Trt)

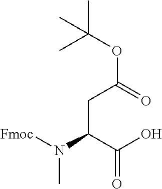

Fmoc-N—Me-Asp(OtBu)-OH
(Fmoc-mAsp(OtBu)-OH)

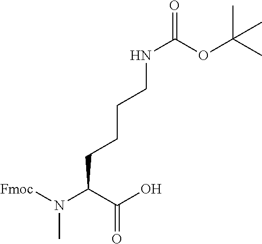

Fmoc-N—Me-Lys(Boc)-OH
(Fmoc-mLys(Boc)-OH)

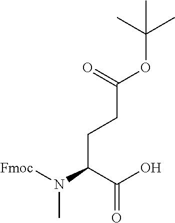

Fmoc-N—Me-Glu(OtBu)-OH
(Fmoc-nGlu(OtBu)-OH)

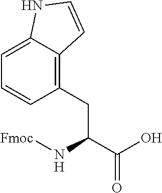

2-(Fmoc-amino)-3-(1H-indol-4-
yl)propanoic acid

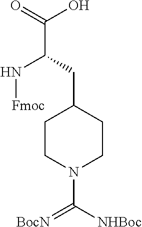

Fmoc-Ala(β-1-
carbamimidoylpiperidin-4-
yl)-OH

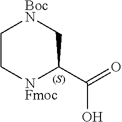

(S)-1-Fmoc-4-Boc-
piperazine-2-carboxylic acid

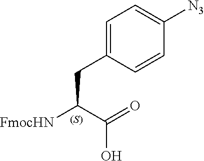

Fmoc-Phe(4-N₃)—OH

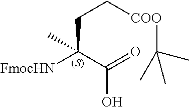

Fmoc-α-Me-Glu(tBu)

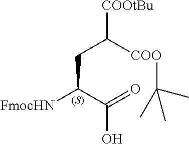

Fmoc-β-COOtBu-Glu(tBu)

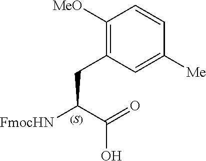

Fmoc-Phe(2-OMe—5-Me)—OH

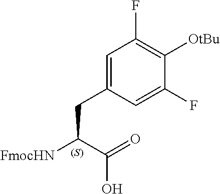

Fmoc-Tyr(tBu, 2,6-di-F)—OH

### Solid Phase Peptide Synthesis and Cyclization of Compounds

The procedures described in this example, either in whole or in part where noted, were used to synthesize the macrocyclic peptides described herein.

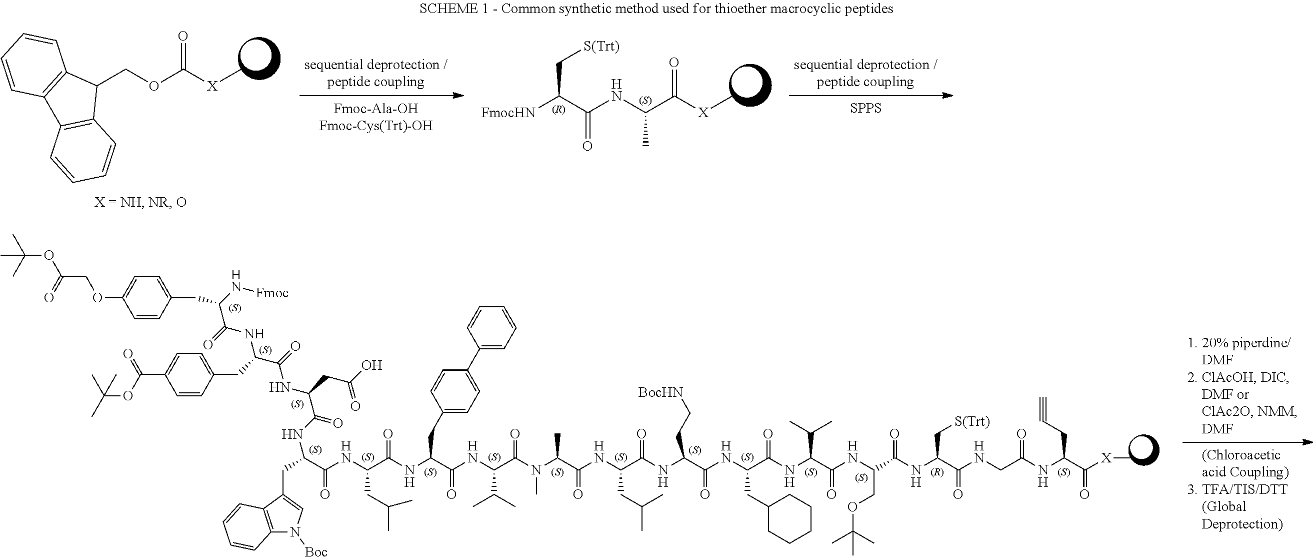

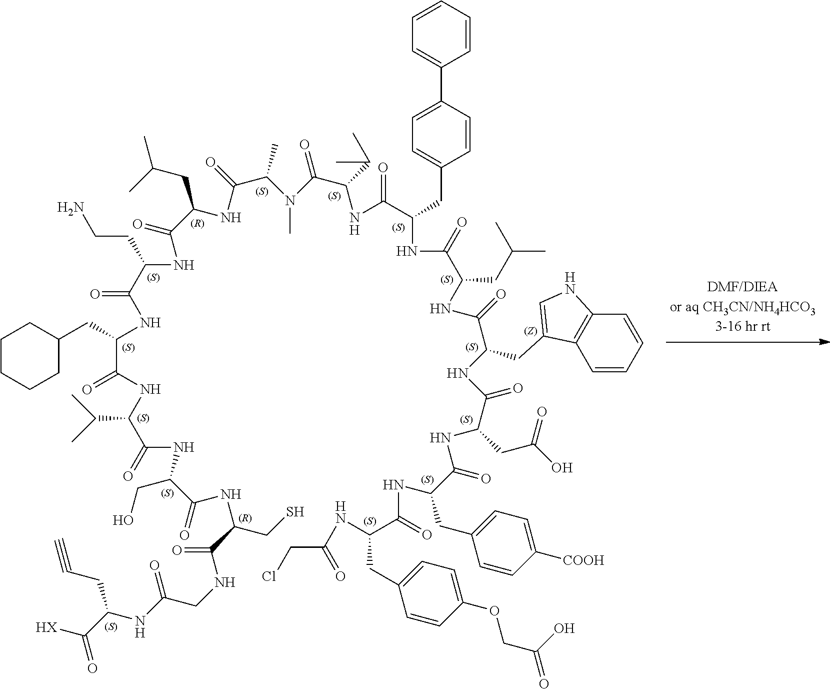

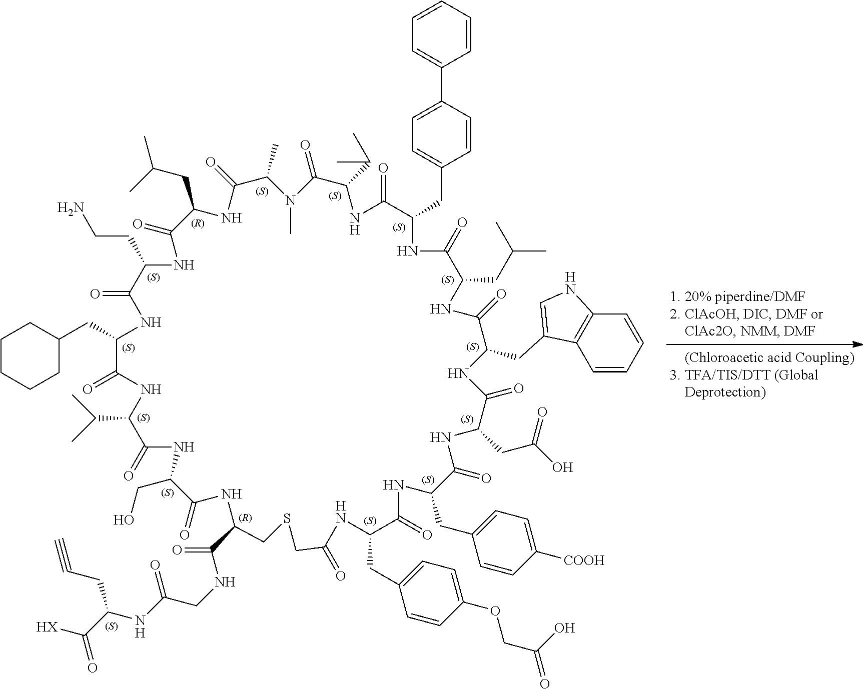

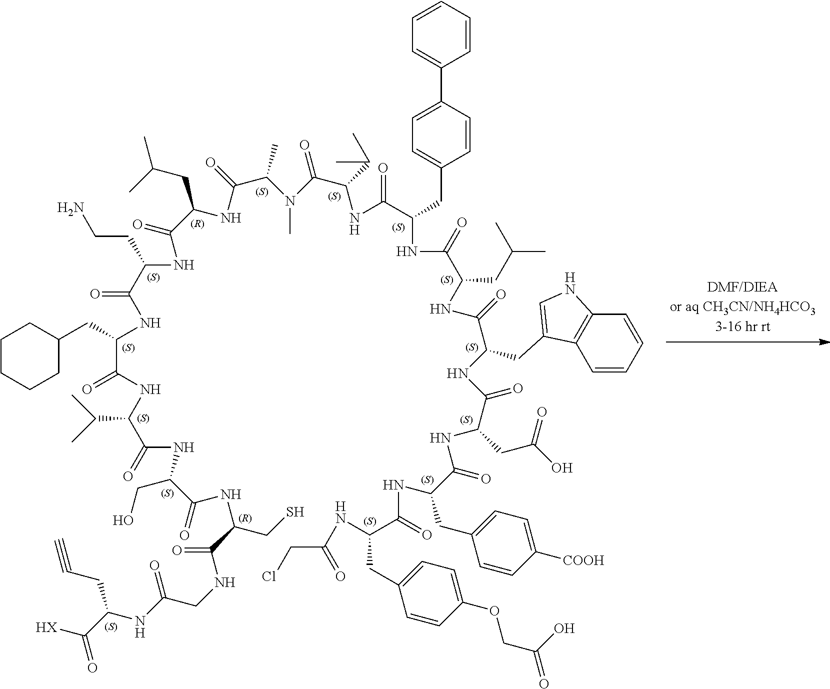

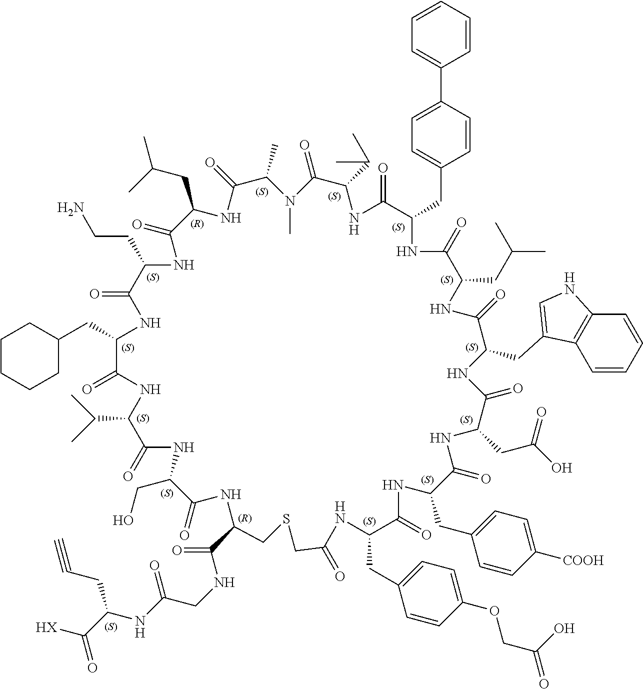

### General Protocol for Solid-Phase Peptide Synthesis (SPPS) and Macrocyclization

On a Symphony Peptide Synthesizer (Protein Technology Inc. Tucson, AZ), Prelude Peptide Synthesizer (Protein Technology Inc. Tucson, AZ), or Symphony X Peptide Synthesizer (Protein Technology Inc. Tucson, AZ), Chlorotrityl resin preloaded with Fmoc-Pra-OH (0.100 mmol) was swelled with CH₂Cl₂ then DMF when mixing under a gentle stream of N2. The solvent was drained and the following method was used to couple the first amino acid: the Fmoc group was removed from the resin-supported building block by washing the resin twice with a solution of 20% piperidine in DMF when mixing with a gentle stream of N2 every 30 seconds. The resin was washed five to six times with DMF. Fmoc-Ala-OH (0.2 M solution in DMF) was then added, followed by coupling activator (i.e., HATU (Chem-Impex Int'l, 0.4M solution in DMF) and base (i.e., N-methyl morpholine (Aldrich, 0.8 M in DMF). The reaction mixture was agitated by a gentle stream of nitrogen for 1-2 h. The reagents were drained from the reaction vessel, and the resin was washed five to six times with DMF. The resulting resin-supported Fmoc-protected dipeptide was then sequentially deprotected and coupled with third amino acid and so forth in an iterative fashion to give the desired resin-supported product.

The Fmoc group was removed from the N-terminus upon by washing the resin twice with a solution of 20% piperidine in DMF by a gentle stream of nitrogen. The resin was washed with DMF (5-6×). To the peptide-resin was treated with choroacetic anhydride (0.2 M in DMF) followed by NMM (0.8 M in DMF). This reaction was repeated. After draining all the reagents and solvents, the resin was washed with DMF and DCM, and then dried.

LCMS analysis was performed on a peptide aliquot, which was cleaved from the resin (analytical amount was treated with a small amount of TFA/TIS/DTT (96:4:1) solution at room temperature to confirm the formation of the desired linear sequence.

The peptide was globally deprotected and cleaved from the resin upon treatment with a TFA/TIS/DTT (96:4:1) solution for 1.5 h. The resin was removed by filtration, washed with a small amount of cleavage cocktail, and the combined filtrates were added to cold Et20. The solution was chilled at 0° C. in order to effect the peptide to precipitate out of solution. The slurry is centrifuged to pellet the solids and the supernatant was decanted. Fresh Et20 was added and the process was repeated three times to wash the solids. To the air-dried solids was added a solution of DIEA/DMF (1-3 mL of DIEA in 40-45 mL of DMF) or 0.1 M NH₄HCO₃/acetonitrile (from 1/1 to 3/1 (v/v)) so that the pH of the solution is greater than 8. The solution was stirred for 16-72 h and monitored by LCMS. The reaction solution was purified by preparative reverse phase HPLC to obtain the desired product.

### General Analytical Protocols and Synthesis Methods

### Analytical Data:

Mass Spectrometry: “ESI-MS(+)” signifies electrospray ionization mass spectrometry performed in positive ion mode; “ESI-MS(−)” signifies electrospray ionization mass spectrometry performed in negative ion mode; “ESI-HRMS(+)” signifies high-resolution electrospray ionization mass spectrometry performed in positive ion mode; “ESI-HRMS(−)” signifies high-resolution electrospray ionization mass spectrometry performed in negative ion mode. The detected masses are reported following the “m z” unit designation. Compounds with exact masses greater than 1000 were often detected as double-charged or triple-charged ions.

The crude material was purified via preparative LC/MS. Fractions containing the desired product were combined and dried via centrifugal evaporation.

### Analytical LCMS Condition A:

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm.

### Analytical LCMS Condition B:

Column: Waters Acquity UPLC BEH C18, 2.1×50 mm, 1.7-μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0-100% B over 3 minutes, then a 0.75-minute hold at 100% B; Flow: 1.0 mL/min; Detection: UV at 220 nm.

### Analytical LCMS Condition C:

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 70° C.; Gradient: 0-100% B over 3 minutes, then a 2.0-minute hold at 100% B; Flow: 0.75 mL/min; Detection: UV at 220 nm.

### Analytical LCMS Condition D:

Column: Waters Acquity UPLC BEH C18, 2.1×50 mm, 1.7-μm particles; Mobile Phase A: 5:95 acetonitrile:water with 0.1% trifluoroacetic acid; Mobile Phase B: 95:5 acetonitrile:water with 0.1% trifluoroacetic acid; Temperature: 70° C.; Gradient: 0-100% B over 3 minutes, then a 2.0-minute hold at 100% B; Flow: 0.75 mL/min; Detection: UV at 220 nm.

### Analytical LCMS Condition E:

Column: Kinetex XB C18, 3.0×75 mm, 2.6-μm particles; Mobile Phase A: 10 mM ammonium formate in water:acetonitrile (98:2); Mobile Phase B: 10 mM ammonium formate in Water:acetonitrile (02:98); Gradient: 20-100% B over 4 minutes, then a 0.6-minute hold at 100% B; Flow: 1.0 mL/min; Detection: UV at 254 nm.

### Analytical LCMS Condition F.

Column: Ascentis Express C18, 2.1×50 mm, 2.7-μm particles; Mobile Phase A: 10 mM ammonium acetate in water:acetonitrile (95:5); Mobile Phase B: 10 mM ammonium acetate in Water:acetonitrile (05:95), Temperature: 50° C.; Gradient: 0-100% B over 3 minutes; Flow: 1.0 mL/min; Detection: UV at 220 nm.

### Analytical LCMS Condition G:

Column: X Bridge C18, 4.6×50 mm, 5-μm particles; Mobile Phase A: 0.1% TFA in water; Mobile Phase B: acetonitrile, Temperature: 35° C.; Gradient: 5-95% B over 4 minutes; Flow: 4.0 mL/min; Detection: UV at 220 nm.

### Analytical LCMS Condition H.

Column: X Bridge C18, 4.6×50 mm, 5-μm particles; Mobile Phase A: 10 mM NH₄OAc; Mobile Phase B: methanol, Temperature: 35° C.; Gradient: 5-95% B over 4 minutes; Flow: 4.0 mL/min; Detection: UV at 220 nm.

### Analytical LCMS Condition I:

Column: X Bridge C18, 4.6×50 mm, 5-μm particles; Mobile Phase A: 10 mM NH₄OAc; Mobile Phase B: acetonitrile, Temperature: 35° C.; Gradient: 5-95% B over 4 minutes; Flow: 4.0 mL/min; Detection: UV at 220 nm.

### Analytical LCMS Condition J.

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 70° C.; Gradient: 0-100% B over 1.5 minutes, then a 2.0-minute hold at 100% B; Flow: 0.75 mL/min; Detection: UV at 254 nm.

### Analytical LCMS Condition K.

Column: Waters Acquity UPLC BEH C18, 2.1×50 mm, 1.7-μm particles; Mobile Phase A: 100% water with 0.05% trifluoroacetic acid; Mobile Phase B: 100% acetonitrile with 0.05% trifluoroacetic acid; Temperature: 50° C.; Gradient: 2-98% B over 1.0 minutes, then at 1.0-1.5 minute hold at 98% B; Flow: 0.80 mL/min; Detection: UV at 220 nm.

### Analytical LCMS Condition L:

Column: Waters Acquity UPLC BEH C18, 2.1×50 mm, 1.7-μm particles; Buffer:10 mM Ammonium Acetate. Mobile Phase A: buffer” CH₃CN (95/5); Mobile Phase B: Mobile Phase B:Buffer:ACN(5:95); Temperature: 50° C.; Gradient: 20-98% B over 2.0 minutes, then at 0.2 minute hold at 100% B; Flow: 0.70 mL/min; Detection: UV at 220 nm.

### Analytical LCMS Condition M:

Column: Waters Acquity UPLC BEH C18, 3.0×50 mm, 1.7-μm particles; Mobile Phase A: 95% water and 5% water with 0.1% trifluoroacetic acid; Mobile Phase B: 95% acetonitrile and 5% water with 0.1% trifluoroacetic acid; Temperature: 50° C.; Gradient: 20-100% B over 2.0 minutes, then at 2.0-2.3 minute hold at 100% B; Flow: 0.7 mL/min; Detection: UV at 220 nm.

### Analytical LCMS Condition N.

Column: Waters Acquity UPLC BEH C18, 2.1×50 mm, 1.7-μm particles; Mobile Phase A: 100% water with 0.05% trifluoroacetic acid; Mobile Phase B: 100% acetonitrile with 0.05% trifluoroacetic acid; Temperature: 50° C.; Gradient: 2-98% B over 5.0 minutes, then at 5.0-5.5 minute hold at 98% B; Flow: 0.80 mL/min; Detection: UV at 220 nm.

### Analytical LCMS Condition O:

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 50° C.; Gradient: 2%-98% B over 2 minutes, then a 0.5-minute hold at 98% B; Flow: 0.8 mL/min; Detection: UV at 220 nm.

### Analytical LCMS Condition P:

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 50° C.; Gradient: 0%-100% B over 3 minutes, then a 0.5-minute hold at 100% B; Flow: 1.0 mL/min; Detection: UV at 220 nm.

### Analytical LCMS Condition Q:

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 50° C.; Gradient: 0%-100% B over 1 minute, then a 0.5-minute hold at 100% B; Flow: 1.0 mL/min; Detection: UV at 220 nm.

### Analytical LCMS Condition R:

Column: Waters Acquity UPLC BEH C18, 2.1×50 mm, 1.7-μm particles; Buffer:10 mM Ammonium Acetate. Mobile Phase A: buffer” CH₃CN (95/5); Mobile Phase B: Mobile Phase B:Buffer:ACN(5:95); Temperature: 50° C.; Gradient: 0%-100% B over 1 minute, then a 0.5-minute hold at 100% B; Flow: 1.0 mL/min; Detection: UV at 220 nm.

### General Procedures:

### Prelude Method:

All manipulations were performed under automation on a Prelude peptide synthesizer (Protein Technologies). Unless noted, all procedures were performed in a 45-mL polypropylene reaction vessel fitted with a bottom frit. The reaction vessel connects to the Prelude peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N2 gas through the bottom frit; the pulse lasts approximately 5 seconds and occurs every 30 seconds. Amino acid solutions were generally not used beyond two weeks from preparation. HATU solution was used within 7-14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl)polystyrene.

Common amino acids used are listed below with side-chain protecting groups indicated inside parentheses: Fmoc-Ala-OH; Fmoc-Arg(Pbf)—OH; Fmoc-Asn(Trt)—OH; Fmoc-Asp(tBu)—OH; Fmoc-Bip-OH; Fmoc-Cys(Trt)—OH; Fmoc-Dab(Boc)—OH; Fmoc-Dap(Boc)—OH; Fmoc-Gln(Trt)—OH; Fmoc-Gly-OH; Fmoc-His(Trt)—OH; Fmoc-Hyp(tBu)—OH; Fmoc-Ile-OH; Fmoc-Leu-OH; Fmoc-Lys(Boc)—OH; Fmoc-Nle-OH; Fmoc-Met-OH; Fmoc-[N-Me]Ala-OH; Fmoc-[N-Me]Nle-OH; Fmoc-Orn(Boc)—OH, Fmoc-Phe-OH; Fmoc-Pro-OH; Fmoc-Sar-OH; Fmoc-Ser(tBu)—OH; Fmoc-Thr(tBu)—OH; Fmoc-Trp(Boc)—OH; Fmoc-Tyr(tBu)—OH; Fmoc-Val-OH and their corresponding D-amino acids.

The procedures of “Prelude Method” describe an experiment performed on a 0.100 mmol scale, where the scale is determined by the amount of Sieber or Rink or 2-chlorotrityl or PL-FMP resin. This scale corresponds to approximately 140 mg of the Sieber amide resin described above. All procedures can be scaled down or up from the 0.100 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)-used the “Double-coupling procedure” described below.

### Resin-Swelling Procedure:

To a 45-mL polypropylene solid-phase reaction vessel was added Sieber amide resin (140 mg, 0.100 mmol). The resin was washed (swelled) two times as follows: to the reaction vessel was added DMF (5.0 mL) through the top of the vessel “DMF top wash” upon which the mixture was periodically agitated for 10 minutes before the solvent was drained through the frit.

### Single-Coupling Procedure:

To the reaction vessel containing the resin from the previous step was added piperidine:DMF (20:80 v/v, 5.0 mL). The mixture was periodically agitated for 5.0 minutes and then the solution was drained through the frit. To the reaction vessel was added piperidine:DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 60-120 minutes, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Double-Coupling Procedure:

To the reaction vessel containing the resin from the previous step was added piperidine:DMF (20:80 v/v, 5.0 mL). The mixture was periodically agitated for 5.0 minutes and then the solution was drained through the frit. To the reaction vessel was added piperidine:DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 1-1.5 hour, then the reaction solution was drained through the frit. The resin was washed successively two times as follows: for each wash, DMF (5.0 mL) was added through the top of the vessel and the resulting mixture was periodically agitated for 1.0 minute before the solution was drained through the frit. To the reaction vessel was added the amino acid (0.2 M in DMF, 5.0 mL, 10 equiv), then HATU (0.4 M in DMF, 2.5 mL, 10 equiv), and finally NN (0.8 M in DMF, 2.5 mL, 20 equiv). The mixture was periodically agitated for 1-1.5 hours, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Single-Coupling Manual Addition Procedure A:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-2 mL) was added manually using a pipette from the top of the vessel while the bottom of the vessel was remain attached to the instrument, then the vessel was closed. The automatic program was resumed and HATU (0.4 M in DMF, 1.3 mL, 4 equiv) and NMM (1.3 M in DMF, 1.0 mL, 8 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel was remain attached to the instrument, followed by the manual addition of HATU (2-4 equiv, same equiv as the unnatural amino acid), and then the vessel was closed. The automatic program was resumed and NMM (1.3 M in DMF, 1.0 mL, 8 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

Peptoid Installation (50 pRnol) Procedure:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 60 seconds before the solution was drained through the frit. To the reaction vessel was added bromoacetic acid (0.4 M in DMF, 2.0 mL, 16 eq), then DIC (0.4 M in DMF, 2.0 mL, 16 eq). The mixture was periodically agitated for 1 hour, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. To the reaction vessel was added the amine (0.4 M in DMF, 2.0 mL, 16 eq). The mixture was periodically agitated for 1 hour, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Chloroacetic Anhydride Coupling:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed twice as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed successively five times as follows: for each wash, DMF (6.0 mL) was added through the top of the vessel and the resulting mixture was periodically agitated for one minute before the solution was drained through the frit. The resin was washed successively four times as follows: for each wash, DCM (6.0 mL) was added through the top of the vessel and the resulting mixture was periodically agitated for one minute before the solution was drained through the frit. The resin was then dried with nitrogen flow for 10 minutes. The resulting resin was used directly in the next step.

### Symphony Method:

All manipulations were performed under automation on a 12-channel Symphony peptide synthesizer (Protein Technologies). Unless noted, all procedures were performed in a 25-mL polypropylene reaction vessel fitted with a bottom frit. The reaction vessel connects to the Symphony peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N2 gas through the bottom frit; the pulse lasts approximately 5 seconds and occurs every 30 seconds. Amino acid solutions were generally not used beyond two weeks from preparation. HATU solution were used within 7-14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading.

PL-FMP resin: (4-Formyl-3-methoxyphenoxymethyl)polystyrene.

Fmoc-glycine-2-chlorotrityl chloride resin, 200-400 mesh, 1% DVB, 0.63 mmol/g loading.

Common amino acids used are listed below with side-chain protecting groups indicated inside parenthesis: Fmoc-Ala-OH; Fmoc-Arg(Pbf)—OH; Fmoc-Asn(Trt)—OH; Fmoc-Asp(tBu)—OH; Fmoc-Bip-OH; Fmoc-Cys(Trt)—OH; Fmoc-Dab(Boc)—OH; Fmoc-Dap(Boc)—OH; Fmoc-Gln(Trt)—OH; Fmoc-Gly-OH Fmoc-Gly-OH; Fmoc-His(Trt)—OH; Fmoc-Hyp(tBu)—OH; Fmoc-Ile-OH; Fmoc-Leu-OH; Fmoc-Lys(Boc)—OH; Fmoc-Nle-OH; Fmoc-Met-OH; Fmoc-[N-Me]Ala-OH; Fmoc-[N-Me]Nle-OH; Fmoc-Orn(Boc)—OH, Fmoc-Phe-OH; Fmoc-Pro-OH; Fmoc-Sar-OH; Fmoc-Ser(tBu)—OH; Fmoc-Thr(tBu)—OH; Fmoc-Trp(Boc)—OH; Fmoc-Tyr(tBu)—OH; Fmoc-Val-OH and their corresponding D-amino acids.

The procedures of “Symphony Method” describe an experiment performed on a 0.05 mmol scale, where the scale is determined by the amount of Sieber or Rink or chlorotrityl linker or PL-FMP bound to the resin. This scale corresponds to approximately 70 mg of the Sieber resin described above. All procedures can be scaled up from the 0.05 mmol scale by adjusting the described volumes by the multiple of the scale.

Prior to the amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)-used the “Double-coupling procedure” described below.

### Resin-Swelling Procedure:

To a 25-mL polypropylene solid-phase reaction vessel was added the resin (0.05 mmol). The resin was washed (swelled) as follows: to the reaction vessel was added DMF (2.0-3.0 mL, 1-2 times), upon which the mixture was periodically agitated for 10 minutes before the solvent was drained through the frit. Sometimes the resin was washed (swelled) as follows: to the reaction vessel was added CH₂Cl₂ (3-5 mL, 2 times) and upone which the mixture was periodically agitated for 30 min and before the solvent was drained through the frit. Then DMF (2.0-3.0 mL, 1-6 times), upon which the mixture was periodically agitated for 2-10 minutes before the solvent was drained through the frit.

### Single-Coupling Procedure:

To the reaction vessel containing the resin from the previous step was added DMF (2.5-3.75 mL) three times, upon which the mixture was agitated for 30 seconds before the solvent was drained through the frit each time. To the resin was added piperidine:DMF (20:80 v/v, 3.0-3.75 mL). The mixture was periodically agitated for 5.0 minutes and then the solution was drained through the frit. To the reaction vessel was added piperidine:DMF (20:80 v/v, 3.0-3.75 mL). The mixture was periodically agitated for 5.0 minutes and then the solution was drained through the frit. Sometimes the deprotection step was performed the third time. The resin was washed successively six times as follows: for each wash, DMF (2.5-3.75 mL) was added to the vessel and the resulting mixture was periodically agitated for 30 seconds before the solution was drained through the frit. To the reaction vessel was added the amino acid (0.2 M in DMF, 2.0-2.5 mL, 8-10 equiv), then HATU (0.4 M in DMF, 1.0-1.25 mL, 8-10 equiv), and finally NN (0.8 M in DMF, 1.0-1.25 mL, 20 equiv). The mixture was periodically agitated for 30-120 minutes, then the reaction solution was drained through the frit. The resin was washed successively six times as follows: for each wash, DMF (2.5-3.0 mL) was added and the resulting mixture was periodically agitated for 30 seconds before the solution was drained through the frit. The resulting resin was used directly in the next step.

### Single-Coupling Manual Addition Procedure:

To the reaction vessel containing the resin from the previous step was added DMF (3.0-3.75 mL) three times, upon which the mixture was agitated for 30 seconds before the solvent was drained through the frit each time. To the resin was added piperidine:DMF (20:80 v/v, 3.0-3.75 mL). The mixture was periodically agitated for 5.0 minutes and then the solution was drained through the frit. To the reaction vessel was added piperidine:DMF (20:80 v/v, 3.0-3.75 mL). The mixture was periodically agitated for 5.0 minutes and then the solution was drained through the frit. The mixture was periodically agitated for 5.0 minutes and then the solution was drained through the frit. The resin was washed successively six times as follows: for each wash, DMF (3.0-3.75 mL) was added to the vessel and the resulting mixture was periodically agitated for 30 seconds before the solution was drained through the frit. To the reaction vessel was added the premixed amino acid (2.0-5.0 equiv) and HATU (0.4 M in DMF, 2.0-5.0 equiv), then NMM (0.8 M in DMF, 4.0-10.0 equiv) and the molar ratio for amino acid, HATU, and NMM is 1:1:2. The mixture was periodically agitated for 2-6 hours, then the reaction solution was drained through the frit. The resin was washed successively four times as follows: for each wash, DMF (3.75 mL) was added and the resulting mixture was periodically agitated for 30 seconds before the solution was drained through the frit. The resulting resin was used directly in the next step.

### Double-Coupling Procedure:

To the reaction vessel containing resin from the previous step was added DMF (2.5-3.75 mL) three times, upon which the mixture was agitated for 30 seconds before the solvent was drained through the frit each time. To the reaction vessel was added piperidine:DMF (20:80 v/v, 3.0-3.75 mL). The mixture was periodically agitated for 5 minutes and then the solution was drained through the frit. To the reaction vessel was added piperidine:DMF (20:80 v/v, 3.0-3.75 mL). The mixture was periodically agitated for 5 minutes and then the solution was drained through the frit. The resin was washed successively six times as follows: for each wash, DMF (3.0-3.75 mL) was added and the resulting mixture was periodically agitated for 30 seconds before the solution was drained through the frit. To the reaction vessel was added the amino acid (0.2 M in DMF, 2.0-2.5 mL, 8-10 equiv), then HATU (0.4 M in DMF, 1.0-1.25 mL, 10 equiv), and finally NN (0.8 M in DMF, 1.0-1.25 mL, 16-20 equiv). The mixture was periodically agitated for 1 hour, then the reaction solution was drained through the frit. The resin was washed twice with DMF (3.0-3.75 mL) and the resulting mixture was periodically agitated for 30 seconds before the solution was drained through the frit each time. To the reaction vessel was added the amino acid (0.2 M in DMF, 2.0-2.5 mL, 8-10 equiv), then HATU (0.4 M in DMF, 1.0-1.25 mL, 8-10 equiv), and finally NMM (0.8 M in DMF, 1.0-1.25 mL, 16-20 eq). The mixture was periodically agitated for 1-2 hours, then the reaction solution was drained through the frit. The resin was successively washed six times as follows: for each wash, DMF (3.0-3.75 mL) was added and the resulting mixture was periodically agitated for 30 seconds before the solution was drained through the frit. The resulting resin was used directly in the next step.

Peptoid Installation (50 μmol) Procedure:

To the reaction vessel containing the resin from the previous step was added piperidine: DMF (20:80 v/v, 3.75 mL). The mixture was periodically agitated for 5 minutes and then the solution was drained through the frit. To the reaction vessel was added piperidine: DMF (20:80 v/v, 3.75 mL). The mixture was periodically agitated for 5 minutes and then the solution was drained through the frit. The resin was washed successively six times as follows: for each wash, DMF (3.75 mL) was added to the vessel and the resulting mixture was periodically agitated for 30 seconds before the solution was drained through the frit. To the reaction vessel was added the bromoacetic acid (0.4 M in DMF, 2.5 mL, 10 eq), then DIC (0.4 M in DMF, 2.5 mL, 10 eq). The mixture was periodically agitated for 60 mins, then the reaction solution was drained through the frit. The resin was washed successively two times as follows: for each wash, DMF (3.75 mL) was added to the vessel and the resulting mixture was periodically agitated for 30 seconds before the solution was drained through the frit. To the reaction vessel was added the amine (0.4 M in DMF, 2.5 mL, 10 eq), the mixture was periodically agitated for 60 mins, and then the reaction solution was drained through the frit. The resin was washed successively five times as follows: for each wash, DMF (3.75 mL) was added to the vessel and the resulting mixture was periodically agitated for 30 seconds before the solution was drained through the frit. The resulting resin was used directly in the next step.

### Chloroacetic Anhydride Coupling:

To the reaction vessel containing resin from the previous step was added DMF (3.0-3.75 mL) three times, upon which the mixture was agitated for 30 seconds before the solvent was drained through the frit each time. To the reaction vessel containing the resin from the previous step was added piperidine:DMF (20:80 v/v, 3.0-3.75 mL). The mixture was periodically agitated for 5 minutes and then the solution was drained through the frit. To the reaction vessel was added piperidine:DMF (20:80 v/v, 3.0-3.75 mL). The mixture was periodically agitated for 5 minutes and then the solution was drained through the frit. The resin was washed successively six times as follows: for each wash, DMF (3.0-3.75 mL) was added through the top of the vessel and the resulting mixture was periodically agitated for 30 seconds before the solution was drained through the frit. To the reaction vessel was added the chloroacetic anhydride solution (0.4 M in DMF, 3.0-3.75 mL, 30 equiv), then NMM (0.8 M in DMF, 2.5 mL, 40 equiv). The mixture was periodically agitated for 15 minutes, then the reaction solution was drained through the frit. The resin was washed once as follows: DMF (5.0-6.25 mL) was added through the top of the vessel and the resulting mixture was periodically agitated for 30 seconds before the solution was drained through the frit. To the reaction vessel was added the chloroacetic anhydride solution (0.4 M in DMF, 3.75 mL, 30 equiv), then NN (0.8 M in DMF, 2.5 mL, 40 equiv). The mixture was periodically agitated for 15 minutes, then the reaction solution was drained through the frit. The resin was washed successively six times as follows: for each wash, DMF (2.5 mL) was added through the top of the vessel and the resulting mixture was periodically agitated for 30 seconds before the solution was drained through the frit. The resin was washed successively four times as follows: for each wash, DCM (2.5 mL) was added through the top of the vessel and the resulting mixture was periodically agitated for 30 seconds before the solution was drained through the frit. The resulting resin was dried using a nitrogen flow for 10 mins before being used directly in the next step.

### Symphony X Methods:

All manipulations were performed under automation on a Symphony X peptide synthesizer (Protein Technologies). Unless noted, all procedures were performed in a 45-mL polypropylene reaction vessel fitted with a bottom frit. The reaction vessel connects to the Symphony X peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N2 gas through the bottom frit; the pulse lasts approximately 5 seconds and occurs every 30 seconds. A “single shot” mode of addition describes the addition of all the solution contained in the single shot falcon tube that is usually any volume less than 5 mL. Amino acid solutions were generally not used beyond two weeks from preparation. HATU solution was used within 14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl)polystyrene.

Common amino acids used are listed below with side-chain protecting groups indicated inside parenthesis: Fmoc-Ala-OH; Fmoc-Arg(Pbf)—OH; Fmoc-Asn(Trt)—OH; Fmoc-Asp(tBu)—OH; Fmoc-Bip-OH; Fmoc-Cys(Trt)—OH; Fmoc-Dab(Boc)—OH; Fmoc-Dap(Boc)—OH; Fmoc-Gln(Trt)—OH; Fmoc-Gly-OH; Fmoc-His(Trt)—OH; Fmoc-Hyp(tBu)—OH; Fmoc-Ile-OH; Fmoc-Leu-OH; Fmoc-Lys(Boc)—OH; Fmoc-Nle-OH; Fmoc-Met-OH; Fmoc-[N-Me]Ala-OH; Fmoc-[N-Me]Nle-OH; Fmoc-Orn(Boc)—OH, Fmoc-Phe-OH; Fmoc-Pro-OH; Fmoc-Sar-OH; Fmoc-Ser(tBu)—OH; Fmoc-Thr(tBu)—OH; Fmoc-Trp(Boc)—OH; Fmoc-Tyr(tBu)—OH; Fmoc-Val-OH and their corresponding D-amino acids.

The procedures of “Symphony X Method” describe an experiment performed on a 0.050 mmol scale, where the scale is determined by the amount of Sieber or Rink or 2-chlorotrityl or PL-FMP bound to the resin. This scale corresponds to approximately 70 mg of the Sieber amide resin described above. All procedures can be scaled beyond or under 0.050 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- or D-Leu used the “Double-coupling procedure” or the “Single-Coupling 2-Hour Procedure” described below. Unless otherwise specified, the last step of automated synthesis is the acetyl group installation described as “Chloroacetyl Anhydride Installation”. All syntheses end with a final rinse and drying step described as “Standard final rinse and dry procedure”.

### Resin-Swelling Procedure:

To a 45-mL polypropylene solid-phase reaction vessel was added Sieber amide resin (70 mg, 0.050 mmol). The resin was washed (swelled) three times as follows: to the reaction vessel was added DMF (5.0 mL) through the top of the vessel “DMF top wash” upon which the mixture was periodically agitated for 3 minutes before the solvent was drained through the frit.

### Single-Coupling Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NN (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling 4 Equivalent Procedure:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 1.0 mL, 4 equiv), then HATU (0.2 M in DMF, 1.0 mL, 4 equiv), and finally NN (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Double-Coupling Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1 hour, then the reaction solution was drained through the frit. The resin was washed successively two times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Double-Coupling 4 Equivalent Procedure:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 1.0 mL, 4 equiv), then HATU (0.2 M in DMF, 1.0 mL, 4 equiv), and finally NN (0.8 M in DMF, 1.0 mL, 16 equiv). The mixture was periodically agitated for 1 hour, then the reaction solution was drained through the frit. The resin was washed successively two times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 1.0 mL, 4 equiv), then HATU (0.2 M in DMF, 1.0 mL, 4 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure A:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel was remain attached to the instrument, then the vessel was closed. The automatic program was resumed and HATU (0.4 M in DMF, 1.0 mL, 8 equiv) and NMM (0.8 M in DMF, 1.0 mL, 16 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel was remain attached to the instrument, followed by the manual addition of HATU (2-4 equiv, same equiv as the unnatural amino acid), then the vessel was closed. The automatic program was resumed and NMM (0.8 M in DMF, 1.0 mL, 16 equiv) was added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure C:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) containing HATU (an equimolor amount relative to the unnatural amino acid), and NMM (4-8 equiv) was added manually using a pipette from the top of the vessel while the bottom of the vessel remained attached to the instrument. The automatic program was resumed and the mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure D:

To the reaction vessel containing the resin from the previous step was added piperidine: DMF (20:80 v/v, 4.0 mL). The mixture was periodically agitated for 5 minutes and then the solution was drained through the frit. To the reaction vessel was added piperidine: DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) containing DIC (an equimolor amount relative to the unnatural amino acid), and HOAt (an equimolor amount relative to the unnatural amino acid),, was added manually using a pipette from the top of the vessel while the bottom of the vessel was remain attached to the instrument. The automatic program was resumed and the mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

Peptoid Installation (50 μmol) Procedure:

To the reaction vessel containing the resin from the previous step was added piperidine: DMF (20:80 v/v, 3.0 mL). The mixture was periodically agitated for 5 minutes and then the solution was drained through the frit. To the reaction vessel was added piperidine: DMF (20:80 v/v, 3.0 mL). The mixture was periodically agitated for 5 minutes and then the solution was drained through the frit. The resin was washed successively six times as follows: for each wash, DMF (3.0 mL) was added through the top of the vessel and the resulting mixture was periodically agitated for 30 seconds before the solution was drained through the frit. To the reaction vessel was added bromoacetic acid (0.4 M in DMF, 1.0 mL, 8 eq), then DIC (0.4 M in DMF, 1.0 mL, 8 eq). The mixture was periodically agitated for 1 hour, then the reaction solution was drained through the frit. The resin was washed successively two times as follows: for each wash, DMF (4.0 mL) was added through the top of the vessel and the resulting mixture was periodically agitated for 30 seconds before the solution was drained through the frit. To the reaction vessel was added the amine (0.4 M in DMF, 2.0 mL, 16 eq). The mixture was periodically agitated for 1 hour, then the reaction solution was drained through the frit. The resin was washed successively five times as follows: for each wash, DMF (3.0 mL) was added through the top of the vessel and the resulting mixture was periodically agitated for 30 seconds before the solution was drained through the frit. The resulting resin was used directly in the next step.

### Chloroacetic Anhydride Coupling:

To the reaction vessel containing the resin from the previous step was added piperidine:DMF (20:80 v/v, 3.0 mL). The mixture was periodically agitated for 3.5 or 5 minutes and then the solution was drained through the frit. To the reaction vessel was added piperidine:DMF (20:80 v/v, 3.0 mL). The mixture was periodically agitated for 5 minutes and then the solution was drained through the frit. The resin was washed successively six times as follows: for each wash, DMF (3.0 mL) was added through the top of the vessel and the resulting mixture was periodically agitated for 30 seconds before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed twice as follows: for each wash, DMF (3.0 mL) was added through the top of the vessel and the resulting mixture was periodically agitated for 1.0 minute before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed successively five times as follows: for each wash, DMF (3.0 mL) was added through the top of the vessel and the resulting mixture was periodically agitated for 1.0 minute before the solution was drained through the frit. The resulting resin was used directly in the next step.

### Final Rinse and Dry Procedure:

The resin from the previous step was washed successively six times as follows: for each wash, DCM (5.0 mL) was added through the top of the vessel and the resulting mixture was periodically agitated for 30 seconds before the solution was drained through the frit. The resin was then dried using a nitrogen flow for 10 minutes. The resulting resin was used directly in the next step.

### Sonata Method:

All procedures unless noted were performed under automation on a Sonata peptide synthesizer (Protein Technologies). All procedures unless noted were performed in a 200 mL-glass reaction vessel that connects to the Sonata peptide synthesizer through both the bottom and the top of the vessel. All reagents are added through the bottom of the vessel, and washes are performed with one single top wash followed by five bottom washes. All solutions are removed through the bottom of the vessel. “Periodic agitation” describes a brief pulse of N2 gas through the bottom frit; the pulse lasts approximately 5 seconds and occurs every 30 seconds. Mechanical agitation is performed during additions and throughout washes and/or reaction cycles. Chloroacetic acid/DIC solutions in DMF were used within 0.25 h of preparation. Amino acid solutions were not used beyond three days from preparation. HATU solutions were used within five days of preparation. DMF=N, N-dimethylformamide; NN=N-methylmorpholine.

The procedures of “Prelude Method” describe an experiment performed on a 2 mmol scale, where the scale is determined by the amount of Sieber or Rink or 2-chlorotrityl or PL-FMP resin. The procedures described below can be scaled up beyond 2 mmol scale simply by adjusting the described volumes by the multiple of the scale. The outline of a general synthesis is as follows: Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)-used the “Double-coupling procedure” described below.
Swelling of resin (automated)
Amino acid coupling steps (repetition of general procedures A & B; automated)
Capping with chloroacetic acid (automated)
Global deprotection (manual)
Cyclization (manual)

### Resin-Swelling and First-Coupling Procedure:

Note: This procedure contains a swelling step and is used as the first coupling cycle.

To a 200-mL glass reaction vessel was added the resin The resin was washed successively four times as follows: for each wash, DMF (9 seconds, ˜45 mL) was added through the top of the vessel and the resulting mixture was mechanically and periodically agitated for 1 minute before the solution was drained through the frit. To the reaction vessel containing resin from the previous step was added piperidine: DMF (20:80 v/v, 10 second delivery ˜50 mL). The mixture was mechanically and periodically agitated for 5 minutes and then the solution was drained through the frit. To the reaction vessel was added piperidine: DMF (20:80 v/v, 10 seconds −50 mL). The mixture was mechanically and periodically agitated for 5 minutes and then the solution was drained through the frit. The resin was washed successively six time as follows: for each wash, DMF (9 seconds, ˜45 mL) was added to top of the vessel and the resulting mixture was mechanically and periodically agitated for 1 minute before the solution was drained through the frit. To the reaction vessel was added the amino acid (0.2 M in DMF, 20 mL, 2 equiv), then HATU (0.4 M in DMF, 10 mL, 2 equiv), and finally NMM (0.8 M in DMF, 10 mL, 4 equiv). The mixture was mechanically and periodically agitated for 30 minutes, then the reaction solution was drained through the frit. The resin was washed successively five times as follows: for each wash, DMF (9 seconds, −45 mL) was added through the top of the vessel and the resulting mixture was mechanically and periodically agitated for 1 minute before the solution was drained through the frit. To the reaction vessel was added over 9 seconds −45 mL of 10%(v/v) acetic anhydride and 10%(v/v) d IPEA in DMF. The mixture was mechanically and periodically agitated for 10 minutes, then the solution was drained through the frit. The resin was washed successively five times as follows: for each wash, DMF (9 seconds, −45 mL) was added through the bottom of the vessel and the resulting mixture was mechanically and periodically agitated for 90 seconds before the solution was drained through the frit. The resulting resin was used directly in the next step.

Single-Coupling with Capping Procedure:

To the reaction vessel containing the resin from the previous step was added piperidine: DMF (20:80 v/v, −50 mL, 10 second delivery). The mixture was mechanically and periodically agitated for 5 minutes and then the solution was drained through the frit. To the reaction vessel was added piperidine: DMF 20:80 v/v, −50 mL, 10 second delivery). The mixture was mechanically and periodically agitated for 5 minutes and then the solution was drained through the frit. The resin was washed successively 6 times as follows: for each wash, DMF (9 seconds, ˜45 mL) was added through the top of the vessel and the resulting mixture was mechanically and periodically agitated for 1 minute before the solution was drained through the frit. To the reaction vessel was added the amino acid (0.2 M in DMF, 20 mL, 2 equiv), then HATU (0.4 M in DMF, 10 mL, 2 equiv), and finally NMM (0.8 M in DMF, 10 mL, 4 equiv). The mixture was mechanically and periodically agitated for 30 minutes, then the reaction solution was drained through the frit. The resin was washed successively five times as follows: for each wash, DMF (9 seconds, −45 mL) was added through the top of the vessel and the resulting mixture was mechanically and periodically agitated for 1 minute before the solution was drained through the frit. To the reaction vessel was added over 9 seconds −45 mL of 10%(v/v) acetic anhydride and 10%(v/v) DIEA in DMF. The mixture was mechanically and periodically agitated for 10 minutes, then the solution was drained through the frit. The resin was washed successively five times as follows: for each wash, DMF (9 seconds, −45 mL) was added through the bottom of the vessel and the resulting mixture was mechanically and periodically agitated for 90 seconds before the solution was drained through the frit. The resulting resin was used directly in the next step.

Double-Coupling with Capping Procedure:

Note: There are two exposures of the amino acid and coupling reagents (“double-coupling”). This procedure is typically used if the reacting terminal amine is secondary rather than primary.

To the reaction vessel containing resin from the previous step was added piperidine: DMF (20:80 v/v, 10 second delivery, −50 mL). The mixture was mechanically and periodically agitated for 5 minutes and then the solution was drained through the frit. To the reaction vessel was added piperidine: DMF (20:80 v/v, 10 seconds, −50 mL). The mixture was mechanically and periodically agitated for 5 minutes and then the solution was drained through the frit. The resin was washed successively six times as follows: for each wash, DMF (9 seconds, 45 mL) was added through the top of the vessel and the resulting mixture was mechanically and periodically agitated for 1 minute before the solution was drained through the frit. To the reaction vessel was added the amino acid (0.2 M in DMF, 20 mL, 2 equiv), then HATU (0.4 M in DMF, 10 mL, 2 equiv), and finally NMM (0.8 M in DMF, 10 mL, 4 equiv). The mixture was mechanically and periodically agitated for 30 minutes, then the reaction solution was drained through the frit. The resin was twice washed as follows: for each wash, DMF (9 seconds, −45 mL) was added through the top of the vessel and the resulting mixture was mechanically and periodically agitated for 1 minute before the solution was drained through the frit. To the reaction vessel was added the amino acid (0.2 M in DMF, 20 mL, 2 equiv), then HATU (0.4 M in DMF, 10 mL, 2 equiv), and finally N N (0.8 M in DMF, 10 mL, 4 eq). The resin was washed successively five times as follows: for each wash, DMF (9 seconds, −45 mL) was added through the top of the vessel and the resulting mixture was mechanically and periodically agitated for 1 minute before the solution was drained through the frit. To the reaction vessel was added over 9 seconds −45 mL of 10%(v/v) acetic anhydride and 10%(v/v) d IPEA in DMF. The mixture was mechanically and periodically agitated for 10 minutes, then the solution was drained through the frit. The resin was washed successively five times as follows: for each wash, DMF (9 seconds, −45 mL) was added through the bottom of the vessel and the resulting mixture was mechanically and periodically agitated for 90 seconds before the solution was drained through the frit. The resulting resin was used directly in the next step.

### Single Coupling, Extended Time Procedure:

Note: The coupling time is extended to two hours.

To the reaction vessel containing resin from the previous step was added piperidine: DMF (20:80 v/v, 10 second delivery, −50 mL). The mixture was mechanically and periodically agitated for 5 minutes and then the solution was drained through the frit. To the reaction vessel was added piperidine: DMF (20:80 v/v, 10 seconds, −50 mL). The mixture was mechanically and periodically agitated for 5 minutes and then the solution was drained through the frit. The resin was washed successively six times as follows: for each wash, DMF (9 seconds, 45 mL) was added through the bottom of the vessel and the resulting mixture was mechanically and periodically agitated for 1 minute before the solution was drained through the frit. To the reaction vessel was added the amino acid (0.2 M in DMF, 20 mL, 2 equiv), then HATU (0.4 M in DMF, 10 mL, 2 equiv), and finally NMM (0.8 M in DMF, 10 mL, 4 equiv). The mixture was mechanically and periodically agitated for 120 minutes, then the reaction solution was drained through the frit. The resin was washed successively five times as follows: for each wash, DMF (9 seconds, −45 mL) was added through the top of the vessel and the resulting mixture was mechanically and periodically agitated for 1 minute before the solution was drained through the frit. To the reaction vessel was added over 9 seconds −45 mL of 10%(v/v) acetic anhydride and 10%(v/v) d IPEA in DMF. The mixture was mechanically and periodically agitated for 10 minutes, then the solution was drained through the frit. The resin was washed successively five times as follows: for each wash, DMF (9 seconds, −45 mL) was added through the bottom of the vessel and the resulting mixture was mechanically and periodically agitated for 90 seconds before the solution was drained through the frit. The resulting resin was used directly in the next step.

### Final Coupling Procedure:

Note: This condition differs from General Procedure B in the fact that it contains a final DCMwash of the peptide to prevent any loss of the terminal FMOC from the linear peptide

To the reaction vessel containing resin from the previous step was added piperidine: DMF (20:80 v/v, 10 second delivery, −50 mL). The mixture was mechanically and periodically agitated for 5 minutes and then the solution was drained through the frit. To the reaction vessel was added piperidine: DMF (20:80 v/v, 10 seconds, −50 mL). The mixture was mechanically and periodically agitated for 5 minutes and then the solution was drained through the frit. The resin was washed successively 1 time as follows: DMF (9 seconds, ˜45 mL) was added through the top of the vessel and the resulting mixture was mechanically and periodically agitated for 1 minute before the solution was drained through the frit. The resin was washed successively 5 times as follows: for each wash, DMF (9 seconds, ˜45 mL) was added through the top of the vessel and the resulting mixture was mechanically and periodically agitated for 1 minute before the solution was drained through the frit. To the reaction vessel was added the amino acid (0.2 M in DMF, 20 mL, 2 equiv), then HATU (0.4 M in DMF, 10 mL, 2 equiv), and finally N N (0.8 M in DMF, 10 mL, 4 equiv). The mixture was mechanically and periodically agitated for 30 minutes, then the reaction solution was drained through the frit. The resin was twice washed as follows: for each wash, DMF (9 seconds, −45 mL) was added through the top of the vessel and the resulting mixture was mechanically and periodically agitated for 1 minute before the solution was drained through the frit. To the reaction vessel was added the amino acid (0.2 M in DMF, 20 mL, 2 equiv), then HATU (0.4 M in DMF, 10 mL, 2 equiv), and finally NMM (0.8 M in DMF, 10 mL, 4 equiv). The resin was washed successively five times as follows: for each wash, DMF (9 seconds, −45 mL) was added to top of the vessel and the resulting mixture was mechanically and periodically agitated for 1 minute before the solution was drained through the frit. To the reaction vessel was added over 9 seconds −45 mL of 10%(v/v) acetic anhydride and 10%(v/v) DIEA in DMF. The mixture was mechanically and periodically agitated for 10 minutes, then the solution was drained through the frit. The resin was washed successively five times as follows: for each wash, DMF (9 seconds, −45 mL) was added through the bottom of the vessel and the resulting mixture was mechanically and periodically agitated for 90 seconds before the solution was drained through the frit. The resin was washed successively seven times as follows: for each wash, DCM (10 seconds, −50 mL) was added through the bottom of the vessel and the resulting mixture was mechanically and periodically agitated for 90 seconds before the solution was drained through the frit. The resulting resin was used directly in the next step.

### Manual Mode Chloroacetic Acid Capping Procedure:

To the reaction vessel containing resin from the previous step was added piperidine: DMF (20:80 v/v, 10 second delivery, −50 mL). The mixture was mechanically and periodically agitated for 5 minutes and then the solution was drained through the frit. To the reaction vessel was added piperidine: DMF (20:80 v/v, 10 seconds, −50 mL). The mixture was mechanically and periodically agitated for 5 minutes and then the solution was drained through the frit. The resin was washed successively six times as follows: for each wash, DMF (9 seconds, 45 mL) was added through the bottom of the vessel and the resulting mixture was mechanically and periodically agitated for 1 minute before the solution was drained through the frit. To an Erlenmeyer flask were added 1.9 g (10 equiv) of chloroacetic acid in 40 mL of DMF and 4.7 mL (15 equiv) of DIC. This prepared solution was added to the 200-mL glass reactor which contains the resin, using a manual mode, 2 seconds, ˜10 mL DMF is added through the bottom of the frit and the resulting mixture was mechanically and periodically agitated for 2.5 days. The reaction time is likely to be much shorter and can be monitored using the Kaiser ninhydrin test. The resin was washed successively five times as follows: for each wash, DMF (9 seconds, −45 mL) was added through the bottom of the vessel and the resulting mixture was mechanically and periodically agitated for 1 minute before the solution was drained through the frit. The resin was washed successively seven times as follows: for each wash, DCM (10 seconds, −50 mL) was added through the bottom of the vessel and the resulting mixture was mechanically and periodically agitated for 1 minute before the solution was drained through the frit.

Alternatively, when 4 equivalents of amino acids were used, acetic anhydrade capping was not performed (no capping) in the above single-coupling and double-couplings steps

Manual Handling and Manipulation for all the following procedures:

Manual handling and manipulation for all following procedures: a “deprotection solution” was prepared by combining in a 200 mL Erlenmeyer: 1% by weight of dithiothreitol (75 mg), 2.5% by volume of triisopropylsilane (1.875 mL), and trifluoroacetic acid (75 mL). The deprotection solution was cooled in an ice water bath to 5° C. prior to addition to the resin. 0.8 mmol-4 g of resin were placed in a 100 mL peptide synthesis vessel, the cold “deprotection solution” was added in one portion, the mixture was capped and shaken on a shaker for 1.5 hours. The filtrate was collected equally in 8×50 mL polypropylene Falcon tubes. 30 mL ether were added to each tube, capped and shaken to provide a white precipitate. The tubes were chilled in a refrigerator for 1 hour prior to centrifugation. Each tube was centrifuged (3 min, 2500 rpm) and the ethereal layers discarded. The precipitate was washed with ether (3×20 mL) and the centrifugation was repeated to provide the crude linear chloroacylated peptide.

7.5 mL of acetonitrile were added to each tube, and then they were diluted to 35 mL with aqueous 0.1 M NH₄HCO₃, shaking the resulting mixture to dissolve all solids. Caution: CO₂ evolves from quenching the excess TFA with ammonium bicarbonate. All solutions were transferred to a freeze drying flask and each Falcon tube was washed with 5 mL of a 1:1 mixture of acetonitrile and aqueous 0.1 M NH₄HCO₃. The resulting solution was carefully adjusted to pH 8 using 1.0 M NaOH. The solution was allowed to stand overnight (18 h). Reaction completion was confirmed by HPLC MS. Then the mixture was frozen in an dry ice/acetone bath and lyophilized to provide a white solid for purification.

### General Deprotection, Cyclization, N-Methylation, Click, Suzuki Procedures: Global Deprotection Method A:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 50-mL falcon tube was added the resin and 2.0-5.0 mL of the cleavage cocktail (TFA:TIS:DTT, v/v/w=95:5:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. To the suspension was added 35-50 mL of cold diethyl ether. The mixture was vigorously mixed upon which a significant amount of a white solid precipitated. The mixture was centrifuged for 3-5 minutes, then the solution was decanted away from the solids and discarded. The solids were suspended in Et20 (30-40 mL); then the mixture was centrifuged for 3-5 minutes; and the solution was decanted away from the solids and discarded. For a final time, the solids were suspended in Et20 (30-40 mL); the mixture was centrifuged for 3-5 minutes; and the solution was decanted away from the solids and discarded to afford the crude peptide as a white to off-white solid together with the cleaved resin after drying under a flow of nitrogen and/or under house vacuum. The crude was used at the same day for the cyclization step.

### Global Deprotection Method B:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 30-mL bio-rad poly-prep chromatography column was added the resin and 2.0-5.0 mL of the cleavage cocktail (TFA:TIS:H₂O:DTT, v/v/w=94:3:3:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. The acidic solution was drained into 40 mL of cold diethyl ether and the resin was washed twice with 0.5 mL of TFA solution. The mixture was centrifuged for 3-5 minutes, then the solution was decanted away from the solids and discarded. The solids were suspended in Et20 (35 mL); then the mixture was centrifuged for 3-5 minutes; and the solution was decanted away from the solids and discarded. For a final time, the solids were suspended in Et20 (35 mL); the mixture was centrifuged for 3-5 minutes; and the solution was decanted away from the solids and discarded to afford the crude peptide as a white to off-white solid after drying under a flow of nitrogen and/or under house vacuum. The crude was used at the same day for the cyclization step.

### Cyclization Method A:

Unless noted, all manipulations were performed manually. The procedure of “Cyclization Method A” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids from the globle deprotection were dissolved in DMF (30-45 mL) in the 50-mL centrifuge tube at room temperature, and to the solution was added DIEA (1.0-2.0 mL) and the pH value of the reaction mixure above was 8. The solution was then allowed to shake for several hours or overnight or over 2-3 days at room temperature. The reaction solution was concentrated to dryness on speedvac or genevac EZ-2 and the crude residue was then dissolved in DMF or DMF/DMSO (2 mL). After filtration, this solution was subjected to single compound reverse-phase HPLC purification to afford the desired cyclic peptide.

### Cyclization Method B:

Unless noted, all manipulations were performed manually. The procedure of “Cyclization Method B” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids in the 50-mL centrifuge tube were dissolved in CH₃CN/0.1 M aqueous solution of ammonium bicarbonate (1:1,v/v, 30-45 mL). The solution was then allowed to shake for several hours at room temperature. The reaction solution was checked by pH paper and LCMS, and the pH can be adjusted to above 8 by adding 0.1 M aqueous ammonium bicarbonate (5-10 mL). After completion of the reaction based on the disappearance of the linear peptide on LCMS, the reaction was concentrated to dryness on speedvac or genevac EZ-2. The resulting residue was charged with CH₃CN:H₂O (2:3, v/v, 30 mL), and concentrated to dryness on speedvac or genevac EZ-2. This procedure was repeated (usually 2 times). The resulting crude solids were then dissolved in DMF or DMF/DMSO or CH₃CN/H₂O/formic acid. After filtration, the solution was subjected to single compound reverse-phase HPLC purification to afford the desired cyclic peptide.

N-Methylation on-Resin MethodA. To the resin (50 μmol) in a Bio-Rad tube was added CH₂Cl₂ (2 mL) and shaken for 5 min at rt. 2-Nitrobenzene-1-sulfonyl chloride (44.3 mg, 200 μmol, 4 equiv) was added followed by the addition of 2,4,6-trimethylpyridine (0.040 mL, 300 μmol, 6 equiv). The reaction was shaken at rt for 2 h. The solvent was drained and the resin was rinsed with CH₂Cl₂ (5 mL×3), DMF (5 mL×3) and then THF (5 mL×3). The resin was added THF (1 mL). Triphenylphosphine (65.6 mg, 250 μmol, 5 equiv), methanol (0.020 mL, 500 μmol, 10 equiv) and Diethyl azodicarboxylate or DIAD (0.040 mL, 250 μmol, 5 equiv) were added. The mixture was shaken at rt for 2-16 h. The reaction was repeated. Triphenylphosphine (65.6 mg, 250 μmol, 5 equiv), methanol (0.020 mL, 500 μmol, 10 equiv) and Diethyl azodicarboxylate or DIAD (0.040 mL, 250 μmol, 5 equiv) were added. The mixture was shaken at rt for 1-16 h. The solvent was drained, and the resin was washed with THE (5 mL×3) and CHCl₃ (5 mL×3). The resin was air dried and used directly in the next step. The resin was shaken in DMF (2 mL). 2-Mercaptoethanol (39.1 mg, 500 μmol) was added followed by DBU (0.038 mL, 250 μmol, 5 equiv). The reaction was shaken for 1.5 h. The solvent was drained. The resin was washed with DMF (4×). Air dried and used directly in the next step.

N-Methylation On-resin Method B (Turner, R. A. et al, Org. Lett., 15(19):5012-5015 (2013)). All manipulations were performed manually unless noted. The procedure of “N-methylation on-resin Method A” describes an experiment performed on a 0.100 mmol scale, where the scale is determined by the amount of Sieber or Rink linker bound to the resin that was used to generate the peptide. This scale is not based on a direct determination of the quantity of peptide used in the procedure. The procedure can be scaled beyond 0.10 mmol scale by adjusting the described volumes by the multiple of the scale. The resin was transferred into a 25 mL fritted syringe. To the resin was added piperidine:DMF (20:80 v/v, 5.0 mL). The mixture was shaken for 3 min. and then the solution was drained through the frit. The resin was washed 3 times with DMF (4.0 mL). To the reaction vessel was added piperidine:DMF (20:80 v/v, 4.0 mL). The mixture was shaken for 3 min. and then the solution was drained through the frit. The resin was washed successively three times with DMF (4.0 mL) and three times with DCM (4.0 mL). The resin was suspended in DMF (2.0 mL) and ethyl trifluoroacetate (0. 119 mL, 1.00 mmol), 1,8-diazabicyclo[5.4.0]undec-7-ene (0.181 mL, 1.20 mmol). The mixture was placed on a shaker for 60 min. The solution was drained through the frit. The resin was washed successively three times with DMF (4.0 mL) and three times with DCM (4.0 mL). The resin was washed three times with dry THE (2.0 mL) to remove any residual water. In an oven dried 4.0 mL vial was added THE (1.0 mL) and triphenylphosphine (131 mg, 0.500 mmol) on dry 4 A molecular sieves (20 mg). The solution was transferred to the resin and diisopropyl azodicarboxylate (0.097 mL, 0.5 mmol) was added slowly. The resin was stirred for 15 min. The solution was drained through the frit and the resin was washed with three times with dry THE (2.0 mL) to remove any residual water. In an oven dried 4.0 mL vial was added THE (1.0 mL), triphenylphosphine (131 mg, 0.50 mmol) on dry 4 A molecular sieves (20 mg). The solution was transferred to the resin and diisopropyl azodicarboxylate (0.097 mL, 0.5 mmol) was added slowly. The resin was stirred for 15 min. The solution was drained through the frit. The resin was washed successively three times with DMF (4.0 mL) and three times with DCM (4.0 mL). The resin was suspended in Ethanol (1.0 mL) and THF (1.0 mL), and sodium borohydride (37.8 mg, 1.000 mmol) was added. The mixture was stirred for 30 min. and drained. The resin was washed successively three times with DMF (4.0 mL) and three times with DCM (4.0 mL).

### N-Alkylation On-resin Procedure Method A:

A solution of the alcohol corresponding to the alkylating group (0.046 g, 1.000 mmol), triphenylphosphine (0.131 g, 0.500 mmol), and DIAD (0.097 mL, 0.500 mmol) in 3 mL of THF was added to nosylated resin (0.186 g, 0.100 mmol), and the reaction mixture was stirred for 16 hours at room temperature. The resin was washed three times with THF (5 mL), and the above procedure was repeated 1-3 times. Reaction progress was monitored by TFA micro-cleavage of small resin samples treated with a solution of 50 μL of TIS in 1 mL of TFA for 1.5 hours.

### N-Alkylation On-resin Procedure Method B:

The nosylated resin (0.100 mmol) was washed three times with N-methylpyrrolidone (NMP) (3 mL). A solution of NMP (3 mL), Alkyl Bromide (20 eq, 2.000 mmol) and DBU (20 eq, 0.301 mL, 2.000 mmol) was added to the resin, and the reaction mixture was stirred for 16 hours at room temperature. The resin was washed with NMP (3 mL) and the above procedure was repeated once more. Reaction progress was monitored by TFA micro-cleavage of small resin samples treated with a solution of 50 μL of TIS in 1 mL of TFA for 1.5 hours.

### N-Nosylate Formation Procedure:

A solution of collidine (10 eq.) in DCM (2 mL) was added to the resin, followed by a solution of Nos-Cl (8 eq.) in DCM (1 mL). The reaction mixture was stirred for 16 hours at room temperature. The resin was washed three times with DCM (4 mL) and three times with DMF (4 mL). The alternating DCM and DMF washes were repeated three times, followed by one final set of four DCM washes (4 mL).

### N-Nosylate Removal Procedure:

The resin (0.100 mmol) was swelled using three washes with DMF (3 mL) and three washes with NMIP (3 mL). A solution of NMIP (3 mL), DBU (0.075 mL, 0.500 mmol) and 2-mercaptoethanol (0.071 mL, 1.000 mmol) was added to the resin and the reaction mixture was stirred for 5 minutes at room temperature. After filtering and washing with NMIP (3 mL), the resin was re-treated with a solution of NMP (3 mL), DBU (0.075 mL, 0.500 mmol) and 2-mercaptoethanol (0.071 mL, 1.000 mmol) for 5 minutes at room temperature. The resin was washed three times with NMP (3 mL), four times with DMF (4 mL) and four times with DCM (4 mL), and was placed back into a Symphony reaction vessel for completion of sequence assembly on the Symphony peptide synthesizer.

General Procedure for Preloading amines on the PL-FMP resin:

PL-FMP resin (Novabiochem, 1.00 mmol/g substitution) was swollen with DMF (20 mL/mmol) at room temperature. The solvent was drained and 10 mL of DMF was added, followed by the addition of the amine (2.5 mmol) and acetic acid (0.3 mL) into the reaction vessel. After 10-min agitation, sodium triacetoxyhydroborate (2.5 mmol) was added. The reaction was allowed to agitate overnight. The resin was washed by DMF (1×), THF/H₂O/AcOH (6:3:1) (2×), DMF (2×), DCM (3×), and dried. The resulting PL-FMP resin preloaded with the amine can be checked by the following method: Took 100 mg of above resin and reacted with benzoyl chloride (5 equiv), and DIEA (10 equiv) in DCM (2 mL) at room temperature for 0.5 h. The resin was washed with DMF (2×), MeOH (1×), and DCM (3×). The sample was then cleaved with 40% TFA/DCM (1 h). The product was collected and analyzed by HPLC and MS. Collected sample was dried and got weight to calculate resin loading.

General Procedure for Preloading Fmoc-Amino Acids on C₁-trityl resin:

To a glass reaction vessel equipped with a frit was added the 2-Chloro-chlorotrityl resin mesh 50-150, (1.54 meq/gram, 1.94 grams, 3.0 mmole) to be swollen in DCM (5 mL) for 5 minutes. A solution of the acid (3.00 mmol, 1.0 eq) in DCM (5 mL) was added to the resin followed by DIPEA (2.61 mL, 15.00 mmol, 5.0 eq). The reaction was shaken at room temperature for 60 minutes. Add in DIEA (0.5 mL) and methanol (3 mL), shaken for an additional 15 minutes. The reaction solution was filtered through the frit and the resin was rinsed with DCM (4×5 mL), DMF (4×5 mL), DCM (4×5 mL), diethyl ether (4×5 mL), and dried using a flow of nitrogen. The resin loading can be determined as follows:

A sample of resin (13.1 mg) was treated with 20% piperidine/DMF (v/v, 2.0 mL) for 10 minutes with shaking. 1 mL of this solution was transferred to a 25.0 mL volumetric flask and diluted with methanol to a total volume of 25.0 mL. A blank solution of 20% piperidine/DMF (v/v, 1.0 mL) was diluted up with methanol in a volumetric flask to 25.0 mL. The UV was set to 301 nm and zero with the blank solution followed by the reading of the solution, Absorbance=1.9411. (1.9411/20 mg)*6.94=0.6736. Loading of the resin was measured to be 0.6736 mmol/g.

### Click Reaction On-Resin Method A:

This procedure describes an experiment performed on a 0.050 mmol scale. It can be scaled beyond or under 0.050 mmol scale by adjusting the described volumes by the multiple of the scale. The alkyne containing resin (50 μmol each) was transferred into Bio-Rad tubes and swell with DCM (2×5 mL×5 mins) and then DMF (2×5 mL×5 mins). In a 200-mL bottle was charged with 30 time of the following: ascorbic acid (vitamin C, 0.026 g, 0.150 mmol), bis(2,2,6,6-tetramethyl-3,5-heptanedionato)copper(II) (10.75 mg, 0.025 mmol), DMF (1.5 mL), 2,6-lutidine (0.058 mL, 0.50 mmol) and THE (1.5 mL), followed by DIEA (0.087 mL, 0.50 mmol) and the corresponding azide used in the examples (1.0-2.0 equiv.). The mixture was stirred until everything was in solution. The DMF in the above Bio-Rad tube was drained, and the above click solution (3 mL each) was added to each Bio-Rad tube. The tubes were shaken overnight on an orbital shaker. Solutions were drained through the frit. The resins were washed with DMF (3×2 mL) and DCM (3×2 mL).

### Click Reaction On-Resin Method B:

This procedure describes an experiment performed on a 0.050 mmol scale. It can be scaled beyond or under 0.050 mmol scale by adjusting the described volumes by the multiple of the scale. The alkyne containing resin (50 μmol each) was transferred into Bio-Rad tubes and swell with DCM (2×5 mL×5 mins) and then DMF (2 5 mL×5 mins). In a separate bottle, nitrogen was bubbled into 4.0 mL of DMSO for 15 mins. To the DMSO was added copper iodide (9.52 mg, 0.050 mmol, 1.0 eq) (sonicated), lutidine (58 μL, 0.500 mmol, 10.0 eq) and DIEA (87 μL, 0.050 mmol, 10.0 eq). The solution was purged with nitrogen again. DCM was drained through the frit. In a separate vial, ascorbic acid (8.8 mg, 0.050 mmol, 1.0 eq) was dissolved into water (600 μL). Nitrogen was bubbled through the solution for 10 mins. Coupling partners were distributed in the tubes (0.050 mmol to 0.10 mmol, 1.0 to 2.0 eq) followed by the DMSO copper and base solution and finally ascorbic acid aqueous solution. The solutions were topped with a blanket of nitrogen and capped. The tube was put onto the rotatory mixer for 16 hours. Solutions were drained through the frit. The resins were washed with DMF (3×2 mL) and DCM (3×2 mL).

### Suzuki Reaction On-Resin Procedure:

In a Bio Rad tube is placed 50 μmoles of dried Rink resin of a N-terminus Fmoc-protected linear polypeptide containing a 4-bromo-phenylalanine side chain. The resin was swelled with DMF (2×5 mL). To this was added a DMF solution (2 mL) of p-tolylboronic acid (0.017 g, 0.125 mmol), potassium phosphate (0.2 mL, 0.400 mmol) followed by the catalyst [1,1′-bis(di-tert-butylphosphino)ferrocene]dichloropalladium(II) [PdCl₂(dtbpf)](3.26 mg, 5.00 μmol). The tube was shaken at rt overnight. The solution was drained and the resin was washed with DMF (5×3 mL) followed by alternating DCM (2×3 mL), then DMF (2×3 mL), and then DCM (5×3 mL). A small sample of resin was micro-cleaved using 235 μL of TIS in 1 mL TFA at rt for 1 h. The rest of the resin was used in the next step of peptide coupling or chloroacetic acid capping of the N-terminus. Solution Phase Click Reaction Method A:

To a 20-mL scintillation vial was added 100-times of sodium ascorbate (sodium (R)-2-((S)-1,2-dihydroxyethyl)-4-hydroxy-5-oxo-2,5-dihydrofuran-3-olate) and copper(II) sulfate pentahydrate (CuSO₄: sodium ascorbate mol ratio: 1:3 to 1:5). The reaction was diluted with water. This solution was shaken at RT for 1-10 min. The resulting yellowish slurry was added to the reaction.

To the vial containing the alkyne and the azide (1.0-2.0 equiv) was added aliquor amount of the above copper solution (CuSO₄: 0.3-1.0 equiv of the alkyne). The mixture was shaken at rt for 1-3 h and the progress was monitored by LC/MS. Additional amounts of azide or copper solution can be added to drive the triazole formation if required. After completion, the mixture was diluted with CH₃CN: aq. NH₄CO₃ solution (v/v 1:1), filtered, and purified at the same day via reverse phase HPLC purification.

### Solution Phase Click Reaction Method B:

A stock solution of CuSO₄ and sodium ascorbate was prepared by diluting a dry 1:2 to 1:3 mol ratio of copper(II) sulfate pentahydrate and soidum ascorbate to a concentration of 0.1-0,3 M with respect to copper sulfate pentahydrate. To a solution of the peptide alkyne in DMF (0.05-0.1 M) was added the corresponding azide used in the examples (1.0-2.0 equiv) followed by the above freshly prepared aqueous copper solution (0.03-1.0 equiv). The mixture was stirred at room temperature and monitored by LCMS. Additional amounts of azide or copper solution can be added to drive the triazole formation if required. Upon full conversion the mixture was diluted, filtered and purified at the same day by reverse phase HPLC.

### Symphony Dde/ivDde Deprotection Procedure:

The reaction vessel containing the resin from the previous step was washed successively two times as follows: for each wash, DMF (2.5 mL) was added through the top of the vessel and the resulting mixture was periodically agitated for 30 seconds before the solution was drained through the frit. The resin was washed successively five times as follows: for each wash, a solution of hydrazine in DMF (2% v/v, 2.5 mL) was added through the top of the vessel and the resulting mixture was periodically agitated for 5 minutes before the solution was drained through the frit. The resin was washed successively six times as follows: for each wash, DMF (2.5 mL) was added through the top of the vessel and the resulting mixture was periodically agitated for 30 seconds before the solution was drained through the frit. The resulting resin was used directly in the next step.

### General Purification Procedures:

The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at certain percentage of B, then a linear increase from this percentage to a higher percentage of B over 20-30 minutes, then a 0-minute hold at 100% B; Flow Rate: 20-40 mL/min; Column Temperature: 25° C.25° C. Fraction collection was triggered by MS and UV signals. Fractions containing the desired product were combined and dried via centrifugal evaporation. If the material was not pure based on the orthogonal analytical data, it was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at certain percentage of B, then a linear increase from the starting percentage of B over 20-30 minutes, then a 0-minute hold at 100% B; Flow Rate: 20-40 mL/min; Column Temperature: 25° C.25° C. Fraction collection was triggered by MS signals. Fractions containing the desired product were combined and dried via centrifugal evaporation. The yield and the purity of the product were determined.

Alternatively, based on the initial analytical data, the crude material was purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at certain percentage of B, then a linear increase from the starting percentage of B over 20 minutes, then a 0-minute hold at 100% B; Flow Rate: 40 mL/min; Column Temperature: 25° C.25° C. Fraction collection was triggered by MS signals. Fractions containing the desired product were combined and dried via centrifugal evaporation. If the material is not pure based on the orthogonal analytical data, it was further purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at certain percentage of B, then a linear increase from this percentage to a higher percentage of B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation. The yield and the purity of the product were determined.

### Fatty Acid Chain Coupling Procedure A:

To the elaborated peptide was added DMF (2.0 mL), the fatty activated ester (0.077 to 0.205 mmol, 1.5 to 4.0 eq) and DIEA (0.036 to 0.072 mL, 0.205 mmol, 4.0-8.0 eq). The reaction was allowed to shake for 1 h. The reaction mixture was neutralized with a few drops of acetic acid and submitted for purification.

### Fatty Acid Chain Coupling Procedure B:

To the elaborated peptide was added DMF (2.0 mL), the fatty activated ester (0.077 to 0.205 mmol, 1.5 to 4.0 eq) and DIEA (0.036 to 0.072 mL, 0.205 mmol, 4.0-8.0 eq). The reaction was allowed to shake for 1 h. The reaction mixture was concentrated to dryness using a Biotage V10 apparatus. To the crude pdt was added 2.0 mL of a solution of TFA/water (90:10, v:v) and the solution was allowed to shake for 20 minutes. The reaction was then concentrated to dryness and redissolved in 2.0 mL of DMF to be submitted for purification

### Unnatural Amino Acid Synthesis:

### Preparation of (S)-2-((((9H-fluoren-9-yl)methoxy)carbonyl)-3-(1-(2-(tert-butoxy)-2-oxoethyl)-1H-indol-3-yl)propanoic acid

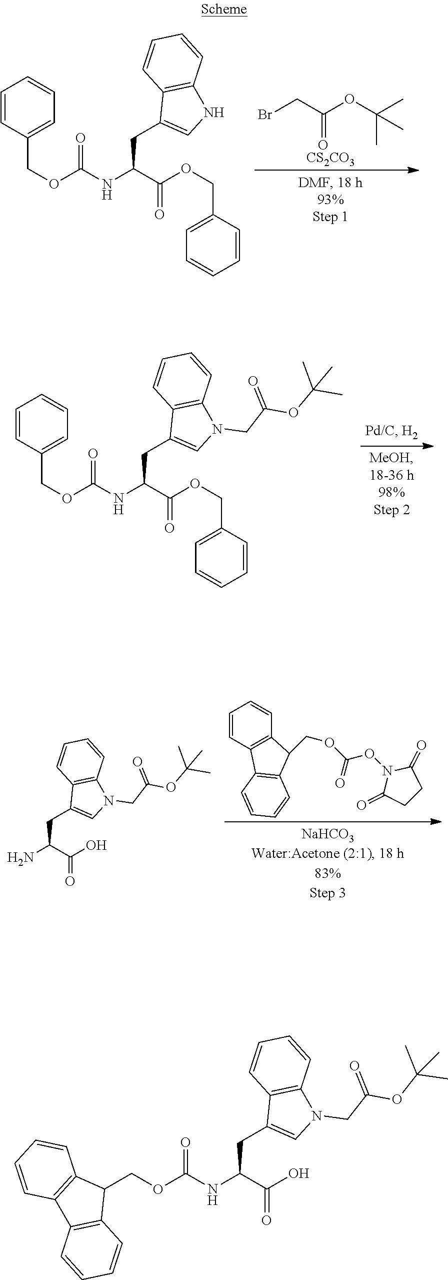

### Step 1:

To a 0° C. solution of (S)-benzyl 2-(((benzyloxy)carbonyl)amino)-3-(1H-indol-3-yl) propanoate (25.0 g, 58.3 mmol) and cesium carbonate (20.9 g, 64.2 mmol) in DMF (200 mL) was added tert-butyl 2-bromoacetate (9.36 mL, 64.2 mmol). The solution was allowed to slowly warm up to RT with stirring for 18 h. The reaction mixture was poured into ice water:aq. 1N HCl (1:1) and then extracted with EtOAc. The organic layer was washed with brine, collected, dried over MgSO₄, filtered, and then concentrated in vacuo. The resulting solid was subjected to flash chromatography (330 g column, 0-50% EtOAc:Hex over 20 column volumes) to afford (S)-benzyl 2-(((benzyloxy)carbonyl)amino)-3-(1-(2-(tert-butoxy)-2-oxoethyl)-1H-indol-3-yl)propanoate as a white solid (29.6 g, 93%).

### Step 2:

H₂ was slowly bubbled through a mixture of (S)-benzyl 2-(((benzyloxy)carbonyl)amino)-3-(1-(2-(tert-butoxy)-2-oxoethyl)-1H-indol-3-yl)propanoate (29.6 g, 54.5 mmol) and Pd-C(1.45 g, 1.36 mmol) in MeOH (200 mL) at RT for 10 min. The mixture was then stirred under positive pressure of H₂ while conversion was monitored by LCMS. After 48 h the reaction mixture was filtered through diatomaceous earth and evaporated to afford crude (S)-2-amino-3-(1-(2-(tert-butoxy)-2-oxoethyl)-1H-indol-3-yl)propanoic acid (17.0 g) which was carried into step three without additional purification.

### Step 3:

To a solution of (S)-2-amino-3-(1-(2-(tert-butoxy)-2-oxoethyl)-1H-indol-3-yl)propanoic acid (5.17 g, 16.2 mmol) and sodium bicarbonate (6.8 g, 81 mmol) in acetone:water (50.0 mL:100 mL) was added (9H-fluoren-9-yl)methyl (2,5-dioxopyrrolidin-1-yl) carbonate (5.48 g, 16.2 mmol). The mixture stirred overnight upon which LCMS analysis indicated complete conversion. The vigorously stirred mixture was acidified via slow addition of aq 1N HCl. Once acidified, the mixture was diluted with DCM (150 mL), and the isolated organic phase was then washed with water, followed by brine. The organic layer was collected, dried over sodium sulfate, and concentrated under vacuum to afford the crude product. The crude material was purified via silica gel chromatography (330 g column, 20-80% EtOAc:Hex over 20 column 25 volumes) to afford (S)-2-((((9H-fluoren-9-yl)methoxy)carbonyl)amino)-3-(1-(2-(tertbutoxy)-2-oxoethyl)-1H-indol-3-yl)propanoic acid as a white foam (7.26 g, 83%). ¹H NMR (500 MHz, methanol-d₄) δ 7.80 (d, J=7.6 Hz, 2H), 7.67-7.60 (m, 2H), 7.39 (t, J=7.5 Hz, 2H), 7.32-7.22 (m, 3H), 7.18 (td, J=7.6, 0.9 Hz, 1H), 7.08 (td, J=7.5, 0.9 Hz, 1H), 7.04 (s, 1H), 4.54 (dd, J=8.4, 4.9 Hz, 1H), 4.36-4.23 (m, 2H), 4.23-4.14 (m, 1H), 30 3.43-3.35 (m, 2H), 3.25-3.09 (m, 1H), 1.55-1.38 (m, 9H). ESI-MS(+) m/z=541.3 (M+H).

### Preparation of (S)-2-((((9H-fluoren-9-yl)methoxy)carbonyl)amino)-3-(4-(2-(tert-butoxy)-2-oxoethoxy)phenyl)propanoic acid

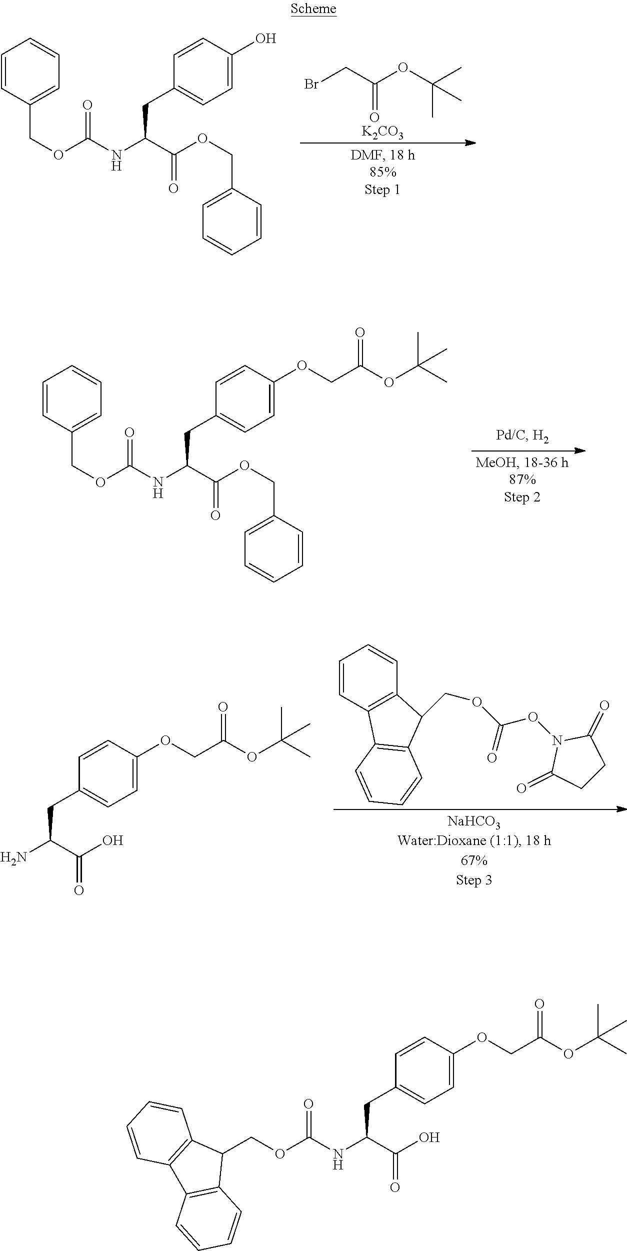

### Step 1:

To a cooled stirred solution of (S)-benzyl 2-(((benzyloxy)carbonyl)amino)-3-(4-hydroxyphenyl)propanoate (70 g, 173 mmol) and K₂CO₃ (35.8 g, 259 mmol) in DMF (350 mL) was added tert-butyl-2-bromoacetate (30.6 mL, 207 mmol) dropwise and the resulting mixture was stirred at RT overnight. The reaction mixture was diluted with 10% brine solution (1000 mL) and extracted with ethyl acetate (2×250 mL). The combined organic layer was washed with water (500 mL), saturated brine solution (500 mL), dried over anhydrous sodium sulfate, filtered, and concentrated to afford colorless gum. The crude compound was purified by flash column chromatography using 20% ethyl acetate in petroleum ether as an eluent to afford a white solid (78 g, 85%).

### Step 2:

The (S)-benzyl 2-(((benzyloxy)carbonyl)amino)-3-(4-(2-(tert-butoxy)-2-oxoethoxy)phenyl)propanoate (73 g, 140 mmol) was dissolved in MeOH (3000 mL) and purged with nitrogen for 5 min. To the above purged mixture was added Pd/C (18 g, 16.91 mmol) and stirred under hydrogen pressure of 3 kg for 15 hours. The reaction mixture was filtered through a bed of diatomaceous earth (Celite©) and washed with methanol (1000 mL). The filtrate was concentrated under vacuum to afford a white solid (36 g, 87%).

### Step 3:

To a stirred solution of (S)-2-amino-3-(4-(2-(tert-butoxy)-2-oxoethoxy)phenyl)propanoic acid (38 g, 129 mmol) and sodium bicarbonate (43.2 g, 515 mmol) in water (440 mL) was added Fmoc-OSu (43.4 g, 129 mmol) dissolved in dioxane (440 mL) d dropwise and the resulting mixture was stirred at RT overnight. The reaction mixture was diluted with 1.5 N HCl (200 mL) and water (500 mL) and extracted with ethyl acetate (2×250 mL). The combined organic layer was washed with water (250 mL), saturated brine solution (250 mL), and dried over Na₂SO₄, filtered, and concentrated to afford a pale yellow gum. The crude compound was purified by column chromatography using 6% MeOH in chloroform as an eluent to afford pale green gum. The gum was further triturated with petroleum ether to afford an off-white solid (45 g, 67%). ¹H NMR (400 MHz, DMSO-d₆) δ 12.86-12.58 (m, 1H), 7.88 (d, J=7.5 Hz, 2H), 7.73-7.61 (m, 3H), 7.58-7.47 (m, 1H), 7.44-7.27 (m, 4H), 7.18 (d, J=8.5 Hz, 2H), 6.79 (d, J=8.5 Hz, 2H), 4.57 (s, 2H), 4.25-4.10 (m, 4H), 3.34 (br s, 3H), 3.02 (dd, J=13.8, 4.3 Hz, 1H), 2.81 (dd, J=14.1, 10.5 Hz, 1H), 1.41 (s, 9H).

### Preparation of (S)-2-((((9H-fluoren-9-yl)methoxy)carbonyl)amino)-3-(4-(tert-butoxycarbonyl)phenyl)propanoic acid

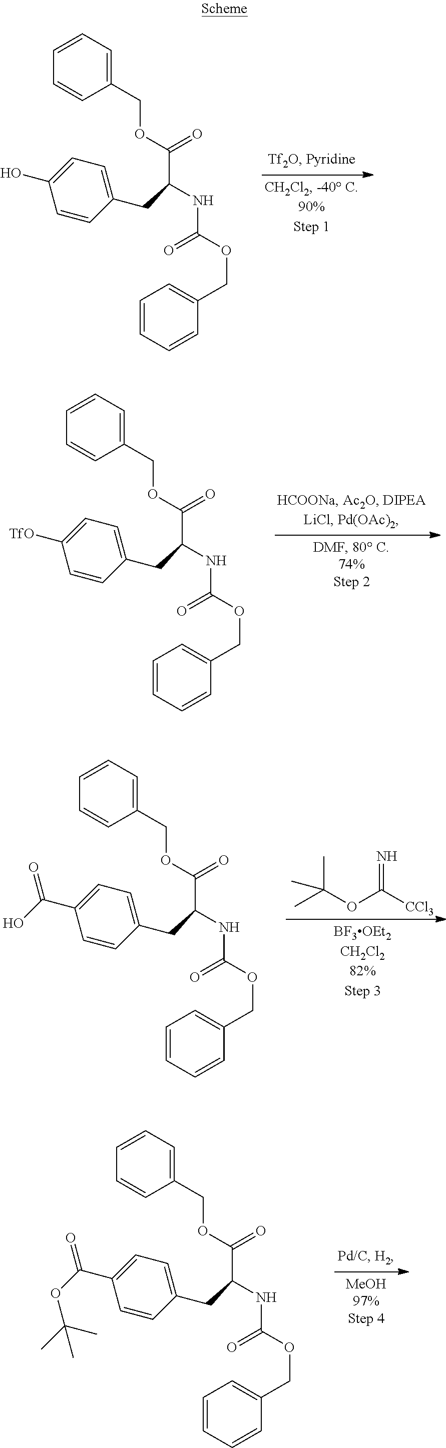

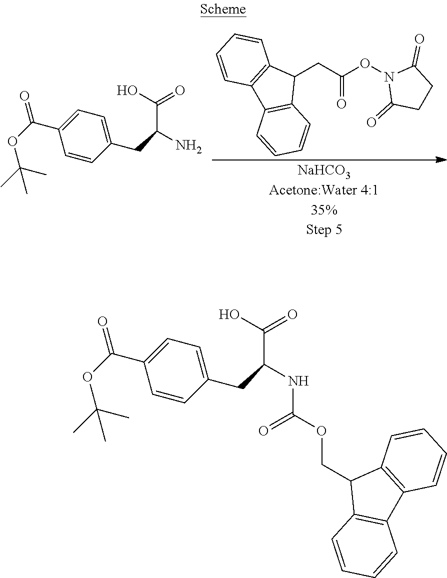 [SMILES: CC(C)(C)OC(=O)c1ccc(C[C@H](N)C(=O)O)cc1 ; O=C(CC1c2ccccc2-c2ccccc21)ON1C(=O)CCC1=O ; CC(C)(C)OC(=O)c1ccc(C[C@H](NC(=O)OCC2c3ccccc3-c3ccccc32)C(=O)O)cc1]

### Step 1.

(S)-Benzyl 2-(((benzyloxy)carbonyl)amino)-3-(4-hydroxyphenyl)propanoate (10 g, 24.66 mmol) was taken in DCM (100 mL) in a 250 mL multi-neck round bottom flask under magnetic stirring with N2 outlet. The reaction mixture was cooled to −40° C., pyridine (5.49 mL, 67.8 mmol) was added slowly and then stirred at the same temperature for 20 minutes, followed by addition of triflic anhydride (11.46 mL, 67.8 mmol) slowly at −40° C. and allowed to stir at -40° C. for 2 hours. The reaction mixture was quenched with water at −10° C., and then added citric acid solution (50 mL). The organic layer was extracted in DCM, and the separated organic layer was dried over anhydrous Na₂SO₄, filtered, and then evaporated to give (S)-benzyl 2-(((benzyloxy)carbonyl)amino)-3-(4-(((trifluoromethyl)sulfonyl)oxy)phenyl)propanoate (11.93 g, 22.20 mmol, 90% yield) as a pale yellow solid.

### Step 2.

A solution of DMF (1500 mL) was purged with nitrogen for 10 min. To this was added sodium formate (114 g, 1676 mmol) and acetic anhydride (106 mL, 1123 mmol). Purging continued and the mixture was cooled to 0° C. DIPEA (194 mL, 1111 mmol) was added and the reaction mixture was allowed to stir for 1 h at RT under nitrogen atmosphere.

To a 10-liter autoclave was added DMF (3200 mL) and the system was purged with nitrogen. Under the nitrogen purging conditions, (S)-benzyl 2-(((benzyloxy)carbonyl)amino)-3-(4-(((trifluoromethyl)sulfonyl)oxy)phenyl)propanoate (300 g, 558 mmol), lithium chloride (71 g, 1675 mmol), 1,3-bis(diphenylphosphino)propane (24.17 g, 58.6 mmol) were added followed by the addition of palladium(II) acetate (12.9 g, 57.5 mmol). To this reaction mixture was added the above prepared solution and heated to 80° C. for 16 h.

The reaction mass was diluted with ethyl acetate and water. The phases were separated and the ethyl acetate layer was washed with water and brine solution, dried over anhydrous sodium sulphate, filtered, and concentrated. The crude material was added to a torrent column and was eluted with petroleum ether and ethyl acetate. The fractions at 30%-65% ethyl acetate in petroleum ether were concentrated to afford a cream solid (300 g), which was dissolved in ethyl acetate (700 mL) and petroleum ether was added slowly. At about 20% ethyl acetate in petroleum ether a white solid precipitated out, which was filtered and washed with 20% ethyl acetate in petroleum ether to obtain a white solid (180 g, yield 74%).

### Step 3.

To a 2000-mL multi-neck round-bottomed flask was charged (S)-4-(3-(benzyloxy)-2-(((benzyloxy)carbonyl)amino)-3-oxopropyl)benzoic acid (130 g, 300 mmol), dichloromethane (260 mL) and cyclohexane (130 mL). To the slurry reaction mixture was added BF₃.OEt₂ (3.80 mL, 30.0 mmol) at room temperature, followed by the addition of tert-butyl 2,2,2-trichloroacetimidate (262 g, 1200 mmol) slowly at room temperature over 30 min. Upon addition, the slurry slowly started dissolving and at the end of the addition it was completely dissolved. The reaction mixture was allowed to stir at room temperature for 16 h. The reaction mixture was diluted with DCM and the remaining solids were removed by filtration. The filtrate was concentrated and purified by flash chromatography. The crude material was purified by Torrent using 1.5 Kg silicycle column. The product spot was eluted at 15% ethyl acetate/petroleum ether mixture. The collected fractions were concentrated to obtain a colorless liquid (120 g, yield 82%).

### Step 4.

(S)-tert-Butyl 4-(3-(benzyloxy)-2-(((benzyloxy)carbonyl)amino)-3-oxopropyl)benzoate (200 g, 409 mmol) was dissolved in MeOH (4000 mL) and N₂ was purged for 10 min. Pd/C (27.4 g, 25.7 mmol) was added. The reaction was shaken under H₂ for 16 h at room temperature. The reaction mass was filtered through celite bed and the bed was washed with methanol. The obtained filtrate was concentrated to obtain a pale yellow solid. The obtained solid was stirred with 5% methanol: diethyl ether mixture for 15 min before being filtered, dried under vacuum to obtain a pale yellow solid. It was made slurry with 5% methanol in diethyl ether and stirred for 15 min, filtered, and dried to give (S)-2-amino-3-(4-(tert-butoxycarbonyl)phenyl)propanoic acid as a white solid (105g, yield 97%). Analysis condition E: Retention time=0.971 min; ESI-MS(+) m/z [M+H]⁺: 266.2.

### Step 5.

(S)-2-Amino-3-(4-(tert-butoxycarbonyl)phenyl)propanoic acid (122 g, 460 mmol) was dissolved in acetone (1000 mL) and then water (260 mL) and sodium bicarbonate (116 g, 1380 mmol) were added. It was cooled to 0° C. and Fmoc-OSu (155 g, 460 mmol) was added portionwise into the reaction mixture. After completion of addition it was stirred at room temperature for 16 h. The reaction mixture was diluted with dichloromethane (2 L) and then water was added (1.5 L). The organic layer was washed with saturated citric acid solution and extracted, and the aqueous layer was again extracted with DCM. The combined organic layer was washed with 10% citric acid solution, brine solution, and dried over Na₂SO₄, and evaporated to dryness. The obtained white solid was made slurry with diethyl ether, filtered, and dried to get the desired product as a white solid (80 g, yield 35%). ¹H NMR (400 MHz, DMSO-d₆) δ 7.87 (d, J=7.5 Hz, 2H), 7.83-7.73 (m, 3H), 7.60 (t, J=8.5 Hz, 2H), 7.51-7.24 (m, 7H), 4.26-4.11 (m, 4H), 3.45-3.27 (m, 4H), 3.17 (br dd, J=13.8, 4.3 Hz, 1H), 2.94 (dd, J=13.5, 11.0 Hz, 1H), 2.52-2.48 (m, 4H), 1.51 (s, 9H).

### Preparation of tert-butyl (R)-2-((((9H-fluoren-9-yl)methoxy)carbonyl)amino)-3-iodopropanoate

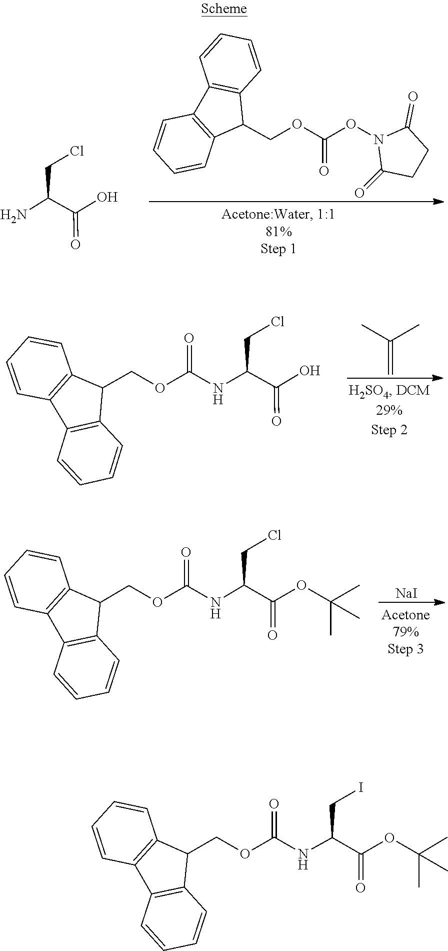

### Step 1.

To a solution of (R)-2-amino-3-chloropropanoic acid hydrochloride (125 g, 781 mmol) in a 1:1 mixture of acetone (1 L) and water (1 L) was added Na₂CO₃ (182 g, 1719 mmol) followed by Fmoc-OSu (250 g, 742 mmol). The reaction was stirred at RT overnight. It was extracted with ethyl acetate (2×500 mL) and the aq. layer was acidified with 5N HCl. The HCl solution was extracted with ethyl acetate (1500 mL, then 2×500 mL). The combined organic layers were dried over anhydrous MgSO₄, filtered, and concentrated to give the crude product (R)-2-((((9H-fluoren-9-yl)methoxy)carbonyl)amino)-3-chloropropanoic acid. The product (220 g) was taken to the next step as such.

### Step 2.

A solution of (R)-2-((((9H-fluoren-9-yl)methoxy)carbonyl)amino)-3-chloropropanoic acid (220 g, 636 mmol) in DCM (2 L) was cooled to −20° C. 2-Methylpropene (200 mL, 636 mmol) was bubbled into the solution for 15 mins, then H₂SO₄ (57.7 mL, 1082 mmol) was added and the mixture was stirred at RT overnight. To the reaction mixture was added water (500 mL). The layers were separated and the aqueous layer was extracted DCM (2×500 mL). The combined organic layers were dried over anhydrous MgSO₄, filtered, and evaporated. The crude was purified by flash chromatography using petroleum ether and ethyl acetate elution solvents. The desired fractions were combined and concentrated to give the product (R)-tert-butyl 2-((((9H-fluoren-9-yl)methoxy)carbonyl)amino)-3-chloropropanoate (83 g, 182 mmol, 29% yield).

### Step 3.

To a solution of (R)-tert-butyl 2-((((9H-fluoren-9-yl)methoxy)carbonyl)amino)-3-chloropropanoate (80 g, 199 mmol) in acetone (1000 mL) was added sodium iodide (119 g, 796 mmol) and the reaction was heated to reflux for 40 hours. Acetone was removed by rotavap and the crude product was diluted with water (1000 mL) and DCM (1000 mL). The layers were separated and the organic layer was washed with aqueous saturated sodium sulphite solution (1000 mL) and brine (1000 mL). The organic layer was dried over anhydrous Na₂SO₄, filtered, and concentrated. The crude was purified by flash chromatography using 7 to 9% of ethyl acetate in petroleum ether. The desired product fractions were combined and concentrated to afford the product (R)-tert-butyl 2-((((9H-fluoren-9-yl)methoxy)carbonyl)amino)-3-iodopropanoate (83 g, 156 mmol, 79%). ¹H NMR (400 MHz, CDCl₃) δ 7.77 (d, J=7.5 Hz, 2H), 7.62 (d, J=7.5 Hz, 2H), 7.45-7.30 (m, 4H), 5.67 (br d, J=7.0 Hz, 1H), 4.54-4.32 (m, 3H), 4.30-4.21 (m, 1H), 3.71-3.50 (m, 2H), 1.56-1.48 (m, 9H).

### Preparation of (S)-2-((((9H-fluoren-9-yl)methoxy)carbonyl)amino)-3-(2-methyl-]H-indol-3-yl)propanoic acid

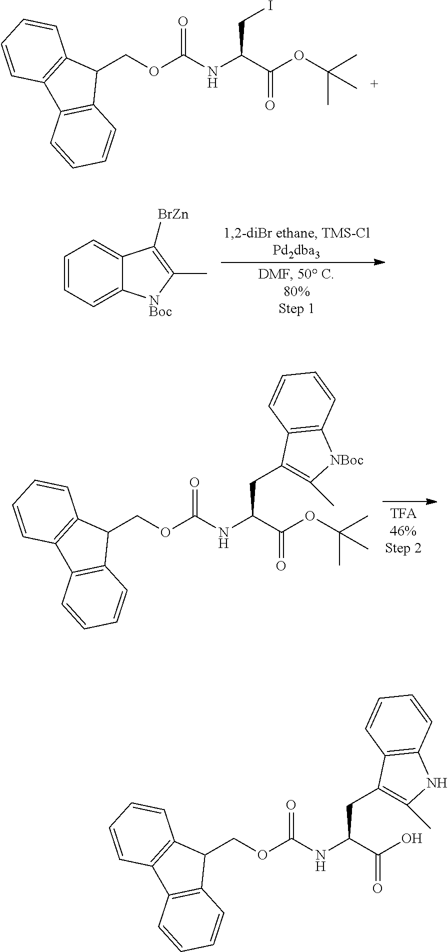

### Step 1.

In a 100-mL three-neck, flame-dried, nitrogen-purged round-bottomed flask, zinc (2.319 g, 35.5 mmol) was added under argon atmosphere and the flask was heated to 150° C. using a hot gun and was purged with argon. To the reaction flask, DMF (50 mL) was added followed by the addition of 1,2-dibromoethane (0.017 mL, 0.20 mmol) and TMS-Cl (0.026 mL, 0.20 mmol) under argon atmosphere and then stirred for 10 min. To the reaction mixture (R)-tert-butyl 2-((((9H-fluoren-9-yl)methoxy)carbonyl)amino)-3-iodopropanoate (5 g, 10.14 mmol) was added and the reaction was stirred for 1 h. The reaction progress was monitored via TLC and LCMS, till the starting iodide was completely converted into the Zn-complex. The solution of organozinc reagent was allowed to cool to room temperature and then tris(dibenzylideneacetone)dipalladium(0) (Pd₂(dba)₃) (0.23 g, 0.25 mmol), dicyclohexyl(2′,6′-dimethoxy-[1,1′-biphenyl]-2-yl)phosphine (SPhos) (0.21 g, 0.51 mmol), and tert-butyl 3-bromo-2-methyl-1H-indole-1-carboxylate (3.77 g, 12.16 mmol) were added. The reaction mixture was allowed to stir at RT under a positive pressure of nitrogen for 1 h and then heated to 50° C. for 6 hrs. The reaction progress was monitored via LCMS. The mixture was diluted with EtOAc (700 mL) and filtered through Celite. The organic phase was washed with sat. NH₄Cl (250 mL), water (2×200 mL), and sat. NaCl (aq) (250 mL), dried over anhydrous Na₂SO₄(s), concentrated, and dried under vacuum to afford the crude compound (19 g). It was purified through ISCO flash chromatography using 330 gredisep column and the product was eluted with 7 to 9% of ethyl acetate in petroleum ether. The above reaction and purification were repeated. The pure fractions were concentrated to give tert-butyl (S)-3-(2-((((9H-fluoren-9-yl)methoxy)carbonyl)amino)-3-(tert-butoxy)-3-oxopropyl)-2-methyl-1H-indole-1-carboxylate as a brownish solid (10.2 g. 95% pure, ca. 80% yield). Analysis condition G: Retention time=4.23 min; ESI-MS(+) m/z [M+2H][M-Boc-tBu+H]⁺: 441.2.

### Step 2.

In a 25-mL multi neck, round-bottomed flask, DCM (65 mL) was added followed by (S)-tert-butyl 3-(2-((((9H-fluoren-9-yl)methoxy)carbonyl)amino)-3-(tert-butoxy)-3-oxopropyl)-2-methyl-1H-indole-1-carboxylate (6.5 g, 10.89 mmol) under nitrogen atmosphere at RT. The reaction mixture was cooled to 0° C., triethylsilane (4.18 mL, 26.1 mmol) was added followed by the addition of TFA (5.87 mL, 76 mmol) dropwise at 0° C. The temperature of the reaction mixture was slowly brought to RT and stirred at RT for 4 h. The reaction progress was monitored by TLC. To the reaction mixture, TFA (5.87 mL, 76 mmol) was added. The reaction mixture was stirred at RT overnight, and concentrated under reduced pressure. The crude material was triturated with hexanes and stored in cold room to give a brown colored solid (crude weight: 6.5 g). It was purified via reverse phase flash chromatography, and the pure fractions were concentrated to obtain the desired final product as an off-white powder (2.3 g, 46%). ¹H NMR (DMSO-d₆): δ ppm: 10.65 (s, 1H), 7.84 (d, J=9.12 Hz, 2H), 7.65 (d, J=9.12 Hz, 2H), 7.42-7.49 (m, 1H), 7.30-7.38 (m, 2H), 7.26-7.29 (m, 2H), 7.17-7.19 (m, 2H), 6.91-6.95 (m, 1H), 6.85-6.88 (t, J=7.85 Hz, 1H), 4-16-4.18 (m, 2H), 4.01-4.06 (m, 1H), 3.09-3.14 (m, 1H), 2.96-2.99 (m, 1H), 2.50 (s, 3H). Analysis condition F: Retention time=1.37 min; ESI-MS(+) m/z [M+2H][M+H]⁺: 441.2.

### Preparation of (S)-2-((((9H-fluoren-9-yl)methoxy)carbonyl)amino)-3-(7-methyl-]H-indol-3-yl)propanoic acid

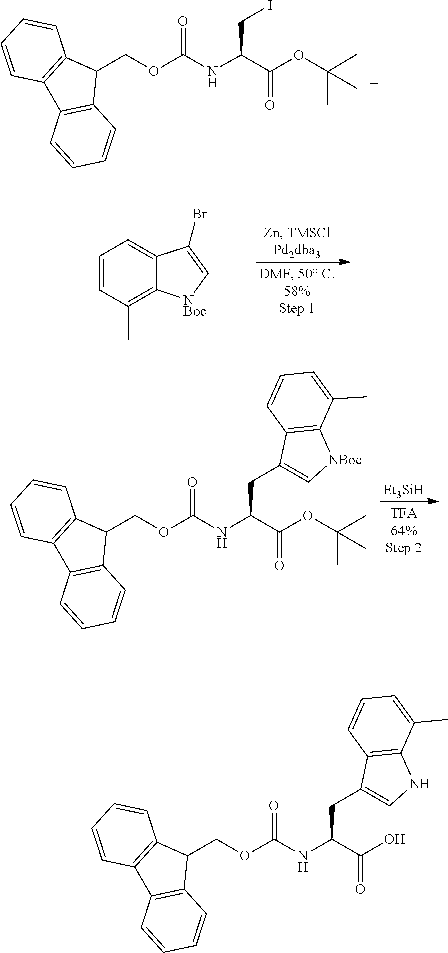

In a 50-mL round-bottomed flask, dry zinc (0.928 g, 14.19 mmol) was charged and flushed with argon three times and then the flask was heated to 150° C. for 5 min and then allowed to cool to room temperature and flushed with argon 3 times. DMF (20 mL) was added followed by the addition of 1,2-dibromoethane (6.99 μl, 0.081 mmol) and TMS-Cl (0.013 mL, 0.10 mmol). Successful zinc insertion was accompanied by a noticeable exotherm. After 5 min (R)-tert-butyl 2-((((9H-fluoren-9-yl)methoxy)carbonyl)amino)-3-iodopropanoate (2.0 g, 4.05 mmol) was added and the reaction was stirred for 30 min. In a 50-mL round-bottomed flask equipped charged with Argon was added the above alkyl zinc reagent, tert-butyl 3-bromo-7-methyl-1H-indole-1-carboxylate (1.26 g, 4.05 mmol) followed by 2-dicyclohexylphosphino-2′,6′-dimethoxybiphenyl (SPhos) (0.083 g, 0.20 mmol) and Pd₂(dba)₃ (0.093 g, 0.101 mmol). After the addition the reaction mixture was heated to 50° C. overnight. Another equivalents of Sphos and Pd₂(dba)₃ was added and heating continued for another 16 h. The reaction mixture was diluted with EtOAc (100 mL) and filtered through Celite. The organic phase was washed with sat. aq. NH₄Cl (100 mL), water (50 mL), and sat NaCl (100 mL), dried over anhydrous Na₂SO₄(s), concentrated, and dried under vacuum. After purification by flash chromatography the desired tert-butyl (S)-3-(2-((((9H-fluoren-9-yl)methoxy)carbonyl)amino)-3-(tert-butoxy)-3-oxopropyl)-2-methyl-1H-indole-]-carboxylate was obtained in 58% yield.

### Step2.

Final product was obtained following the same procedure of (S)-2-((((9H-fluoren-9-yl)methoxy)carbonyl)amino)-3-(2-methyl-]H-indol-3-yl)propanoic acid. TFA hydrolysis with triethylsilane afforded the desired (S)-2-((((9H-fluoren-9-yl)methoxy)carbonyl)amino)-3-(7-methyl-]H-indol-3-yl)propanoic acid as an off white solid in 64% yield after purification by reverse phase flash chromatography. Analysis condition E: Retention time=2.16 min; ESI-MS(+) m/z [M+H]⁺: 441.1. ¹H NMR (300 MHz, DMSO-d₆) Shift 12.70 (br s, 1H), 10.81 (br s, 1H), 7.88 (d, J=7.6 Hz, 2H), 7.76-7.56 (m, 2H), 7.49-7.21 (m, 5H), 7.17 (d, J=2.3 Hz, 1H), 6.94-6.84 (m, 2H), 4.29-4.13 (m, 3H), 4.07 (br s, 1H), 3.19 (br dd, J=14.7, 4.5 Hz, 1H), 3.01 (br dd, J=14.5, 9.6 Hz, 1H), 2.47-2.40 (m, 3H), 0.02-0.06 (m, 1H).

### Preparation of (S)-2-((((9H-fluoren-9-yl)methoxy)carbonyl)amino)-3-(quinolin-6-yl)propanoic acid

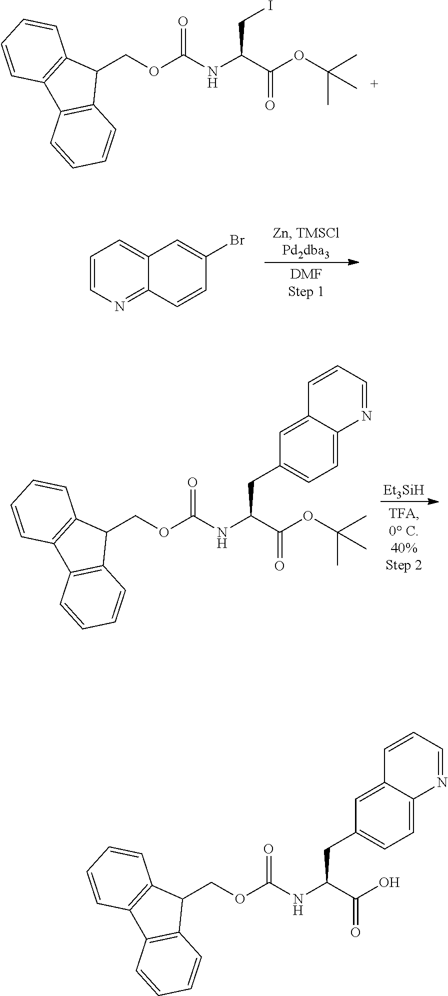

### Step 1.

In a 25-mL round bottom flask, dry zinc (2.32 g, 35.5 mmol) was charged and argon was flashed three times. The flask was heated to 150° C. for 5 min and then allowed to cool to room temp and flushed with argon 3 times. DMF (50 mL) was added followed by the addition of 1,2-dibromoethane (0.017 mL, 0.20 mmol) and TMS-Cl (0.032 mL, 0.25 mmol). Successful zinc insertion was accompanied by a noticeable exotherm. After 5 min (R)-tert-butyl 2-((((9H-fluoren-9-yl)methoxy)carbonyl)amino)-3-iodopropanoate (5.0 g, 10.14 mmol) was added and the reaction was stirred for 30 min.

In a 250-mL round bottom flask purged with Argon was added DMF (50 mL), 6-bromoquinoline (2.53 g, 12.16 mmol), previously prepared solution of alkyl zinc reagent, (R)-tert-butyl 2-((((9H-fluoren-9-yl)methoxy)carbonyl)amino)-3-iodopropanoate (5.0 g, 10.14 mmol) followed by 2-dicyclohexylphosphino-2′,6′-diisopropoxy-1,1′-biphenyl (RuPhos) (0.24 g, 0.51 mmol) and Pd₂(dba)₃ (0.23 g, 0.25 mmol). The reaction mixture was allowed to stir at rt for 5 h and then heated to 50° C. for 16 h. It was cooled to rt and filtered over celite and rinsed with ethyl acetate. The solution was concentrated on rotovap. Purification by flash chromatography gave the desired compound as a thick brown liquid in quantitative yields. Analysis condition E: Retention time=3.47 min; ESI-MS(+) m/z [M+H]⁺: 495.2.

### Step 2.

The final product was obtained following the same procedure of (S)-2-((((9H-fluoren-9-yl)methoxy)carbonyl)amino)-3-(2-methyl-1H-indol-3-yl)propanoic acid. TFA hydrolysis with triethylsilane afforded the desired (S)-2-((((9H-fluoren-9-yl)methoxy)carbonyl)amino)-3-(quinolin-6-yl)propanoic acid as a beige solid in 40% yield after solid-liquid extraction with diethyl ether and water. ¹H NMR (300 MHz, DMSO-d₆) δ 8.94 (br d, J=4.5 Hz, 1H), 8.49 (d, J=8.7 Hz, 1H), 8.01-7.92 (m, 2H), 7.85-7.79 (m, 3H), 7.65 (dd, J=8.3, 4.5 Hz, 1H), 7.55 (dd, J=7.2, 4.2 Hz, 2H), 7.36 (t, J=7.4 Hz, 2H), 7.26-7.14 (m, 2H), 4.32 (dd, J=10.6, 4.5 Hz, 1H), 4.18-4.08 (m, 3H), 3.38-3.29 (m, 2H), 3.11 (br d, J=10.6 Hz, 1H), 2.72 (s, 1H), 1.07 (t, J=7.0 Hz, 1H), −0.02 (s, 1H). Analysis condition E: Retention time=1.54 min; ESI-MS(+) m/z [M+H]⁺: 439.0.

### Preparation of (S)-2-((((9H-fluoren-9-yl)methoxy)carbonyl)amino)-3-(isoquinolin-6-yl)propanoic acid

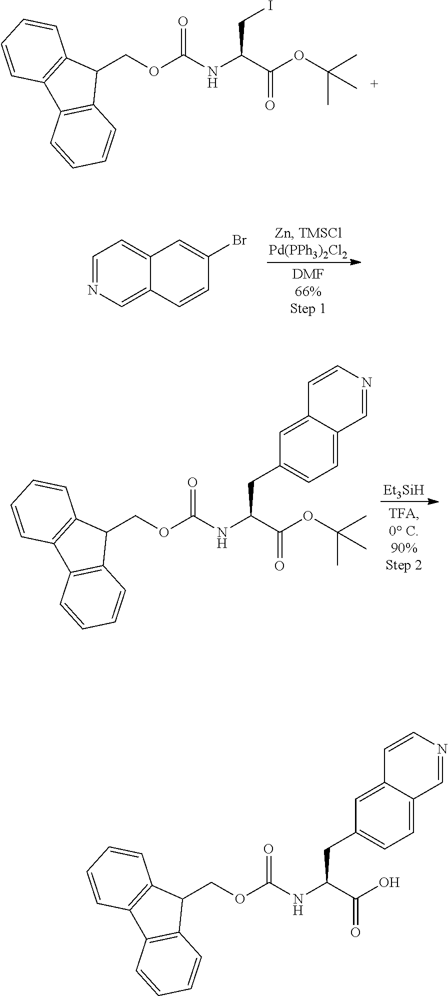

### Step 1.

In a 50-mL three neck flame-dried round bottom flask zinc (1.392 g, 21.28 mmol) was added under argon atmosphere and the flask was heated to 150° C. using a hot gun and was purged with argon. To the reaction DMF (30 mL) was added followed by the addition of 1,2-dibromoethane (10.48 μl, 0.12 mmol) and TMS-Cl (0.016 mL, 0.12 mmol) under argon. The reaction was stirred for 10 minutes. To the reaction mixture (R)-tert-butyl 2-((((9H-fluoren-9-yl)methoxy)carbonyl)amino)-3-iodopropanoate (3.0 g, 6.08 mmol) was added and the reaction was stirred for 1 hr To the reaction mixture 6-bromoisoquinoline (1.52 g, 7.30 mmol) and bis-(triphenylphosphino)-palladous chloride (0.20 g, 0.30 mmol) were added and the reaction was stirred for 16 h. The reaction mixture was diluted with ethyl acetate (50 mL), filtered through celite and washed with ethyl acetate (50 mL). The filtrate was concentrated under reduced pressure to afford the crude product as a red thick gum. The crude was purified by flash chromatography using 40 to 42% EtOAc in petroleum ether. After concentration on rotovap tert-butyl (S)-2-((((9H-fluoren-9-yl)methoxy)carbonyl)amino)-3-(isoquinolin-6-yl)propanoate (2.0 g, 66%) was obtained as a yellow gum. Analysis condition B: Retention time=2.46 min; ESI-MS(+) m/z [M+H]⁻: 495.3.

### Step 2.

The final product was obtained following the same procedure of (S)-2-((((9H-fluoren-9-yl)methoxy)carbonyl)amino)-3-(2-methyl-1H-indol-3-yl)propanoic acid. TFA hydrolysis with triethylsilane afforded the desired (S)-2-((((9H-fluoren-9-yl)methoxy)carbonyl)amino)-3-(isoquinolin-6-yl)propanoic acid as a grey solid in 90% yield after recrystallization in EtOAc and hexanes. ¹H NMR (400 MHz, METHANOL-d₄) δ 9.55 (s, 1H), 8.46 (d, J=6.5 Hz, 1H), 8.33 (d, J=8.5 Hz, 1H), 8.17 (d, J=6.0 Hz, 1H), 8.08 (s, 1H), 7.99-7.86 (m, 1H), 7.78 (dd, J=7.5, 4.0 Hz, 2H), 7.66-7.48 (m, 2H), 7.43-7.30 (m, 2H), 7.30-7.17 (m, 2H), 4.68 (dd, J=10.0, 4.5 Hz, 1H), 4.32-4.13 (m, 2H), 4.12-3.84 (m, 1H), 3.61 (dd, J=13.8, 4.8 Hz, 1H), 3.32-3.26 (m, 1H), 1.46 (s, 1H). Analysis condition B: Retention time=2.77 min; ESI-MS(+) m/z [M+H]⁺: 439.2.

### Preparation of (S)-2-((((9H-fluoren-9-yl)methoxy)carbonyl)amino)-3-(isoquinolin-4-yl)propanoic acid

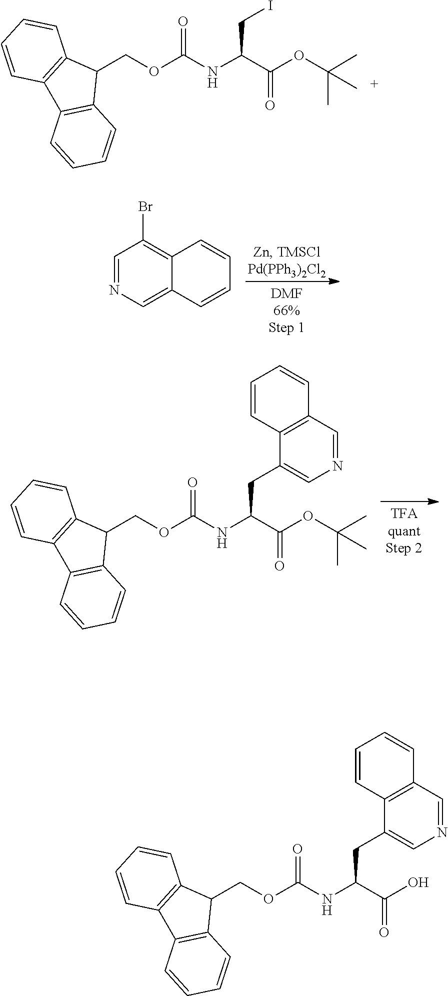 [SMILES: CC(C)(C)OC(=O)[C@H](CI)NC(=O)OCC1c2ccccc2-c2ccccc21 ; Brc1cncc2ccccc12 ; CC(C)(C)OC(=O)[C@H](Cc1cncc2ccccc12)NC(=O)OCC1c2ccccc2-c2ccccc21 ; O=C(N[C@@H](Cc1cncc2ccccc12)C(=O)O)OCC1c2ccccc2-c2ccccc21]

### Step 1.

To a stirred mixture of zinc (2.319 g, 35.5 mmol) in DMF (50 mL) was added dibromomethane (0.071 mL, 1.014 mmol) and TMS-Cl (0.130 mL, 1.014 mmol). Exotherm was observed. The reaction mixture was for 10 min. (R)-tert-butyl 2-((((9H-fluoren-9-yl)methoxy)carbonyl)amino)-3-iodopropanoate (5 g, 10.14 mmol) was added and again exotherm was observed. The reaction was allowed to stir for 1 h at room temperature. 2-Dicyclohexylphosphino-2′,6′-dimethoxybiphenyl (0.21 g, 0.51 mmol), tris(dibenzylideneacetone)dipalladium(0) (0.23 g, 0.25 mmol) and 4-bromoisoquinoline (2.11 g, 10.14 mmol) were added sequentially and the reaction was heated to 50° C. for 16 h. The reaction mixture was cooled to rt and treated with saturated ammonium chloride solution (200 mL). The crude was diluted with the ethyl acetate (300 mL). Layers were separated and the organic layer was washed with brine and dried over anhydrous sodium sulphate. After filtration and concentration the crude product was purified by flash chromatography eluting with 30% of ethyl acetate in petroleum ether to afford tert-butyl (S)-2-((((9H-fluoren-9-yl)methoxy)carbonyl)amino)-3-(isoquinolin-4-yl)propanoate (2.5 g, 50%). Analysis condition E: Retention time=3.44 min; ESI-MS(+) m/z [M+H]⁺: 495.2. Step 2.

The final product was obtained following the same procedure of (S)-2-((((9H-fluoren-9-yl)methoxy)carbonyl)amino)-3-(2-methyl-1H-indol-3-yl)propanoic acid. TFA hydrolysis afforded the desired (S)-2-((((9H-fluoren-9-yl)methoxy)carbonyl)amino)-3-(isoquinolin-4-yl)propanoic acid as an off white solid in quantitative yield after purification diethyl ether trituration. ¹H NMR (400 MHz, DMSO-d₆) δ 9.55 (s, 1H), 8.52 (s, 1H), 8.44-8.24 (m, 2H), 8.18-8.00 (m, 1H), 7.95-7.80 (m, 4H), 7.59 (br d, J=7.5 Hz, 1H), 7.56 (br d, J=7.5 Hz, 1H), 7.47-7.34 (m, 2H), 7.34-7.24 (m, 2H), 4.46-4.30 (m, 1H), 4.25-4.02 (m, 3H), 3.69 (dd, J=14.1, 4.5 Hz, 1H), 3.37 (dd, J=14.1, 10.5 Hz, 1H), 0.10-0.11 (m, 1H). Analysis condition E: Retention time=1.57 min; ESI-MS(+) m/z [M+H]⁺: 441.2.

### Preparation of (S)-2-((((9H-fluoren-9-yl)methoxy)carbonyl)amino)-3-(4-(tert-butoxy)-3,5-difluorophenyl)propanoic acid

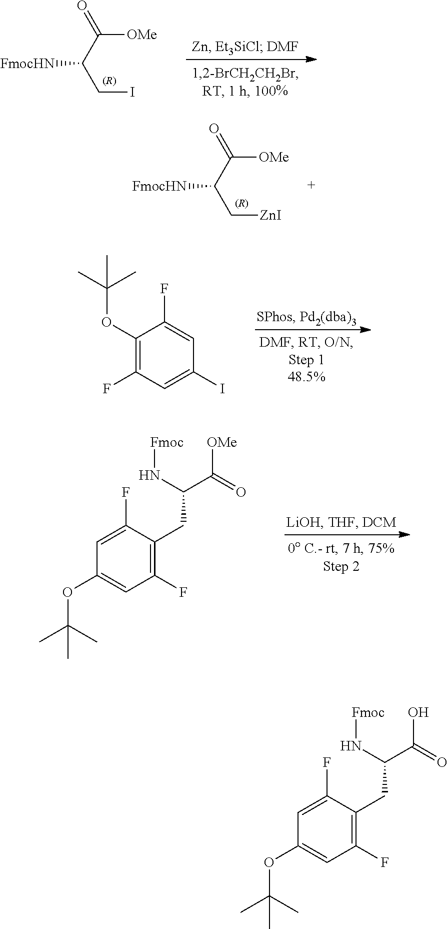

### Step 1.

The compound was prepared following the same procedure of tert-butyl (S)-2-((((9H-fluoren-9-yl)methoxy)carbonyl)amino)-3-(isoquinolin-4-yl)propanoate. First Negishi coupling with methyl (R)-2-((((9H-fluoren-9-yl)methoxy)carbonyl)amino)-3-iodopropanoate at 50° C. afforded the desired methyl (S)-2-((((9H-fluoren-9-yl)methoxy)carbonyl)amino)-3-(4-(tert-butoxy)-2,6-difluorophenyl)propanoate (5.5 g, 48.5% yield) after purification by flash chromatography. Analysis condition E: Retention time=3.99 min; ESI-MS(+) m/z [M+NH₄]⁺: 527.2.

### Step 2.

In a multi-neck round bottom flask methyl (S)-2-((((9H-fluoren-9-yl)methoxy)carbonyl)amino)-3-(4-(tert-butoxy)-3,5-difluorophenyl)propanoate (11 g, 21.59 mmol) was added followed by the addition of tetrahydrofuran (132 mL) under nitrogen atmosphere at RT. The reaction mixture was cooled to 0° C. and LiGH (1.09 g, 45.3 mmol) in water (132 mL) solution was added. The reaction was stirred for 3 h. It was concentrated under reduced pressure below 38° C. to remove the solvent. The crude compound was cooled to 0° C., sat. Citric acid solution was added to adjust the pH to 4-5. It was extracted with ethyl acetate (3×250 mL). The combined organic layer was washed with water (200 mL) followed by brine (200 mL). The organic layer dried over sodium sulphate, filtered and concentrated under reduced pressure to give the crude (12 g) as a colorless thick mass. The crude compound was purified through ISCO using 120 g redisep column, the product was eluted with 20% of ethyl acetate in petroleum ether. The reactions were concentrated to give (S)-2-((((9H-fluoren-9-yl)methoxy)carbonyl)amino)-3-(4-(tert-butoxy)-3,5-difluorophenyl)propanoic acid (9.0 g, 82%, HPLC purity 97%) as a white fluffy solid. Analysis condition E: Retention time=3.62 min; ESI-MS(+) m/z [M+H]⁺: 513.2. ¹H NMR (CDCl₃, 400 MHz) d 7.75 (d, J=7.6 Hz, 2H), 7.60 (m, 2H), 7.39 (t, J=7.6 Hz, 2H), 7.30 (m, 2H), 6.71 (d, J=7.6 Hz, 2H), 5.26 (m, 1H), 4.65 (m, 1H), 4.48-4.38 (m, 2H), 4.20 (m, 1H), 3.14-2.99 (m, 1H), 1.35 (s, 9H).

### Preparation of (S)-2-((((9H-fluoren-9-yl)methoxy)carbonyl)amino)-3-(isoquinolin-8-yl)propanoic acid

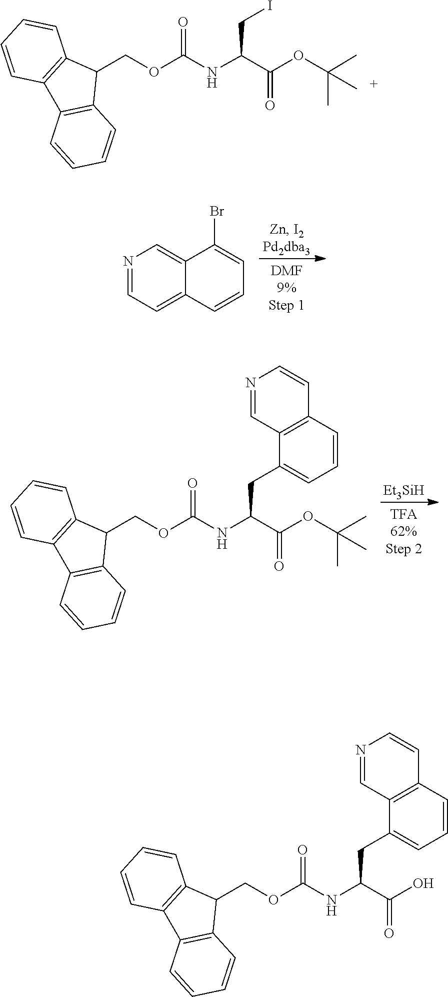

### Step 1.

Zinc (0.79 g, 12.00 mmol) was added to a flame-dried, nitrogen-purged side arm round-bottomed flask. DMF (5 mL) was added via syringe, followed by a catalytic amount of iodine (0.16 g, 0.63 mmol). A color change of the DMF was observed from colorless to yellow and back again. Protected (R)-tert-butyl 2-((((9H-fluoren-9-yl)methoxy)carbonyl)amino)-3-iodopropanoate (1.97 g, 4.00 mmol) was added immediately, followed by a catalytic amount of iodine (0.16 g, 0.63 mmol). The solution was stirred at room temperature; successful zinc insertion was accompanied by a noticeable exotherm. The solution of organozinc reagent was allowed to cool to room temperature and then Pd₂(dba)₃ (0.088 g, 0.096 mmol), dicyclohexyl(2′,6′-dimethoxy-[1,1′-biphenyl]-2-yl)phosphine (0.082 g, 0.200 mmol) and 8-bromoisoquinoline (1.082 g, 5.20 mmol) were added sequentially. The reaction mixture was stirred at 50° C. for 4 h. under a positive pressure of nitrogen. The reaction mixture was cooled to rt, diluted with EtOAc (200 mL) and passed through Celite. The organic solvent was washed with sat. aq. NH₄Cl (200 mL), water (150 mL), and sat. aq. NaCl (200 mL), dried over Na₂SO₄, concentrated, and dried under vacuum to afford the crude compound. It was purified using ISCO combiflash column chromatography (24 g silica gel column, hexanes/ethyl acetate as the eluents) to afford (S)-tert-butyl 2-((((9H-fluoren-9-yl)methoxy)carbonyl)amino)-3-(isoquinolin-8-yl)propanoate (380 mg, 0.768 mmol, 19.21% yield). Analysis condition G: Retention time=2.59 min; ESI-MS(+) m/z [M+H]⁺: 495.3.

### Step2.

(S)-tert-Butyl 2-((((9H-fluoren-9-yl)methoxy)carbonyl)amino)-3-(isoquinolin-8-yl)propanoate (380 mg, 0.768 mmol) was placed in 50-mL round bottom flask and was dissolved in DCM (8 mL). Triethylsilane (0.31 mL, 1.92 mmol) was added followed by trifluoroacetic acid (2.66 mL, 34.6 mmol). The reaction mixture was stirred at room temperature for 5 h. The solvents were evaporated, and the residue was dissolved in diethyl ether. The product was precipitated by the addition of petroleum ether. The resulting powder was then triturated with petroleum ether to yield (S)-2-((((9H-fluoren-9-yl)methoxy)carbonyl)amino)-3-(isoquinolin-8-yl)propanoic acid (320 mg, 0.712 mmol, 93% yield) as an off white solid. ¹H-NMR: (400 MHz, DMSO-d₆) δ ppm: 12.98 (bs, 1H), 9.79 (s, 1H), 8.62 (d, J=9.42 Hz, 1H), 8.22 (d, J=9.42 Hz, 1H), 8.06 (d, J=9.42 Hz, 1H), 7.84-7.93 (m, 4H), 7.74-7.76 (m, 1H), 7.56-7.58 (m, 1H), 7.38-7.42 (m, 2H), (m, 3H), 7.26-7.30 (m, 2H), 4.41 (m, 1H), 4.10-4.15 (m, 3H), 3.731-3.66 (m, 1H), 3.47-3.50 (m, 1H). Analysis condition G: Retention time=2.012 min; ESI-MS(+) m/z [M+H]⁺: 439.2 with 97.5% purity.

### Preparation of (S)-2-((((9H-fluoren-9-yl)methoxy)carbonyl)amino)-3-(7-fluoro-]H-indol-3-yl)propanoic acid

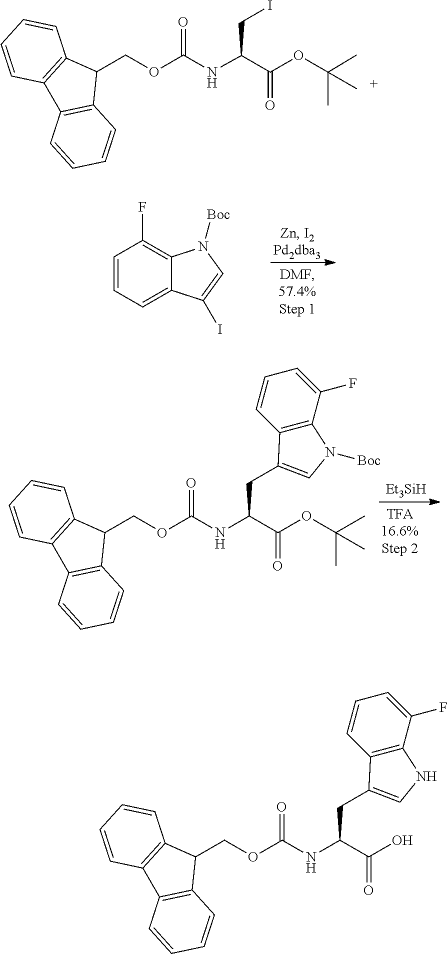

### Step 1.

Synthesis of tert-butyl 6-fluoro-3-iodo-]H-indole-1-carboxylate from 6-fluoro-1H-indole: A solution of iodine (3.76 g, 14.80 mmol) in DMF (15 mL) was dropped to the solution of 6-fluoro-1H-indole (2 g, 14.80 mmol) and potassium hydroxide (2.076 g, 37.0 mmol) in DMF (15 mL) at room temperature and the mixture was stirred for 45 min. The reaction mixture was then poured on 200 mL of ice water containing 0.5% ammonia and 0.1% sodium disulfite. The mixture was placed in a refrigerator to ensure the complete precipitation. The precipitate was filtered, washed with 100 mL ice water and dried in vacuo to obtain 3.80 g. The solid was suspended in dichloromethane (25 mL). 4-Dimethylaminopyridine (160 mg, 10 mol %) and di-tert-butyl dicarbonate (4.84 g, 22.20 mmol) were dissolved in dichloromethane (15 mL), and were added to the reaction. The resulting mixture was stirred for 30 min at room temperature, washed with 0.1 N HCl (25 mL) and the aqueous phase was extracted with dichloromethane (3×35 mL, monitored by TLC). The combined organic layers were dried with sodium sulfate, the solvents were removed under reduced pressure to obtain tert-butyl 6-fluoro-3-iodo-1H-indole-1-carboxylate (4.16 g, 11.52 mmol, 78% yield) as an orange solid. ¹H-NMR (CDCl₃) δ ppm: 7.82 (d, J=8.23 Hz, 1H), 7.68(s 1H), 7.30-7.34 (m, 1H), 7.03-7.08 (m, 1H), 1.66 (s, 9H) Step 2.

Compound was prepared following the same procedure of (S)-tert-butyl 2-((((9H-fluoren-9-yl)methoxy)carbonyl)amino)-3-(isoquinolin-8-yl)propanoate. First Negishi coupling at 50° C. afforded the desired tert-butyl (S)-3-(2-((((9H-fluoren-9-yl)methoxy)carbonyl)amino)-3-(tert-butoxy)-3-oxopropyl)-7-fluoro-]H-indole-1-carboxylate (690 mg, 1.149 mmol, 57.4% yield) after purification by flash chromatography. Analysis condition H: Retention time=3.885 min; ESI-MS(+) m/z [M-Boc-tBu+H]⁺: 445.2

### Step 3

Final product was obtained following the same procedure of (S)-2-((((9H-fluoren-9-yl)methoxy)carbonyl)amino)-3-(isoquinolin-8-yl)propanoic acid. TFA hydrolysis afforded the desired (S)-2-((((9H-fluoren-9-yl)methoxy)carbonyl)amino)-3-(7-fluoro-]H-indol-3-yl)propanoic acid as an off white powder (96 mg, 0.191 mmol, 16.63% yield) after purification by reverse phase prep HPLC (Column: 80 g size, Silisep C18, 19X150 mm,5 m, Mobile phases: A=10 mM ammonium acetate in water, B=MeoH.15 mL/min flow Gradient: 0-20 min, 5-30% B, 20-55 min, 30-80% B, 55-60 min, 80-100% B, held at 100% B for 5 min. Compound was eluted at 75% B) followed by lyophilization. Analysis condition F: Retention time=1.367 min; ESI-MS(+) m/z [M+H]⁺: 445.3. ¹H-NMR (400 MHz, DMSO-d₆) δ ppm: 11.22 (s, 1H), 7.86 (d, J=8.72 Hz, 2H), 7.62-7.65 (m, 1H), 7.52-7.55 (m, 3H), 7.40-7.42 (m, 2H), 7.26-7.38 (m, 2H), 6.78-6.83 (m, 2H), 4.12-4.21 (m, 4H), 3.15-3.18 (m, 1H), 2.97-3.03 (m, 1H).

### Preparation of (2S,3S)-2-((((9H-fluoren-9-yl)methoxy)carbonyl)amino)-3-(1-(tert-butoxycarbonyl)-]H-indol-3-yl)butanoic acid

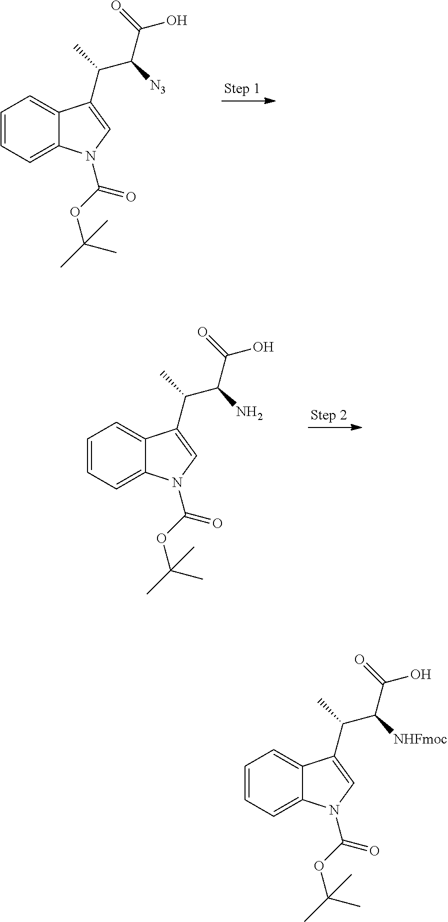

Compound (2S,3S)-2-azido-3-(1-(tert-butoxycarbonyl)-1H-indol-3-yl)butanoic acid was prepared following the procedure reported in Tetrahedron Letters 2001, 42, 4601-4603. The azide reduction step used different conditions as detailed below. Step 1.

To a solution of (2S,3S)-2-azido-3-(1-(tert-butoxycarbonyl)-]H-indol-3-yl)butanoic acid (1000 mg, 2.90 mmol) in THE (58 mL) was added platinum(IV) oxide (132 mg, 0.58 mmol). The reaction mixture was evacuated and filled with hydrogen. The reaction mixture was allowed to stir at room temperature with a hydrogen balloon for 2 h. The reaction mixture was evacuated and back filled with nitrogen three times. The solution was filtered through Celite©. The solvent was removed under vacuum and the crude residue was redissolved in EtOH. This solution was filtered through Celite® to give a clear solution which was concentrated under vacuum (0.89 g 96% yield). ¹H NMR (400 MHz, METHANOL-d₄) δ 8.13 (br d, J=8.0 Hz, 1H), 7.75 (d, J=7.8 Hz, 1H), 7.61 (s, 1H), 7.46-7.18 (m, 2H), 4.89 (s, 2H), 3.80 (d, J=6.5 Hz, 1H), 3.58 (t, J=7.2 Hz, 1H), 1.68 (s, 9H), 1.53 (d, J=7.3 Hz, 3H). Analysis condition B: Retention time=0.93 min; ESI-MS(+) m/z [M+H]⁺: 319.1.

### Step 2.

To a solution of (2S,3S)-2-amino-3-(1-(tert-butoxycarbonyl)-]H-indol-3-yl)butanoic acid (3.96 g, 12.44 mmol) in MeOH (25 mL) was added (9H-fluoren-9-yl)methyl 2,5-dioxopyrrolidine-1-carboxylate (888 mg, 2.76 mmol) followed by Et3N (0.385 mL, 2.76 mmol). The reaction was stirred for 2 h at room temperature. The solvent was removed under vacuum and the residue was redissolved in EtOAc and washed with 1 N HCl aqueous solution then brine. The organic layer was collected, dried over anhydrous sodium sulfate, and concentrated under vacuum to give the desired product (1.3 g, 89% yield) which was not purified further. ¹H NMR (500 MHz, DMSO-d₆) δ 12.78 (br s, 1H), 8.07-7.80 (m, 2H), 7.76-7.48 (m, 4H), 7.46-7.15 (m, 6H), 5.75 (s, 1H), 4.44 (t, J=8.2 Hz, 1H), 4.33-4.22 (m, 1H), 4.19-4.07 (m, 2H), 1.56 (s, 9H), 1.39-1.27 (m, 3H).

### Preparation of (S)-2-((((9H-fluoren-9-yl)methoxy)carbonyl)amino)-3-(6-(o-tolyl)pyridin-3-yl)propanoic acid

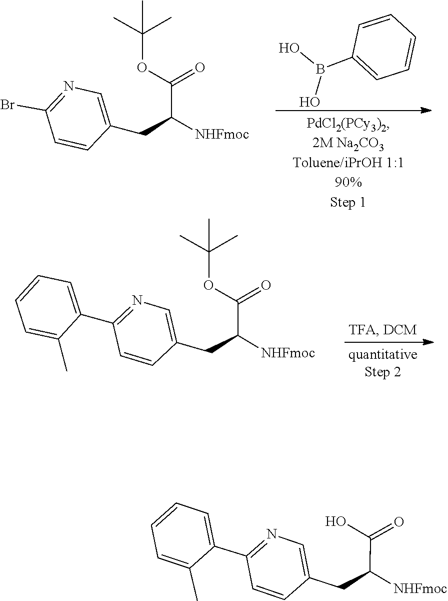

### Step 1.

To a stirred solution of tert-butyl (S)-2-((((9H-fluoren-9-yl)methoxy)carbonyl)amino)-3-(6-bromopyridin-3-yl)propanoate (1750 mg, 3.35 mmol) in toluene/iPrOH (1:1, v:v, 50 mL) was added o-tolylboronic acid (911.6 mg, 6.7 mmol) and 2M Na₂CO₃ aqueous solution (25.0 mL). The mixture was purged with argon three times. Dichlorobis(tricyclohexylphosphine)palladium(II) (123.6 mg, 0.167 mmol) was added and the reaction mixture was purged twice with argon. The reaction was heated to 80° C. for 20 h. The reaction was cooled to room temperature and iPrOH was removed by rotovap. The crude was partitioned between water and EtOAc. The aqueous phase was extracted with EtOAc. Organic phases were combined and dried over anhydrous MgSO₄. After filtration and concentration the crude product was obtained as a brown oil. Purification by flash chromatography using EtOAc:DCM (1:9) as eluant lead to tert-butyl (S)-2-((((9H-fluoren-9-yl)methoxy)carbonyl)amino)-3-(6-(o-tolyl)pyridin-3-yl)propanoate (1.81 g, 3.39 mmol, 90%) as a colorless oil.

### Step 2.

(S)-2-((((9H-Fluoren-9-yl)methoxy)carbonyl)amino)-3-(6-(o-tolyl)pyridin-3-yl)propanoate (1750 mg, 3.19 mmol) was dissolved in trifluoroacetic acid (5.00 mL) and the reaction was allowed to stir at room temperature for two hours. The reaction was brought to dryness on rotovap and the crude product was dissolved in diethyl ether and 1M HCl in diethyl ether. The mixture was sonicated for 2 hours to give a white solid. The product was isolated by filtration and washed with water to give (S)-2-((((9H-fluoren-9-yl)methoxy)carbonyl)amino)-3-(6-(o-tolyl)pyridin-3-yl)propanoic acid (1.91 g, 3.99 mmol, 100%) as a white solid. ¹H NMR (499 MHz, DMSO-d₆) δ 8.90 (s, 1H), 8.48 (br d, J=8.0 Hz, 1H), 7.96 (t, J=6.9 Hz, 2H), 7.89 (d, J=7.5 Hz, 2H), 7.64 (dd, J=7.2, 4.8 Hz, 2H), 7.52-7.45 (m, 1H), 7.43-7.29 (m, 7H), 4.46 (ddd, J=10.7, 8.9, 4.5 Hz, 1H), 4.25-4.15 (m, 3H), 3.45-3.34 (m, 1H), 3.18-3.10 (m, 1H), 3.08-3.00 (m, 1H), 2.27-2.20 (m, 3H).

### Preparation of (S)-2-((((9H-fluoren-9-yl)methoxy)carbonyl)amino)-3-(4′-acetamido-[,1′-biphenyl]-4-yl)propanoic acid

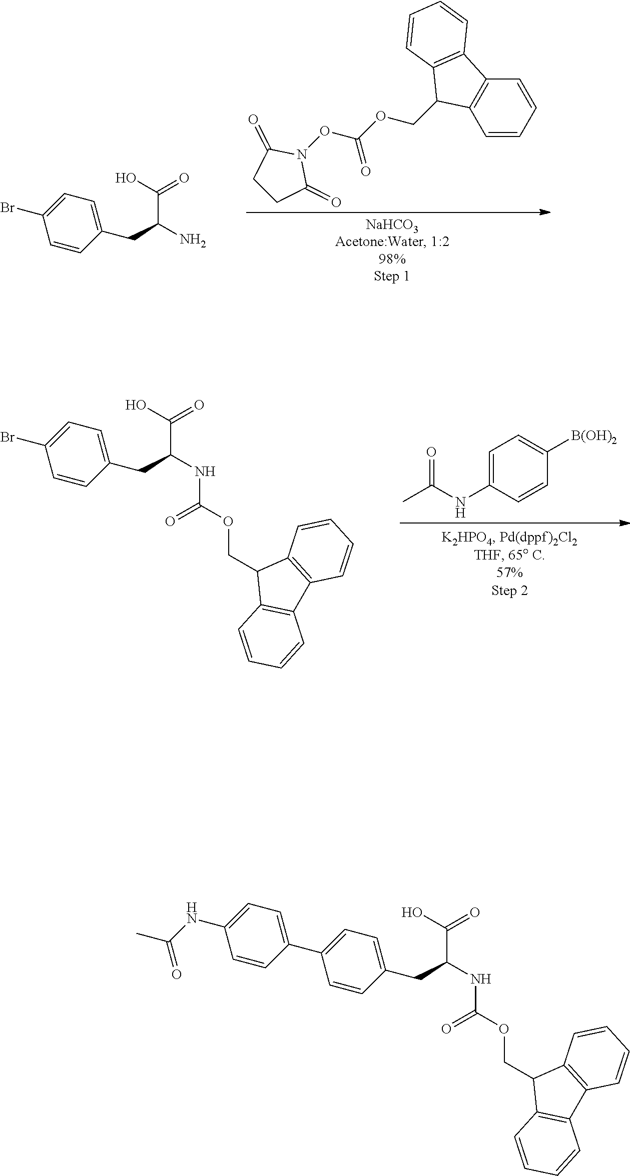 [SMILES: N[C@@H](Cc1ccc(Br)cc1)C(=O)O ; O=C(OCC1c2ccccc2-c2ccccc21)ON1C(=O)CCC1=O ; O=C(N[C@@H](Cc1ccc(Br)cc1)C(=O)O)OCC1c2ccccc2-c2ccccc21 ; CC(=O)Nc1ccc(B(O)O)cc1 ; CC(=O)Nc1ccc(-c2ccc(C[C@H](NC(=O)OCC3c4ccccc4-c4ccccc43)C(=O)O)cc2)cc1]

### Step 1.

A 5.0-1 multi-neck round-bottomed flask was charged with (S)-2-amino-3-(4-bromophenyl)propanoic acid (150.0 g, 615 mmol), Fmoc-OSu (207 g, 615 mmol) in acetone (1500 mL), a solution of sodium bicarbonate (258 g, 3073 mmol) in water (3000 mL) in one lot and allowed to stir at room temperature for 16 h. The reaction mixture was slowly acidified with 10 N HCl solution to pH 1 and stirred for 15 min. The slurry was filtered and dried under vacuum and the cake was washed with water (3.0 L). Solids were dried for 16 h. The desired product was obtained as a white solid (280 g, 98%) and the product was taken to the next stage. Analysis condition E: Retention time=2.17 min; ESI-MS(+) m/z [M+H]⁺: 466.2.

### Step 2.

To a stirred solution of (S)-2-((((9H-fluoren-9-yl)methoxy)carbonyl)amino)-3-(4-bromophenyl)propanoic acid (1.0 g, 2.144 mmol) and (4-acetamidophenyl)boronic acid (0.576 g, 3.22 mmol) with THF (50 mL) in 150-mL pressure tube, Argon was purged for 5 min. Potassium phosphate, tribasic (1.366 g, 6.43 mmol) was then added and the purging was continued for another 5 min. 1,1′-bis(di-tert-butylphosphino)ferrocene palladium dichloride (0.140 g, 0.214 mmol) was then added, and the purging was continued for another 5 min. The reaction mixture was heated to 65° C. for 26 h. The reaction mass was diluted with EtOAc (25 mL) and washed with 10% citric acid aqueous solution (10 mL) and then brine solution to get the crude product. It was triturated with 20% DCM, stirred for 10 min and filtered with a buchner funnel, and then dried for 10 min. The crude was purified by flash chromatography to give 0.7 g (57%) of the desired product as a brown solid. Analysis condition E: Retention time=1.79 min; ESI-MS(+) m/z [M+H]⁺: 519.0. ¹H NMR (400 MHz, DMSO-d₆) δ 12.75 (br s, 1H), 9.99 (s, 1H), 7.87 (d, J=7.5 Hz, 2H), 7.77-7.49 (m, 9H), 7.47-7.22 (m, 7H), 4.26-4.13 (m, 4H), 3.11 (br dd, J=13.8, 4.3 Hz, 1H), 2.91 (dd, J=13.8, 10.8 Hz, 1H), 2.12-2.01 (m, 4H).

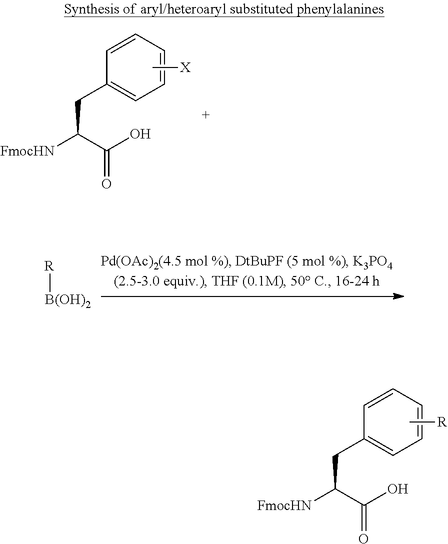

General procedures for Suzuki-Miyaura coupling (SMC) reactions in Scheme 1. To a N₂-flushed 20-mL scintillation vial equipped with a magnetic stir bar was added Fmoc-halo-Phe-OH (0.5 mmol), boronic acid (1.5-2.5 equiv.), and anhydrous THE (6 mL). The suspension was degassed by bubbling N₂ into the vial for several minutes. Palladium(II) acetate (4.5 mol %), DtBuPF (5 mol %), and then anhydrous K₃PO₄ (2.5 equiv.) were added. The suspension was degassed for several minutes, and then the vial was capped with a septum. The reaction mixture was stirred at 50° C. for 16 h. After cooling, 20% aqueous citric acid solution was added to acidify the reaction. The organic layer was separated, and the aqueous layer was extracted with EtOAc (2×). Silica gel was added to the combined organic layers, and the mixture was concentrated to dryness. The residue was dry-loaded on a silica gel column (ISCO system) and eluted with hexanes/EtOAc to give the desired product. Sometimes for compounds which are tailing in a Hexanes/EtOAc system, further eluting with MeOH/CH₂Cl₂ is also needed.

### Preparation of (S)-2-((((9H-fluoren-9-yl)methoxy)carbonyl)amino)-3-(4′-(tert-butoxycarbonyl)-[1,1′-biphenyl]-4-yl)propanoic acid

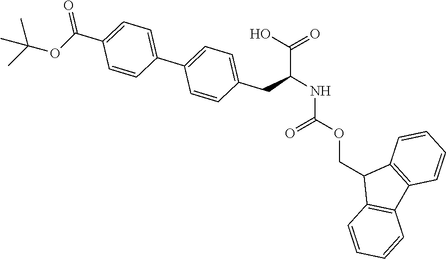 [SMILES: CC(C)(C)OC(=O)c1ccc(-c2ccc(C[C@H](NC(=O)OCC3c4ccccc4-c4ccccc43)C(=O)O)cc2)cc1]

(S)-2-((((9H-Fluoren-9-yl)methoxy)carbonyl)amino)-3-(4′-(tert-butoxycarbonyl)-[1,1′-biphenyl]-4-yl)propanoic acid was prepared according to the SMC general procedure. Yield: 78% (439 mg); colorless solids. ¹H NMR (400 MHz, methanol-d₄) δ 7.94 (d, J=8.3 Hz, 2H), 7.74 (d, J=7.6 Hz, 2H), 7.56 (d, J=8.4 Hz, 4H), 7.51 (d, J=8.1 Hz, 2H), 7.38-7.28 (m, 4H), 7.28-7.17 (m, 2H), 4.56-4.38 (m, 1H), 4.29 (dd, J=10.5, 7.0 Hz, 1H), 4.17 (dd, J=10.5, 7.1 Hz, 1H), 4.08 (t, J=7.0 Hz, 1H), 3.29-3.21 (m, 1H), 2.98 & 2.80 (dd, J=13.8, 9.6 Hz, total 1H), 1.59 (s, 9H). ESI-HRMS: Calcd for C₃₅H₃₄NO₆ [M+H]⁺ 564.23806, found 564.23896, mass difference 1.588 ppm.

### Preparation of (S)-2-((((9H-fluoren-9-yl)methoxy)carbonyl)amino)-3-(3′-(tert-butoxycarbonyl)-[1,1′]-biphenyl]-4-yl)propanoic acid

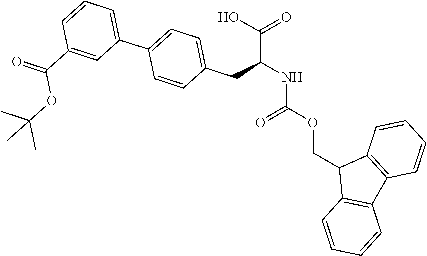 [SMILES: CC(C)(C)OC(=O)c1cccc(-c2ccc(C[C@H](NC(=O)OCC3c4ccccc4-c4ccccc43)C(=O)O)cc2)c1]

(S)-2-((((9H-Fluoren-9-yl)methoxy)carbonyl)amino)-3-(4′-(tert-butoxycarbonyl)-[1,1′-biphenyl]-4-yl)propanoic acid was prepared according to the SMC general procedure. Yield: 85% (240 mg); off-white solids. ¹H NMR (500 MHz, DMSO-d₆) δ 8.08 (t, J=1.8 Hz, 1H), 7.86 (dd, J=7.7, 1.4 Hz, 3H), 7.83 (d, J=8.1 Hz, 1H), 7.64 (d, J=7.7 Hz, 1H), 7.63 (d, J=7.5 Hz, 1H), 7.58-7.48 (m, 3H), 7.41-7.35 (m, 2H), 7.31 (d, J=7.8 Hz, 2H), 7.30-7.23 (m, 2H), 4.31-4.10 (m, 4H), 4.05 (td, J=8.2, 4.5 Hz, 1H), 3.13 & 2.9 (dd, J=13.6, 4.5 Hz, total 1H), 2.94 & 2.76 (dd, J=13.6, 8.7 Hz, total 1H), 1.56 (s, 9H). ESI-HRMS: Calcd for C₃₅H₃₇N₂O₆ [M+NH₄]⁺ 581.26461, found at 581.26474, mass difference 0.218 ppm.

### Preparation of (S)-2-((((9H-fluoren-9-yl)methoxy)carbonyl)amino)-3-(4-boronophenyl)propanoic acid

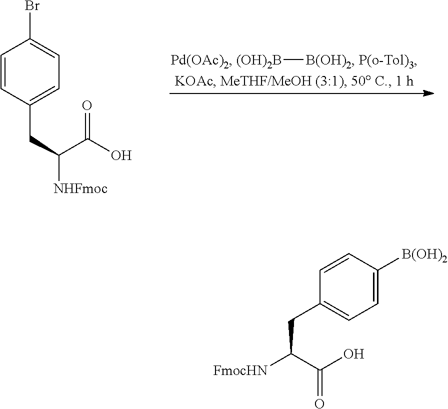

To a 75-mL pressure bottle (S)-2-((((9H-fluoren-9-yl)methoxy)carbonyl)amino)-3-(4-bromophenyl)propanoic acid (6.0 g, 12.87 mmol) and 2-methyl THE (250 mL) were charged, and the solution was purged with argon for 5 min. Tri-o-tolylphosphine (0.31 g, 1.03 mmol), tetrahydroxydiboron (2.31 g, 25.7 mmol), potassium acetate (3.79 g, 38.6 mmol) were added every in 10-min interval followed by the addition of MeOH (100 mL) and Pd(OAc)₂ (0.12 g, 0.52 mmol), and argon was purged for 10 min. The reaction was heated at 50° C. overnight. The reaction mixture was transferred into a 1-liter separatory funnel, diluted with 2-methyl-THF, and acidified with 1.5 N HCl to pH=2. The organic layer was washed with brine, dried (sodium sulphate), passed through celite, and concentrated to give black crude material. The crude was treated with petroleum ether to give a solid (10 g) which was dissolved with 2-methyl-THF and charcoal (2 g) was added. The mixture was heated on a rotovap without vacuum at 50° C. After filtration, the filtrate was passed through celite, concentrated. The resulting solid was treated with 30% ethyl acetate in petroleum ether, filtered to give 8 g of the crude as a fine off-white solid, which was further purified via flash chromatography then trituration with petroleum ether to give (S)-2-((((9H-fluoren-9-yl)methoxy)carbonyl)amino)-3-(4-boronophenyl)propanoic acid (4.0 g, 9.28 mmol, 72.1% yield) as a white solid. LCMS: 432.1 (M+H), tr=0.82 min. ¹H NMR (500 MHz, DMSO-d₆) δ 7.88 (d, J=7.6 Hz, 2H), 7.85-7.77 (m, 1H), 7.71 (br d, J=7.9 Hz, 3H), 7.68-7.60 (m, 2H), 7.41 (br d, J=6.6 Hz, 2H), 7.35-7.20 (m, 4H), 4.30-4.11 (m, 5H), 3.16-3.03 (m, 1H), 2.95-2.83 (m, 1H).

### Preparation of (S)-2-((((9H-fluoren-9-yl)methoxy)carbonyl)amino)-3-(4′-fluoro-[, 1′-biphenyl]-4-yl)propanoic acid

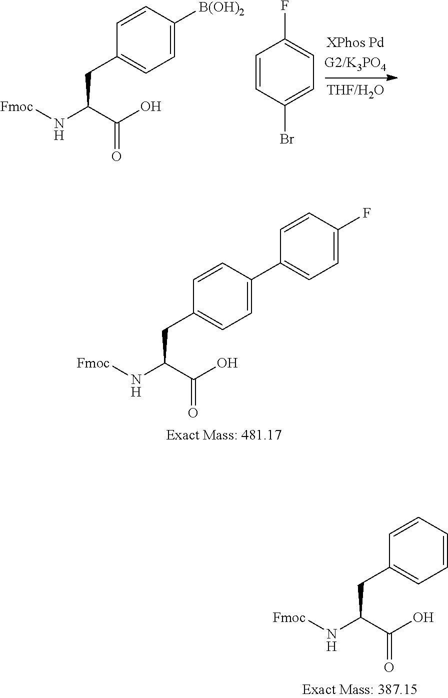

To a stirred solution of (S)-2-((((9H-fluoren-9-yl)methoxy)carbonyl)amino)-3-(4-boronophenyl)propanoic acid (217.5 mg, 0.504 mmol), 1-bromo-4-fluorobenzene (0.083 mL, 0.757 mmol) and XPhos Pd G2 (9.7 mg, 0.012 mmol) in THF (1 mL) at rt was added 0.5 M aqueous K₃PO₄ (2 mL, 1.000 mmol). N₂ was purged with vacuum three times and the mixture was stirred at 80° C. for 16 h. The mixture was cooled to rt. To the reaction was added 10% citric acid until pH<6. It was partitioned between EtOAc and H₂O, and the organic phase was separated, washed with brine, and dried over sodium sulfate. The mixture was filtered, SiO₂ (5 g) was added and concentrated. The material was then purified by flash chromatography (Teledyne ISCO CombiFlash Rf, gradient of 0% to 20% MeOH/CH₂Cl₂ over 15 column volumes, RediSep SiO₂ 40 g). Fractions containing the desired product were collected and concentrated to give (S)-2-((((9H-fluoren-9-yl)methoxy)carbonyl)amino)-3-(4′-fluoro-[,1′-biphenyl]-4-yl)propanoic acid (206.1 mg, 0.43 mmol, 85% yield) as a cream solid: HPLC: RT=1.04 min (Waters Acquity UPLC BEH C18 1.7 μm 2.1×50 mm, CH₃CN/H₂O/0.05% TFA, 1 min. gradient, wavelength=254 nm); MS (ES): m z=482 [M+H]⁺. ¹H NMR (499 MHz, DMSO-d₆) δ 12.78 (br s, 1H), 7.88 (d, J=7.5 Hz, 3H), 7.71-7.61 (m, 5H), 7.53 (d, J=8.1 Hz, 2H), 7.39 (q, J=7.3 Hz, 3H), 7.36-7.23 (m, 8H), 4.24-4.13 (m, 5H), 3.12 (dd, J=14.0, 4.5 Hz, 1H), 2.91 (dd, J=13.6, 10.3 Hz, 1H).

### Preparation of (S)-2-((((9H-fluoren-9-yl)methoxy)carbonyl)amino)-3-(3′,5′-difluoro-[, 1′-biphenyl]-4-yl)propanoic acid

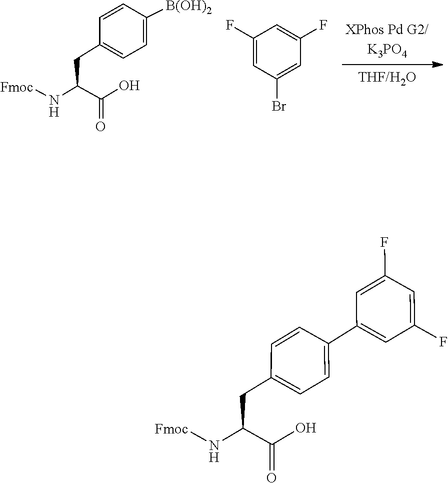

The final product was obtained following the same procedure of (S)-2-((((9H-fluoren-9-yl)methoxy)carbonyl)amino)-3-(4′-fluoro-[1,1′-biphenyl]-4-yl)propanoic acid. The Suzuki coupling reaction afforded the desired (S)-2-((((9H-fluoren-9-yl)methoxy)carbonyl)amino)-3-(3′,5′-difluoro-[1,1′-biphenyl]-4-yl)propanoic acid (197.1 mg, 0.40 mmol, 78% yield) as a colorless solid after purification by flash chromatography. HPLC: RT=1.06 min (Waters Acquity UPLC BEH C18 1.7 μm 2.1×50 mm, CH₃CN/H₂O/0.05% TFA, 1 min. gradient, wavelength=254 nm); MS (ES): m z=500 [M+H]⁺. ¹H NMR (499 MHz, DMSO-d₆) δ 12.90-12.67 (m, 1H), 7.87 (d, J=7.5 Hz, 2H), 7.69-7.61 (m, 4H), 7.45-7.35 (m, 6H), 7.33-7.27 (m, 2H), 7.22-7.16 (m, 1H), 4.25-4.18 (m, 3H), 4.17-4.12 (m, 1H), 3.14 (dd, J=13.8, 4.4 Hz, 1H), 2.92 (dd, J=13.7, 10.6 Hz, 1H).

### Preparation of (S)-2-((((9H-fluoren-9-yl)methoxy)carbonyl)amino)-3-(3′,4′,5′-trifluoro-[1,1′-biphenyl]-4-yl)propanoic acid

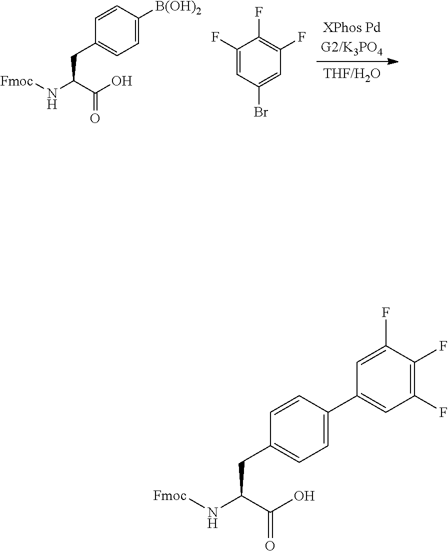

The final product was obtained following the same procedure of (S)-2-((((9H-fluoren-9-yl)methoxy)carbonyl)amino)-3-(4′-fluoro-[1,1′-biphenyl]-4-yl)propanoic acid. The Suzuki coupling reaction afforded the desired (S)-2-((((9H-fluoren-9-yl)methoxy)carbonyl)amino)-3-(3′,4′,5′-trifluoro-[1,1′-biphenyl]-4-yl)propanoic acid (218.5 mg, 0.422 mmol, 84% yield) as a colourless solid after purification by flash chromatography. HPLC: RT=1.466 min (Shimadzu UPLC with Waters Acquity BEH C18 1.7 μm 2.1×50 mm column, CH₃CN/H₂O/0.1% TFA, 3 min. gradient, wavelength=254 nm); MS (ES): m z=556. ¹H NMR (499 MHz, DMSO-d₆) δ 12.79 (br s, 1H), 7.87 (d, J=7.6 Hz, 2H), 7.75 (d, J=8.6 Hz, 1H), 7.69-7.58 (m, 6H), 7.44-7.35 (m, 4H), 7.33-7.25 (m, 2H), 4.27-4.17 (m, 3H), 4.17-4.10 (m, 1H), 3.14 (dd, J=13.8, 4.4 Hz, 1H), 2.92 (dd, J=13.7, 10.7 Hz, 1H).

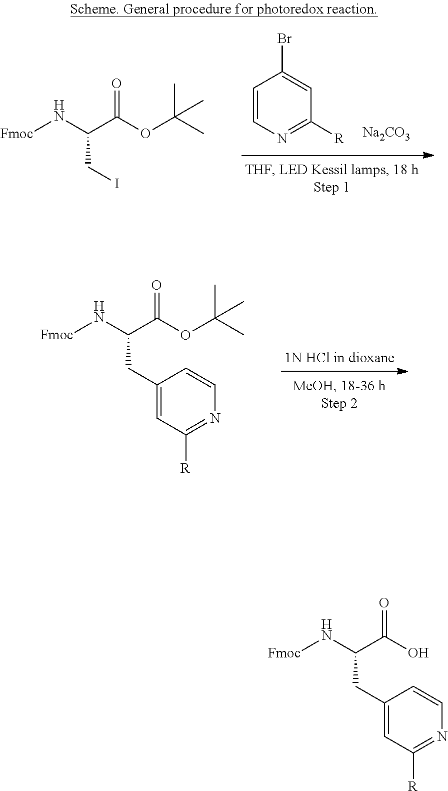

Ir[dF(CF₃)ppy₂]₂(dtbbpy)PF₆ (0.018 g, 0.016 mmol, 1 mol %), tert-butyl (R)-2-((((9H-fluoren-9-yl)methoxy)carbonyl)amino)-3-iodopropanoate (1.181 g, 2.393 mmol, 1.5 equiv), bromo-pyridine derivative (1.596 mmol, 1.00 equiv), pulverized Na₂CO₃ (0.338 g, 3.19 mmol, 2.00 equiv), and tris(trimethylsilane)silane (0.278 g, 1.596 mmol, 1.00 equiv) were charged into an oven-dried 40-mLlpressure-relief screw cap vial. The vial was capped, purged with nitrogen, diluted with THF (45.0 mL), and then sonicated. In a seperate vial were charged NiCl₂-glyme (18 mg, 0.080 mmol, 5 mol %) and di-tertbutylbipyridine (18 mg, 0.096 mmol, 6 mol %) in 1 mL dioxane. The vial was purged with nitrogen for 10 min. The Nickel-ligand complexe solution was transferred to the main reaction vial and the mixture was degassed with gentle nitrogen flow for 20 min. The reactor was sealed with parafilm and placed between 2 34 W blue LED Kessil lamps (ca. 7 cm away) and allowed to stir vigorously. After 16 h, the reaction was monitored by LCMS analysis. The resulting oil was dissolved into 4 M HCl dioxane solution (15 mL). After 16 h, the reaction mixture was brought to dryness on rotovap. The crude product was dissolved in a minimum amount of methanol and dry loaded on silica gel column for purification.

### Preparation of (2S)-2-({[(9H-fluoren-9-yl)methoxy]carbonyl}amino)-3-(2-methoxypyridin-4-yl)propanoic acid

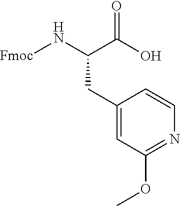

The mixture was rotovaped onto silica gel, purified by isco using 10% to 80% EtOAc/Hexanes. The fractions were pooled, concentrated to obtain the desired product as a clear oil (237 mg, 100%) Analysis conditions D: Retention time 1.74 min; ES+475.1.

### Preparation of ((S)-2-((((9H-fluoren-9-yl)methoxy)carbonyl)amino)-3-(4-(trifluoromethoxy)phenyl)propanoic acid

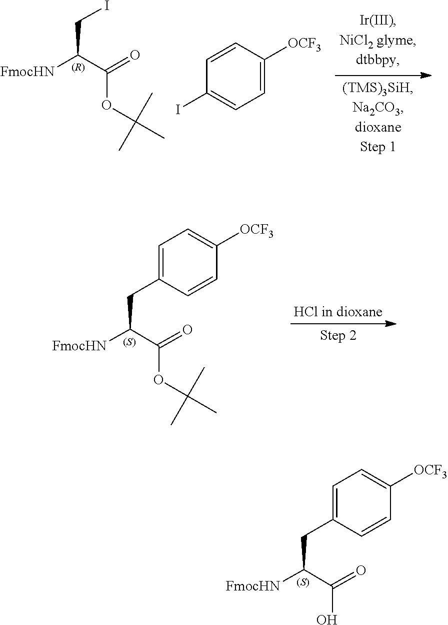

### Step 1

In 4 separate 40-mL vials was placed Ir(dF(CF₃)ppy)₂(dtbbpy)PF₆ (5.6 mg, 4.99 μmol) and Na₂CO₃ (249 mg, 2.35 mmol) in dioxane (18 mL), and was fitted with a teflon screw cap and a stir bar. To the mixture was added 1-iodo-4-(trifluoromethoxy)benzene (0.16 mL, 1.02 mmol) stirred briefly, then tris(trimethylsilyl)silane (0.23 mL, 0.75 mmol) was added via syringe, and the suspension was degassed (cap on) with nitrogen for 5 min. To a separate 40-mL vial was added nickel(II) chloride ethylene glycol dimethyl ether complex (22 mg, 0.10 mmol) and 4,4′-di-tert-butyl-2,2′-bipyridine (33 mg, 0.12 mmol)ioxane (10 mL) was added and this solution was degassed (cap on) with nitrogen gas for 10 min and stirred. To the Ir mixture was added 2.5 mL of the Ni solution, and 5 mL of a solution of the iodo alanine, tert-butyl (R)-2-((((9H-fluoren-9-yl)methoxy)carbonyl)amino)-3-iodopropanoate (987 mg, 2.0 mmol) in dioxane (20 mL), and then the mixture was further degassed with nitrogen gas for another 5 min (cap on). The vials were sealed with parafilm, placed in the round photoredox reactor with light and fan on, stirred for 40 h. The eactions were removed from the illumination/reactor. The blackish reaction mixtures of each vial were poured into a 500-mL erlenmeyer flask into which was added EtOAc (200 mL). The mixture was filtered through celite, washed with EtOAc, and concentrated. The residue was purified by flash chromatography (Teledyne ISCO CombiFlash Rf, gradient of 0% using solvent A/B=CH₂Cl₂/EtOAcover 10 column volumes, RediSep SiO₂ 80 gloaded as DCM solution). The fractions containing the desired product were collected and concentrated to obtained the product tert-butyl (S)-2-((((9H-fluoren-9-yl)methoxy)carbonyl)amino)-3-(4-(trifluoromethoxy)phenyl)propanoate (865.2 mg, 1.64 mmol, 82% yield, only about 73% HPLC purityas a colourless oiland was used as was in the deprotection step: HPLC: RT=1.62 min (Waters Acquity UPLC BEH C18 1.7 μm 2.1×50 mm, CH₃CN/H₂O/0.05% TFA, 1 min. gradient, wavelength=254 nm); MS (ES): m z=550 [M+23]⁺.

### Step 2.

To a stirred solution of tert-butyl (S)-2-((((9H-fluoren-9-yl)methoxy)carbonyl)amino)-3-(4-(trifluoromethoxy)phenyl)propanoate (865.2 mg, 1.64 mmol) in dichloromethane (8.2 mL) at rt was added HCl (4M in dioxane, 8.20 mL, 32.8 mmol). The mixture was stirred at rt for 18 h. The mixture was concentrated in vacuo then dried under vacuum. The residue was dissolved in DMF (4 mL), purified on ISCO ACCQ Prep over 2 injections. The fractions containing the desire product were combined and partially concentrated on rotovap, then blown air over mixture over weekend. The residue was dissolved in CH₃CN, diluted with water, frozen, and lyophilized. To obtained the product (S)-2-((((9H-fluoren-9-yl)methoxy)carbonyl)amino)-3-(4-(trifluoromethoxy)phenyl)propanoic acid (344.1 mg, 0.73 mmol, 44.5% yield) as a colorless solid. HPLC: RT=1.38 min (Waters Acquity UPLC BEH C18 1.7 μm 2.1×50 mm, CH₃CN/H₂O/0.05% TFA, 1.5 min. gradient, wavelength=254 nm); MS (ES): m z=472 [M+1]⁺. ¹H NMR (499 MHz, DMSO-d₆) ppm δ 7.88 (d, J=7.5 Hz, 2H), 7.63 (d, J=7.4 Hz, 2H), 7.44-7.37 (m, 2H), 7.35-7.25 (m, 4H), 7.19 (br d, J=7.6 Hz, 3H), 4.30-4.20 (m, 1H), 4.21-4.13 (m, 2H), 4.04 (br d, J=3.5 Hz, 1H), 3.11 (br dd, J=13.6, 4.4 Hz, 1H), 2.91 (br dd, J=13.6, 9.1 Hz, 1H).

### Preparation of (S)-2-((((9H-fluoren-9-yl)methoxy)carbonyl)amino)-3-(2,5-dimethylphenyl)propanoic acid

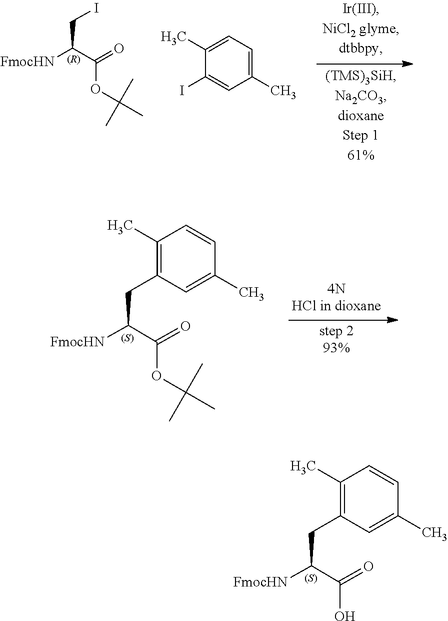

### Step 1.

Compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2,5-dimethylphenyl)propanoate (140.5 mg, 0.298 mmol, 61.1% yield) after purification by flash chromatography. HPLC: RT=1.21 min (Waters Acquity UPLC BEH C18 1.7 μm 2.1×50 mm, CH₃CN/H₂O/0.05% TFA, 1 min. gradient, wavelength=254 nm); Analysis condition F: Retention time=1.21 min; ESI-MS(+) m/z [M-tBu+H]⁺: 416. ¹H NMR (499 MHz, CHLOROFORM-d) δ 7.78 (d, J=7.5 Hz, 2H), 7.63-7.56 (m, 2H), 7.42 (t, J=7.4 Hz, 2H), 7.37-7.30 (m, 2H), 7.07 (d, J=7.7 Hz, 1H), 6.98 (d, J=7.7 Hz, 1H), 6.96 (s, 1H), 4.58-4.51 (m, 1H), 4.39 (dd, J=10.5, 7.3 Hz, 1H), 4.34 (dd, J=10.5, 7.2 Hz, 1H), 4.24-4.19 (m, 1H), 3.10-3.01 (m, 2H), 2.34 (s, 3H), 2.28 (s, 3H), 1.40 (s, 8H).

### Step 2.

Final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2,5-dimethylphenyl)propanoic acid (115.2 mg, 0.277 mmol, 93% yield) as a cream solid after purification by reverse phase flash chromatography. IPLC: RT=1.03 min (Waters Acquity UPLC BEH C18 1.7 μm 2.1×50 mm, CH₃CN/H₂O/0.05% TFA, 1 min. gradient, wavelength=254 nm); MS (ES): m z=416 [M+H]⁺. ¹H NMR (499 MHz, CHLOROFORM-d) δ 7.88 (d, J=7.4 Hz, 2H), 7.79 (br d, J=8.6 Hz, 1H), 7.67 (d, J=7.4 Hz, 1H), 7.64 (d, J=7.5 Hz, 1H), 7.41 (td, J=7.3, 4.2 Hz, 3H), 7.35-7.29 (m, 2H), 7.29-7.25 (m, 1H), 7.02 (br d, J=8.9 Hz, 2H), 6.91 (br d, J=7.4 Hz, 1H), 4.21-4.10 (m, 5H), 3.07 (dd, J=14.1, 4.4 Hz, 1H), 2.80 (dd, J=14.1, 10.3 Hz, 1H), 2.24 (s, 3H), 2.18 (s, 3H).

### Preparation of (S)-2-((((9H-fluoren-9-yl)methoxy)carbonyl)amino)-3-(4-fluoro-3-methylphenyl)propanoic acid

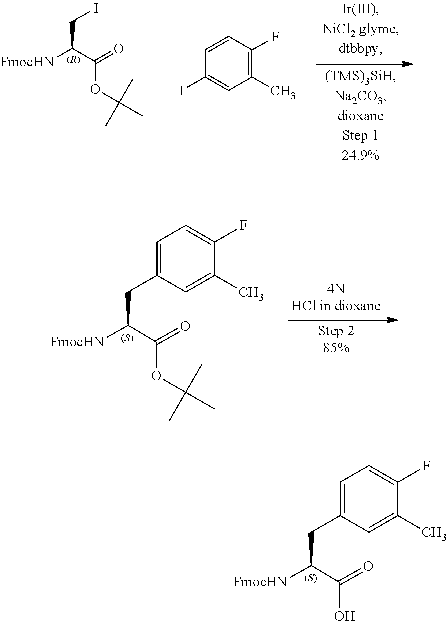

### Step 1.

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(4-fluoro-3-(trifluoromethyl)phenyl)propanoate (66.3 mg, 0.13 mmol, 24.9% yield) as a colourless solid after purification by flash chromatography. HPLC: RT=1.19 min (Waters Acquity UPLC BEH C18 1.7 μm 2.1×50 mm, CH₃CN/H₂O/0.05% TFA, 1 min. gradient, wavelength=254 nm); MS (ES): m z=474 [M-tBu]⁺. ¹H NMR (499 MHz, CHLOROFORM-d) δ 7.80 (d, J=7.5 Hz, 2H), 7.60 (dd, J=7.6, 3.3 Hz, 2H), 7.47-7.39 (m, 3H), 7.38-7.32 (m, 2H), 7.16-7.09 (m, 1H), 5.34 (br d, J=7.7 Hz, 1H), 4.57-4.47 (m, 2H), 4.40 (dd, J=10.3, 6.9 Hz, 1H), 4.26-4.21 (m, 1H), 3.14 (br d, J=4.9 Hz, 2H), 1.44 (s, 9H).

### Step 2.

Final product was obtained following the same procedure of (S)-2-((((9H-fluoren-9-yl)methoxy)carbonyl)amino)-3-(4-(trifluoromethoxy)phenyl)propanoic acid. Removal of the tBu ester with HCl/dioxane afforded the desired (S)-2-((((9H-fluoren-9-yl)methoxy)carbonyl)amino)-3-(4-fluoro-3-methylphenyl)propanoic acid (58.3 mg, 0.139 mmol, 85% yield) as a cream solid after purification by reverse phase flash chromatography. HPLC: RT=1.02 min (Waters Acquity UPLC BEH C18 1.7 μm 2.1×50 mm, CH₃CN/H₂O/0.05% TFA, 1 min. gradient, wavelength=254 nm); MS (ES): m z=420 [M+H+. ¹H NMR (499 MHz, DMSO-d₆) 6 12.86-12.66 (m, 1H), 7.89 (d, J=7.5 Hz, 2H), 7.73 (d, J=8.3 Hz, 1H), 7.65 (t, J=7.5 Hz, 2H), 7.42 (t, J=7.5 Hz, 2H), 7.35-7.26 (m, 2H), 7.17 (br d, J=7.5 Hz, 1H), 7.14-7.08 (m, 1H), 7.06-6.99 (m, 1H), 4.24-4.11 (m, 4H), 3.03 (dd, J=13.7, 4.3 Hz, 1H), 2.82 (dd, J=13.6, 10.6 Hz, 1H), 2.17 (s, 3H).

### Preparation of (S)-2-((((9H-fluoren-9-yl)methoxy)carbonyl)amino)-3-(2,4-difluoro-5-methoxyphenyl)propanoic acid

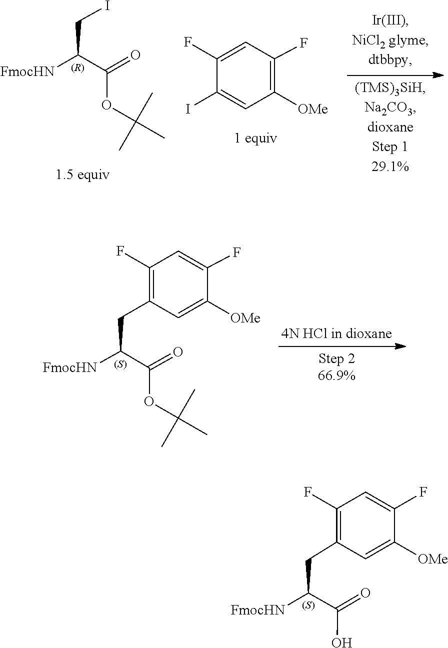

### Step 1.

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2,4-difluoro-5-methoxyphenyl)propanoate (77.1 mg, 0.151 mmol, 29.1% yield as a colourless solid after purification by flash chromatography. HPLC: RT=1.15 min (Waters Acquity UPLC BEH C18 1.7 μm 2.1×50 mm, CH₃CN/H₂O/0.05% TFA, 1 min. gradient, wavelength=254 nm); MS (ES): m z=454 [M-t-Bu]⁺. ¹H NMR (499 MHz, CHLOROFORM-d) δ 7.79 (d, J=7.4 Hz, 2H), 7.59 (t, J=6.4 Hz, 2H), 7.43 (t, J=7.3 Hz, 2H), 7.33 (td, J=7.5, 1.1 Hz, 3H), 6.85 (dd, J=10.8, 9.3 Hz, 1H), 6.83-6.79 (m, 1H), 5.40 (br d, J=8.1 Hz, 1H), 4.58-4.51 (m, 1H), 4.38 (dd, J=7.0, 4.5 Hz, 2H), 4.25-4.20 (m, 1H), 3.82 (s, 3H), 3.18-3.05 (m, 2H), 1.45 (s, 9H).

### Step 2.

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2,4-difluoro-5-methoxyphenyl)propanoic acid (45.9 mg, 0.101 mmol, 66.9% yield) as a cream solid after purification by reverse phase flash chromatography. HPLC: RT=0.99 min (Waters Acquity UPLC BEH C18 1.7 μm 2.1×50 mm, CH₃CN/H₂O/0.05% TFA, 1 min. gradient, wavelength=254 nm); MS (ES): m z=454 [M+1]⁺. ¹H NMR (499 MHz, DMSO-d₆) δ 12.92 (br s, 1H), 7.89 (d, J=7.5 Hz, 2H), 7.71-7.65 (m, 1H), 7.63 (d, J=7.5 Hz, 2H), 7.41 (t, J=7.5 Hz, 2H), 7.34-7.25 (m, 2H), 7.24-7.15 (m, 2H), 4.24-4.12 (m, 4H), 3.77 (s, 3H), 3.16 (br dd, J=13.8, 4.6 Hz, 1H), 2.82 (dd, J=13.6, 10.7 Hz, 1H).

### Preparation of (S)-2-((((9H-fluoren-9-yl)methoxy)carbonyl)amino)-3-(2,3-dimethylphenyl)propanoic acid

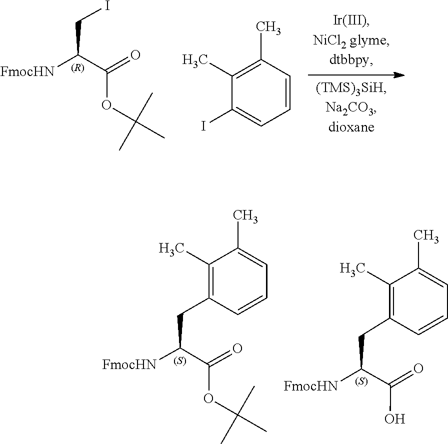

### Step 1.

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2,3-dimethylphenyl)propanoate (107.5 mg, 0.228 mmol, 55.5% yield) as a tan viscous oil after purification by flash chromatography. HPLC: RT=1.21 min (Waters Acquity UPLC BEH C18 1.7 μm 2.1×50 mm, CH₃CN/H₂O/0.05% TFA, 1 min. gradient, wavelength=254 nm); MS (ES): m z=416 [M-t-Bu]⁺. ¹H NMR (499 MHz, CHLOROFORM-d) δ 7.79 (d, J=7.5 Hz, 2H), 7.61-7.56 (m, 2H), 7.42 (t, J=7.5 Hz, 2H), 7.35-7.31 (m, 2H), 7.09-7.06 (m, 1H), 7.02 (t, J=7.5 Hz, 1H), 7.00-6.96 (m, 1H), 5.30 (br d, J=8.3 Hz, 1H), 4.53 (q, J=7.4 Hz, 1H), 4.39 (dd, J=10.6, 7.3 Hz, 1H), 4.34 (dd, J=10.4, 7.0 Hz, 1H), 4.21 (t, J=7.2 Hz, 1H), 3.15 (dd, J=14.2, 7.0 Hz, 1H), 3.08 (dd, J=14.1, 7.3 Hz, 1H), 2.29 (s, 3H), 2.28 (s, 3H), 1.40 (s, 9H).

### Step 2.

The final product was obtained following the same procedure of (S)-2-((((9H-fluoren-9-yl)methoxy)carbonyl)amino)-3-(4-(trifluoromethoxy)phenyl)propanoic acid. Removal of the tBu ester with HCl/dioxane afforded the desired (S)-2-((((9H-fluoren-9-yl)methoxy)carbonyl)amino)-3-(2,3-dimethylphenyl)propanoic acid (72.9 mg, 0.175 mmol, 77% yield) as a cream solid after purification by reverse phase flash chromatography. HPLC: RT=1.03 min (Waters Acquity UPLC BEH C18 1.7 μm 2.1×50 mm, CH₃CN/H₂O/0.05% TFA, 1 min. gradient, wavelength=254 nm); MS (ES): m z=416 [M+H]⁺. ¹H NMR (499 MHz, DMSO-d₆) δ 12.76 (br d, J=1.8 Hz, 1H), 7.89 (d, J=7.5 Hz, 2H), 7.79-7.71 (m, 1H), 7.66 (dd, J=13.6, 7.6 Hz, 2H), 7.42 (td, J=7.2, 4.1 Hz, 2H), 7.35-7.27 (m, 2H), 7.07 (d, J=7.3 Hz, 1H), 7.04-6.99 (m, 1H), 6.99-6.94 (m, 1H), 4.24-4.14 (m, 3H), 4.13-4.05 (m, 1H), 3.15 (dd, J=14.1, 4.1 Hz, 1H), 2.85 (dd, J=13.9, 10.4 Hz, 1H), 2.22 (s, 3H), 2.19 (s, 3H).

### Preparation of (S)-2-((((9H-fluoren-9-yl)methoxy)carbonyl)amino)-3-(2-fluoro-3-methylphenyl)propanoic acid

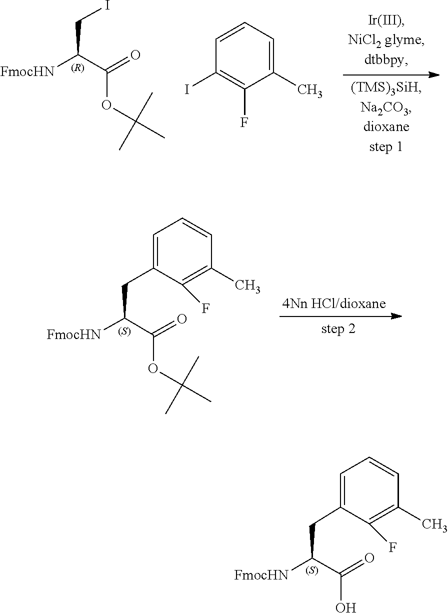

### Step 1

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2-fluoro-3-methylphenyl)propanoate (136.9 mg, LCMS showed 77% product and 23% impurity) as a viscous oil after purification by flash chromatography. Used as is, purify at after tBu hydrolysis.

### Step 2

The final product was obtained following the same procedure of (S)-2-((((9H-fluoren-9-yl)methoxy)carbonyl)amino)-3-(4-(trifluoromethoxy)phenyl)propanoic acid Removal of tBu ester with HCl/dioxane afforded the desired (S)-2-((((9H-fluoren-9-yl)methoxy)carbonyl)amino)-3-(2-fluoro-3-methylphenyl)propanoic acid (79.7 mg, 0.190 mmol, 66.0% yield) as a cream solid after purification by reverse phase flash chromatography. HPLC: RT=1.02 min (Waters Acquity UPLC BEH C18 1.7 μm 2.1×50 mm, CH₃CN/H₂O/0.05% TFA, 1 min. gradient, wavelength=254 nm); MS (ES): m z=420 [M+1]⁺. ¹H NMR (499 MHz, DMSO-d₆) δ 12.79 (br s, 1H), 7.89 (d, J=7.7 Hz, 2H), 7.78 (d, J=8.6 Hz, 1H), 7.65 (dd, J=11.6, 7.5 Hz, 2H), 7.44-7.39 (m, 3H), 7.37-7.25 (m, 3H), 7.14 (br t, J=7.4 Hz, 2H), 7.01-6.96 (m, 1H), 4.24-4.12 (m, 4H), 3.17 (dd, J=13.8, 4.8 Hz, 1H), 2.86 (dd, J=13.6, 10.8 Hz, 1H), 2.21 (s, 3H). ¹H NMR and LCMS showed a 14% impurity.

### Preparation of ((S)-2-((((9H-fluoren-9-yl)methoxy)carbonyl)amino)-3-(2-fluoro-5-methylphenyl)propanoic acid

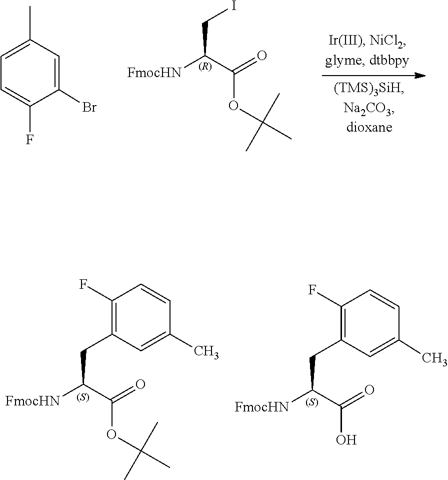

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2-fluoro-5-methylphenyl)propanoate (148.1 mg, 0.311 mmol, 65.4% yield) as a colourless gum after purification by flash chromatography. HPLC: RT=1.19 min (Waters Acquity UPLC BEH C18 1.7 μm 2.1×50 mm, CH₃CN/H₂O/0.05% TFA, 1 min. gradient, wavelength=254 nm); MS (ES): m z=420 [M-t-Bu]⁺. ¹H NMR (499 MHz, CHLOROFORM-d) δ 7.79 (d, J=7.6 Hz, 2H), 7.60 (t, J=7.2 Hz, 2H), 7.42 (t, J=7.4 Hz, 2H), 7.37-7.30 (m, 2H), 7.06-6.99 (m, 2H), 6.97-6.90 (m, 1H), 5.41 (br d, J=8.1 Hz, 1H), 4.60-4.54 (m, 1H), 4.43 (dd, J=10.4, 7.2 Hz, 1H), 4.30 (dd, J=10.1, 7.5 Hz, 1H), 4.26-4.21 (m, 1H), 3.16 (dd, J=13.9, 6.7 Hz, 1H), 3.10 (dd, J=13.9, 6.4 Hz, 1H), 2.28 (s, 3H), 1.44 (s, 9H).

### Step 2

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2-fluoro-5-methylphenyl)propanoic acid (98.1 mg, 0.23 mmol, 75% yield) as a colourless solid after purification by reverse phase flash chromatography. HPLC: RT=1.01 min (Waters Acquity UPLC BEH C18 1.7 μm 2.1×50 mm, CH₃CN/H₂O/0.05% TFA, 1 min. gradient, wavelength=254 nm); MS (ES): m z=420 [M+1]⁺. ¹H NMR (499 MHz, DMSO-d₆) δ 12.82 (br s, 1H), 7.89 (d, J=7.5 Hz, 2H), 7.78 (d, J=8.6 Hz, 1H), 7.67 (d, J=7.4 Hz, 1H), 7.64 (d, J=7.4 Hz, 1H), 7.42 (td, J=7.4, 3.0 Hz, 2H), 7.34-7.27 (m, 2H), 7.16-7.11 (m, 1H), 7.08-6.97 (m, 2H), 4.26-4.12 (m, 5H), 3.15 (dd, J=13.8, 4.9 Hz, 1H), 2.83 (dd, J=13.8, 10.3 Hz, 1H), 2.20 (s, 3H).

### Preparation of (S)-2-((((9H-fluoren-9-yl)methoxy)carbonyl)amino)-3-(2-fluoro-5-methoxyphenyl)propanoic acid

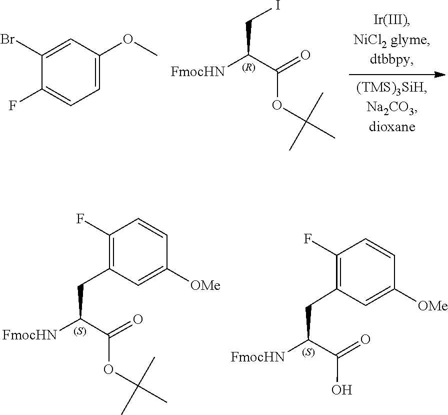

### Step 1

The compound was prepared following the same procedure of tert-butyl (S)-2-((((9H-fluoren-9-yl)methoxy)carbonyl)amino)-3-(2-fluoro-5-methoxyphenyl)propanoate (117.7 mg, 0.24 mmol, 50.4% yield) as a colourless solid after purification by flash chromatography. HPLC: RT=1.15 min (Waters Acquity UPLC BEH C18 1.7 μm 2.1×50 mm, CH₃CN/H₂O/0.05% TFA, 1 min. gradient, wavelength=254 nm); MS (ES): m z=436 [M-t-Bu]⁺.

¹H NMR (499 MHz, CHLOROFORM-d) δ 7.78 (d, J=7.5 Hz, 2H), 7.63-7.56 (m, 2H), 7.42 (t, J=7.4 Hz, 2H), 7.37-7.30 (m, 2H), 7.01-6.93 (m, 1H), 6.79-6.72 (m, 2H), 5.41 (br d, J=8.2 Hz, 1H), 4.62-4.55 (m, 1H), 4.41 (dd, J=10.4, 7.3 Hz, 1H), 4.31 (dd, J=10.5, 7.4 Hz, 1H), 4.26-4.20 (m, 1H), 3.75 (s, 3H), 3.17 (dd, J=13.9, 6.7 Hz, 1H), 3.11 (dd, J=14.4, 6.6 Hz, 1H), 1.45 (s, 9H).

### Step 2

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2-fluoro-5-methoxyphenyl)propanoic acid (79.5 mg, 0.183 mmol, 76% yield) as a colourless solid after purification by flash chromatography. HPLC: RT=0.98 min (Waters Acquity UPLC BEH C18 1.7 μm 2.1×50 mm, CH₃CN/H₂O/0.05% TFA, 1 min. gradient, wavelength=254 nm); MS (ES): m z=436 [M+1]⁺. Base peak of 214=fully deprotected amino acid fragment was also observed. ¹H NMR (499 MHz, DMSO-d₆) δ 12.84 (br s, 1H), 7.89 (d, J=7.5 Hz, 2H), 7.79 (d, J=8.6 Hz, 1H), 7.64 (t, J=8.4 Hz, 2H), 7.45-7.38 (m, 2H), 7.34-7.25 (m, 2H), 7.07 (t, J=9.2 Hz, 1H), 6.94 (dd, J=6.1, 3.2 Hz, 1H), 6.80 (dt, J=8.9, 3.6 Hz, 1H), 4.25-4.13 (m, 4H), 3.69 (s, 3H), 3.17 (dd, J=13.9, 4.6 Hz, 1H), 2.83 (dd, J=13.7, 10.7 Hz, 1H).

### Preparation of (S)-2-((((9H-fluoren-9-yl)methoxy)carbonyl)amino)-3-(2-methoxy-5-methylphenyl)propanoic acid

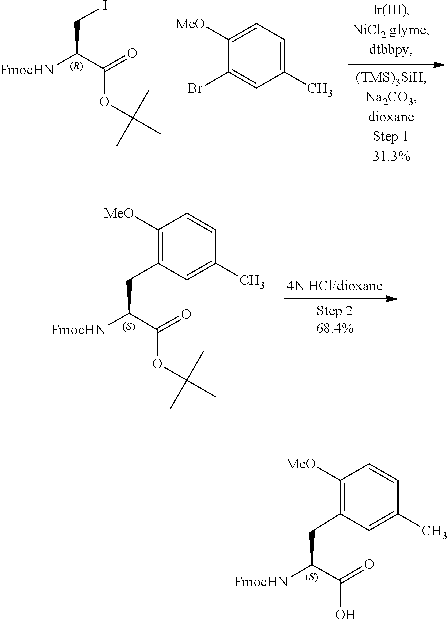

### Step 1.

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2-methoxy-5-methylphenyl)propanoate (73.9 mg, 0.15 mmol, 31.3% yield) as a colourless film after purification by flash chromatography. HIPLC: RT=1.20 min (Waters Acquity UPLC BEH C18 1.7 μm 2.1×50 mm, CH₃CN/H₂O/0.05% TFA, 1 min. gradient, wavelength=254 nm); MS (ES): m z=488 [M-tBu+H]⁺. ¹H NMR (499 MHz, CHLOROFORM-d) δ 7.78 (d, J=7.6 Hz, 2H), 7.61-7.54 (m, 2H), 7.41 (t, J=7.4 Hz, 2H), 7.34-7.30 (m, 2H), 7.05 (dd, J=8.1, 1.5 Hz, 1H), 6.98 (d, J=1.4 Hz, 1H), 6.79 (d, J=8.3 Hz, 1H), 5.70 (br d, J=7.7 Hz, 1H), 4.49 (q, J=7.4 Hz, 1H), 4.33 (d, J=7.4 Hz, 2H), 4.25-4.18 (m, 1H), 3.82 (s, 3H), 3.10-3.02 (m, 2H), 2.26 (s, 3H), 1.43 (s, 9H).

### Step 2.

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2-methoxy-5-methylphenyl)propanoic acid (44.7 mg, 0.104 mmol, 68.4% yield) as a colourless solid after purification by flash chromatography. HPLC: RT=1.02 min (Waters Acquity UPLC BEH C18 1.7 μm 2.1×50 mm, CH₃CN/H₂O/0.05% TFA, 1 min. gradient, wavelength=254 nm); MS (ES): m z=432 [M+H]⁺. ¹H NMR (499 MHz, DMSO-d₆) δ 12.61 (br s, 1H), 7.89 (d, J=7.5 Hz, 2H), 7.67 (d, J=7.5 Hz, 1H), 7.63 (d, J=7.5 Hz, 1H), 7.60 (br d, J=8.1 Hz, 1H), 7.42 (td, J=7.2, 3.5 Hz, 2H), 7.32 (td, J=7.5, 1.0 Hz, 1H), 7.30-7.26 (m, 1H), 7.02-6.97 (m, 2H), 6.84 (d, J=8.9 Hz, 1H), 4.26-4.10 (m, 4H), 3.75 (s, 3H), 3.12 (dd, J=13.5, 4.8 Hz, 1H), 2.72 (dd, J=13.4, 10.2 Hz, 1H), 2.16 (s, 3H).

### Preparation of (S)-2-((((9H-fluoren-9-yl)methoxy)carbonyl)amino)-3-hydroxy-3-methylbutanoic acid

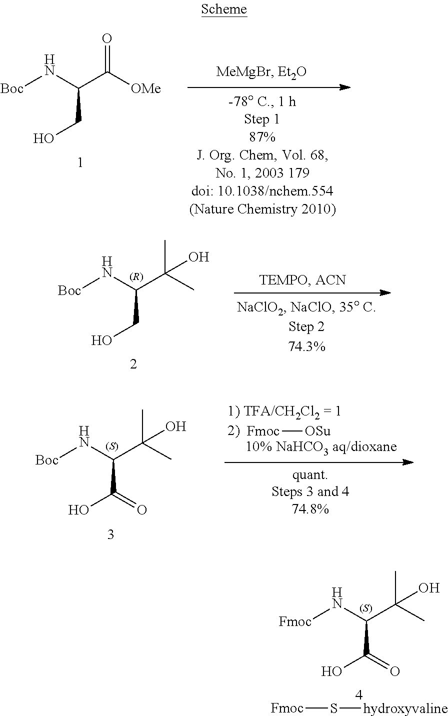

### Step 1.

To a 10-L multi-neck round-bottomed flask was charged methyl (tert-butoxycarbonyl)-D-serinate (50 g, 228 mmol), diethyl ether (4200 mL). The mixture was cooled to −78° C. and methylmagnesium bromide (456 mL, 1368 mmol) was added dropwise over 30 min. The reaction was stirred at RT for 1 h. It was cooled to 0° C. and saturated NH₄Cl solution (1500 mL), was added dropwise and stirred for 10 min. The organic layer was separated and the aqueous layer was extracted with ethyl acetate (3×2000 mL). The combined organic layer was washed with brine, dried over Na₂SO₄, and concentrated at 40° C. to give a colorless thick liquid. The crude was purified by I2PAC. Desired fractions were eluted at 50% EtOAc:petroleum ether mixture, and were collected and concentrated at 40° C. to give tert-butyl (R)-(J,3-dihydroxy-3-methylbutan-2-yl)carbamate (43.5 g, 87%) as a white solid. ¹H NMR (MeOD, 300 MHz) δ 3.70 (m, 1H), 3.48 (m, 1H), 3.21 (m, 1H), 1.35 (s, 9H), 1.13 (s, 3H), 1.05 (s, 3H).

### Step 2.

A 50-mL single neck round-bottomed flask was charged with tert-butyl (R)-(1,3-dihydroxy-3-methylbutan-2-yl)carbamate (43.0 g, 196 mmol), acetonitrile (650 mL) and was stirred till solution became clear. Sodium phosphate buffer (460 mL, 196 mmol) (pH=6.7, 0.67 M), (diacetoxyiodo)benzene (4.48 g, 13.92 mmol), and TEMPO (2.206 g, 14.12 mmol) were added sequentially and then the reaction was cooled to 0° C. and sodium chlorite (19.95 g, 221 mmol) was added. The color of the reaction turned black. The reaction was allowed to stir at 0° C. for 2 h. then at RT overnight. The orange colored reaction was quenched with saturated ammonium chloride solution (1000 mL) and the pH meter was used to adjust the pH=2 using 1.5 N HCl (330 mL). The aqueous solution was saturated with solid NaCl and extracted with ethyl acetate. The combined organic layer was washed with brine, dried over Na₂SO₄, and concentrated to obtain crude (S)-2-((tert-butoxycarbonyl)amino)-3-hydroxy-3-methylbutanoic acid (34.0 g, 74.3% yield) as an off-white solid and was taken directly to the next stage. ¹H NMR (MeOD, 300 MHz) δ 3.98 (s, 1H), 1.35 (s, 9H), 1.19 (s, 3H), 1.16 (9s, 3H).

### Step 3.

A 2000-mL single neck flask was charged with (S)-2-((tert-butoxycarbonyl)amino)-3-hydroxy-3-methylbutanoic acid (90 g, 386 mmol)dioxane (450 mL) and was cooled to 0° C. 4N HCl in Dioxane (450 mL, 1800 mmol) was added dropwise over 10 min. The reaction was allowed to stir at RT for 3 h. It was concentrated and azetroped with toluene (2×) then stirred with ethyl acetate for 10 min. It was filtered and dried under vacuum to obtain crude (S)-2-amino-3-hydroxy-3-methylbutanoic acid, HCl (70 g, 107% yield) as a white solid and was taken directly to the next step.

### Step 4

To a 3000-mL multi-neck round-bottomed flask was charged (S)-2-amino-3-hydroxy-3-methylbutanoic acid, HCl (70 g, 413 mmol), dioxane (1160 mL) and water (540 mL) The stirred solution became clear and a solution of sodium bicarbonate (104 g, 1238 mmol) in water (1160 mL) was added in one portion at RT. The reaction mass was allowed to stir at RT for 30 min. A solution of Fmoc-OSu (139 g, 413 mmol) in 1,4-dioxane (1460 mL) was added in one portion at RT. The reaction was allowed to stir at RT for 16 h. The reaction was concentrated to remove dioxane. To the resulting solution water was added and washed with ethyl acetate (3×1000 mL). The aqueous solution was acidified to pH 1-2 and extracted with ethyl acetate. The combined organic layer was washed with water, followed by brine, finally dried over Na₂SO₄, and concentrated to give an off-white solid (135.7 g). To remove the trapped dioxane and ethyl acetate the following proceture was followed: the solid was dissolved in ethyl acetate (1200 mL) and was stripped off with n-hexane (3000 mL). The slurry obtained was stirred for 10 min, filtered, dried under vacuum to give (S)-2-((((9H-fluoren-9-yl)methoxy)carbonyl)amino)-3-hydroxy-3-methylbutanoic acid (112.0 g, 74.8 yield for two steps) as a white solid.

### Preparation of (S)-2-((((9H-fluoren-9-yl)methoxy)carbonyl)amino)-3-(3,4,5-trifluorophenyl)propanoic acid

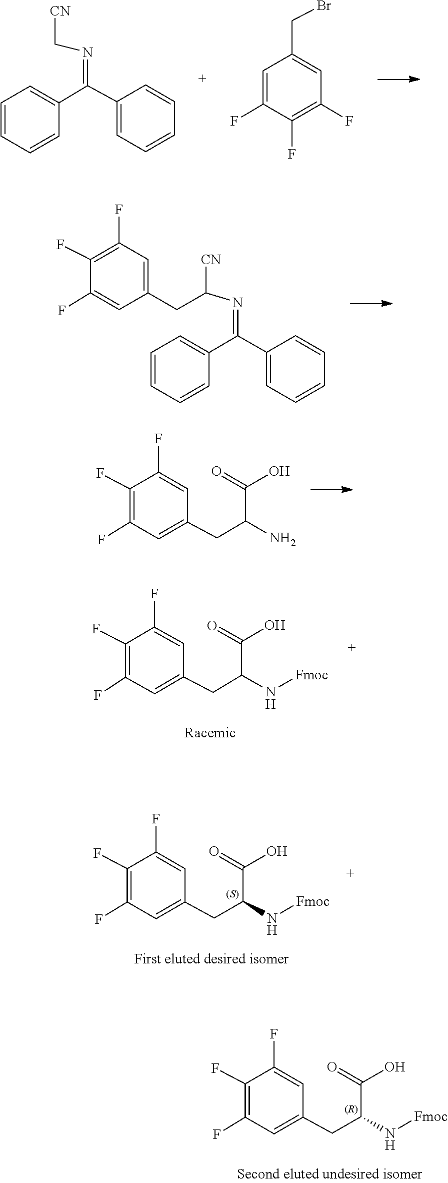

### Step 1.

To a stirred solution of 2-((diphenylmethylene)amino)acetonitrile (100 g, 454 mmol) in DCM (1000 mL), 5-(bromomethyl)-1,2,3-trifluorobenzene (66.5 mL, 499 mmol) and benzyltrimethylammonium chloride (16.86 g, 91 mmol) was added. To this, 10 M NaOH (136 mL, 1362 mmol) solution was added and stirred at rt overnight. After 26 h, the reaction mixture was diluted with water (500 mL) and the DCM layer was separated. The aqueous layer was further extracted with DCM (2×250 mL). The organic layer was combined, washed with water and brine solution, dried over Na₂SO₄, filtered, and concentrated under vacuum. The crude compound was purified by flash column chromatography (1.5 kg, silica gel, 0-10% ethylacetate/petroleum ether mixture) and the desired fractions were collected and concentrated to afford 2-((diphenylmethylene)amino)-3-(3,4,5-trifluorophenyl)propanenitrile (140 g, 384 mmol, 85% yield) as a yellow solid. Analysis condition E: Retention time=3.78 min; ESI-MS(+) m/z [M+H]⁺: 365.2.

### Step 2.

To a stirred solution of 2-((diphenylmethylene)amino)-3-(3,4,5-trifluorophenyl)propanenitrile (80 g, 220 mmol) in 1,4-dioxane (240 mL), was added conc. HCl (270 mL, 3293 mmol) and the mixture was stirred at 90° C. for 16 h. The reaction mixture was taken as such for next step. Step 3.

To the crude aqueous dioxane solution from the previous was added 10 N NaOH solution until the solution was neutral. Na₂CO₃ (438 mL, 438 mmol) was then added, followed by the addition of Fmoc-OSu (81 g, 241 mmol). The mixture was stirred at rt overnight. The aqueous solution was acidified with 1.5 N HCl till pH=2 and the solid formed was filtered, dried to afford the crude compound. It was slurried initially with 5% EtOAc/petroleum ether for 30 min and filtered. racemic 2-((((9H-fluoren-9-yl)methoxy)carbonyl)amino)-3-(3,4,5-trifluorophenyl)propanoic acid (90 g, 204 mmol, 93% yield) as an off-white solid. This racemic compound was separated into two isomers by SFC purification (column: Chiralcel OD-H, MeOH as co-solvent) to get the desired isomers. After concentration of the desired isomer, it was slurried with 5% EtOAc/petroleum ether and filtered to get (S)-2-((((9H-fluoren-9-yl)methoxy)carbonyl)amino)-3-(3,4,5-trifluorophenyl)propanoic acid (43 g, 95 mmol, 43.3% yield) as an off-white solid. ¹H NMR (MeOD, 400 MHz) δ 7.78 (d, J=7.2 Hz, 2H), 7.60 (t, J=8.0 Hz, 2H), 7.38 (t, J=8.0 Hz, 2H), 7.28 (t, J=7.6 Hz, 2H), 7.01 (t, J=7.8 Hz, 2H), 4.48-4.26 (m, 3H), 4.18 (m, 1H), 3.18 (m, 1H), 2.91 (m, 1H). ¹⁹F (MeOD, 376 MHz) δ-137.56 (d, J=19.6 Hz, 2F), -166.67 (t, J=19.6 Hz, 1F). Analysis condition E: Retention time=3.15 min; ESI-MS(+) m/z [M+H]⁺: 442.2.

The other fraction was concentrated to get (R)-2-((((9H-fluoren-9-yl)methoxy)carbonyl)amino)-3-(3,4,5-trifluorophenyl)propanoic acid (40 g, 91 mmol, 41.4% yield) as an off-white solid.

### Preparation of (S)-2-((((9H-fluoren-9-yl)methoxy)carbonyl)amino)-4-(tert-butoxy)-3,3-dimethyl-4-oxobutanoic acid

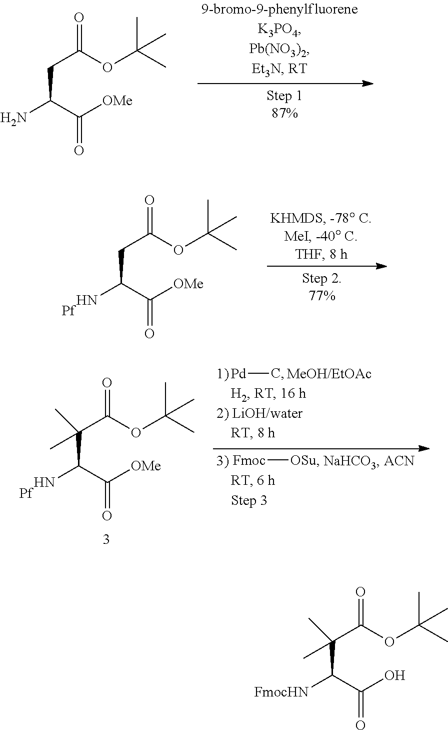

### Step 1.

To a stirred solution of 4-(tert-butyl) 1-methyl L-aspartate, HCl salt (34 g, 142 mmol) in acetonitrile (550 mL), was added lead(II) nitrate (47.0 g, 142 mmol), potassium phosphate (66.2 g, 312 mmol), and TEA (19.77 mL, 142 mmol) under nitrogen atmosphere. The mixture was cooled to 0° C. then a solution of 9-bromo-9-phenylfluorene (43.3 g, 135 mmol) in acetonitrile (100 mL) was added. The reaction mixture was stirred at RT for 48 h and the reaction progress was monitored by TLC (50% EA in PE) and LCMS. The reaction mixture was filtered over celite, washed with chloroform, and evaporated to get thick pale yellow liquid, to which ethyl acetate (3500 mL) was added. The EtOAc layer was washed with 5% citric acid solution (500 mL) followed by brine solution. The organic layer was dried over sodium sulfate and evaporated under reduced pressure to get pale yellow thick liquid, which was scratched with petroleum ether and filtered to obtain 4-(tert-butyl) 1-methyl (9-phenyl-9H-fluoren-9-yl)-L-aspartate (55 g, 124 mmol, 87% yield) as a white solid. Analysis condition L: Retention time=1.73 min; ESI-MS(+) m/z [M+Na]⁺: 466.40.

### Step 2.

A solution of 4-(tert-butyl) 1-methyl (9-phenyl-9H-fluoren-9-yl)-L-aspartate (22.5 g, 50.7 mmol) was cooled to −78° C. under Ar and a solution of KHMDS (127 mL, 127 mmol, 1 M in THF) was added over 30 min while stirring. The reaction was allowed to warm to −40° C., and methyl iodide (9.52 mL, 152 mmol) was added dropwise. The reaction was stirred at −40° C. for 5 h. The reaction was monitored by TLC and LCMS. Saturated NH₄Cl (400 mL) was added followed by H₂O (100 mL). The resulting mixture was extracted with EtOAc (3×) and the combined organic extracts were washed with 2% citric acid (200 mL), aq. NaHCO₃ (200 mL), and brine. The organic layer was dried over anhydrous Na₂SO₄, evaporated in vacuo, and recrystallized from hexanes to give 1-(tert-butyl) 4-methyl (S)-2,2-dimethyl-3-((9-phenyl-9H-fluoren-9-yl)amino)succinate (18.5 g, 39.2 mmol, 77% yield) as a white solid, which was taken for next step. Analysis condition L: Retention time=2.04 min; ESI-MS(+) m/z [M+Na]⁺: 494.34.

### Step 3.

A stirred solution of 1-(tert-butyl) 4-methyl (S)-2,2-dimethyl-3-((9-phenyl-9H-fluoren-9-yl)amino)succinate (24 g, 50.9 mmol) in methanol (270 mL) and ethyl acetate (100 mL) was degassed with nitrogen. Pd-C(2.71 g, 2.54 mmol) (10% by weight) was added, and the mixture was flushed with hydrogen gas and then stirred at RT in 1-liter capacity autoclave with 50 psi overnight. The reaction mixture was filtered through celite pad, washed with a mixture of methanol and ethyl acetate. The combined solvents were evaporated to dryness and the precipitated white solid was removed by filtration to obtain a pale yellow liquid 1-(tert-butyl) 4-methyl (S)-3-amino-2,2-dimethylsuccinate (11.7 g) which was taken as such for the next step.

### Step 4.

To a stired solution of 1-(tert-butyl) 4-methyl (S)-3-amino-2,2-dimethylsuccinate (11.0 g, 47.6 mmol)cooled in an ice bath, was added lithium hydroxide (428 mL, 86 mmol, 0.2 M solution in water) and the reaction was slowly brought to RT. The reaction was monitored by TLC and LCMS. The reaction mixture was evaporated and directly taken to the next step. To a stirred solution of (S)-2-amino-4-(tert-butoxy)-3,3-dimethyl-4-oxobutanoic acid (15 g, 69.0 mmol) (which was in water from the previous batch) in acetonitrile (200 mL) cooled to 0° C., was added sodium bicarbonate (5.80 g, 69.0 mmol) and Fmoc-OSu (46.6 g, 138 mmol). The reaction mixture was stirred at RT overnight. It was acidified with 2 N HCl to pH=4, then extracted with ethyl acetate (3×500 mL), and the combined organic layer was washed with brine, dried over sodium sulfate, and evaporated to get an off-white solid, which was purified by ISCO flash chromatography with 20% EA in petroleum ether to get (S)-2-((((9H-fluoren-9-yl)methoxy)carbonyl)amino)-4-(tert-butoxy)-3,3-dimethyl-4-oxobutanoic acid (12.2 g, 26.9 mmol, 39.0% yield) as a white solid. ¹HNMR (CDCl₃, 400 MHz) δ 7.77 (d, J=7.6 Hz, 2H), 7.60 (m, 2H), 7.42 (t, J=8.0 Hz, 2H), 7.33 (t, J=7.6 Hz, 2H), 4.65 (m, 2H), 4.34 (m, 1H), 4.25 (m, 1H), 3.18 (m, 1H), 1.40-1.27 (m, 6H). Analysis condition E: Retention time=1.90 min; ESI-MS(+) m/z [M+H]⁺: 440.2.

### Preparation of (S)-2-((((9H-fluoren-9-yl)methoxy)carbonyl)amino)-3-(3-(tert-butoxycarbonyl)phenyl)propanoic acid

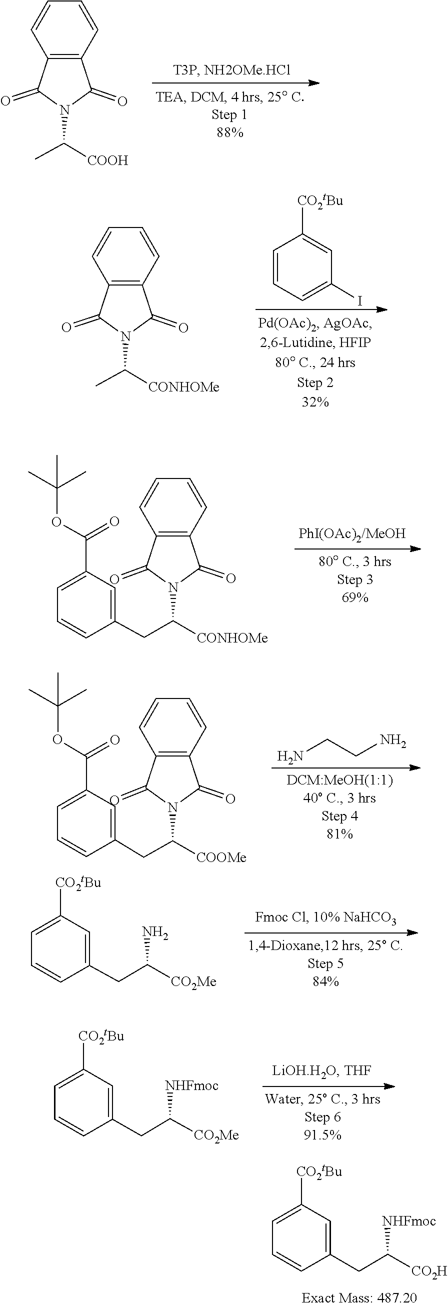

### Step 1.

To a solution of (S)-2-(1,3-dioxoisoindolin-2-yl)propanoic acid (80 g, 365 mmol), O-methylhydroxylamine hydrochloride (36.6 g, 438 mmol) in CH₂Cl₂ (2000 mL), was added TEA (153 mL, 1095 mmol) at RT. The reaction was cooled to 0° C., 1-propanephosphonic anhydride (326 mL, 547 mmol) was added dropwise. The reaction was stirred at RT for 2 h. It was quenched with saturated ammonium chloride (500 mL) and extracted with EtOAc (3×300 mL). The combined organic layers were washed with saturated brine, dried over Na₂SO₄, and concentrated under reduced pressure. The crude product was purified via combiflash using 120 g silica column with 38 to 45% EtOAc in petroleum ether to give (S)-2-(1,3-dioxoisoindolin-2-yl)-N-methoxypropanamide (80 g, 322 mmol, 88% yield). ¹H NMR (DMSO-d₆, 400 MHz) δ 11.36 (s, 1H), 7.91-7.85 (m, 4H), 4.75-4.69 (m, 1H), 3.56 (s, 3H), 1.51 (d, J=7.6 Hz, 3H).

### Step 2.

To a solution of (S)-2-(1,3-dioxoisoindolin-2-yl)-N-methoxypropanamide (20 g, 81 mmol), palladium(II) acetate (1.809 g, 8.06 mmol), silver acetate (26.9 g, 161 mmol) placed in a 1000-mL seal tube, was added tert-butyl 3-iodobenzoate (36.8 g, 121 mmol), 2,6-Lutidine (2.395 mL, 24.17 mmol), HFIP (300 mL) at 25° C. under N₂ atmosphere. The reaction was stirred for 15 min at 25° C. under N₂ and then heated up to 80° C. for 24 h with vigorous stirring. The reaction mixture was filtered through CELITE and washed with DCM (200 mL). The combined organic layer was concentrated under reduced pressure. The crude product was purified via combiflash using 220 g silica column eluting with 25 to 30% EtOAc:CHCl₃ to obtain the desired product tert-butyl (S)-3-(2-(1,3-dioxoisoindolin-2-yl)-3-(methoxyamino)-3-oxopropyl)benzoate (11 g, 25.9 mmol, 32.2% yield). Analysis condition E: Retention time=2.52 min; ESI-MS(+) m/z [M−H]⁺: 423.2. ¹H NMR (DMSO-d₆, 400 MHz) δ 11.46 (s, 1H), 7.82 (m, 4H), 7.63 (d, J=7.6 Hz, 1H), 7.54 (s, 1H), 7.40 (d, J=7.6 Hz, 1H), 7.30 (t, J=7.6 Hz, 1H), 4.93-4.89 (m, 1H), 3.59 (s, 3H), 3.56-3.49 (m, 1H), 3.36-3.27 (m, 1H), 1.40 (s, 9H).

### Step 3.

To a solution of tert-butyl (S)-3-(2-(1,3-dioxoisoindolin-2-yl)-3-(methoxyamino)-3-oxopropyl)benzoate (15 g, 35.3 mmol) in methanol (200 mL), (diacetoxyiodo)benzene (12.52 g, 38.9 mmol) was added at RT. The temperature was slowly raised to 80° C. and stirred for 3 h at 80° C. The Reaction was concentrated under reduced pressure to get the crude product. It was purified with silica gel chromatography (100-200 mesh eluting with 20% EA: hexane) to obtain the desired compound tert-butyl (S)-3-(2-(1,3-dioxoisoindolin-2-yl)-3-methoxy-3-oxopropyl)benzoate (10 g, 24.42 mmol, 69.1% yield. ¹H NMR (CDCl₃, 400 MHz) δ 7.80-7.76 (m, 4H), 7.72-7.68 (m, 2H), 7.34-7.26 (m, 1H), 7.25-7.23 (m, 1H), 5.14 (dd, J=10.8, 5.6 Hz, 1H), 3.76 (s, 3H), 3.65-3.49 (m, 2H), 1.50 (s, 9H).

### Step 4.

To a solution of tert-butyl (S)-3-(2-(1,3-dioxoisoindolin-2-yl)-3-methoxy-3-oxopropyl)benzoate (15 g, 36.6 mmol) in methanol (25 mL) ethylenediamine (12.25 mL, 183 mmol) was added at RT. The reaction temperature was slowly raised to 40° C. and stirred for 3 h at 40° C. The mixture was concentrated under reduced pressure to get the crude product. It was purified with silica gel chromatography (100-200 mesh eluting with 20% EA: hexane) to obtain the desired compound tert-butyl (S)-3-(2-amino-3-methoxy-3-oxopropyl)benzoate (8.3 g, 29.7 mmol, 81% yield). ¹H NMR (DMSO-d₆, 400 MHz) δ 8.32 (s, 1H), 7.77-7.72 (m, 2H), 7.46-7.38 (m, 1H), 3.61-3.57 (m, 4H), 2.96-2.91 (m, 1H), 2.85-2.82 (m, 1H), 1.79 (br. s, 2H), 1.55 (s, 9H).

### Step 5.

To a solution of tert-butyl (S)-3-(2-amino-3-methoxy-3-oxopropyl)benzoate (10 g, 35.8 mmol) in dioxane (150 mL), sodium bicarbonate (6.01 g, 71.6 mmol) was added followed by the addition of 9-fluorenylmethyl chloroformate (13.89 g, 53.7 mmol) at RT. The reaction was stirred for 12 h at RT. It was diluted with water and extracted with ethyl acetyate. The organic layer was concentrated under reduced pressure to get the crude product. It was purified via silica gel chromatography (100-200 mesh eluting with 20% EA: hexane) to obtain the desired compound tert-butyl (S)-3-(2-((((9H-fluoren-9-yl)methoxy)carbonyl)amino)-3-methoxy-3-oxopropyl)benzoate (15 g, 29.9 mmol, 84% yield).

### Step 6.

To a solution of tert-butyl (S)-3-(2-((((9H-fluoren-9-yl)methoxy)carbonyl)amino)-3-methoxy-3-oxopropyl)benzoate (18.00 g, 35.9 mmol) in THE (150 mL) and H₂O (150 mL) at RT, lithium hydroxide monohydrate (1.66 g, 39.5 mmol) was added. The reaction was stirred for 2 h at RT. The reaction was concentrated under reduced pressure to remove THF. In the basic medium the mixture was extracted with diethyl ether to remove the non polar impurities. The aqueous layer was acidified with aqueous citric acid solution and extracted with ethyl acetate. The organic layer was dried over sodium sulphate and concentrated under reduced to get the desired compound as a gummy solid which was further lyopholized to give off-white solids. the desired compound Lot 1: (S)-2-((((9H-fluoren-9-yl)methoxy)carbonyl)amino)-3-(3-(tert-butoxycarbonyl)phenyl)propanoic acid (11 g, 22.56 mmol, 62.9% yield). And lot 2: (S)-2-((((9H-fluoren-9-yl)methoxy)carbonyl)amino)-3-(3-(tert-butoxycarbonyl)phenyl)propanoic acid (5 g, 10.26 mmol, 28.6% yield). 7.86 (t, J=7.6 Hz, 2H), 7.75 (d, J=7.6 Hz, 1H), 7.66-7.59 (m, 2H), 7.52 (m, 2H), 7.41-7.37 (m, 3H), 7.31-7.24 (m, 2H), 4.21-4.16 (m, 4H), 3.17 (m, 1H), 2.96 (m, 1H), 1.53 (br, s. 9H). Analysis condition E: Retention time=3.865 min; ESI-MS(+) m/z [M−H]⁺: 486.2.

### Preparation of (S)-2-((((9H-fluoren-9-yl)methoxy)carbonyl)amino)-3-(m-tolyl)propanoic acid

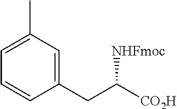

Compound was synthesized following the similar procedures of (S)-2-((((9H-fluoren-9-yl)methoxy)carbonyl)amino)-3-(3-(tert-butoxycarbonyl)phenyl)propanoic acid. Analysis condition E: Retention time=3.147 min; ESI-MS(+) m/z [M+H]⁺: 402.0. ¹H NMR (DMSO-d6, 300 MHz) δ 7.88 (d, J=7.5 Hz, 2H), 7.64 (t, J=6.8 Hz, 2H), 7.44 (t, J=7.5 Hz, 2H), 7.36-7.28 (m, 2H), 7.18 (t, J=7.5 Hz, 1H), 7.09-7.02 (m, 3H), 4.24-4.17 (m, 4H), 3.21-3.04 (m, 1H), 2.89-2.81 (m, 1H), 2.26 (s, 3H) ppm.

### Preparation ethyl (S)-5-((tert-butoxycarbonyl)amino)-2-(((S)-mesitylsulfinyl)amino)-3, 3-dimethylpentanoate

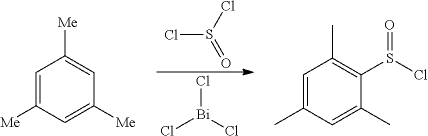

### Step 1:

The compound was synthesized using similar procedure described in reference: To a 1000-mL flask equipped with a septum inlet and magnetic stirring bar was added bismuth(III) chloride (5.25 g, 16.64 mmol). The flask was connected to an argon line and thionyl chloride (501 mL, 6864 mmol) were added by syringe. To the suspension was added mesitylene (100 g, 832 mmol). The flask was equipped with a condenser, connected to an oil bubbler and the reaction mixture was heated in an oil bath at 60° C. for 5 h. During this time the color of the solution became red-orange and HCl evolved from the solution. The reaction was monitored by LCMS. The flask was cooled in an ice bath and the excess of thionyl chloride was removed under reduced pressure yielding to an orange liquid. In order to remove the catalyst, 2000 mL of pentane were added, stirred and filtered through celite, and the bed was washed with pentane (2×500 mL). The organic phase was collected and evaporated under reduced pressure to give 2,4,6-trimethylbenzenesulfinic chloride (151 g, 745 mmol, 90% yield) as a pale yellow solid. The compound was taken to the next step without further purification. ¹H NMR (400 MHz, CDCl₃) δ 7.07-6.76 (m, 2H), 2.66 (s, 6H), 2.38-2.24 (m, 3H) ppm.

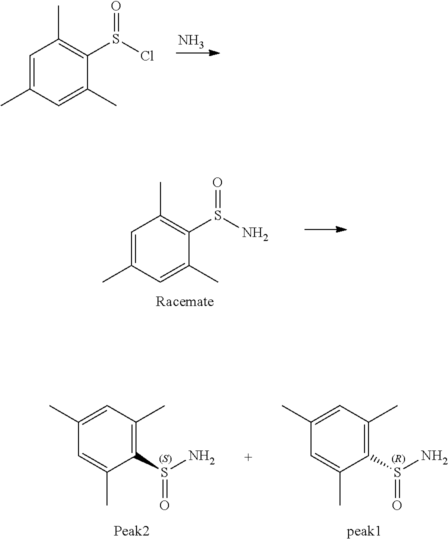

### Step 2.

The compound was synthesized using similar procedure described in reference: To a stirred solution of 2,4,6-trimethylbenzenesulfinic chloride (155 g, 765 mmol) in diethyl ether (1500 mL). After it had been cooled to −40° C. In a separate setup, (2L multi neck RBF) taken in diethyl ether (900 mL) ammonia gas was bubbled 30 minutes at −40° C., this purged solution was added to above reaction mass at −40° C. After it had warmed to rt the reaction mixture was stirred for 2 hours and monitored by open access LCMS starting material was absent. The reaction was stirred at room temperature overnight according to given procedure. The reaction was monitored by TLC and open access LCMS (A1CE6-344-01), TLC wise starting material was absent. Workup: The reaction mixture was diluted with ethyl acetate (3000 mL) and washed with water(2000 mL), the organic layer was separated and the aqueous phase was again extracted with ethyl acetate(1×500 mL).The combined organic layer washed with brine(1×800 mL). The combined organic layer, dried (Na₂SO₄), filtered, and concentrated under reduced pressure to obtained (235g) as a pale brown solid. The product (235 g) was recrystallized from 10% ethyl acetate/petroleum ether (500 mL), stirred, filtered, and dried to afford mesitylenesulphinamid (125 g) racemate as a white solid. The compound was submitted for the SFC method development. Two peaks were collected from SFC. The solvent was concentrated to give Peak-1 (Undesired): (R)-2,4,6-trimethylbenzenesulfinamide (51.6 g, 265 mmol, 34.6% yield) as a white colour solid. ¹H NMR (400 MHz, DMSO-d₆) δ 7.01-6.68 (m, 2H), 6.23-5.77 (m, 2H), 2.52-2.50 (m, 6H), 2.32-1.93 (m, 3H) and Peak-2 (desired): (S)-2, 4,6-trimethylbenzenesulfinamide (51.6 g, 267 mmol, 35.0% yield) as a white colour solid. ¹H NMR (400 MHz, DMSO-d₆) δ 6.87 (s, 2H), 6.16-5.82 (m, 2H), 2.53-2.50 (m, 6H), 2.34-1.93 (m, 3H).

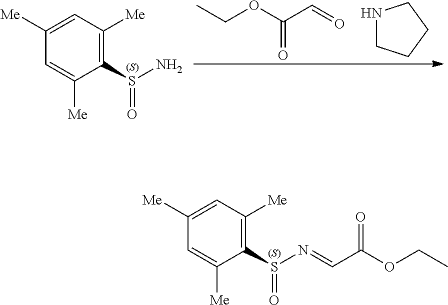

### Step 3.

The compound was synthesized using similar procedure described in reference: To a well stirred solution of (S)-2,4,6-trimethylbenzenesulfinamide (15.5 g, 85 mmol) in dichloromethane (235 mL) and 4A molecular sieves (84.5 g), was added ethyl 2-oxoacetate in toluene (25.9 mL, 127 mmol) and pyrrolidine (0.699 mL, 8.46 mmol). The reaction mixture was stirred at room temperature for overnight. The reaction was repeated and the two batches were combined together for work up. The reaction was mass was filtered throw the celite and the bed was washed with DCM. The solvents were removed under reduced pressure to obtained the crude (55 g) as a brownish color mass. The crude compound was purified by ISCO (Column size: 300 g silica column. Adsorbent: 60-120 silica mesh, Mobile phase:40% EtOAc/Pet ether) and the product was collected at 15-20% of EtOAc. The fractions were concentrated to obtain ethyl (S,E)-2-((mesitylsulfinyl)imino)acetate (16.5 g, 57.4 mmol, 67.9% yield) as a colorless liquid. The compound slowly solidified as an off white solid. ¹H NMR (400 MHz, CDCl₃) δ=8.27 (s, 1H), 7.04-6.70 (m, 2H), 4.59-4.21 (m, 2H), 2.55-2.44 (m, 6H), 2.36-2.23 (m, 3H), 1.51-1.30 (m, 3H). 2.670 min. 268.2 (M+H).

### Step 4.

General procedure for the synthesis of TCNHPI redox-active esters as in reference ACIE: TCNHPI esters were prepared according to the previously reported general procedure (ACIE paper and references therein): A round-bottom flask or culture tube was charged with carboxylic acid (1.0 equiv), N-hydroxytetrachlorophthalimide (1.0-1.1 equiv) and DMAP (0.1 equiv). Dichloromethane was added (0.1-0.2 M), and the mixture was stirred vigorously. Carboxylic acid (1.0 equiv) was added. DIC (1.1 equiv) was then added dropwise via syringe, and the mixture was allowed to stir until the acid was consumed (determined by TLC). Typical reaction times were between 0.5 h and 12 h. The mixture was filtered (through a thin pad of Celite®, SiO₂, or frit funnel) and insed with additional CH₂Cl₂/Et20. The solvent was removed under reduced pressure, and purification of the crude mixture by column chromatography afforded the desired TCNHPI redox-active ester. If necessary, the TCNHPI redox-active ester could be further recrystallized from CH₂Cl₂/MeOH.

### Step 5.

4,5,6, 7-tetrachloro-1, 3-dioxoisoindolin-2-yl-4-((tert-butoxycarbonyl)amino)-2,2-dimethylbutanoate was obtained as a white solid following General Procedure for the synthesis of TCNHPI redox-active esters on 5.00 mmol scale. Purification by column (silica gel, gradient from CH₂Cl₂ to 10:1 CH₂Cl₂:Et20) afforded 2.15g (84%) of the title compound. ¹H NMR (400 MHz, CDCl₃): δ 4.89 (br s, 1H), 3.30 (q, J=7.0 Hz, 2H), 1.98 (t, J=7.6 Hz, 2H), 1.42 (s, 15H) ppm. ¹³C NMR (151 MHz, CDCl₃): δ 173.1, 157.7, 156.0, 141.1, 130.5, 124.8, 79.3, 40.8, 40.2, 36.8, 28.5, 25.2 ppm. HRMS (ESI-TOF): calc'd for C₁₉H₂₀Cl₄N₂NaO₆ [M+Na]⁺: 534.9968, found: 534.9973.

### Step 6.

Ethyl (S)-5-((tert-butoxycarbonyl)amino)-2-(((S)-mesitylsulfinyl)amino)-3,3-dimethylpentanoate was made using the General procedures for decarboxylative Amino acid synthesis in reference ACIE. A culture tube was charged with TCNHPI redox-active ester A (1.0 mmol), sulfinimine B (2.0 mmol), Ni(OAc)2.4H₂O (0.25 mmol, 25 mol %), Zinc (3 mmol, 3 equiv). The tube was then evacuated and backfilled with argon (three times). Anhydrous NMP (5.0 mL, 0.2 M) was added using a syringe. The mixture was stirred overnight at rt. Then, the reaction mixture was diluted with EtOAc, washed with water,brine and dried over MgSO₄. Upon filtration, the organic layer was concentrated under reduced pressure (water bath at 30° C.), and purified by flash column chromatography (silica gel) to provide the product. Purification by column (2:1 hexanes:EtOAc) afforded 327.6 mg (72%) of the title compound ethyl (S)-5-((tert-butoxycarbonyl)amino)-2-(((S)-mesitylsulfinyl)amino)-3,3-dimethylpentanoate as a colorless oil. ¹H NMR (600 MHz, CDCl₃): δ 6.86 (s, 2H), 5.04 (d, J=10.1 Hz, 1H), 4.47 (s, 1H), 4.28-4.16 (m, 2H), 3.66 (d, J=10.1 Hz, 1H), 3.27-3.05 (m, 2H), 2.56 (s, 6H), 2.28 (s, 3H), 1.54-1.46 (m, 2H), 1.43 (s, 9H), 1.30 (t, J=7.2 Hz, 3H), 0.96 (s, 6H) ppm. ¹³C NMR (151 MHz, CDCl₃): δ 172.5, 155.9, 141.1, 137.9, 136.9, 131.0, 79.4, 65.5,61.7, 38.8, 37.1, 36.5, 28.5, 23.9, 23.6, 21.2, 19.4, 14.3 ppm. HRMS (ESI-TOF): calc'd for C₂₃H₃₉N₂O₅S [M+H]⁺: 455.2574, found: 455.2569.

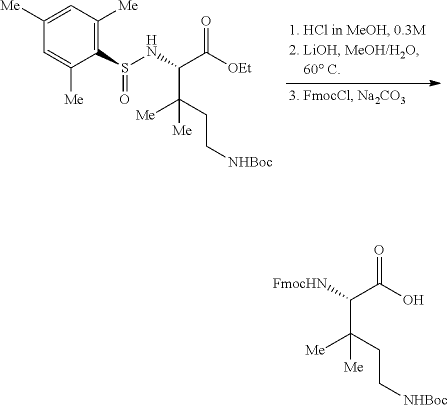

### Step 7.

### 2-((((9H-fluoren-9-yl)methoxy)carbonyl)amino)-5-((tert-butoxycarbonyl)amino)-3,3

dimethylpentanoic acid: A culture tube was charged with ethyl (S)-5-((tert-butoxycarbonyl)amino)-2-(((S)-mesitylsulfinyl)amino)-3,3-dimethylpentanoate (0.5 mmol, 1.0 equiv), HCl (4.0 equiv) in MeOH (0.3 M) was added via syringe and the resulting mixture was stirred at RT for ca. 10 min (screened by TLC). After the reaction, Et3N was added until pH=7 and the solvents were removed under reduced pressure. LiOH (2 equiv) in MeOH/H₂O (2:1, 0.04 M) was added to the crude mixture. The reaction was stirred at 60° C. overnight. On completion, HCl in MeOH (0.3 M) was added until pH=7 and the solvents were removed under reduced pressure. The crude mixture was dissolved in 9% aqueous Na₂CO₃ (5 mL) and dioxane (2 mL). It was slowly added at 0° C. to a solution of Fmoc-OSu (1.2 equiv) in dioxane (8 mL). The mixture was stirred at 0° C. for 1 h and then allowed to warm to rt. After 10 h, the reaction mixture was quenched with HCl (0.5 M), reaching pH 3, and then diluted with EtOAc. The aqueous phase was extracted with EtOAc (3×15 mL), and the combined organic layers were washed with brine, dried over Na₂SO₄, filtered, and the solvent was removed under reduced pressure. The crude mixture was then purified by flash column chromatography (silica gel, 2:1 hexanes:EtOAc) to afford the product ethyl (S)-5-((tert-butoxycarbonyl)amino)-2-(((S)-mesitylsulfinyl)amino)-3,3-dimethylpentanoate in 68% overall yield and 95% ee as a colorless oil. ¹H NMR (600 MHz, CDCl₃): δ 7.76 (d, J=7.5 Hz, 2H), 7.63-7.54 (m, 2H), 7.39 (td, J=7.3, 2.6 Hz, 2H), 7.33-7.28 (m, 2H), 5.50 (br s, 1H), 4.68 (br s, 1H), 4.45-4.43 (m, 1H), 4.38-4.35 (m, 1H), 4.30 (d, J=7.9 Hz, 1H), 4.21 (t, J=6.8 Hz, 1H), 3.27 (br s, 1H), 3.16 (br s, 1H), 1.63-1.50 (m, 2H), 1.43 (s, 9H), 1.09-0.76 (m, 6H) ppm. ¹³C NMR (151 MHz, CDCl₃): δ 185.8, 174.3, 156.5, 144.0, 143.9, 141.5, 127.9, 127.2, 125.24, 125.21, 120.2, 120.1, 79.8, 67.2, 60.9, 47.4, 39.2, 36.8, 29.9, 28.6, 23.9 ppm. HRMS (ESI-TOF): calc'd for C₂₇H₃₅N₂O₆ [M+H]⁺: 483.2490, found: 483.2489.

### Preparation of (S)-2-((((9H-fluoren-9-yl)methoxy)carbonyl)amino)-3-(4,4-difluorocyclohexyl)propanoic acid

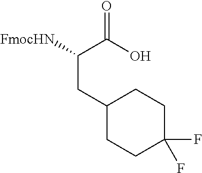

Final product was obtained following similar procedures of ethyl (S)-5-((tert-butoxycarbonyl)amino)-2-(((S)-mesitylsulfinyl)amino)-3,3-dimethylpentanoate. The synthesis afforded the desired (S)-2-((((9H-fluoren-9-yl)methoxy)carbonyl)amino)-3-(4,4-difluorocyclohexyl)propanoic acid (60 mg, 0.14 mmol, 27.9% yield) as a white solid after purification by reverse phase HPLC. ¹H NMR (500 MHz, CDCl₃) δ 7.79 (br d, J=7.5 Hz, 2H), 7.61 (br s, 2H), 7.43 (s, 2H), 7.36-7.31 (m, 2H), 5.24-5.06 (m, 1H), 4.57-4.36 (m, 3H), 4.29-4.16 (m, 1H), 2.19-1.99 (m, 2H), 1.97-1.18 (m, 9H).

### Preparation of (2S)-5-(tert-butoxy)-2-({[(9H-fluoren-9-yl)methoxy]carbonyl}amino)-3,3-dimethyl-5-oxopentanoic acid

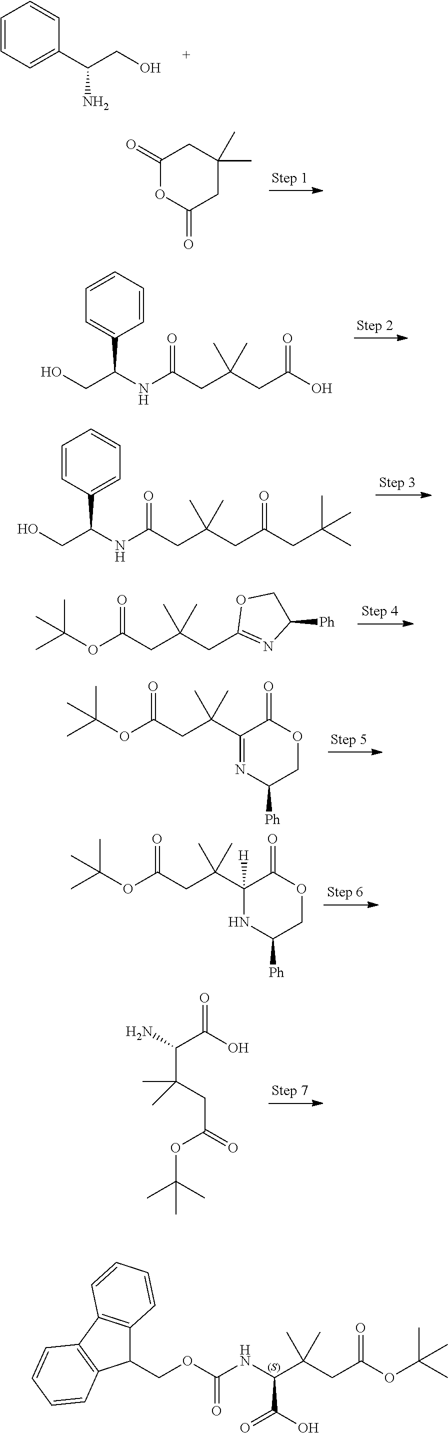

### Step 1.

A solution of 4,4-dimethyldihydro-2H-pyran-2,6(3H)-dione (8.29 g, 58.3 mmol) in dry toluene (100 mL) was slowly added to a solution of (R)-2-amino-2-phenylethan-1-ol (10 g, 72.9 mmol) in dry toluene (100 mL) and CH₂Cl₂ (20 mL) at room temperature. The reaction mixture was then heated to 60° C. and reacted for 12 h. It was cooled to room temperature until a white solid was formed. The solid was filtered and washed with 1:1 EtOAc/CH₂Cl₂ to afford the crude desired compound (R)-5-((2-hydroxy-1-phenylethyl)amino)-3,3-dimethyl-5-oxopentanoic acid (11.9 g, 41.0 mmol, 56.2% yield) without further purification. ¹H NMR (300 MHz, DMSO-d₆) δ 8.41 (br d, J=7.9 Hz, 1H), 7.44-7.32 (m, 2H), 7.32-7.27 (m, 4H), 7.26-7.18 (m, 1H), 4.89-4.80 (m, 1H), 4.14-3.98 (m, 1H), 3.63-3.43 (m, 3H), 2.27-2.18 (m, 4H), 2.08 (s, 1H), 1.99 (s, 1H), 1.17 (t, J=7.2 Hz, 1H), 1.00 (d, J=4.5 Hz, 6H), 0.92 (s, 1H).

### Step 2.

(R)-5-((2-Hydroxy-1-phenylethyl)amino)-3,3-dimethyl-5-oxopentanoic acid (12 g, 43.0 mmol) was dissolved in a solution of benzyltrimethylammonium chloride (8.93 g, 48.1 mmol) in DMA (250 mL). K₂CO₃ (154 g, 1117 mmol) was added to the above solution followed by the addition of 2-bromo-2-methylpropane (235 mL, 2091 mmol). The reaction mixture was stirred at 55° C. for 24 h. The reaction mixture was then diluted with EtOAc (100 mL), washed with H₂O (50 mL×3), and brine (50 mL). The organic phase was dried over Na₂SO₄, concentrated under vacuo, and purified by flash column chromatography on silica gel (CH₂Cl₂/MeOH, 15:1) to give tert-butyl (R)-5-((2-hydroxy-1-phenylethyl)amino)-3,3-dimethyl-5-oxopentanoate (6.0 g, 17.89 mmol, 41.6% yield). Analytical LC/MS Condition M: 1.96 min, 336.3 [M+H]⁺. ¹H NMR (300 MHz, DMSO-d₆) d=8.14 (br d, J=8.3 Hz, 1H), 7.33-7.25 (m, 4H), 7.25-7.17 (m, 1H), 4.90-4.77 (m, 2H), 3.52 (br t, J=5.7 Hz, 2H), 3.34 (s, 1H), 2.94 (s, 1H), 2.78 (s, 1H), 2.20 (d, J=14.0 Hz, 4H), 1.97 (d, J=9.8 Hz, 2H), 1.41-1.31 (m, 9H), 1.00 (d, J=1.1 Hz, 6H).

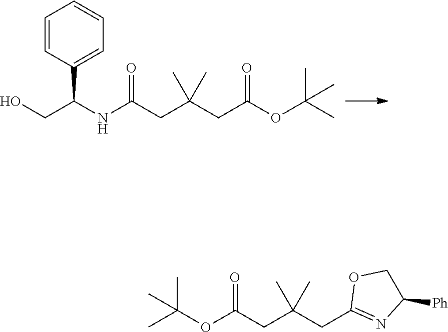

### Step 3.

tert-Butyl (R)-5-((2-hydroxy-1-phenylethyl)amino)-3,3-dimethyl-5-oxopentanoate (6 g, 17.89 mmol) and 2,3-dichloro-5,6-dicyano-p-benzoquinone (6.09 g, 26.8 mmol) was dissolved in dry dichloromethane (70 mL) under Ar. Triphenylphosphine (7.04 g, 26.8 mmol) was added to the above solution. The reaction mixture was stirred at room temperature for 2 h. The crude product was then concentrated under vacuo and purified by flash column chromatography on silica gel (EtOAc/Hexanes, 1: 5) to give tert-butyl (R)-3,3-dimethyl-4-(4-phenyl-4,5-dihydrooxazol-2-yl)butanoate (5.6 g, 17.64 mmol, 99% yield). ESI-MS(+) m/z: 318.3 [M+H]⁺. ¹H NMR (300 MHz, DMSO-d₆) d=7.41-7.18 (m, 5H), 5.18 (t, J=9.1 Hz, 1H), 4.59 (dd, J=8.7, 10.2 Hz, 1H), 3.94-3.85 (m, 1H), 3.94-3.85 (m, 1H), 3.95-3.84 (m, 1H), 4.10-3.84 (m, 1H), 2.43-2.22 (m, 4H), 1.40 (s, 9H), 1.09 (d, J=1.9 Hz, 6H).

### Step 4.

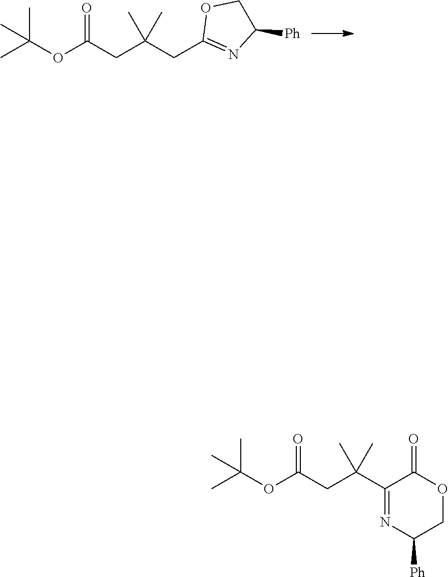 [SMILES: CC(C)(CC(=O)OC(C)(C)C)CC1=N[C@H](c2ccccc2)CO1 ; CC(C)(C)OC(=O)CC(C)(C)C1=N[C@H](c2ccccc2)COC1=O]

A solution of tert-butyl (R)-3,3-dimethyl-4-(4-phenyl-4,5-dihydrooxazol-2-yl)butanoate (5.6 g, 17.64 mmol) in EtOAc (250 mL) was added selenium dioxide (4.89 g, 44.1 mmol) and refluxed for 2 h. The reaction mixture was then cooled to room temperature and stirred for 12 h. The crude product was then concentrated in vacuo and purified by flash column chromatography on silica gel (EtOAc/Hexanes, 1:7) to afford tert-butyl (R)-3-methyl-3-(2-oxo-5-phenyl-5,6-dihydro-2H-1,4-oxazin-3-yl)butanoate (1.3 g, 3.92 mmol, 22.23% yield) as a colorless liquid. ESI-MS(+) m/z: 332.2 [M+H]⁺. 1H NMR (CDCl₃) δ 1.37 (s, 3H), 1.42 (s, 9H), 1.44 (s, 3H), 2.59 (d, J=15.5 Hz, 1H), 3.12 (d, J=15.5 Hz, 1H), 4.32 (t, J=11.1 Hz, 1H), 4.47 (dd, J=4.3 Hz, J=6.7 Hz, 1H), 4.80 (dd, J=4.3 Hz, J=6.7 Hz, 1H), 7.35-7.39 (m, 5H). ¹³C NMR (CD3Cl) 6 26.40, 27.29, 28.00, 40.84, 45.94, 59.72, 70.88, 80.63, 127.13, 127.92, 128.65, 137.58, 155.07, 167.46, 171.95.

### Step 5.

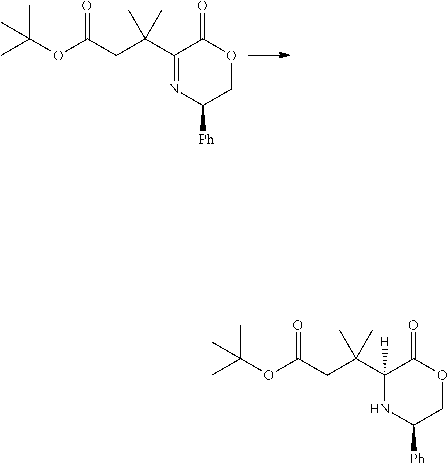 [SMILES: CC(C)(C)OC(=O)CC(C)(C)C1=N[C@H](c2ccccc2)COC1=O ; CC(C)(C)OC(=O)CC(C)(C)[C@@H]1N[C@H](c2ccccc2)COC1=O]

Platinum(IV) oxide monohydrate (130 mg, 0.530 mmol) was added to a solution of tert-butyl (R)-3-methyl-3-(2-oxo-5-phenyl-5,6-dihydro-2H-1,4-oxazin-3-yl)butanoate (1.3 g, 3.92 mmol) in methanol (50 mL). The reaction flask was purged with H₂ (3×) and stirred under H₂ for 24 h. After venting the vessel, the reaction mixture was filtered through Celite, and the filtrate was washed with EtOAc. The crude product was concentrated under vacuo and purified by flash column chromatography on silica gel (EtOAc/Hexanes, 1:8) to give tert-butyl 3-methyl-3-((3S,5R)-2-oxo-5-phenylmorpholin-3-yl)butanoate (1.2 g, 3.33 mmol, 85% yield). ¹H NMR (300 MHz, DMSO-d₆) δ 7.52-7.42 (m, 2H), 7.41-7.26 (m, 3H), 4.30-4.20 (m, 2H), 4.13 (d, J=10.6 Hz, 1H), 3.80 (d, J=7.6 Hz, 1H), 3.07-2.98 (m, 1H), 2.47 (br s, 1H), 2.27 (d, J=13.6 Hz, 1H), 1.43-1.35 (m, 9H), 1.17-1.07 (m, 5H).

### Step 6.

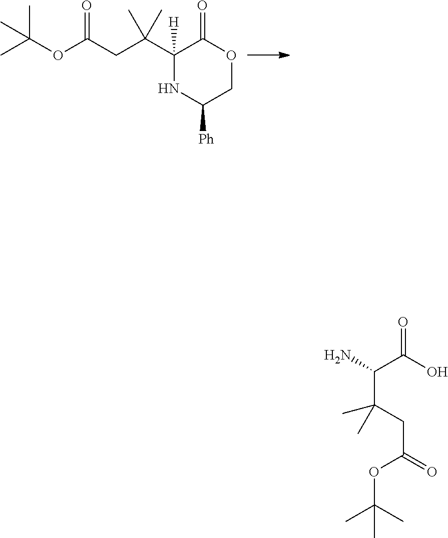 [SMILES: CC(C)(C)OC(=O)CC(C)(C)[C@@H]1N[C@H](c2ccccc2)COC1=O ; CC(C)(C)OC(=O)CC(C)(C)[C@H](N)C(=O)O]

Pearlman's catalyst Pd(OH)₂ on carbon (1.264 g, 1.799 mmol, 20% w w) was added to a solution of tert-butyl 3-methyl-3-((3S,5R)-2-oxo-5-phenylmorpholin-3-yl)butanoate (1.2 g, 3.60 mmol) in methanol (50 mL)/water (3.13 mL)/TFA (0.625 mL) (40:2.5:0.5, v v v). The vessel was purged with H₂ and stirred under H₂ for 24 h. After venting the vessel, the reaction mixture was filtered through Celite, and the filtrate was washed with MeOH. The crude product ((S)-2-amino-5-(tert-butoxy)-3,3-dimethyl-5-oxopentanoic acid (0.83 g, 3.59 mmol, 100% yield)) was concentrated under vacuo. This crude was taken for the next step without further purification. Analytical LC/MS Condition M: 1.13 min, 232.2 [M+H]⁺.

### Step 7.

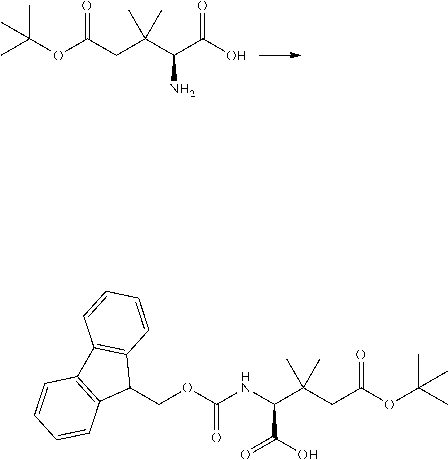 [SMILES: CC(C)(C)OC(=O)CC(C)(C)[C@H](N)C(=O)O ; CC(C)(C)OC(=O)CC(C)(C)[C@H](NC(=O)OCC1c2ccccc2-c2ccccc21)C(=O)O]

The crude product (S)-2-amino-5-(tert-butoxy)-3,3-dimethyl-5-oxopentanoic acid (1 g, 4.32 mmol) dissolved in water (30 mL). Na₂CO₃ (0.916 g, 8.65 mmol) was then added to the above solution. To this solution, fmoc n-hydroxysuccinimide ester (1.458 g, 4.32 mmol) in dioxane (30 mL) was added drop wise at 0° C. and stirred at room temperature for 16 h. The reaction mixture was acidified to pH -2 by 1N HCl and extracted with EtOAc (50 mL×3), dried over Na₂SO₄, concentrated under vacuo and purified by flash column chromatography on silica gel (EtOAc/petroleum ether, 35 to 39%) to give (S)-2-((((9H-fluoren-9-yl)methoxy)carbonyl)amino)-5-(tert-butoxy)-3,3-dimethyl-5-oxopentanoic acid (0.73 g, 1.567 mmol, 36.2% yield) as a white solid. LCMS, Analytical LC/MS Condition E, MS (ESI) t_{R}=2.135 min, m/z 452.2 [M−H]. H NMR (400 MHz, DMSO-d₆) δ 12.78-12.64 (m, 1H), 7.90 (d, J=7.5 Hz, 2H), 7.77 (dd, J=4.5, 7.0 Hz, 2H), 7.65 (br d, J=9.5 Hz, 1H), 7.46-7.39 (m, 2H), 7.37-7.29 (m, 2H), 4.32-4.15 (m, 4H), 2.39-2.31 (m, 1H), 2.30-2.21 (m, 1H), 1.39 (s, 9H), 1.12-1.00 (m, 6H).

### Preparation of (2S)-2-({[(9H-fluoren-9-yl)methoxy]carbonyl}amino)-3-(morpholin-4-yl)propanoic acid

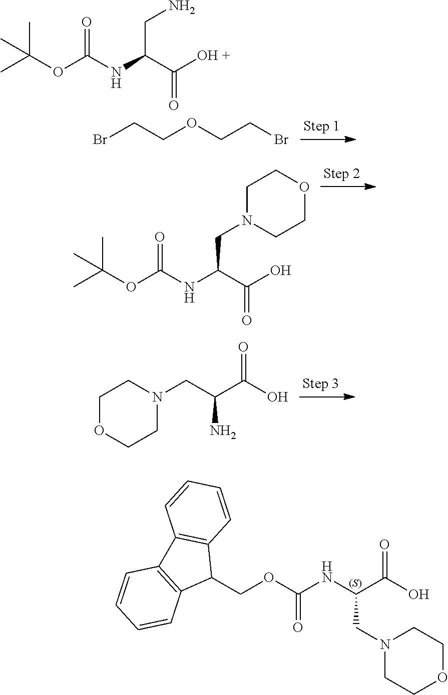

### Step 1.

In a 2-L multi-necked round-bottomed flask fitted with a thermo pocket was added (S)-3-amino-2-((tert-butoxycarbonyl)amino)propanoic acid (50 g, 245 mmol), dioxane (500 mL), followed by 1-bromo-2-(2-bromoethoxy)ethane (30.8 mL, 245 mmol) at rt. NaOH (367 mL, 734 mmol) solution was added and the resulting yellow clear solution was heated to 110° C. (external temperature, 85° C. internal temperature) for 12 h. An aliquot of clear solution was subjected to LCMS (Polar method) which showed completion, and then the dioxane was evaporated to get light red solution which was acidified to pH 3. The resulting mixture was concentrated under high vacuum pump (˜4 mbar) at 60° C. to get (S)-2-((tert-butoxycarbonyl)amino)-3-morpholinopropanoic acid (67 g, 244 mmol, 100% yield) pale yellow solid. Analytical LC/MS Condition M:” 0.56 min, 27 5.2 [M+H]⁺.

### Step 2.

To a stirred suspension of (S)-2-((tert-butoxycarbonyl)amino)-3-morpholinopropanoic acid (100 g, 365 mmol) in dioxane (400 mL) at 0-5° C. was added HCl in dioxane (911 mL, 3645 mmol) slowly over 20 min. The resulting mixture was stirred at rt fr12 h. The volatile was evaporated to get pale yellow sticky crude (S)-2-amino-3-morpholinopropanoic acid (16 g, 92 mmol, 97% yield), This crude was taken for next step without further purification. MS (ESI) m/z 175.2 [M+H]⁺.

### Step 3.

The crude product (S)-2-amino-3-morpholinopropanoic acid (11 g, 63.1 mmol) dissolved in water (250 mL)Na₂CO₃ (13.39 g, 126 mmol) was then added to the above solution. To this solution. Fmoc N-hydroxvsuccinimide ester (21.30 ma 63.1 mmol) was added dropwise a 0° C. and stirred at room temperature for 16 h. The reaction mixture was acidified to pH -2 by 1N HCl and extracted with EtOAc (500 mL×3), dried over Na₂SO₄, concentrated under vacuo, and purified by flash column chromatography on silica gel (petroleum ether/EtOAc, 0-100% then MeOH/CHCl₃ 0-15%) to get (S)-2-((((9H-fluoren-9-yl)methoxy)carbonyl)amino)-3-morpholinopropanoic acid (23 g, 55.9 mmol, 89% yield) as a brown solid. Analytical LC/MS Condition E: 1.43 min, 397.2 [M+H]⁺. ¹H NMR (400 MHz, METHANOL-d₄) δ 7.78 (br d, J=7.5 Hz, 2H), 7.71-7.57 (m, 2H), 7.42-7.34 (m, 2H), 7.34-7.26 (m, 2H), 4.71 (br s, 1H), 4.54-4.32 (m, 2H), 4.29-4.17 (m, 1H), 3.90 (br s, 4H), 3.76-3.62 (m, 1H), 3.58-3.47 (m, 1H), 3.41 (br s, 2H), 3.36-3.32 (m, 2H), 3.31-3.26 (m, 1H).

### Preparation of (2S,3S)-3-{[(tert-butoxy)carbonyl]amino}-2-({[(9H-fluoren-9-yl)methoxy]carbonyl}amino)butanoic acid

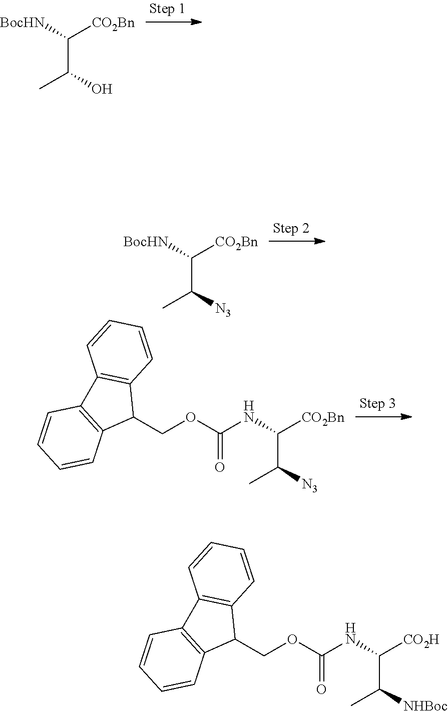

### Step 1.

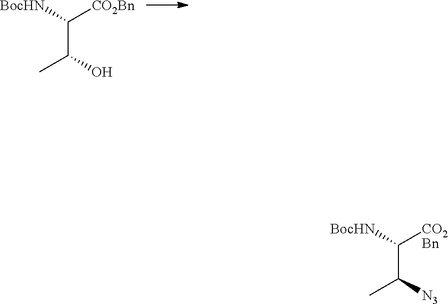 [SMILES: C[C@@H](O)[C@H](NC(=O)OC(C)(C)C)C(=O)OCc1ccccc1 ; C[C@H](N=[N+]=[N-])[C@H](NC(=O)OC(C)(C)C)C(=O)OCc1ccccc1]

To a solution of the benzyl (tert-butoxycarbonyl)-L-threoninate (22 g, 71.1 mmol) in CH₂Cl₂ (600 mL) at −78° C. was sequentially added trifluoromethanesulfonic anhydride (24.08 g, 85 mmol) dropwise and then 2,6-lutidine (10.77 mL, 92 mmol) slowly. After stirring at the same temperature for 1.5 h and monitoring by TLC (Hex:EtOAc 8:2), tetrabutylammonium azide (50.6 g, 178 mmol) was added in portions. After stirring at −78° C. for 1 h, the cooling bath was removed and the reaction mixture was allowed to reach 23° C. for 1.5 h. The reaction was repeated. A saturated aqueous solution of NaHCO₃ was added, and the aqueous phase extracted with EtOAc. The crude product was purified by flash chromatography over silica gel (Hex:EtOAc 95:5 a 9:1) to give benzyl (2S,3S)-3-azido-2-((tert-butoxycarbonyl)amino)butanoate (20 g, 59.8 mmol, 84% yield) as colorless liquid. Analytical LC/MS Condition E: 3.13 min, 333.2 [M−H]⁻.

### Step 2.

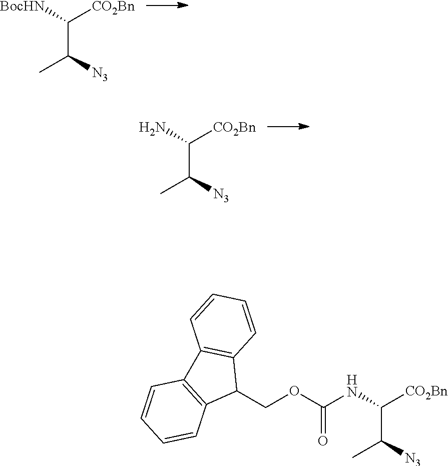 [SMILES: C[C@H](N=[N+]=[N-])[C@H](NC(=O)OC(C)(C)C)C(=O)OCc1ccccc1 ; C[C@H](N=[N+]=[N-])[C@H](N)C(=O)OCc1ccccc1 ; C[C@H](N=[N+]=[N-])[C@H](NC(=O)OCC1c2ccccc2-c2ccccc21)C(=O)OCc1ccccc1]

A solution of benzyl (2S,3S)-3-azido-2-((tert-butoxycarbonyl)amino)butanoate (20 g, 59.8 mmol), dichloromethane (300 mL) and TFA (50 mL, 649 mmol) was stirred for 2 h at 23° C. and then evaporated to dryness to give the corresponding amine. The above amine was redisolved in water (200 mL) and tetrahydrofuran (200 mL). At 0° C., DIPEA (11.49 mL, 65.8 mmol) was added followed by Fmoc chloride (17.02 g, 65.8 mmol). The mixture was warmed up to rt and stirred for 3 h. It was extracted with EtOAc and washed with 0.5 M HCl solution and then brine solution. It was concentrated to get crude liquid. The above crude was purifirf by silica gel column chromatography. The product was eluted at 20% EtOAc in petrolium ether. The fractions were concentrated to get benzyl (2S,3S)-2-((((9H-fluoren-9-yl)methoxy)carbonyl)amino)-3-azidobutanoate (23 g, 50.4 mmol, 84% yield) as a colorless liquid. Analytical LC/MS Condition E: 3.70 min, 479.3 [M+Na]⁺.

### Step 3.

To a multi-neck round-bottled flask was charged benzyl (2S,3S)-2-((((9H-fluoren-9-yl)methoxy)carbonyl)amino)-3-azidobutanoate (40 g, 88 mmol) in tetrahydrofuran (1200 mL). Pd/C (9.32 g, 8.76 mmol) was added under nitrogen and the reaction was stirred under hydrogen for 12 h. Sodium bicarbonate (11.04 g, 131 mmol) in water 6 (mL) was added followed by Boc-anhydride (30.5 mL, 131 mmol). The mixture was stirring under nitrogen for 12 h. The reaction mass was filtered through cellite bed, washed the bed with THF/Water mixture. The mother liquid was concentrated and washed with EtOAc. Then pH of water layer was adjusted to 7-6 using 1.5 N HCl solution. The resulting white solid was extracted with ethylacetate. The above reaction was repeated three more times. The combined organics were washed with water and brine solution, dried over sodium sulphate, and concentrated to afford (2S,3S)-2-((((9H-fluoren-9-yl)methoxy)carbonyl)amino)-3-((tert-butoxycarbonyl)amino)butanoic acid as a white solid (28 g). This was mixed with a previously obtained batch (8 g) in DCM (200 mL). n-Hexane (1L) was added to the above solution and sonicated for 2 min. The solids were filtered, rinsed with hexanes and dired overnight to give (2S,3S)-2-((((9H-fluoren-9-yl)methoxy)carbonyl)amino)-3-((tert-butoxycarbonyl)amino)butanoic acid (36 g, 81 mmol, 92% yield) as a white powder. Analytical LC/MS Condition E: 1.90 min, 439.2 [M−H]. H NMR (400 MHz, DMSO-d₆) δ 7.90 (d, J=7.6 Hz, 2H), 7.75 (d, J=7.2 Hz, 2H), 7.43 (t, J=7.2 Hz, 2H), 7.34 (t, J=Hz, 6.71 (br. d. J=7.6 Hz, 1H), 4.29-4.26 (m, 2H), 4.25-4.21 (m, 1H), 3.94-3.90 (m, 1H), 1.37 (s, 9H), 1.02 (d, J=6.8 Hz, 3H). 13C NMR (101 Hz, DMSO-d₆) δ 171.9, 156.3, 154.8, 143.7, 140.6, 127.6, 127.0, 125.3, 120.0, 77.7, 65.8, 57.8, 47.0, 46.6, 28.2, 16.2.

### Preparation of (S)-2-((((9H-fluoren-9-yl)methoxy)carbonyl)amino)-2-(1-(((tert-butoxycarbonyl)amino)methyl)cyclopropyl)acetic acid

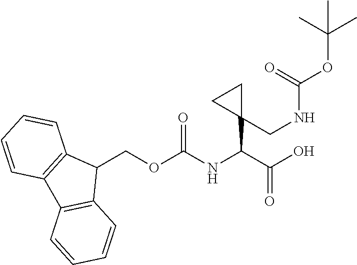 [SMILES: CC(C)(C)OC(=O)NCC1([C@H](NC(=O)OCC2c3ccccc3-c3ccccc32)C(=O)O)CC1]

Final product was obtained following similar procedures of ethyl (S)-5-((tert-butoxycarbonyl)amino)-2-(((S)-mesitylsulfinyl)amino)-3,3-dimethylpentanoate. The synthesis afforded the desired product (0.65 g, 22% yield) as a white solid after purification by flash column chromatography (Red Sep, 40 g, SiO₂, 35 to 40% EtOAc:hexanes (compound ELSD active)). Analytical LC/MS Condition E: 2.04 min, 465.2 [M−H]-. ¹H NMR (300 MHz, DMSO-d₆) δ 7.90 (d, J=7.6 Hz, 2H), 7.71 (m, 3H), 7.47-7.27 (m, 2H), 6.98-6.71 (m, 2H), 4.30-4.17 (m, 3H), 3.94-3.82 (m, 1H), 3.20-2.90 (m, 2H), 1.44-1.30 (m, 9H), 0.48 (br s, 4H).

### Preparation of (S)-2-((((9H-fluoren-9-yl)methoxy)carbonyl)amino)-2-(1-(tert-butoxycarbonyl)azetidin-3-yl)acetic acid

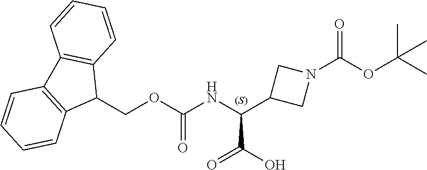

Final product was obtained following similar procedures of ethyl (S)-5-((tert-butoxycarbonyl)amino)-2-(((S)-mesitylsulfinyl)amino)-3,3-dimethylpentanoate. The synthesis afforded the desired product (2.66 g, 20% yield) as a slightly tan solid after purification by reverse-phase HPLC. Analytical LC/MS Condition E: 1.87 min, 467.2 [M−H]. H NMR (400 MHz, DMSO-d₆) δ 7.89 (d, J=7.6 Hz, 2H), 7.69 (m, 2H), 7.41 (t, J=7.2 Hz, 2H), 7.34-7.31 (m, 2H), 6.71 (br. d. J=7.6 Hz, 1H), 4.29-4.23 (m, 3H), 3.77-3.70 (m, 5H), 2.80 (m, 1H), 1.36 (s, 9H).

### Linker Tail Synthesis:

### Preparation of perfluorophenyl dodecanoate, (C12)

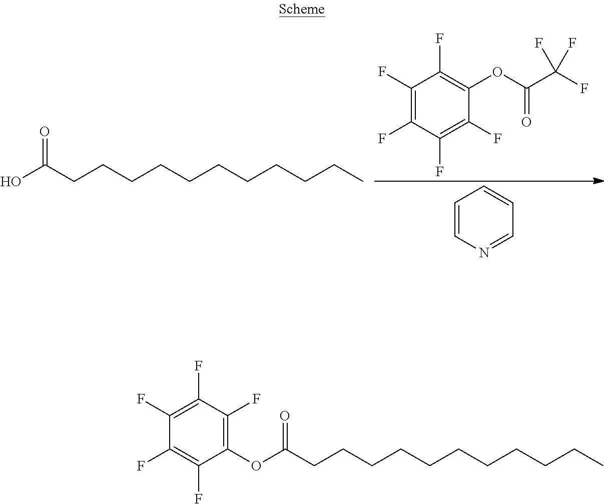

To dodecanoic acid (1310 mg, 6.54 mmol) was added DMF (5 mL), perfluorophenyl 2,2,2-trifluoroacetate (1.454 mL, 8.50 mmol) and pyridine (1.322 mL, 16.35 mmol). The vial was capped and shaken for 2 hours at room temperature. The reaction mixture was concentrated and the crude product was used in the next step as such.

### Preparation of perfluorophenyl tetradecanoate, (C14)

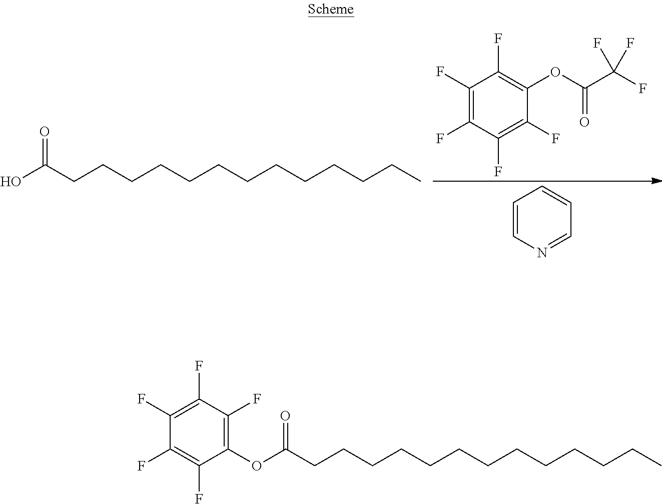

To tetradecanoic acid (345 mg, 1.511 mmol) was added DMF (5 mL), perfluorophenyl 2,2,2-trifluoroacetate (0.388 mL, 2.266 mmol) and pyridine (0.269 mL, 3.32 mmol). The vial was capped and shaken for 2 hours at room temperature. The reaction mixture was concentrated and the crude product was used in the next step as such.

### Preparation of perfluorophenyl pentadecanoate, (CJ5)

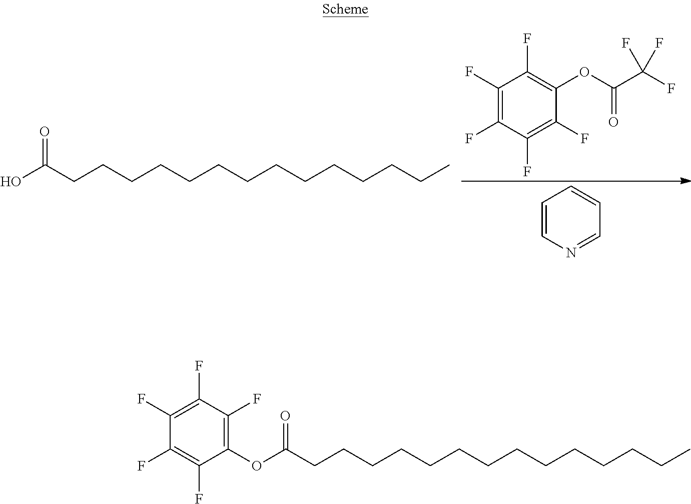

To pentadecanoic acid (324 mg, 1.337 mmol) was added DMF (5 mL), perfluorophenyl 2,2,2-trifluoroacetate (0.343 mL, 2.005 mmol), and pyridine (0.238 mL, 2.94 mmol). The vial was capped and the mixture was shaken for 2 hours at room temperature. The reaction mixture was concentrated and the crude product was used in the next step as such.

### Preparation of per fluorophenyl palmitate, (C16)

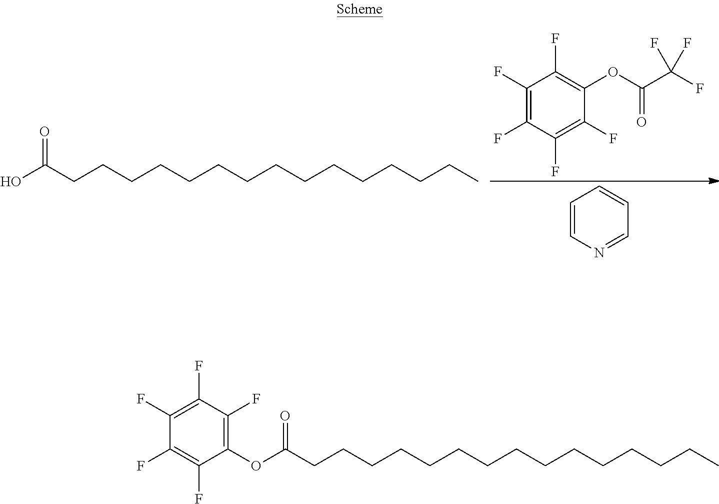

To a pressure seal vial was added palmitic acid (1.026 g, 4 mmol, 1.0 eq) d MF (5 mL), perfluorophenyl 2,2,2-trifluoroacetate (1.369 mL, 8.00 mmol, 2.0 eq), and pyridine (0.712 mL, 8.80 mmol, 2.2 eq). The reaction mixture was kept under a blanket of nitrogen and stirred at room temperature for 16 hours. The reaction was then poured into a saturated citric acid solution and extracted with CH₂Cl₂ 3 times. The organic layers were combined and washed with brine, dried over anhydrous Na₂SO₄, filtered and evaporated in vacuo. The crude material was purified by chromatography on silica gel (80 g) and eluted with 100% hexanes to 5% ethyl acetate/hexanes. The appropriate fractions were combined to obtain perfluorophenyl palmitate (1.47 g, 0.348 mmol, 87% yield). ¹H NMR (400 MHz, CHLOROFORM-d) δ 2.68 (t, J=7.4 Hz, 2H), 1.80 (quin, J=7.5 Hz, 2H), 1.44 (br. s., 3H), 1.29 (s, 21H), 0.93-0.87 (m, 3H).

### Preparation of perfluorophenyl heptadecanoate, (C17)

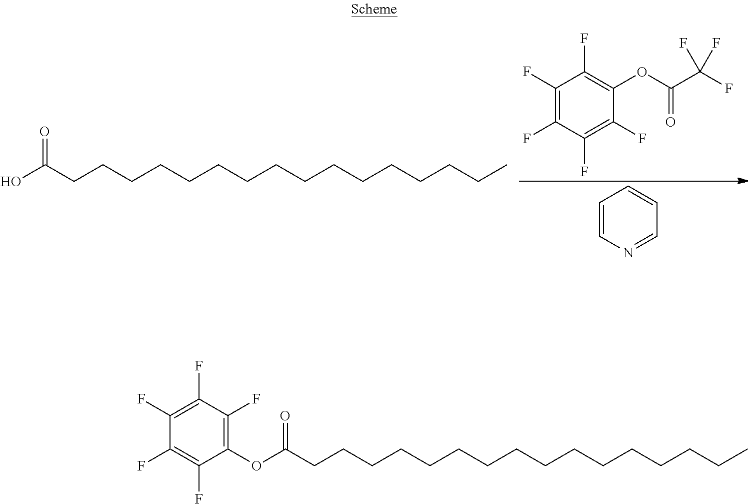

To heptadecanoic acid (304 mg, 1.124 mmol) was added DMF (5 mL), perfluorophenyl 2,2,2-trifluoroacetate (0.288 mL, 1.686 mmol) and pyridine (0.200 mL, 2.473 mmol). The vial was capped and shaken for 2 hours at room temperature. The reaction mixture was concentrated and the crude product was used in the next step as such.

### Preparation of perfluorophenyl stearate, (C18)

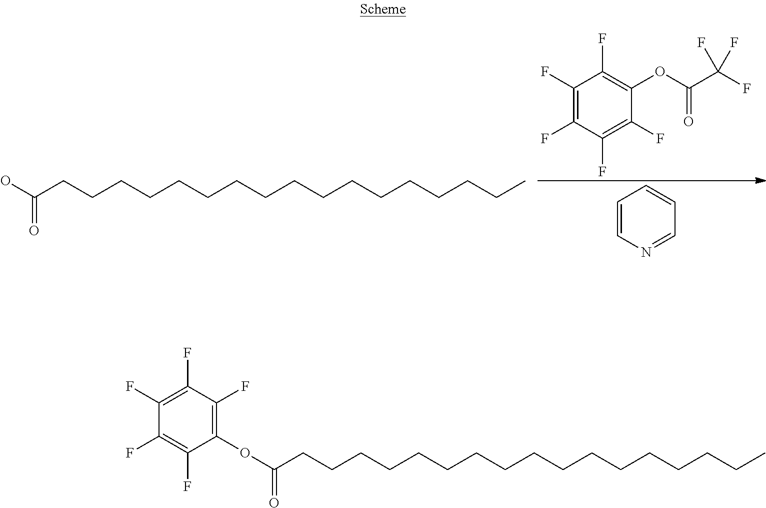

To stearic acid (297 mg, 1.044 mmol) was added DMF (5 mL), perfluorophenyl 2,2,2-trifluoroacetate (0.268 mL, 1.566 mmol) and pyridine (0.186 mL, 2.297 mmol). The vial was capped and shaken for 2 hours at room temperature. The reaction mixture was concentrated and the crude product was used in the next step as such.

### Preparation of perfluorophenyl (R)-4-((3R,5R,8R,9S,10S,13R,14S,17R)-3-hydroxy-10,13-dimethylhexadecahydro-]H-cyclopenta[a]phenanthren-17-yl)pentanoate

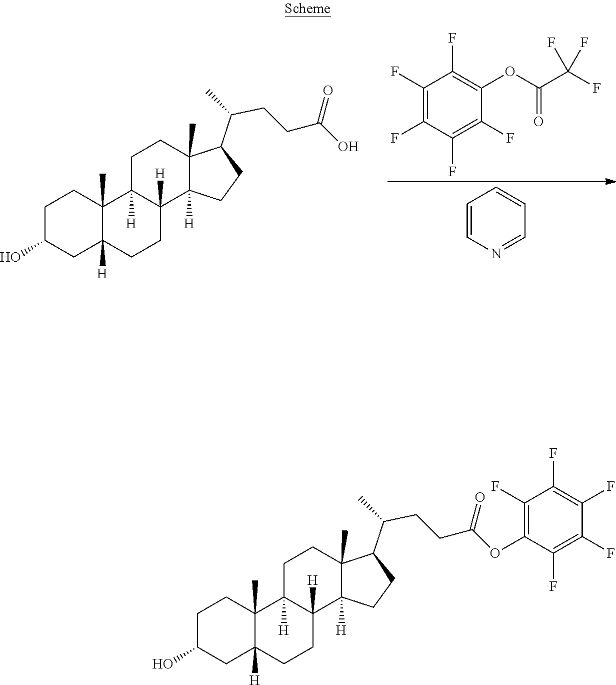

To (R)-4-((3R,5R,8R,9S,1OS,13R,14S,17R)-3-hydroxy-10,13-dimethylhexadecahydro-1H-cyclopenta[a]phenanthren-17-yl)pentanoic acid (300 mg, 0.797 mmol) was added DMF (5 mL), perfluorophenyl 2,2,2-trifluoroacetate (0.204 mL, 1.195 mmol) and pyridine (0.142 mL, 1.753 mmol). The vial was capped and shaken for 2 hours at room temperature. The reaction mixture was concentrated and the crude product was used in the next step as such.

### Preparation of 1-tert-butyl 2,3,4,5,6-pentafluorophenyl tetradecanedioate (C14)

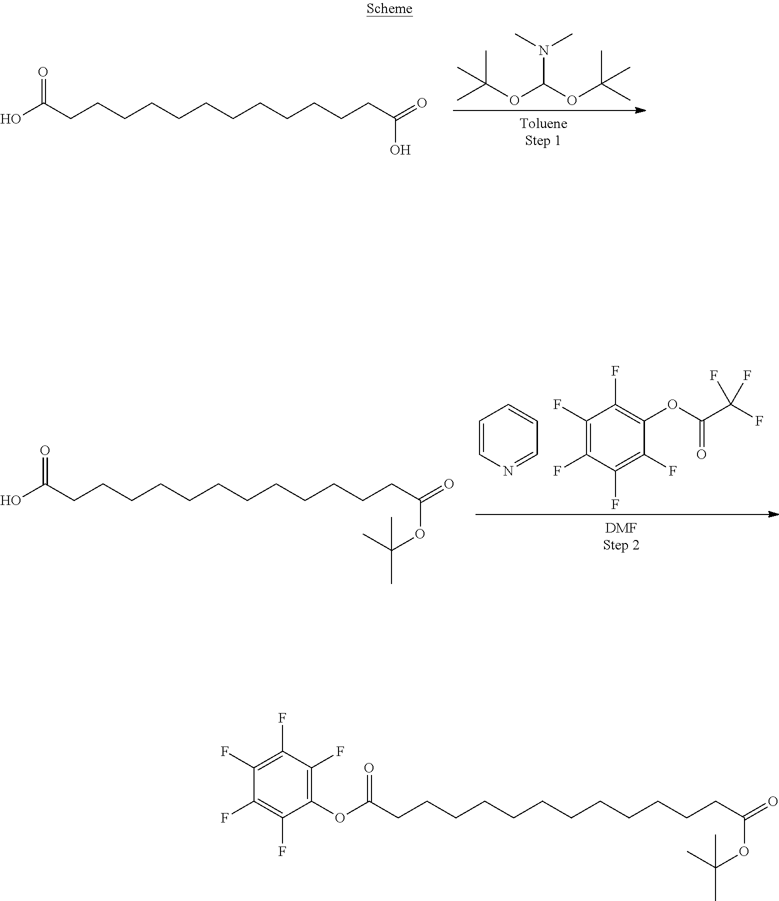

Step 1: 14-(tert-butoxy)-14-oxotetradecanoic acid

N,N-dimethylformamide di-tert-butyl acetal (24.78 mL, 103 mmol) was added to a suspension of tetradecanoic acid (10 g, 38.7 mmol) in Toluene (62.5 mL) at 120° C. over 4 hours. Then, it was heated at reflux (128° C.) for 16 hours. Cooled to rt, all volatiles were removed in vacuo and the residue was suspended in DCM/EtOAc (1:1, 130 mL) at room temperature for 30 minutes. The solids were collected by filtration and triturated with DCM (50 mL). After filtration, 14-(tert-butoxy)-14-oxotetradecanoic acid was obtained (4.02 g, 33% yield). ¹H NMR (400 MHz, DMSO-d₆) δ 11.89 (br s, 1H), 2.18 (br t, J=7.2 Hz, 5H), 1.49 (br s, 5H), 1.39 (br s, 1H), 1.25 (br s, 19H).

Step 2: 1-tert-butyl 2,3,4,5,6-pentafluorophenyl tetradecanedioate

To a 250 mL round bottom flask was added 14-(tert-butoxy)-14-oxotetradecanoic acid (5.27 g, 16.76 mmol), DMF (33.5 mL), pentafluorophenyl trifluoroacetate (5.76 mL, 33.5 mmol), and pyridine (3.39 mL, 41.9 mmol). The flask was sealed with a septum and kept under a nitrogen atmosphere and stirred overnight at room temperature. The reaction mixture was poured into a saturated citric acid solution and extracted with DCM three times. The organic layers were combined and washed with brine, dried over MgSO₄, filtered and evaporated in vacuo. The crude product was purified on silica gel chromatography eluting with 0% ethyl acetate/100% hexanes to 55% ethyl acetate/45% hexanes. The pure fractions were combined and evaporated in vacuo affording 1-tert-butyl 14-(perfluorophenyl) tetradecanedioate (8.05 g, 16.76 mmol, 100% yield). ¹H NMR (400 MHz, CHLOROFORM-d) δ 2.67 (t, J=7.4 Hz, 2H), 2.22 (t, J=7.5 Hz, 2H), 1.84-1.72 (m, 2H), 1.58 (t, J=7.2 Hz, 2H), 1.45 (s, 9H), 1.44-1.37 (m, 2H), 1.28 (br. s., 14H). 1-Tert-butyl 2,3,4,5,6-pentafluorophenyl dodecanedioate (C12) was made following the above procedures.

### Preparation of 1-(tert-butyl) 15-(perfluorophenyl) pentadecanedioate (CJ5)

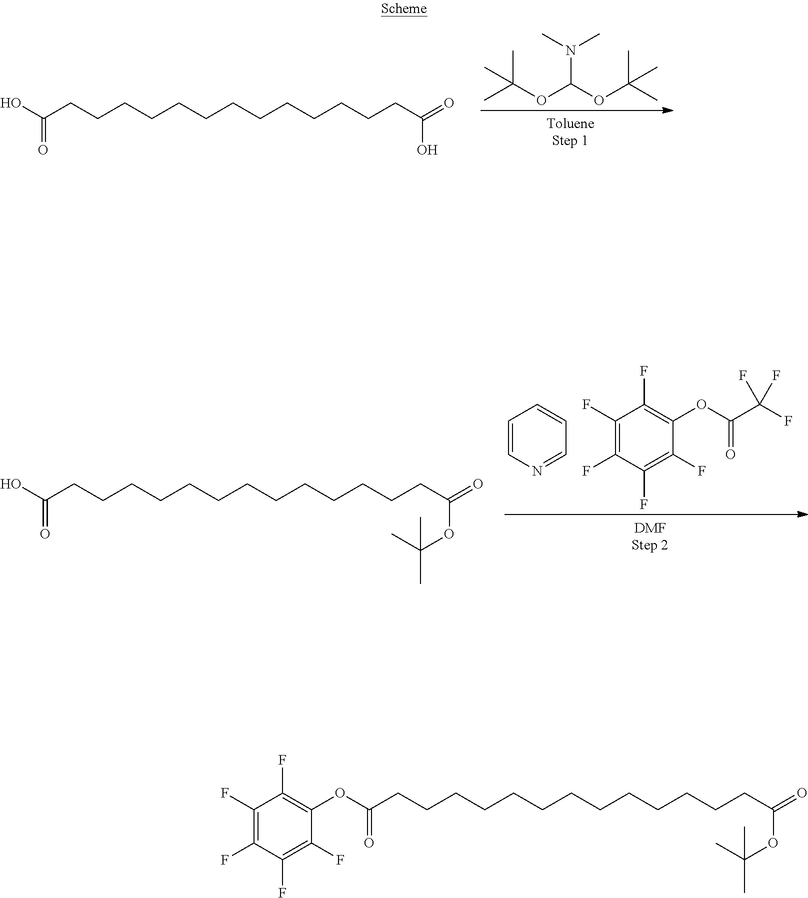

Step 1:]5-(tert-butoxy)-]5-oxopentadecanoic acid

A three neck flask was charged with pentadecanedioic acid (5.00 g, 18.36 mmol) and toluene (36.7 mL) to give a suspension. The mixture was heated to reflux until all solids were in solution. While heating continued 1,1-di-tert-butoxy-N,N-dimethylmethanamine (8.80 mL, 36.7 mmol) was added dropwise over 30 minutes. The mixture was heated under refluxing conditions overnight. The reaction mixture was cooled to room temperature and transferred to a 250 mL round bottom flask. The volatiles were removed under reduced pressure to give an off-white solid. 150 mL of dichloromethane was added to the flask and the suspension was stirred for 2 hours. The suspension was filtered by vacuum filtration. The collected solids were once again stirred with dichloromethane for 30 minutes and filtered. The filtrate was concentrated under reduced pressure to yield a white solid. The product was taken up in 200 mL dichloromethane and concentrated. This procedure was performed twice. The resulting white solid was placed under vacuum over for 72 hours to afford 15-(tert-butoxy)-15-oxopentadecanoic acid (3.94 g, 65.3%). ¹H NMR (400 MHz, METHANOL-d₄) δ 2.28 (t, J=7.4 Hz, 2H), 2.21 (t, J=7.3 Hz, 2H), 1.63-1.55 (m, 4H), 1.45 (s, 9H), 1.31 (s, 18H).

Step 2: 1-(tert-butyl)]5-(perfluorophenyl) pentadecanedioate

To a 50 mL round bottom flask was added 15-(tert-butoxy)-15-oxopentadecanoic acid (0.97 g, 2.95 mmol), DMF (11.81 mL), pyridine (0.836 mL, 10.34 mmol) and pentafluorophenyl trifluoroacetate (1.272 mL, 7.38 mmol). The flask was sealed with a septum and kept under a blanket of nitrogen and stirred overnight at room temperature. The next day the reaction was poured into a saturated citric acid solution and extracted with DCM three times. The organic layers were combined and washed with brine, dried over Na₂SO₄ and evaporated in vacuo. The crude product 1-tert-butyl 15-(perfluorophenyl) pentadecanedioate was used as is without purification.

### Preparation of 1-(tert-butyl) 16-(perfluorophenyl) hexadecanedioate (C16)

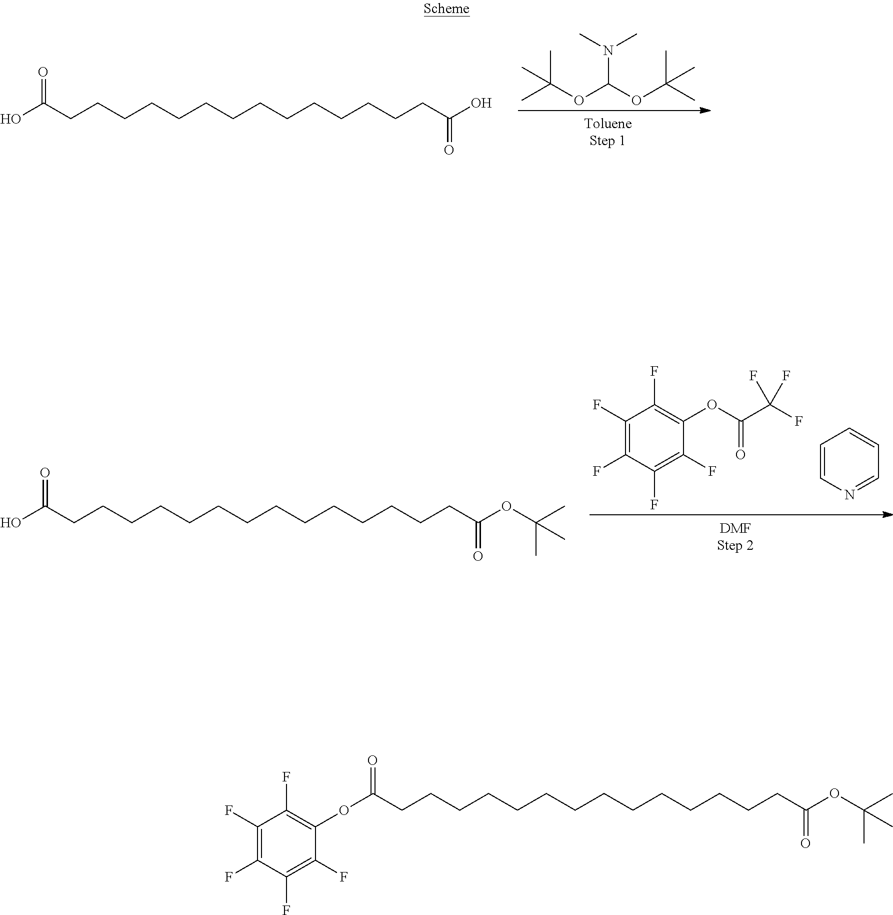

Step 1: Preparation of 16-(tert-butoxy)-16-oxohexadecanoic acid

Hexadecanedioic acid (4.5 g, 15.71 mmol) was suspended in toluene (28.1 mL) and the mixture was heated to reflux. 1,1-di-tert-butoxy-N,N-dimethylmethanamine (10.10 mL, 42.1 mmol) was added dropwise over 30 min. The mixture was put under refluxing conditions overnight. The solvent was removed in vacuo at 50° C. and the crude material was suspended in DCM/EtOAc (75 mL. 1:1) ans stirred for 15 min. The solids were removed by filtration and washed with DCM (25 mL). The filtration was evaporated in vacuo. The resulting material was suspended in DCM (6 mL), cooled with ice for 10 mins, and filtered. The solvent was removed in vacuo to leave crude product which was purified by flash chromatography eluting with EtOAc/Hexanes. After evaporation of the desired fractions 16-(tert-butoxy)-16-oxohexadecanoic acid (2.56 g, 7.47 mmol, 47.6% yield) was obtained. ¹H NMR (500 MHz, METHANOL-d₄) δ 2.35-2.14 (m, 4H), 1.65-1.54 (m, 4H), 1.50-1.42 (m, 11H), 1.36-1.21 (m, 18H).

Step 2: Preparation of 1-(tert-butyl) 16-(perfluorophenyl) hexadecanedioate

To a 1 dram vial was added 16-(tert-butoxy)-16-oxohexadecanoic acid (100 mg, 0.292 mmol), DMF (0.8 mL), perfluorophenyl 2,2,2-trifluoroacetate (164 mg, 0.584 mmol) and pyridine (0.052 mL, 0.642 mmol). The vial was sealed with a septum and stirred overnight at room temperature. The next day the crude reaction mixture was loaded onto a silica gel column and purified, eluting with a 5% EtOAC/95% Hexanes to 30% EtoAc/70% Hexanes. The pure fractions were combined and evaporated in vacuo affording 1-tert-butyl 16-(perfluorophenyl) hexadecanedioate (132 mg, 0.260 mmol, 89% yield) as a white solid. ¹H NMR (400 MHz, CHLOROFORM-d) δ 2.68 (t, J=7.4 Hz, 2H), 2.22 (t, J=7.5 Hz, 2H), 1.79 (quin, J=7.4 Hz, 3H), 1.51-1.34 (m, 15H), 1.29 (br s, 16H).

### Preparation of 1-(tert-butyl) 18-(per fluorophenyo) octadecanedioate (C18)

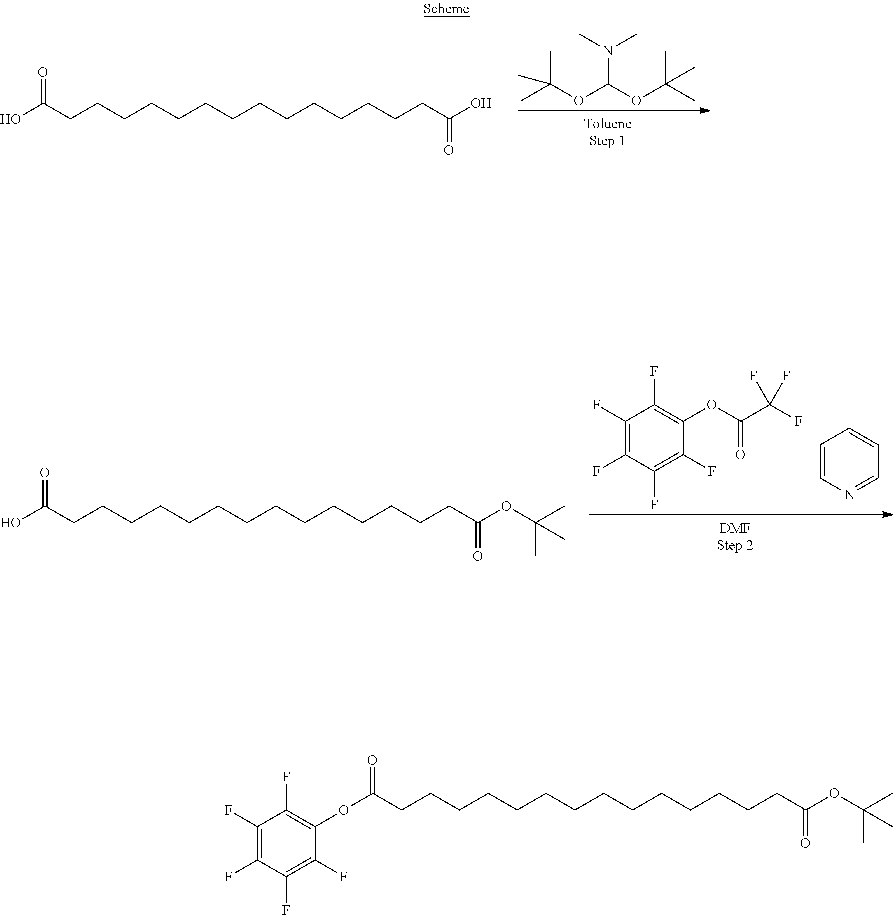

Step]: Preparation of]18-(tert-butoxy)-]8-oxooctadecanoic acid

Octadecanedioic acid (5.0 g, 15.90 mmol) was suspended in toluene (28.4 mL) and the mixture was heated to reflux.. 1,1-di-tert-butoxy-N,N-dimethylmethanamine (10.22 mL, 42.6 mmol) was added dropwise over 30 minutes. The mixture was put under refluxing conditions overnight. The solvent was removed in vacuo at 50° C. and the crude material was suspended in DCM/EtOAc (75 mL. 1:1) and stirred for 15 min. The solids were removed by filtration and washed with DCM (25 mL). The filtration was evaporated in vacuo. The crude product was purified by flash chromatography (0 to 25% Acetone/DCM) to get 18-(tert-butoxy)-18-oxooctadecanoic acid (1.72 g, 4.64 mmol, 29.2% yield). ¹H NMR (500 MHz, METHANOL-d₄) Shift 2.32-2.17 (m, 4H), 1.66-1.54 (m, 4H), 1.50-1.30 (m, 32H).

Step 2: Preparation of 1-(tert-butyl) 18-(perfluorophenyl) octadecanedioate

To a 50 mL round bottom flask was added 18-(tert-butoxy)-18-oxooctadecanoic acid (807 mg, 2.178 mmol), N,N-Dimethylformamide (8 mL), pyridine (379 mg, 4.79 mmol), and perfluorophenyl 2,2,2-trifluoroacetate (1220 mg, 4.36 mmol). The flask was sealed with a septum and kept under a blanket of nitrogen and stirred overnight at room temperature. The next day the reaction was poured into a saturated citric acid solution and extracted with DCM three times. The organic layers were combined and washed with brine, dried over Na₂SO₄ and evaporated in vacuo. The crude product 1-tert-butyl 18-(perfluorophenyl) octadecanedioate (1.1 g, 2.050 mmol, 94% yield) was used as is without purification. ¹H NMR (400 MHz, CHLOROFORM-d) δ 2.70-2.63 (m, 2H), 2.22 (t, J=7.5 Hz, 2H), 1.78 (quin, J=7.5 Hz, 2H), 1.63-1.52 (m, 2H), 1.48-1.39 (m, 12H), 1.37-1.20 (m, 21H).

### Preparation of (S)-I-tert-butyl 5-(perfluorophenyl) 2-(18-(tert-butoxy)-18-oxooctadecanamido)pentanedioate

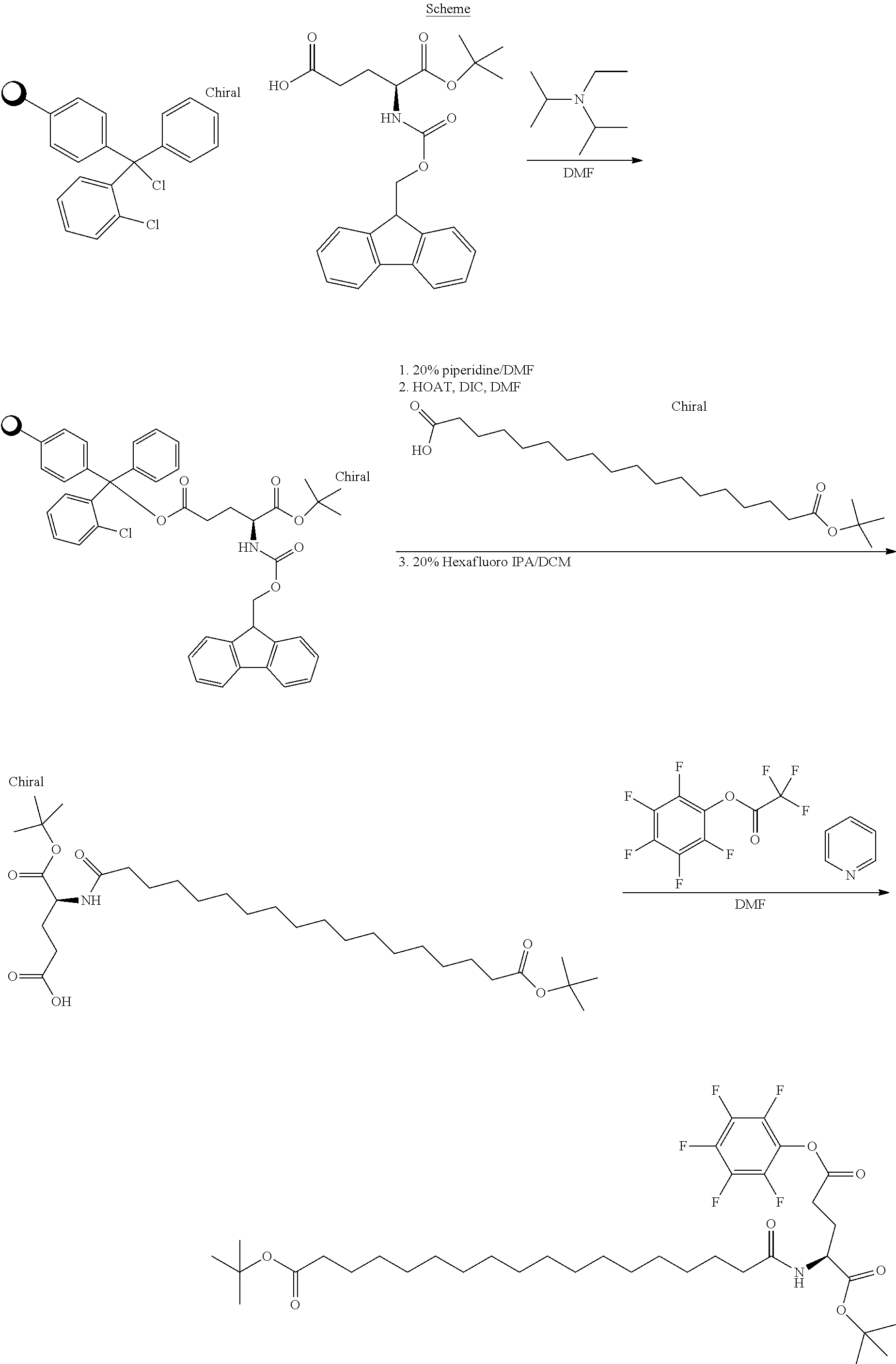

Step Step 1: Preparation of resin bound-(S)-4-((((9H-fluoren-9-yl)methoxy)carbonyl)amino)-5-(tert-butoxy)-5-oxopentanoic acid

To a glass reaction vessel equipped with a frit was added the 2-chloro-chlorotrityl resin mesh 50-150, (1.54 meq/gram, 1.94 grams, 3.0 mmole) to be swollen in DCM (5 mL) for 5 minutes. A solution of Fmoc-Glu-OtBu (1.276 g, 3.00 mmol, 1.0 eq) in DCM (5 mL) was added to the resin followed by DIPEA (2.61 mL, 15.00 mmol, 5.0 eq). The reaction was shaken at room temperature for 60 minutes. Add in DIEA (0.5 mL) and Methanol (3 mL), shaken for an additional 15 minutes. The reaction solution was filtered through the frit and the resin was rinsed with DCM (4×5 mL), DMF (4×5 mL), DCM (4×5 mL), diethyl ether (4×5 mL), and dried using a flow of nitrogen.

A sample of resin (13.1 mg) was treated with 20% piperidine/DMF (v/v, 2.0 mL) for 10 minutes with shaking. 1 mL of this solution was transferred to a 25.0 mL volumetric flask and diluted with methanol to a total volume of 25.0 mL. A blank solution of 20% piperidine/DMF (v/v, 1.0 mL) was diluted up with methanol in a volumetric flask to 25.0 mL. The UV was set to 301 nm and zero with the blank solution followed by the reading of the solution, Absorbance=1.9411 (1.9411/20 mg)*6.94=0.6736. Loading of the resin was measured to be 0.6736 mmol/g.

Step 2: Preparation of (S)-5-(tert-butoxy)-4-(18-(tert-butoxy)-18-oxooctadecanamido)-5-oxopentanoic acid

In a glass reaction vessel equipped with a frit was added the previous pre-loaded resin and a solution of 20% piperidine/DMF (v/v, 5 mL) and the suspension was allowed to shake for 5 mins. The solution was filtered off and the resin was treated again with a solution of 20% piperidine/DMF (v/v, 5 mL) for another 5 mins. The reaction solution was filtered through the frit and the resin was washed with DMF (6×5 mL×1 minute shaking). A solution of 18-(tert-butoxy)-18-oxooctadecanoic acid (593 mg, 1.6 mmol, 1.6 eq) and 1-hydroxy-7-azabenzotriazole (218 mg, 1.600 mmol, 1.6 eq) in DMF (4 mL) was added to the resin followed by N,N′-diisopropylcarbodiimide (251 μl, 1.600 mmol, 1.6 eq). The reaction was shaken at room temperature for 16 hours. The resin was washed with DMF (4×5 mL×1 minute shaking).

The protected peptide was cleaved off the resin with 20% HexaFluoro IPA/DCM (v/v, 30 mL) for 2 hours at room temperature. The cleavage solution containing the crude product was obtained by filtration. The resin was rinsed with DCM (2×5 mL). The combined filtrate were evaporated, chased with DCM (2×5 mL) to afford (S)-5-(tert-butoxy)-4-(18-(tert-butoxy)-18-oxooctadecanamido)-5-oxopentanoic acid as an oil (378 mg, 0.680 mmol, 68%). Analysis condition E: Retention time=1.32 min; ESI-MS(+) m/z [M+H]⁺: 557.5.

Step 3: Preparation of 1-(tert-butyl) 5-(perfluorophenyl) (18-(tert-butoxy)-18-oxooctadecanoyl)-L-glutamate

To a pressure seal vial was added (S)-5-(tert-butoxy)-4-(18-(tert-butoxy)-18-oxooctadecanamido)-5-oxopentanoic acid (378 mg, 0.680 mmol, 1.0 eq) d MF (5 mL), perfluorophenyl 2,2,2-trifluoroacetate (233 μl, 1.360 mmol, 2.0 eq), and pyridine (121 μl, 1.496 mmol, 2.2 eq). The reaction mixture was kept under a blanket of nitrogen and stirred for 16 hours at room temperature. The reaction was poured into a saturated citric acid solution and extracted with CH₂Cl₂ (3×). The organic layers were combined and washed with brine, dried over Na₂SO₄ and evaporated in vacuo. The crude material was purified by chromatography on silica gel (40 g) and eluted with 100 hexanes to 30% ethyl acetate/hexanes. The appropriate fractions were combined to obtain (S)-1-tert-butyl 5-(perfluorophenyl) 2-(18-(tert-butoxy)-18-oxooctadecanamido)pentanedioate (249 mg, 0.345 mmol, 50.7% yield). ¹H NMR (400 MHz, CHLOROFORM-d) δ 6.08 (d, J=7.8 Hz, 1H), 4.63 (td, J=7.8, 5.0 Hz, 1H), 2.89-2.65 (m, 2H), 2.43-2.31 (m, 1H), 2.29-2.18 (m, 4H), 2.14-2.02 (m, 1H), 1.59-1.43 (m, 18H), 1.37-1.22 (m, 24H).

### Preparation of (R)-18-((3-azido-1-carboxypropyl)amino)-18-oxooctadecanoic acid (N₃(Dab)-FDA18)

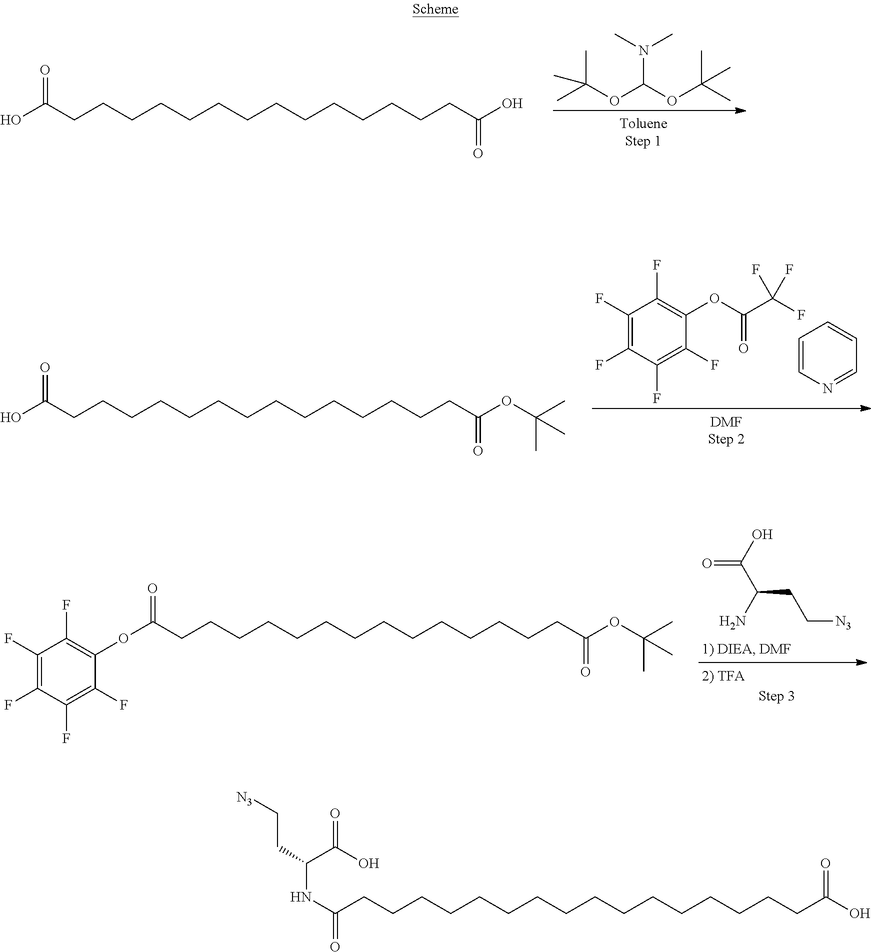

Step 1: Preparation of 18-(tert-butoxy)-18-oxooctadecanoic acid

Octadecanedioic acid (5.0 g, 15.90 mmol) was suspended in toluene (28.4 mL) and the mixture was heated to reflux. 1,1-Di-tert-butoxy-N,N-dimethylmethanamine (10.22 mL, 42.6 mmol) was added dropwise over 30 min. The mixture was put under refluxing conditions overnight. The solvent was removed in vacuo at 50° C. and the crude material was suspended in DCM/EtOAc (75 mL. 1:1) and stirred for 15 min. The solids were removed by filtration and washed with DCM (25 mL). The filtration was evaporated in vacuo. The crude product was purified by flash chromatography (0-25% acetone/DCM) to get 18-(tert-butoxy)-18-oxooctadecanoic acid (1.72 g, 4.64 mmol, 29.2% yield). ¹H NMR (500 MHz, METHANOL-d₄) δ 2.32-2.17 (m, 4H), 1.66-1.54 (m, 4H), 1.50-1.30 (m, 32H).

Step 2: Preparation of 1-(tert-butyl) 18-(perfluorophenyl) octadecanedioate

To a 50-mL round-bottomed flask was added 18-(tert-butoxy)-18-oxooctadecanoic acid (807 mg, 2.178 mmol), N,N-Dimethylformamide (8 mL), pyridine (379 mg, 4.79 mmol), and perfluorophenyl 2,2,2-trifluoroacetate (1220 mg, 4.36 mmol). The flask was sealed with a septum and kept under a blanket of nitrogen and stirred at room temperature overnight. The reaction was poured into a saturated citric acid solution and extracted with DCM (3×). The organic layers were combined and washed with brine, dried over Na₂SO₄, and evaporated in vacuo. The crude product 1-tert-butyl 18-(perfluorophenyl) octadecanedioate (1.1 g, 2.050 mmol, 94% yield) was used as is without purification. ¹H NMR (400 MHz, CHLOROFORM-d) δ 2.70-2.63 (m, 2H), 2.22 (t, J=7.5 Hz, 2H), 1.78 (quin, J=7.5 Hz, 2H), 1.63-1.52 (m, 2H), 1.48-1.39 (m, 12H), 1.37-1.20 (m, 21H).

Step 3: Preparation of (R)-18-((3-azido-1-carboxypropyl)amino)-18-oxooctadecanoic acid

To a solution of 1-tert-butyl 18-(perfluorophenyl) octadecanedioate (110 mg, 0.205 mmol) and (R)-2-amino-4-azidobutanoic acid (29.5 mg, 0.205 mmol) in DMF (7 mL) was added N-ethyl-N-isopropylpropan-2-amine (26.5 mg, 0.205 mmol). The reaction mixture was stirred at room temperature for 1 h. The reaction mixture was concentrated in vacuo and purified via ISCO to give the desired (R)-18-((3-azido-1-carboxypropyl)amino)-18-oxooctadecanoic acid (80 mg, 89%).

Preparation of (S)-1-azido-]6-carboxy-13,18-dioxo-3, 6,9-trioxa-12,17-diazapentatriacontan-35-oic acid, (Azide-Peg3-yGlu-FDA18)

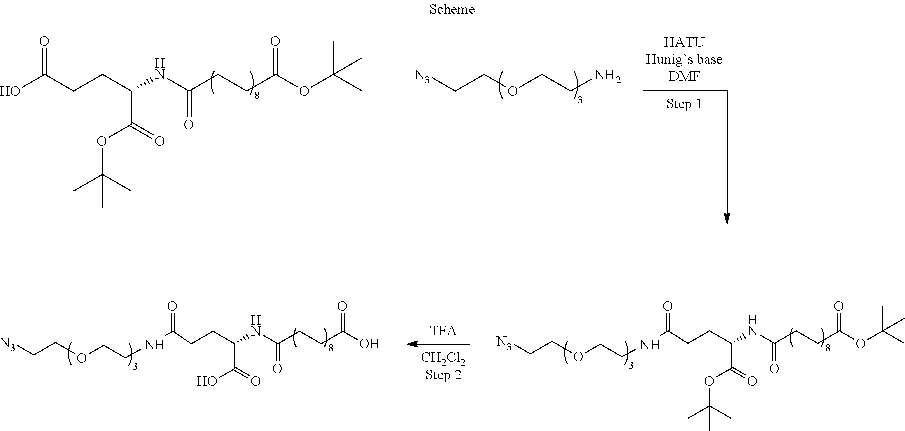

Step 1: Preparation of tert-butyl (S)-1-azido-]6-(tert-butoxycarbonyl)-13,18-dioxo-3,6,9-trioxa-12,17-diazapentatriacontan-35-oate

To a solution of 2-(2-(2-(2-azidoethoxy)ethoxy)ethoxy)ethanamine (113 mg, 0.517 mmol, 1.1 eq) in DMF (4498 μL) was added Hunig'sBase (314 μL, 1.799 mmol, 4.0 eq), then (S)-5-(tert-butoxy)-4-(18-(tert-butoxy)-18-oxooctadecanamido)-5-oxopentanoic acid (250 mg, 0.450 mmol). HATU (342 mg, 0.900 mmol, 2.0 eq) was then added, and the resulting solution was stirred at rt. DMF was removed on high vacuum, and then the residue was applied to silica gel (40 g) and eluted with DCM (100 mL), then a gradient to 75% DCM/acetone over 540 mL and finally a hold at 75% DCM/acetone for 150 mL. The desired fractions were combined. The material was taken into the next step as-is.

Step 2: Preparation of (S)-1-azido-]6-carboxy-13,18-dioxo-3, 6,9-trioxa-12,17-diazapentatriacontan-35-oic acid

To a solution of (S)-tert-butyl 1-azido-16-(tert-butoxycarbonyl)-13,18-dioxo-3,6,9-trioxa-12,17-diazapentatriacontan-35-oate (414.0 mg, 0.548 mmol) in DCM (5476 μL) was added TFA (1266 μL, 16.43 mmol, 30.0 eq). LC/MS indicated a slow reaction, so another 14 eq TFA was added and the mixture was stirred further. After another 6 h, LC/MS indicated a nearly complete reaction. Solvents were removed in vacuo. The mixture was taken up in Hunig's base/MeOH (˜1%). The reaction mixture was purified by PREP HPLC in 5 injections: (30×100 mm HPLC Luna Axia C18 50 to 100% A:B over 10 min, 5 min at 100% B (A is 90:10:0.1 water:MeOH:TFA; B is 90:10:0.1 MeOH:water:TFA)). The desired fractions were combined and concentrated to afford (S)-1-azido-16-carboxy-13,18-dioxo-3,6,9-trioxa-12,17-diazapentatriacontan-35-oic acid (112.4 mg, 0.124 mmol, 22.64% yield). LC/MS: (M+H)⁺=644.45.

### Preparation of (S)-1-azido-22-carboxy-19,24-dioxo-3, 6,9,12,15-pentaoxa-18,23-diazahentetracontan-41-oic acid (ELN)

### 17-{[(IS)-3-[(17-azido-3,6,9,12,15-pentaoxaheptadecan-1-yl)carbamoyl]-1-carboxypropyl]carbamoyl}heptadecanoic acid (IUPAC) (Azide-Peg5-7Glu-FDA18)

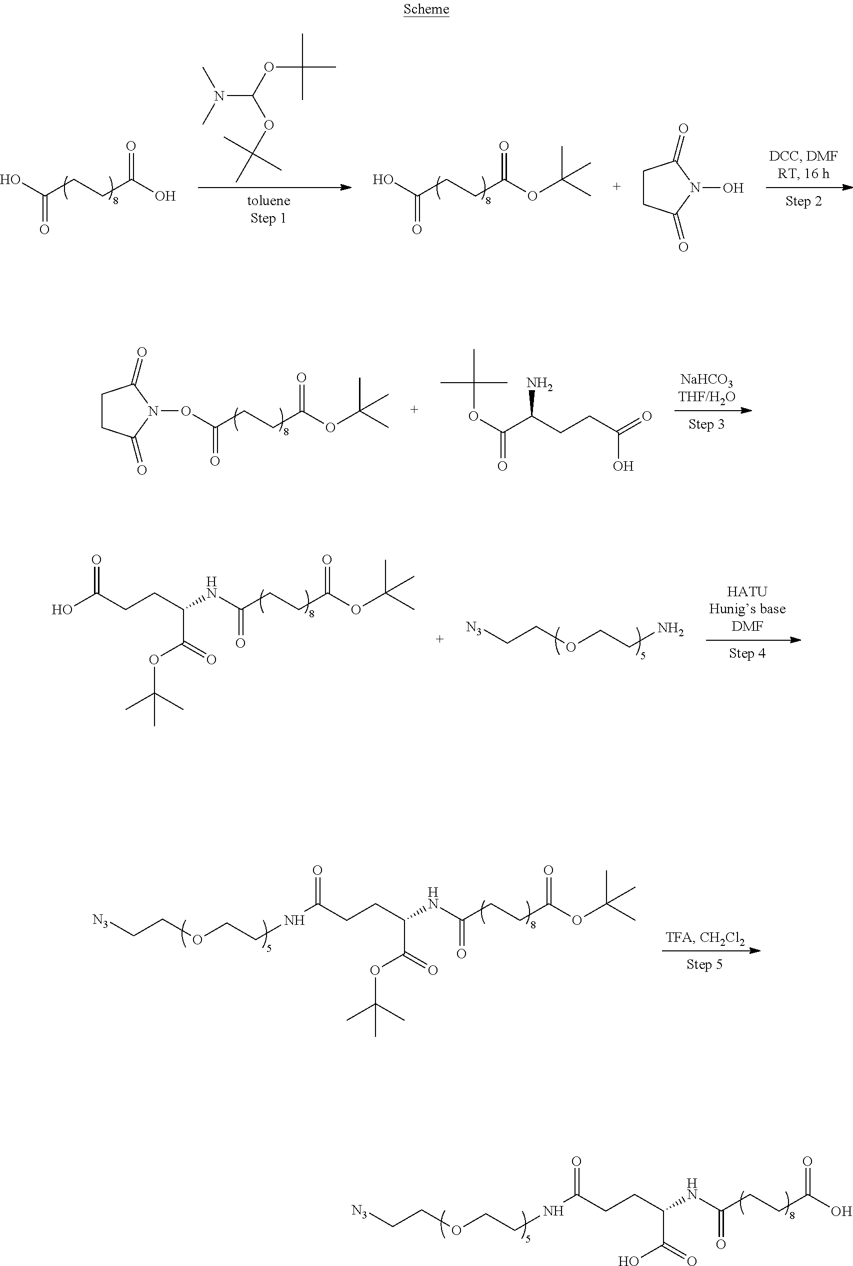

Step 1: Preparation of 18-(tert-butoxy)-18-oxooctadecanoic acid

Octadecanedioic acid (7.5 g, 23.85 mmol) was suspended in toluene (42.6 mL) and the mixture was heated to reflux. 1,1-Di-tert-butoxy-N,N-dimethylmethanamine (15.33 mL, 63.9 mmol, 2.7 eq) was added drop-wise over 30 min. The mixture was reflux overnight. The solvent was removed in vacuo at 50° C. and the crude material was suspended in CH₂Cl₂/EtOAc (110 mL. 1:1) and stirred for 15 min. The solids were removed by filtration and washed with CH₂Cl₂ (40 mL). The filtration was evaporated in vacuo. The crude product was purified by flash chromatography (0-25% acetone/CH₂Cl₂) to get 18-(tert-butoxy)-18-oxooctadecanoic acid (3.95 g, 10.66 mmol, 44.7% yield). Analysis condition D: Retention time=5.04 min; ESI-MS(+) m/z 297.3 [M-OC(CH₃)₃]. ¹H NMR (500 MHz, METHANOL-d₄) δ 2.29 (t, J=7.5 Hz, 2H), 2.22 (t, J=7.4 Hz, 2H), 1.67-1.53 (m, 4H), 1.50-1.42 (m, 9H), 1.40-1.25 (m, 24H).

Step 2: Preparation of 1-tert-butyl 18-(2,5-dioxopyrrolidin-1-yl) octadecanedioate

DCC (5.11 mL, 5.11 mmol, 1.1 eq) was added to a solution of 18-(tert-butoxy)-18-oxooctadecanoic acid (1.72 g, 4.64 mmol) and 1-hydroxypyrrolidine-2,5-dione (0.588 g, 5.11 mmol, 1.1 eq) in DMF (48 mL). The mixture was stirred at rt overnight. The mixture was filtered and concentrated to get 1-tert-butyl 18-(2,5-dioxopyrrolidin-1-yl) octadecanedioate which was used as is in the next step.

Step 3: Preparation of (S)-5-(tert-butoxy)-4-(18-(tert-butoxy)-18-oxooctadecanamido)-5-oxopentanoic acid

Water (5.80 mL) was added to a mixture of (S)-4-amino-5-(tert-butoxy)-5-oxopentanoic acid (1.038 g, 5.11 mmol, 1.1 eq), 1-tert-butyl 18-(2,5-dioxopyrrolidin-1-yl) octadecanedioate (2.171 g, 4.64 mmol), sodium bicarbonate (0.468 g, 5.57 mmol, 1.2 eq) in THE (17.41 mL). The resulting clear solution was stirred at rt for 4 h. The solvents were removed. HCl (6.04 mL, 6.04 mmol, 1.3 eq) was added and the pH was adjusted to 2-3 at 0° C. The resulting suspension was extracted with CH₂Cl₂ (3×). The ordanic layer was concentrated. The resulting crude product was purified by flash chromatography (acetone/CH₂Cl₂ 0-25%) to afford (S)-5-(tert-butoxy)-4-(18-(tert-butoxy)-18-oxooctadecanamido)-5-oxopentanoic acid (2.29 g, 4.12 mmol, 89% yield) as a white solid. Analysis condition D: Retention time=2.74 min; ESI-MS(+) m/z 555.6 (M+H)⁺. ¹H NMR (500 MHz, METHANOL-d₄) δ 4.32 (dd, J=9.0, 5.3 Hz, 1H), 2.45-2.33 (m, 2H), 2.30-2.06 (m, 5H), 1.99-1.82 (m, 3H), 1.78-1.53 (m, 2H), 1.53-1.44 (m, 18H), 1.44-1.26 (m, 24H).

Step 4: Preparation of (S)-tert-butyl I-azido-22-(tert-butoxycarbonyl)-19,24-dioxo-3, 6,9,12,15-pentaoxa-18,23-diazahentetracontan-4]-oate

To a solution of (S)-5-(tert-butoxy)-4-(18-(tert-butoxy)-18-oxooctadecanamido)-5-oxopentanoic acid (225 mg, 0.405 mmol) in DMF (4048 μL) was added Hunig'sBase (212 μL, 1.214 mmol, 3.0 eq) and HATU (308 mg, 0.810 mmol, 2.0 eq). 17-Azido-3,6,9,12,15-pentaoxaheptadecan-1-amine, HCl (139 mg, 0.405 mmol, 1.0 eq) was then added, and the solution was stirred at rt. The crude product was purified by flash chromatography (220 g, silica gel, 10-60% acetone/CH₂Cl₂) to get (S)-tert-butyl 1-azido-22-(tert-butoxycarbonyl)-19,24-dioxo-3,6,9,12,15-pentaoxa-18,23-diazahentetracontan-41-oate (330 mg, 0.391 mmol, 97% yield). Analysis condition D: Retention time=2.88 min; ESI-MS(+) m/z 844.7 (M+H)⁺. ¹H NMR (500 MHz, METHANOL-d₄) δ 4.30-4.23 (m, 1H), 3.74-3.60 (m, 18H), 3.60-3.52 (m, 2H), 3.43-3.35 (m, 4H), 2.34-2.28 (m, 2H), 2.28-2.19 (m, 4H), 2.15-2.08 (m, 1H), 1.98-1.87 (m, 1H), 1.69-1.53 (m, 4H), 1.52-1.44 (m, 18H), 1.41-1.27 (m, 24H).

Step 5: Preparation of (S)-1-azido-22-carboxy-19,24-dioxo-3, 6,9,12,15-pentaoxa-18,23-diazahentetracontan-41-oic acid

The mixture of (S)-tert-butyl 1-azido-22-(tert-butoxycarbonyl)-19,24-dioxo-3,6,9,12,15-pentaoxa-18,23-diazahentetracontan-41-oate (330 mg, 0.391 mmol) and TFA (0.422 mL, 5.47 mmol, 14 eq) in DCM (3.0 mL) was stirred at rt for 2 h. The resulting crude product was purified by Prep-HPLC (Solvent A=0% MeOH -90% H₂O -0.1% TFA, Solvent B=90% MeOH -10% H₂O -0.1% TFA. Column: phenomenex luna 30×150 mm, S10, Flow rate: 40 mL/min, 50-100% B, 10 min and stop at 13 min) to obtain (S)-1-azido-22-carboxy-19,24-dioxo-3,6,9,12,15-pentaoxa-18,23-diazahentetracontan-41-oic acid (101 mg, 0.138 mmol, 35.3% yield). Analysis condition D: Retention time=2.42 min; ESI-MS(+) m/z 732.5 (M+H)⁺.

Preparation of (S)-1-azido-28-carboxy-25,30-dioxo-3, 6,9,12,15,18,21-heptaoxa-24, 29-diazaheptatetracontan-47-oic acid, (Azide-Peg7-2Glu-FDA18)

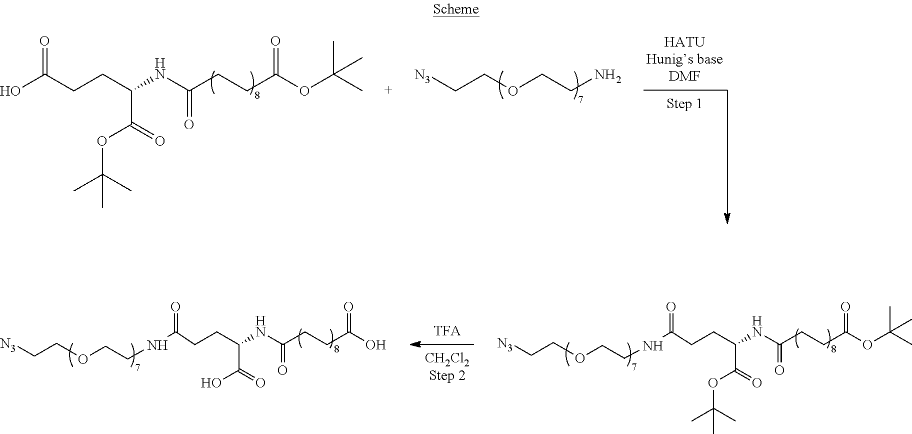

Step 1: Preparation of tert-butyl (S)-1-azido-28-(tert-butoxycarbonyl)-25,30-dioxo-3, 6,9,12,15,18, 21-heptaoxa-24, 29-diazaheptate tracontan-47-oate

To a solution of 23-azido-3,6,9,12,15,18,21-heptaoxatricosan-1-amine (204 mg, 0.517 mmol, 1.15 eq) in DMF (4498 μL) was added Hunig'sBase (314 μL, 1.799 mmol, 4.0 eq), then (S)-5-(tert-butoxy)-4-(18-(tert-butoxy)-18-oxooctadecanamido)-5-oxopentanoic acid (250 mg, 0.450 mmol). HATU (342 mg, 0.900 mmol, 2.0 eq) was then added, and the resulting solution was stirred at rt. DMF was removed on high vacuum, then the residue was applied to silica gel (40 g) and eluted with DCM (90 mL), then a gradient to 75% DCM/acetone over 540 mL and finally a hold at 75% DCM/acetone for 150 mL. The desired fractions were combined to obtain (S)-tert-butyl 1-azido-28-(tert-butoxycarbonyl)-25,30-dioxo-3,6,9,12,15,18,21-heptaoxa-24,29-diazaheptatetracontan-47-oate (394.2 mg, 0.423 mmol, 94% yield).

Step 2: Preparation of (S)-1-azido-28-carboxy-25,30-dioxo-3, 6,9,12,15,18,21-heptaoxa-24,29-diazaheptatetracontan-47-oic acid

To a solution of (S)-tert-butyl 1-azido-28-(tert-butoxycarbonyl)-25,30-dioxo-3,6,9,12,15,18,21-heptaoxa-24,29-diazaheptatetracontan-47-oate (394.2 mg, 0.423 mmol) in DCM (4229 μL) was added TFA (456 μL, 5.92 mmol, 14 eq). Another 14 eq of TFA was added and the mixture was stirred further. After another 6 h, LC/MS indicated a nearly complete reaction. Solvents were removed in vacuo. The mixture was taken up in MeOH. The reaction mixture was purified by PREP HPLC in 7 injections: (30×100 mm HPLC Luna Axia C18 50 to 100% A:B over 10 min, 5 min at 100% B (A is 90:10:0.1 water:MeOH:TFA; B is 90:10:0.1 MeOH:water:TFA)) to give (S)-1-azido-28-carboxy-25,30-dioxo-3,6,9,12,15,18,21-heptaoxa-24,29-diazaheptatetracontan-47-oic acid (121.0 mg, 0.148 mmol, 34.9% yield) after concentration. LC/MS: (M+H)⁺=820.60. ¹H NMR (500 MHz, CHLOROFORM-d) δ 7.30 (d, J=6.4 Hz, 1H), 7.04 (t, J=5.2 Hz, 1H), 4.51 (q, J=6.2 Hz, 1H), 3.72-3.64 (m, 27H), 3.63-3.59 (m, 2H), 3.56-3.49 (m, 1H), 3.47-3.39 (m, 3H), 2.62-2.54 (m, 1H), 2.48-2.40 (m, 1H), 2.36 (t, J=7.4 Hz, 2H), 2.26 (t, J=7.6 Hz, 2H), 2.19-2.08 (m, 2H), 1.65 (quin, J=7.4 Hz, 4H), 1.39-1.24 (m, 25H).

### Preparation of (S)-1-azido-40-carboxy-37,42-dioxo-3, 6,9,12,15,18,21, 24,27, 30,33-undecaoxa-36,41-diazanonapentacontan-59-oic acid (ELN)

### 17-{[(JS)-3-[(35-azido-3,6,9,12,15,18,21,24,27,30,33-undecaoxapentatriacontan-1-yl)carbamoyl]-1-carboxypropyl]carbamoyl}heptadecanoic acid (IUPAC) (Azide-Peg11-yGlu-FDA18)

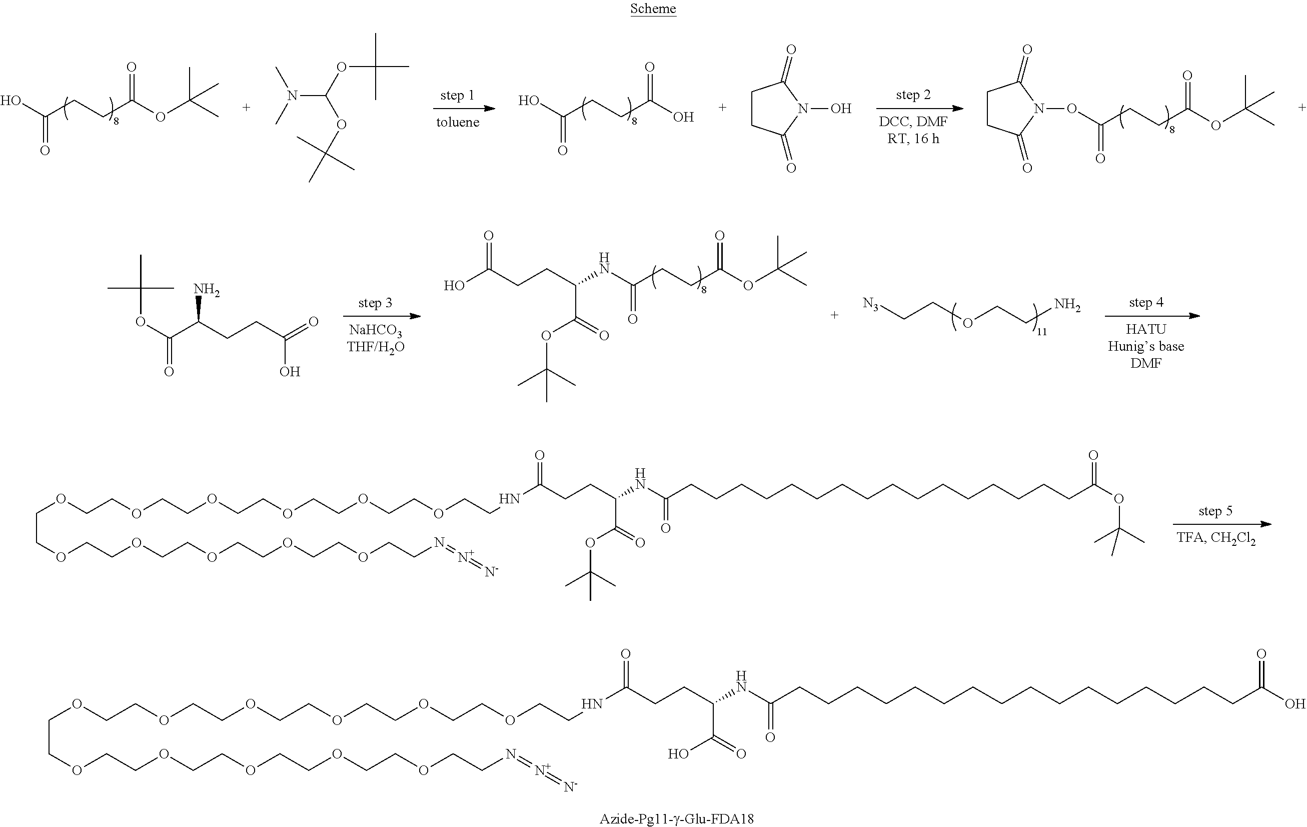

Step 1: Preparation of 18-(tert-butoxy)-18-oxooctadecanoic acid

Octadecanoic acid (7.5 g, 23.85 mmol) was suspended in toluene (42.6 mL) and the mixture was heated to reflux. 1,1-Di-tert-butoxy-N,N-dimethylmethanamine (15.33 mL, 63.9 mmol) was added drop-wise over 30 min. The mixture was reflux overnight. The solvent was removed in vacuo at 50° C. and the crude material was suspended in CH₂Cl₂/EtOAc (110 mL. 1:1) and stirred for 15 min. The solids were removed by filtration and washed with CH₂Cl₂ (40 mL). The filtration was evaporated in vacuo. The crude product was purified by flash chromatography (SG, 0-25% acetone/CH₂Cl₂) to get 18-(tert-butoxy)-18-oxooctadecanoic acid (3.95 g, 10.66 mmol, 44.7% yield). Analysis condition D: Retention time=5.04 min; ESI-MS(+) m/z 297.3 [M -OC(CH₃)₃]. ¹H NMR (500 MHz, METHANOL-d₄) 2.29 (t, J=7.5 Hz, 2H), 2.22 (t, J=7.4 Hz, 2H), 1.67-1.53 (m, 4H), 1.50-1.42 (m, 9H), 1.40-1.25 (m, 24H).

Step 2: Preparation of 1-tert-butyl 18-(2,5-dioxopyrrolidin-1-yl) octadecanedioate

DCC (5.11 mL, 5.11 mmol, 1.1 eq) was added to a solution of 18-(tert-butoxy)-18-oxooctadecanoic acid (1.72 g, 4.64 mmol) and 1-hydroxypyrrolidine-2,5-dione (0.588 g, 5.11 mmol, 1.1 eq) in DMF (48 mL). The mixture was stirred at rt overnight. The mixture was filtered and concentrated to get 1-tert-butyl 18-(2,5-dioxopyrrolidin-1-yl) octadecanedioate which was used as is in the next step.

Step 3: Preparation of (S)-5-(tert-butoxy)-4-(18-(tert-butoxy)-18-oxooctadecanamido)-5-oxopentanoic acid

Water (5.80 mL) was added to a mixture of (S)-4-amino-5-(tert-butoxy)-5-oxopentanoic acid (1.038 g, 5.11 mmol), 1-tert-butyl 18-(2,5-dioxopyrrolidin-1-yl) octadecanedioate (2.171 g, 4.64 mmol, 1.10 eq), sodium bicarbonate (0.468 g, 5.57 mmol, 1.2 eq) in THE (17.41 mL). The resulting clear solution was stirred at rt for 4 h. The solvent was removed in vacuo. HCl (6.04 mL, 6.04 mmol, 1.3 eq) was added and the pH was adjusted to 2-3 at 0° C. The resulting suspension was extracted with CH₂Cl₂ (3×). The organic fractions were combined, dried over anhydrous sodium sulfate, and filtered. The organic solution was concentrated on rotovap. The resulting crude product was purified by flash chromatography (acetone/CH₂Cl₂ 0-25%) to afford (S)-5-(tert-butoxy)-4-(18-(tert-butoxy)-18-oxooctadecanamido)-5-oxopentanoic acid (2.29 g, 4.12 mmol, 89% yield) as a white solid. Analysis condition D: Retention time=2.74 min; ESI-MS(+) m/z 555.6 (M+H)⁺. ¹H NMR (500 MHz, METHANOL-d₄) δ 4.32 (dd, J=9.0, 5.3 Hz, 1H), 2.45-2.33 (m, 2H), 2.30-2.06 (m, 5H), 1.99-1.82 (m, 3H), 1.78-1.53 (m, 2H), 1.53-1.44 (m, 18H), 1.44-1.26 (m, 24H).

Step 4: Preparation of (S)-tert-butyl 1-azido-40-(tert-butoxycarbonyl)-37,42-dioxo-3,6,9,12,15,18,21,24,27,30,33-undecaoxa-36,41-diazanonapentacontan-59-oate

To a solution of (S)-5-(tert-butoxy)-4-(18-(tert-butoxy)-18-oxooctadecanamido)-5-oxopentanoic acid (438 mg, 0.789 mmol, 1.5 eq) in DMF (1593 μL) was added Hunig'sBase (275 μL, 1.577 mmol, 3.0 eq) and HATU (400 mg, 1.051 mmol, 2.0 eq). 35-azido-3,6,9,12,15,18,21,24,27,30,33-undecaoxapentatriacontan-1-amine (300 mg, 0.526 mmol) was then added, and the solution stirred at rt overnight. The mixture was poured into water and extracted with CH₂Cl₂ (3×). The combined organic extracts were dried over MgSO₄, filtered, and concentrated in vacuo to get (S)-tert-butyl 1-azido-40-(tert-butoxycarbonyl)-37,42-dioxo-3,6,9,12,15,18,21,24,27,30,33-undecaoxa-36,41-diazanonapentacontan-59-oate, which is used as is in the next step. Analysis condition D: Retention time=2.84 min; ESI-MS(+) m/z 1109.1 (M+H)⁺.

Step 5: Preparation of 17-{[(1 S)-3-[(35-azido-3,6,9,12,15,18,21,24,27,30,33-undecaoxapentatriacontan-1-yl)carbamoyl]-1-carboxypropyl]carbamoyl}heptadecanoic acid

The mixture of (S)-tert-butyl 1-azido-40-(tert-butoxycarbonyl)-37,42-dioxo-3,6,9,12,15,18,21,24,27,30,33-undecaoxa-36,41-diazanonapentacontan-59-oate (280 mg, 0.253 mmol) and TFA (3 mL, 38.9 mmol) in DCM (3.0 mL) was stirred at rt for 2 h. The resulting crude product was purified by Prep-HPLC (Solvent A=10% MeOH-90% H₂O-0.1⁰% TFA, Solvent B=90% MeOH-10% H₂O -0.1% TFA. Column: phenomenex luna 30×100 mm, S10, Flow rate: 40 mL/min, 50-100% B, 10 min and stop at 12 min) to obtain (S)-1-azido-40-carboxy-37,42-dioxo-3,6,9,12,15,18,21,24,27,30,33-undecaoxa-36,41-diazanonapentacontan-59-oic acid (124 mg, 0.124 mmol, 49.3% yield). Analysis condition D: Retention time=2.43 min; ESI-MS(+) m/z 996.9 (M+H)⁺. ¹H NMR (500 MHz, METHANOL-d₄) δ 4.44-4.35 (m, 1H), 3.84-3.27 (m, 48H), 2.39-2.11 (m, 7H), 2.04-1.87 (m, 1H), 1.71-1.55 (m, 4H), 1.44-1.18 (m, 24H).

Preparation of functionalized tails

### Compound Fmoc-DapB-FDA14

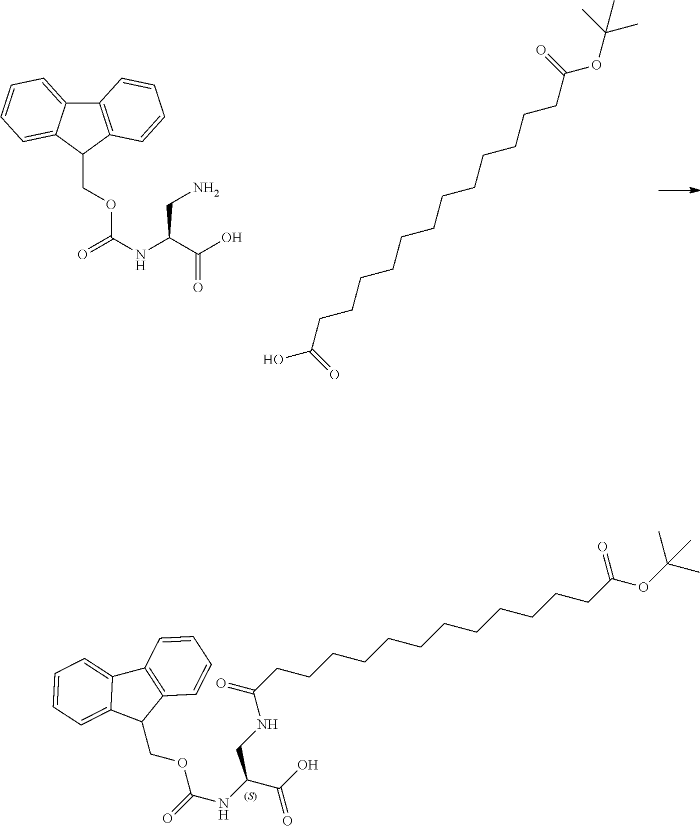

To a solution of 14-(tert-butoxy)-14-oxododecanoic acid (500 mg, 1.59 mmol) in 10 mL of DMF was added HATU (725 mg, 1.908 mmol) and half portion of N-methylmorpholine (0.262 mL, 2.38 mmol). The reaction mixture was stirred at rt for 30 minutes, followed by (S)-2-((((9H-fluoren-9-yl)methoxy)carbonyl)amino)-3-aminopropanoic acid (519 mg, 1.590 mmol) and the rest of base. After 2h stirring, the solvent was evaporated under pressure and purified via ISCO on 40g cartridge, eluted with 10-20% MeOH in CH₂Cl₂. Fractions were combined and solvent was evaporated under pressure to give the desired product (Analysis condition S: Retention time=1.22 min; ESI-MS(+) m/z [M+2H]²⁺: 623.6).

### Compound Fmoc-DapB-FDA18

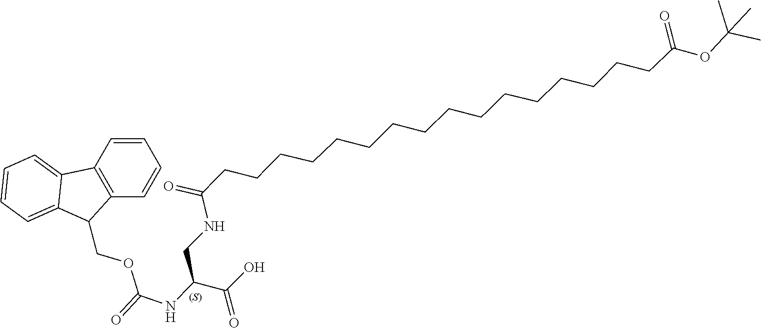

Prepared following the procedure described for Fmoc-DapB-FDA14 above. Analysis condition S: Retention time=1.35 min; ESI-MS(+) m/z [M+H]⁺: 679.5.

### Compound Fmoc-DapB-FDA12

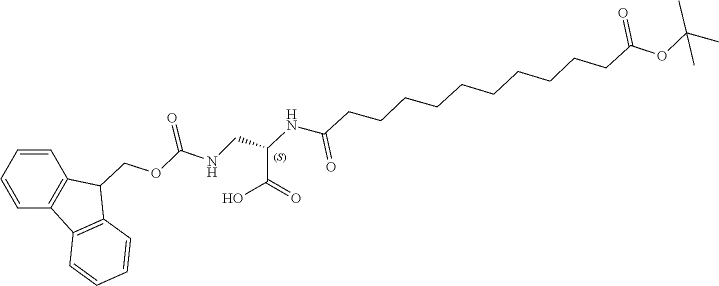

Prepared following the procedure described for Fmoc-DapB-FDA14 above. Prepared following the procedure described for Fmoc-DabG-FDA18 above. Analysis condition S: Retention time=1.19 min; ESI-MS(+) m/z [M+H]⁺: 595.4.

### Compound Fmoc-DabG-FA15

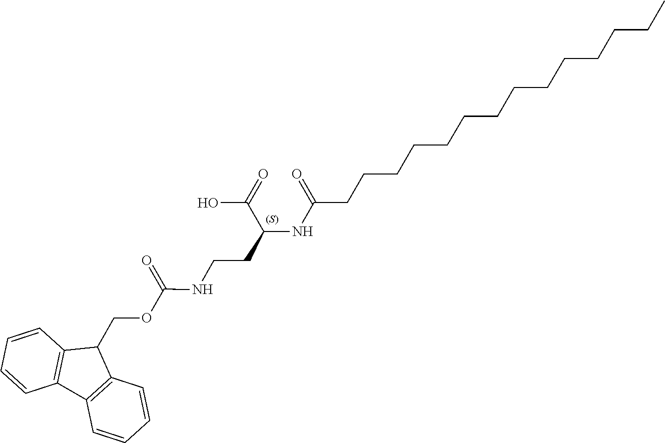

Prepared following the procedure described for Fmoc-DapB-FDA14 above. Analysis condition S: Retention time=1.36 min; ESI-MS(+) m/z [M+H]⁺: 565.5.

### Compound Fmoc-DabG-FDA18

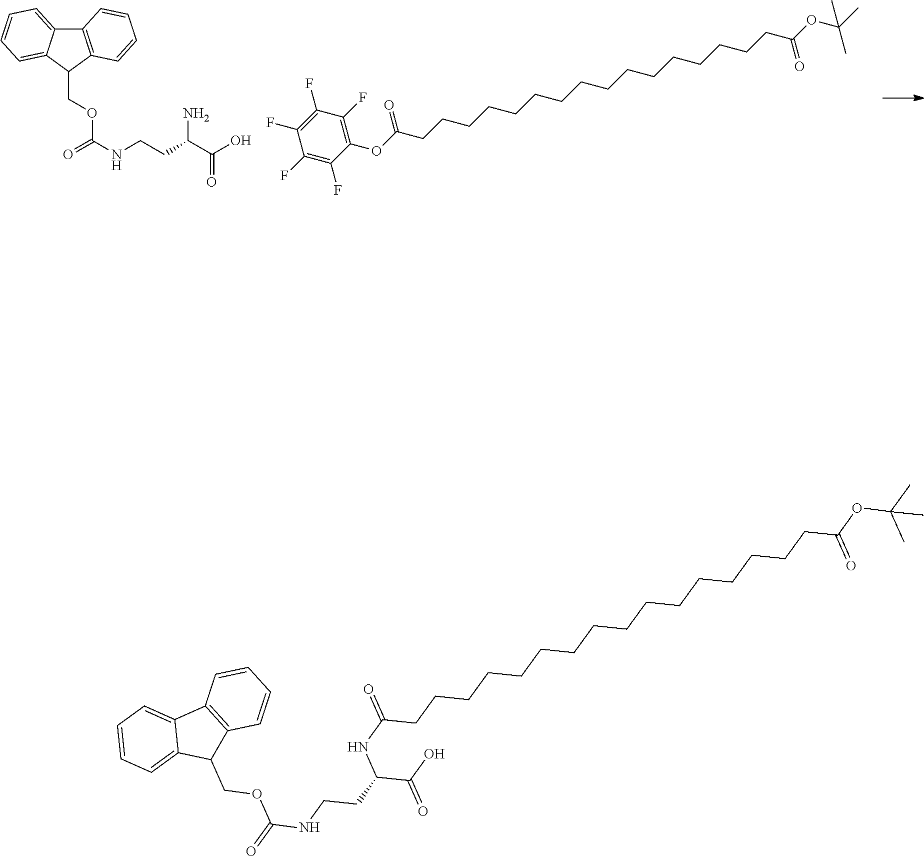 [SMILES: N[C@@H](CCNC(=O)OCC1c2ccccc2-c2ccccc21)C(=O)O ; CC(C)(C)OC(=O)CCCCCCCCCCCCCCCCC(=O)Oc1c(F)c(F)c(F)c(F)c1F ; CC(C)(C)OC(=O)CCCCCCCCCCCCCCCCC(=O)N[C@@H](CCNC(=O)OCC1c2ccccc2-c2ccccc21)C(=O)O]

To a solution of (S)-4-((((9H-fluoren-9-yl)methoxy)carbonyl)amino)-2-aminobutanoic acid (2 g, 5.88 mmol) in DMF (50 mL) was added 1-(tert-butyl) 18-(perfluorophenyl) octadecanedioate (3.15 g, 5.88 mmole, A1FA2-017-01 which described above), followed by DIEA (3.08 mL, 17.63 mmol). The reaction mixture was stirred at rt for 3h. DMF was removed in vac and the desired was purified via ISCO using 10-30% MeOH in DCM on 80g silica gel column, the desired was eluted out at 10% MeOH/DCM and gave S)-4-((((9H-fluoren-9-yl)methoxy)carbonyl)amino)-2-(18-(tert-butoxy)-18-oxooctadecanamido)butanoic acid (3.8 g, 5.48 mmol, 93% yield). Analysis condition S: Retention time=1.31 min; ESI-MS(+) m/z [M+H]⁺: 693.6

### Compound Fmoc-DabG-FDA17

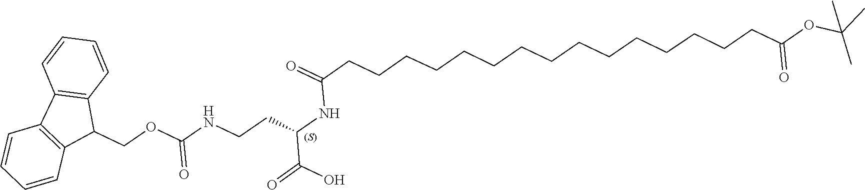

Prepared following the procedure described for Fmoc-DabG-FDA18 above. Analysis condition T: Retention time=2.33 min; ESI-MS(+) m/z [M+H]⁺: 679.5.

### Compound Fmoc-DabG-FDA15

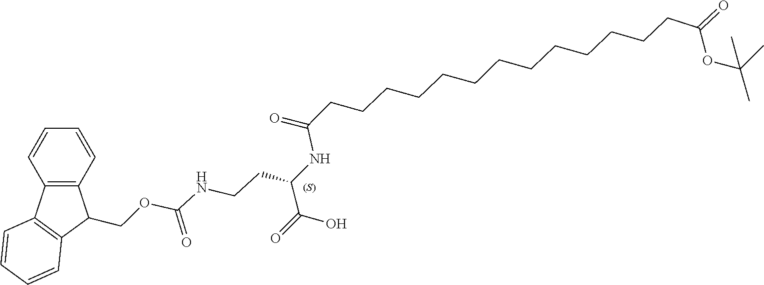

Prepared following the procedure described for Fmoc-DabG-FDA18 above. Analysis condition U: Retention time=1.91 min; ESI-MS(−) m/z [M+H]⁺: 649.4.

### Compound Fmoc-DapB-FDA12

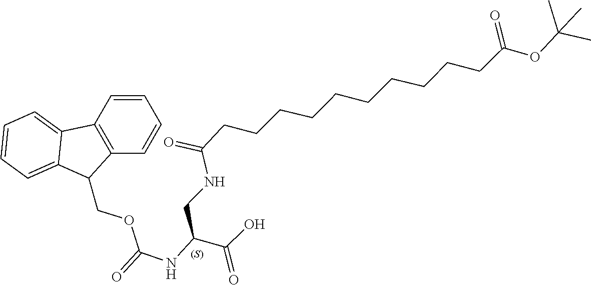

Prepared following the procedure described for Fmoc-DabG-FDA18 above. Analysis condition S: Retention time=1.19 min; ESI-MS(+) m/z [M+H]⁻: 595.4.

### Compound Fmoc-DapB-FDA14

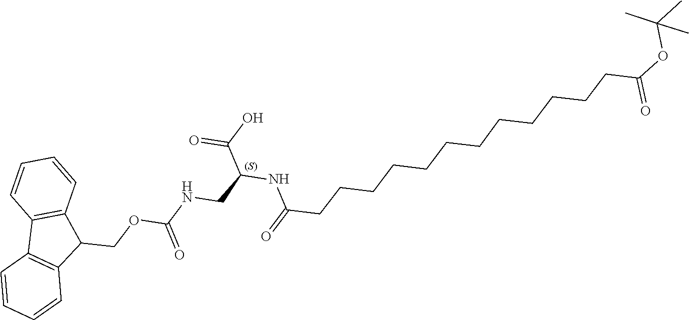

Prepared following the procedure described for Fmoc-DabG-FDA18 above. Analysis condition S: Retention time=1.23 min; ESI-MS(+) m/z [M+2H]²⁺: 624.8.

### Compound Fmoc-DabG-FDA14

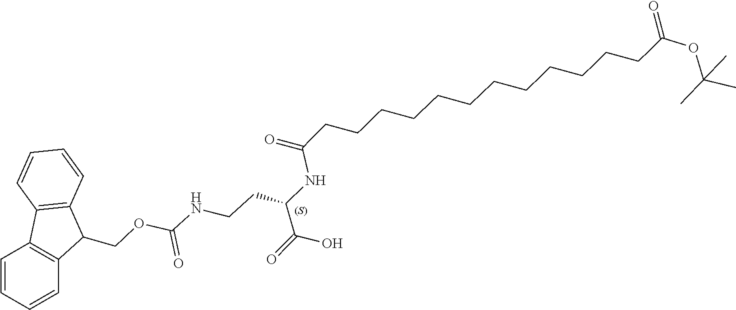

Prepared following the procedure described for Fmoc-DabG-FDA18 above. Analysis condition S: Retention time=1.24 min; ESI-MS(+) m/z [M+H]⁺: 637.8.

### Preparation of FDA functionalized resins

### Compound Fmoc-DabG-FDA14-C₁-Trt Resin

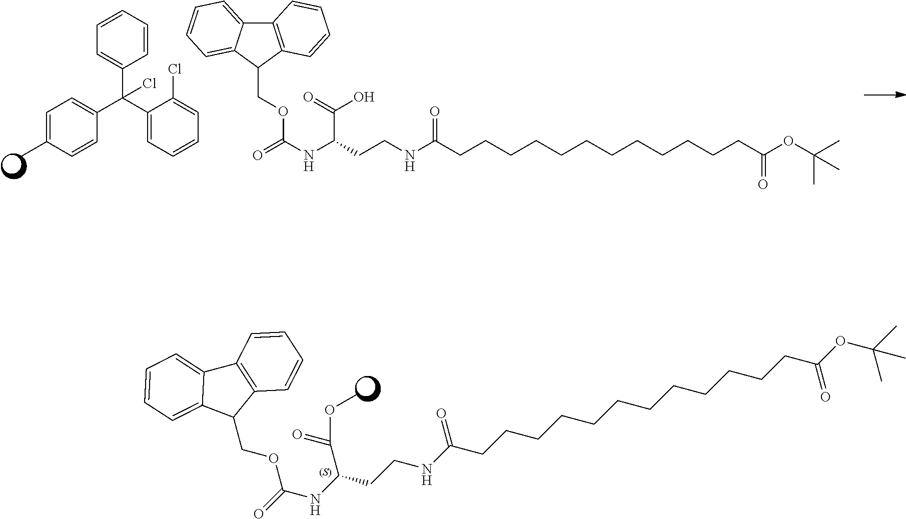

Swell C1-Trt resin(5 g, 7.50 mmol) in DCM for 10 min, then drain. To pre-swelled 1-chloro-2-(chloro(phenyl)(p-tolyl)methyl)benzene (5 g, 7.50 mmol) in 30 mL of DCM was added (S)-2-((((9H-fluoren-9-yl)methoxy)carbonyl)amino)-4-(14-(tert-butoxy)-14-oxotetradecanamido)butanoic acid (5.25 g, 8.25 mmol), followed by DIEA (3.93 mL, 22.50 mmol). The mixture was shaked for 16 h at room temperature. Small (5 mL) amount of MeH and DIEA (20 mL) was added to the mixture reaction and shake for extra 10 minutes, drained and wash with DMF (50 mL×4), DCM (50 mL×4) and Ethylether (50 mL×1). Pump to dryness. Analysis condition S: Retention time=1.24 min; ESI-MS(+) m/z [M+H]⁺: 637.8.

### Compound Fmoc-DapB-FDA14-C₁-Trt Resin

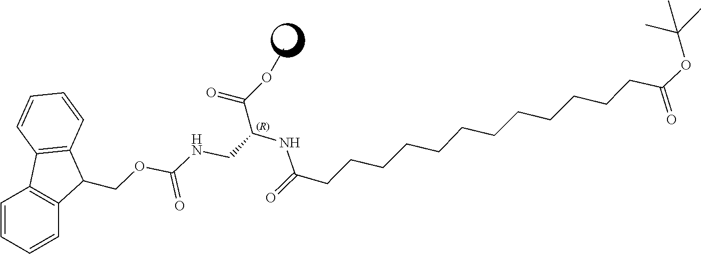

Prepared following the procedure described for Fmoc-DabG-FDA14-C₁-Trt Resin above. Analysis condition S: Retention time=1.23 min; ESI-MS(+) m/z [M+2H]²⁺: 624.8).

### Compound Fmoc-DabG-FDA12 C₁-Trt Resin

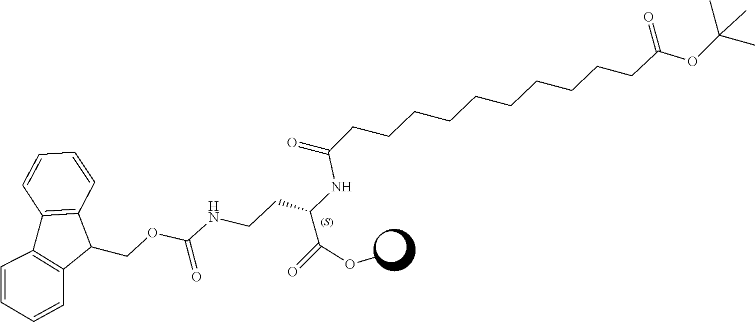

Prepared following the procedure described for Fmoc-DabG-FDA14-C₁-Trt Resin above. Analysis condition S: Retention time=1.20 min; ESI-MS(+) m/z [M+H]⁺: 609.7.

### Compound Fmoc-DapB-FDA12 C₁-Trt Resin

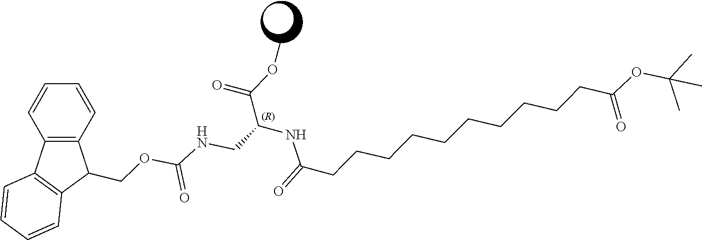

Prepared following the procedure described for Fmoc-DabG-FDA14-C₁-Trt Resin above. Analysis condition S: Retention time=1.19 min; ESI-MS(+) m/z [M+H]⁻: 595.4.

### Compound Fmoc-DapB-FDA14-C₁-Trt Resin

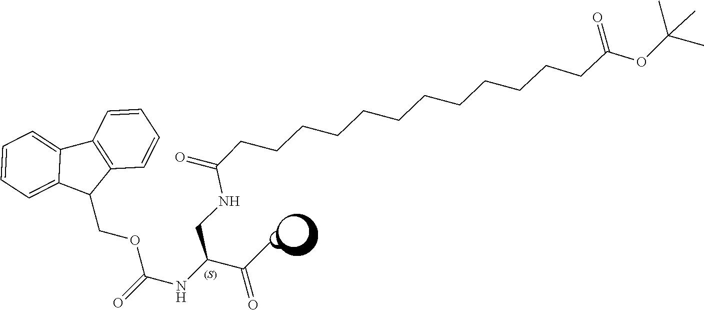

Prepared following the procedure described for Fmoc-DabG-FDA14-C₁-Trt Resin above. Analysis condition B: Retention time=1.22 min; ESI-MS(+) m/z [M+2H]²⁺: 623.6).

### Compound Fmoc-DapB-FDA18-C₁-Trt Resin

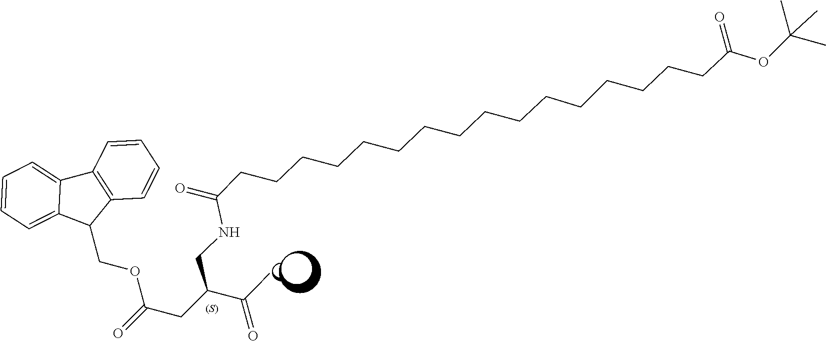

Prepared following the procedure described for Fmoc-DabG-FDA14-C₁-Trt Resin above. Analysis condition S: Retention time=1.35 min; ESI-MS(+) m/z [M+H]⁺: 679.9.

### Compound Fmoc-DabG-FDA18-C₁-Trt Resin

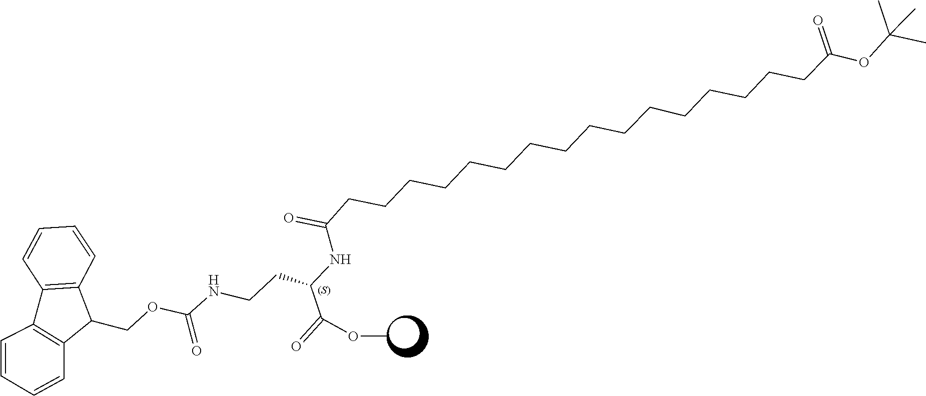

Prepared following the procedure described for Fmoc-DabG-FDA14-C₁-Trt Resin above. Analysis condition S: Retention time=1.34 min; ESI-MS(+) m/z [M+H]⁻: 693.6.

### Compound Fmoc-DabG-FA15-C₁-Trt Resin

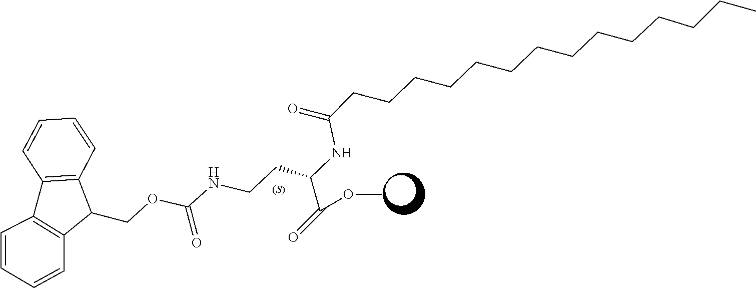

Prepared following the procedure described for Fmoc-DabG-FDA14-C₁-Trt Resin above. Analysis condition B: Retention time=1.36 min; ESI-MS(+) m/z [M+H]⁺: 565.7.

### Compound Fmoc-DabG-FDA15-C₁-Trt Resin

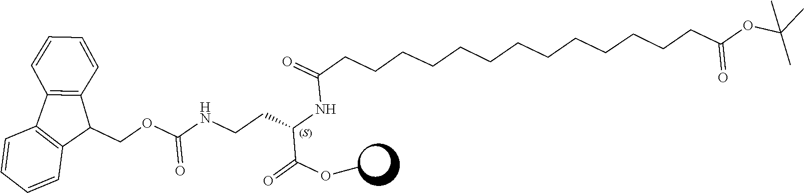

Prepared following the procedure described for Fmoc-DabG-FDA14-C₁-Trt Resin above. Analysis condition U: Retention time=1.91 min; ESI-MS(−) m/z [M+H]⁺: 649.4.

### Compound Fmoc-DabG-FDA17-Cl Trt Resin

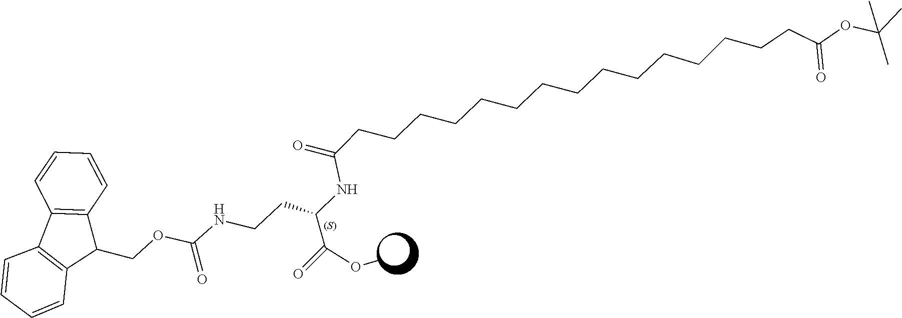

Prepared following the procedure described for Fmoc-DabG-FDA14-C₁-Trt Resin above. Analysis condition T: Retention time=2.33 min; ESI-MS(+) m/z [M+H]⁺: 679.5.

### Final Compound Examples

### Preparation of Compound 100]

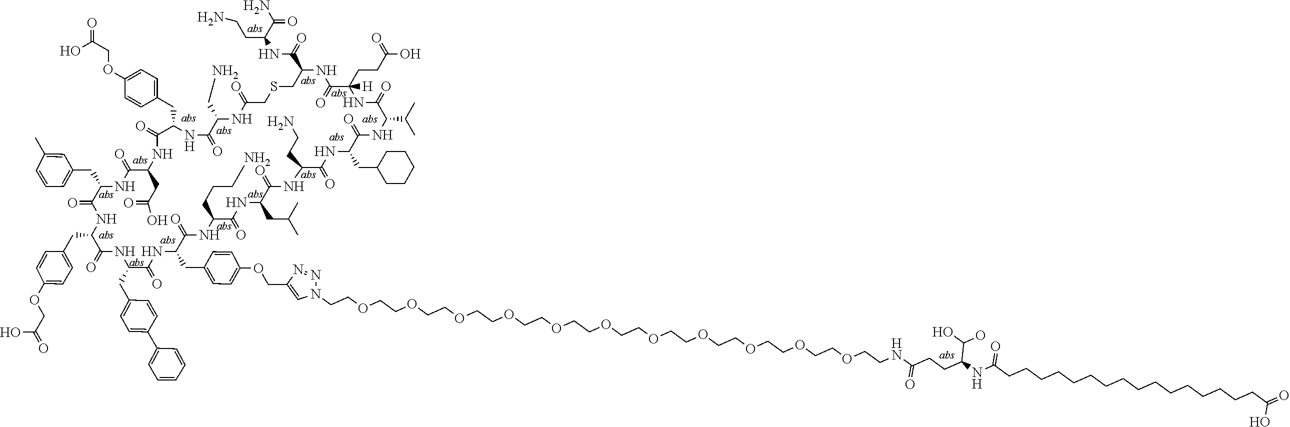

The compound was synthesized via “On-Resin Click Method A”. To a small solid phase reactor was added the Sieber resin of the following linear sequence: ClAc-Dap(Boc)-Tyr(CH₂COOtBu)-Asp(OtBu)-Phe(3-Me)-Tyr(CH₂COOtBu)-Bip-Tyr(CH₂-propargyl)-Orn(Boc)-dLeu-Dab(Boc)-Cha-Val-Glu(OtBu)-Cys(Trt)-Dab(Boc)-Sieber resin (50 μmol). The linear sequence was assembled using Symphony X peptide synthesizer following the general procedures composing of the following general procedures:

“Symphony X Resin-Swelling procedure”, “Symphony X Single-Coupling procedure”, “Symphony X Chloroacetic Anhydride coupling procedure” wherein 10 equiv. of amino acid, 1 h-2 h coupling time was used for all the amino acid coupling steps. The resin was swelled first with DCM (2×5 mL, shaking for 5 min each wash) and then DMF (3×5 mL, shaking for 5 min each wash).

In a separate vial was charged vitamin C (26.4 mg, 150 μmol), bis(2,2,6,6-tetramethyl-3,5-heptanedionato)copper(II) (10.75 mg, 25.00 μmol), DMF (1.5 mL), 2,6-lutidine (0.058 mL, 500 μmol), and THE (1.500 mL). To this stirring solution was added DIPEA (0.087 mL, 500 μmol) followed by (S)-1-azido-40-carboxy-37,42-dioxo-3,6,9,12,15,18,21,24,27,30,33-undecaoxa-36,41-diazanonapentacontan-59-oic acid, N₃-Pegl 1-γGLu-FDA18 (50-75 μmol) as a solid. The resulting mixture was stirred until everything was in solution.

DMF was drained through the frit of the above solid phase reactor and the above click cocktail solution was added to the resin. The reactor was shaken for 16 h. The solution was drained and the resin was washed with DMF (5×) and DCM (5×).

“Global Deprotection Method A” was then followed. The resin in each 40 mL falcon tube was treated with 5 mL of 95% TFA 0.5% DTT and 4.5% TIS at rt for 1.5 h. The linear peptide was crashed out with 45 mL of Et20. After cooling for 1 h in a freezer, it was centrifuged and decanted. Washed the residue one more time with 40 mL of Et20. It was centrifuged, decanted, and then air-dried at rt for 60 min,

“Cyclization Method A” was then followed. To the residue was added DMF (40 mL containing 10% by volume of DIEA). The reaction was shaken at rt overnight. LCMS indicates desired product. It was concentrated to dryness on Genevac, and then diluted with DMSO or DMF (2 mL), filtered and submitted to single compound purification.

The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 20% B, 20-70% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 25.6 mg, and its estimated purity by LCMS analysis was 91.2%. Analysis condition A: Retention time=1.78 min; ESI-MS(+) m/z [M+3H]³⁺: 1065.2.

Compounds 1002-1033 were prepared according to the synthetic procedure for Compound 1001.

### Preparation of Compound 1002

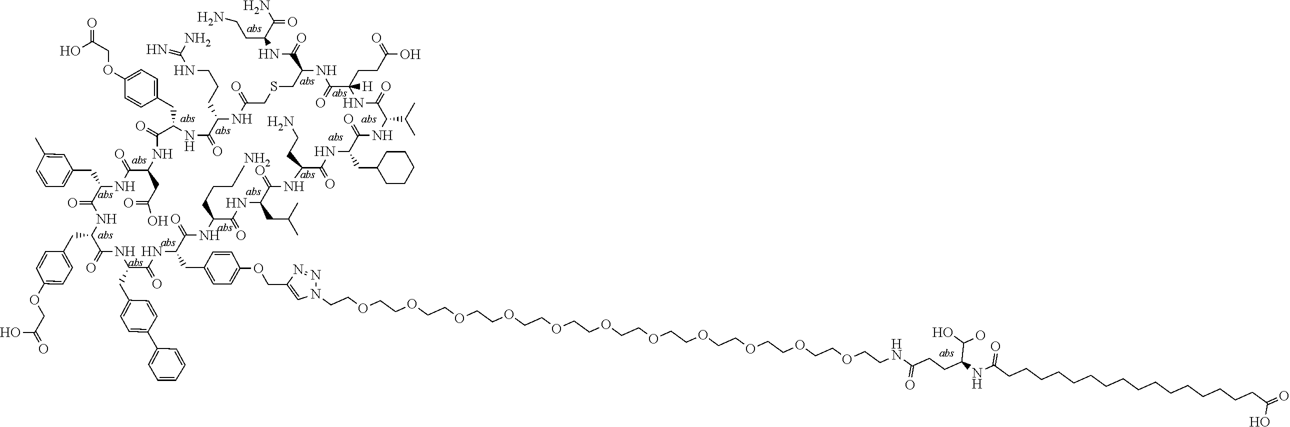

Compound 1002 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 20% B, 20-69% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 13.6 mg, and its estimated purity by LCMS analysis was 95.7%. Analysis condition B: Retention time=1.98 min; ESI-MS(+) m/z [M+3H]³⁺: 1089.1.

### Preparation of Compound 1003

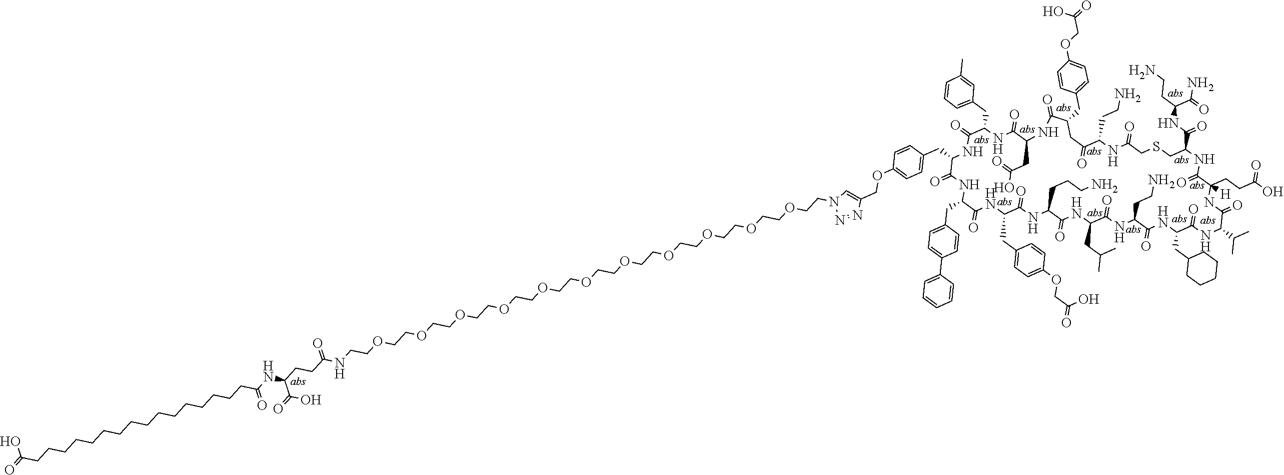

Compound 1003 was prepared on a 50 [μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 15% B, 15-70% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 1 mg, and its estimated purity by LCMS analysis was 85.7%. Analysis condition A: Retention time=1.79 min; ESI-MS(+) m/z [M+3H]f*: 1070.2.

### Preparation of Compound 1004

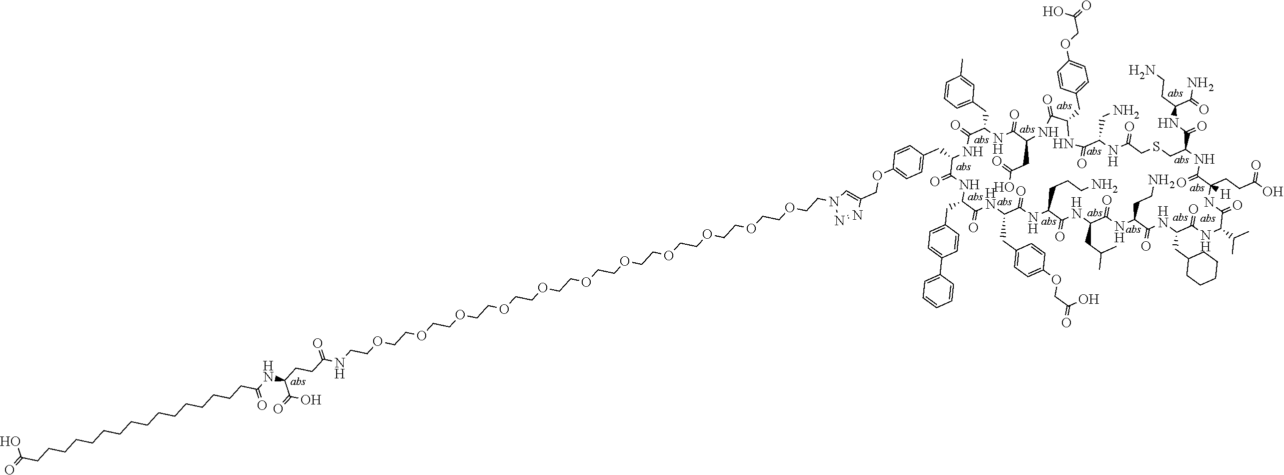

Compound 1004 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 18% B, 18-720% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 15 mg, and its estimated purity by LCMS analysis was 87.7%. Analysis condition A: Retention time=1.78 min; ESI-MS(+) m/z [M+2H]²⁺: 1598.

### Preparation of Compound 1005

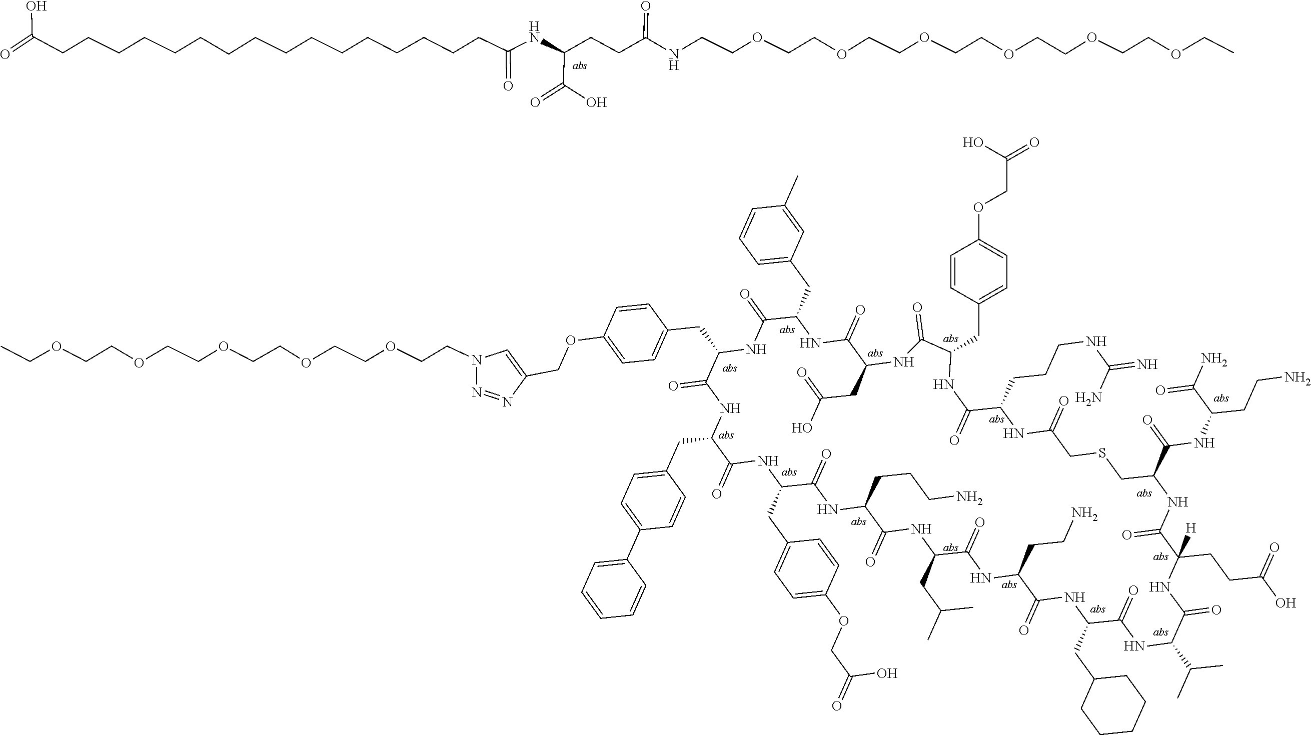

Compound 1005 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 15% B, 15-68% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 23% B, 23-63% B over 20 minutes, then a 2-minute hold at 100% B; Flow Rate: 40 mL/min; Column Temperature: 25° C. Fraction collection was triggered by MS signals. Fractions containing the desired product were combined and dried via centrifugal evaporation. The yield of the product was 4.6 mg, and its estimated purity by LCMS analysis was 97.3%. Analysis condition A: Retention time=1.74 min; ESI-MS(+) m/z [M+3H]³⁺: 1089.3.

### Preparation of Compound 1006

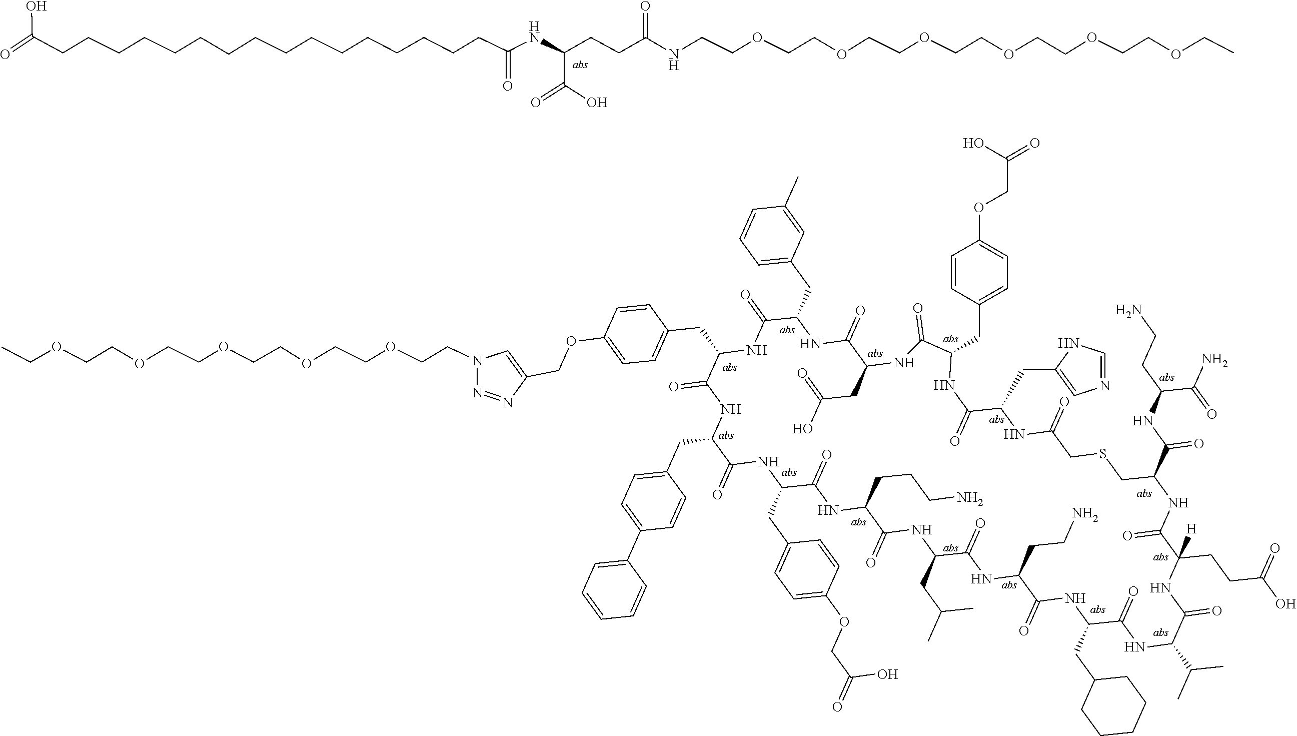

Compound 1006 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 20% B, 20-70% B over 27 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 22% B, 22-62% B over 20 minutes, then a 2-minute hold at 100% B; Flow Rate: 40 mL/min; Column Temperature: 25° C. Fraction collection was triggered by MS signals. Fractions containing the desired product were combined and dried via centrifugal evaporation. The yield of the product was 4.9 mg, and its estimated purity by LCMS analysis was 98.1%.

Analysis condition A: Retention time=1.76 min; ESI-MS(+) m/z [M+3H]³⁺: 1082.9.

### Preparation of Compound 1007

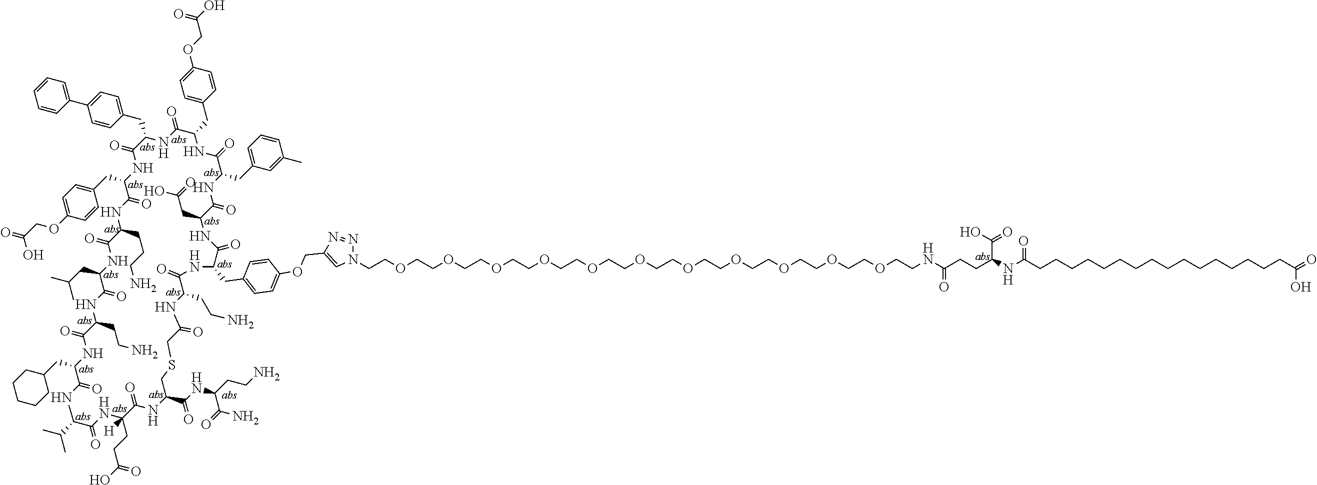 [SMILES: Cc1cccc(C[C@@H]2NC(=O)[C@H](CC(=O)O)NC(=O)[C@H](Cc3ccc(OCc4cn(CCOCCOCCOCCOCCOCCOCCOCCOCCOCCOCCOCCNC(=O)CC[C@H](NC(=O)CCCCCCCCCCCCCCCCC(=O)O)C(=O)O)nn4)cc3)NC(=O)[C@H](CCN)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1007 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 19% B, 19-72% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 25% B, 25-65% B over 20 minutes, then a 4-minute hold at 100% B; Flow Rate: 40 mL/min; Column Temperature: 25° C. Fraction collection was triggered by MS signals. Fractions containing the desired product were combined and dried via centrifugal evaporation. The yield of the product was 2.1 mg, and its estimated purity by LCMS analysis was 98.9%. Analysis condition A: Retention time=1.84 min; ESI-MS(+) m/z [M+3H]³⁺: 1070.

### Preparation of Compound 1008

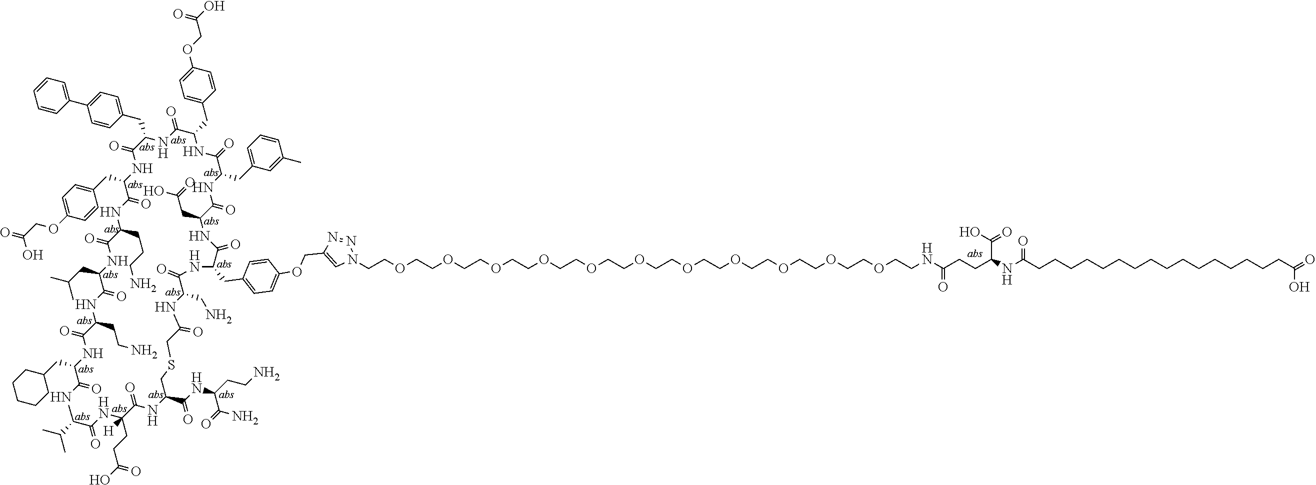 [SMILES: Cc1cccc(C[C@@H]2NC(=O)[C@H](CC(=O)O)NC(=O)[C@H](Cc3ccc(OCc4cn(CCOCCOCCOCCOCCOCCOCCOCCOCCOCCOCCOCCNC(=O)CC[C@H](NC(=O)CCCCCCCCCCCCCCCCC(=O)O)C(=O)O)nn4)cc3)NC(=O)[C@H](CN)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1008 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 19% B, 19-72% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 25% B, 25-65% B over 20 minutes, then a 2-minute hold at 100% B; Flow Rate: 40 mL/min; Column Temperature: 25° C. Fraction collection was triggered by MS signals. Fractions containing the desired product were combined and dried via centrifugal evaporation. The yield of the product was 5.6 mg, and its estimated purity by LCMS analysis was 98.7%. Analysis condition A: Retention time=1.84 min; ESI-MS(+) m/z [M+2H]²⁺: 1597.6.

### Preparation of Compound 1009

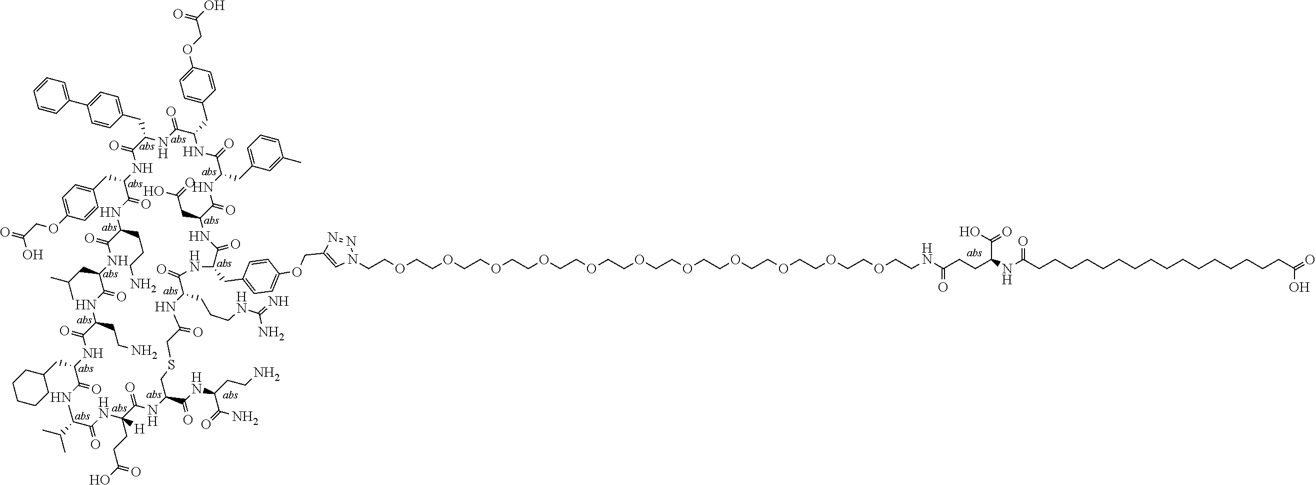 [SMILES: Cc1cccc(C[C@@H]2NC(=O)[C@H](CC(=O)O)NC(=O)[C@H](Cc3ccc(OCc4cn(CCOCCOCCOCCOCCOCCOCCOCCOCCOCCOCCOCCNC(=O)CC[C@H](NC(=O)CCCCCCCCCCCCCCCCC(=O)O)C(=O)O)nn4)cc3)NC(=O)[C@H](CCCNC(=N)N)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1009 was prepared on a 50 [μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.0500 trifluoroacetic acid; Gradient: a 0-minute hold at 20% B, 20-70% B over 26 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 24% B, 24-64% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 4.4 mg, and its estimated purity by LCMS analysis was 99.300.

Analysis condition A: Retention time=1.78 min: ESI-MS(+1 m z [M+3H]³⁺: 1089.3.

### Preparation of Compound 1010

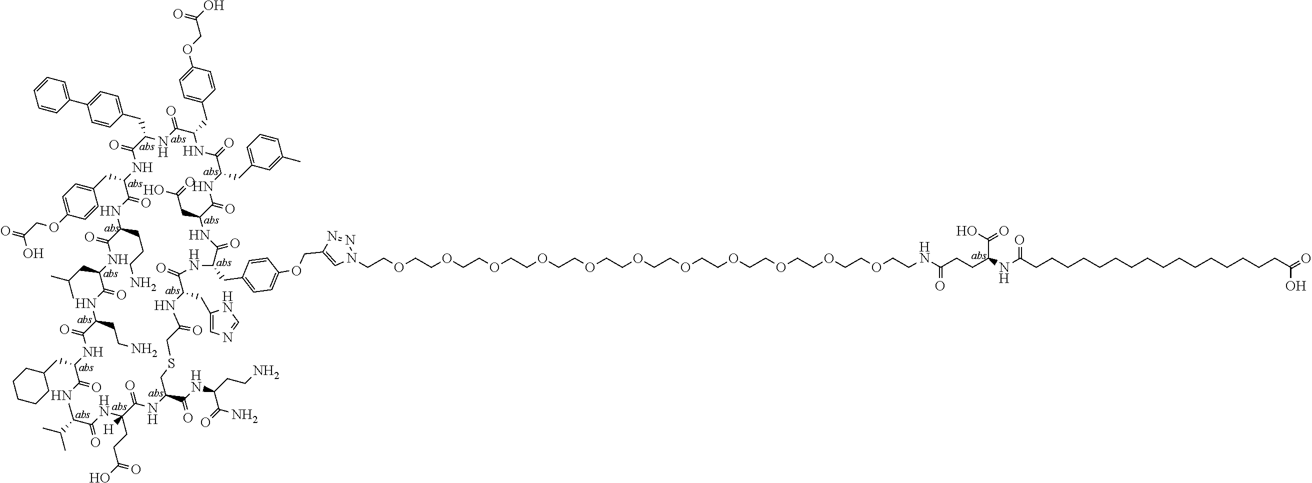 [SMILES: Cc1cccc(C[C@@H]2NC(=O)[C@H](CC(=O)O)NC(=O)[C@H](Cc3ccc(OCc4cn(CCOCCOCCOCCOCCOCCOCCOCCOCCOCCOCCOCCNC(=O)CC[C@H](NC(=O)CCCCCCCCCCCCCCCCC(=O)O)C(=O)O)nn4)cc3)NC(=O)[C@H](Cc3cnc[nH]3)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1010 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 20% B, 20-70% B over 27 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 23% B, 23-63% B over 20 minutes, then a 2-minute hold at 100% B; Flow Rate: 40 mL/min; Column Temperature: 25° C. Fraction collection was triggered by MS signals. Fractions containing the desired product were combined and dried via centrifugal evaporation. The yield of the product was 4.9 mg, and its estimated purity by LCMS analysis was 97.7%. Analysis condition A: Retention time=1.75 min; ESI-MS(+) m/z [M+3H]³⁺: 1083.

### Preparation of Compound 1011

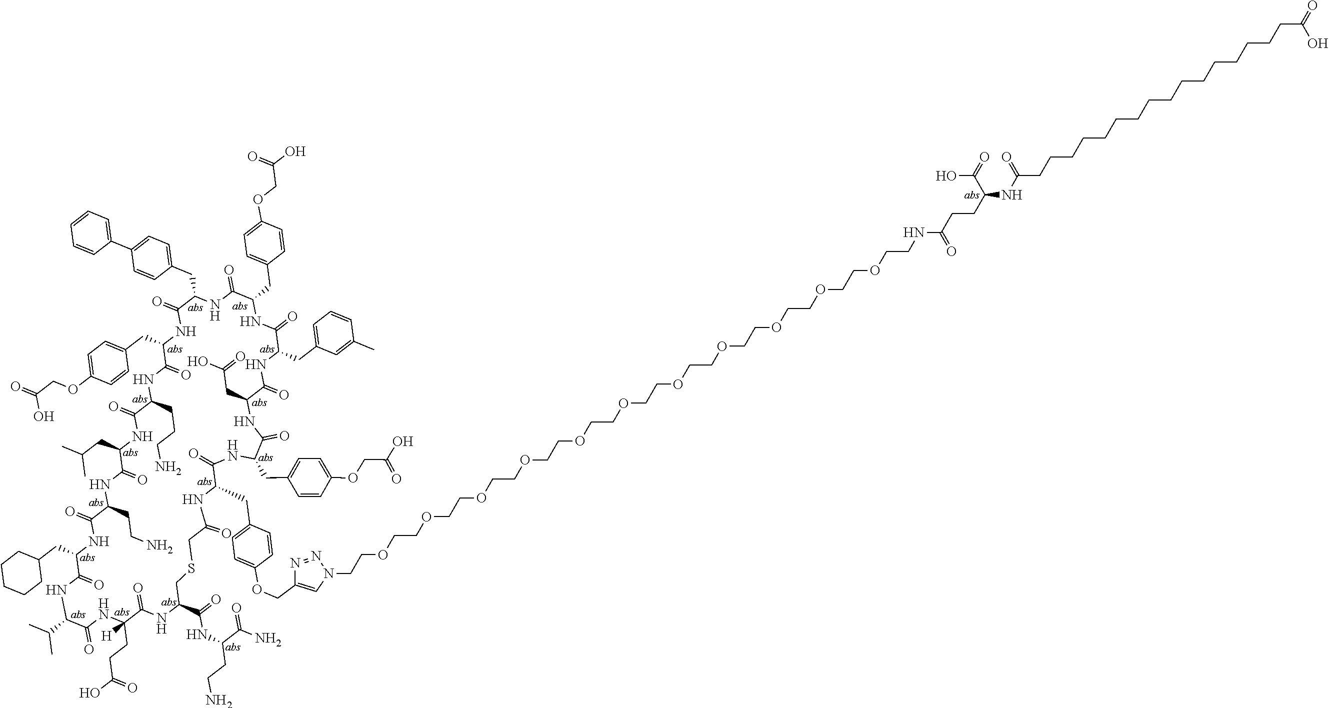 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCc4cn(CCOCCOCCOCCOCCOCCOCCOCCOCCOCCOCCOCCNC(=O)CC[C@H](NC(=O)CCCCCCCCCCCCCCCCC(=O)O)C(=O)O)nn4)cc3)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1011 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 20% B, 20-75% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 14% B, 14-54% B over 20 minutes, then a 2-minute hold at 100% B; Flow Rate: 40 mL/min; Column Temperature: 25° C. Fraction collection was triggered by MS signals. Fractions containing the desired product were combined and dried via centrifugal evaporation. The yield of the product was 5.1 mg, and its estimated purity by LCMS analysis was 97.500.

Analysis condition A: Retention time=1.67 min: ESI-MS(+1 m z [M+3H]³⁺: 1110.9.

### Preparation of Compound 1012

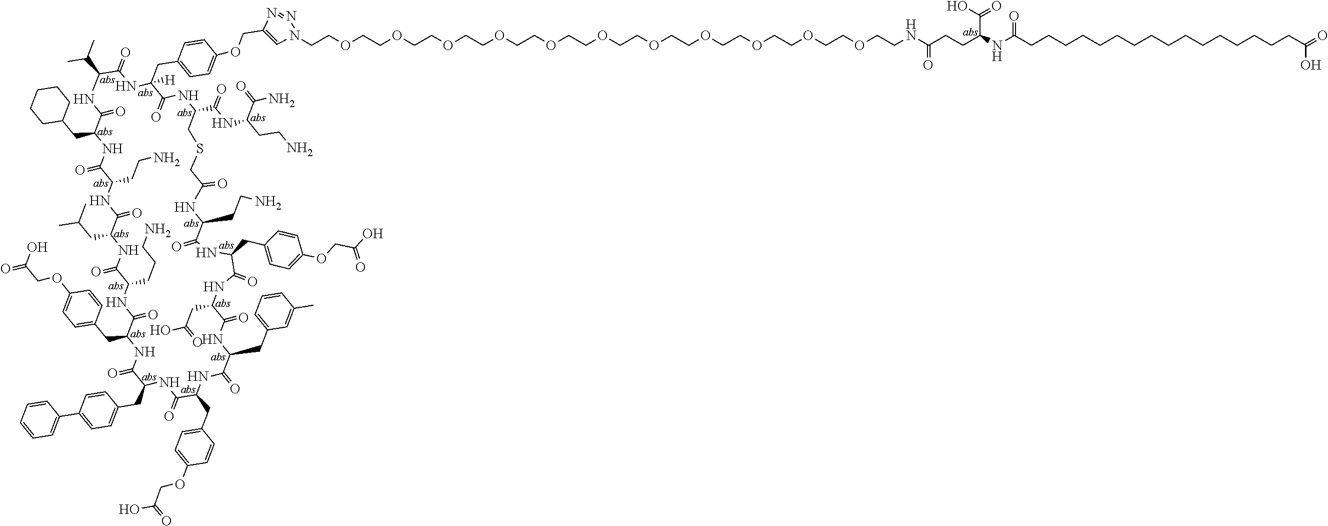 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(N)=O)NC(=O)[C@H](Cc3ccc(OCc4cn(CCOCCOCCOCCOCCOCCOCCOCCOCCOCCOCCOCCNC(=O)CC[C@H](NC(=O)CCCCCCCCCCCCCCCCC(=O)O)C(=O)O)nn4)cc3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1012 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 19% B, 19-72% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 26% B, 26-66% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 9.1 mg, and its estimated purity by LCMS analysis was 85.8%. Analysis condition B: Retention time=1.94 min; ESI-MS(+) m/z [M+3H]³⁺: 1101.1.

### Preparation of Compound 1013

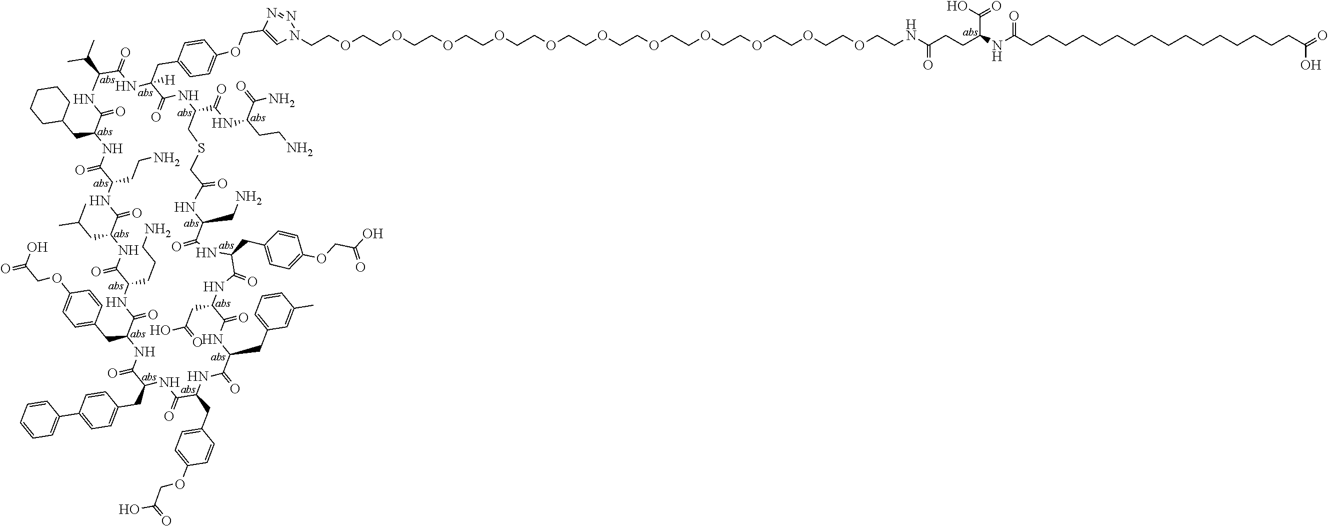 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(N)=O)NC(=O)[C@H](Cc3ccc(OCc4cn(CCOCCOCCOCCOCCOCCOCCOCCOCCOCCOCCOCCNC(=O)CC[C@H](NC(=O)CCCCCCCCCCCCCCCCC(=O)O)C(=O)O)nn4)cc3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1013 was prepared on a 50 [μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 190% B, 19-72% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile: water with 0.050% trifluoroacetic acid; Mobile Phase B: 95:5 acetonitrile: water with 0.050% trifluoroacetic acid; Gradient: a 0-minute hold at 31% B, 31-71% B over 20 minutes, then a 4-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 19% B, 19-59% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 5.4 mg, and its estimated purity by LCMS analysis was 99.200.

Analysis condition A: Retention time=1.76 min: ESI-MS(+1 m z [M+3H]³⁺: 1096.3.

### Preparation of Compound 1014

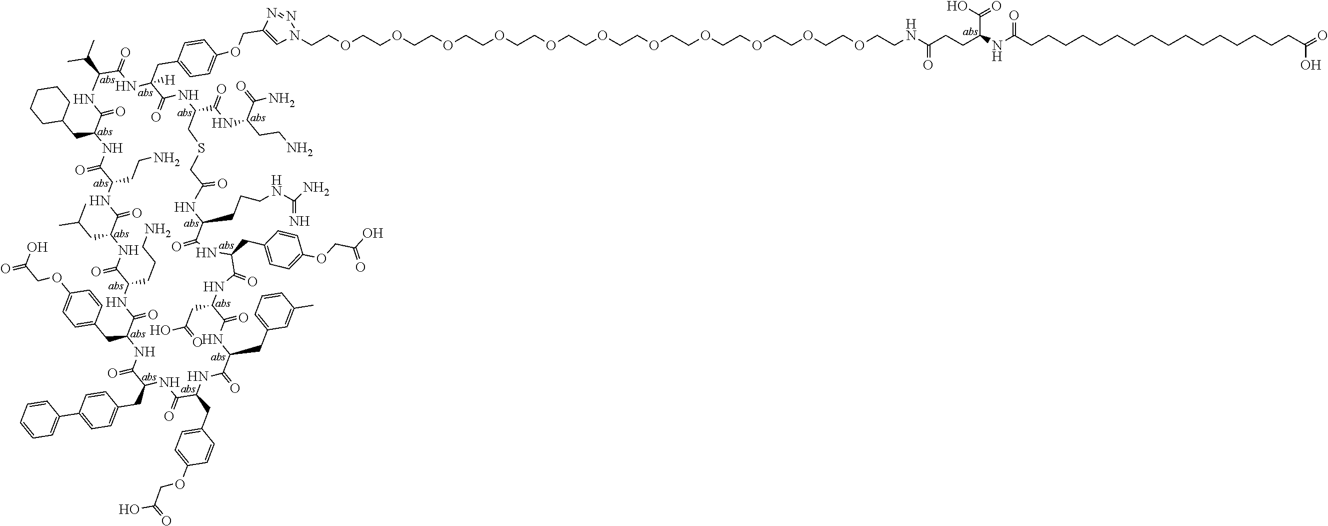 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(N)=O)NC(=O)[C@H](Cc3ccc(OCc4cn(CCOCCOCCOCCOCCOCCOCCOCCOCCOCCOCCOCCNC(=O)CC[C@H](NC(=O)CCCCCCCCCCCCCCCCC(=O)O)C(=O)O)nn4)cc3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1014 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 15% B, 15-70% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 24% B, 24-64% B over 20 minutes, then a 2-minute hold at 100% B; Flow Rate: 40 mL/min; Column Temperature: 25° C. Fraction collection was triggered by MS signals. Fractions containing the desired product were combined and dried via centrifugal evaporation. The yield of the product was 7.2 mg, and its estimated purity by LCMS analysis was 97.8%.

Analysis condition A: Retention time=1.82 min; ESI-MS(+) m/z [M+3H]³⁺: 1120.

### Preparation of Compound 1015

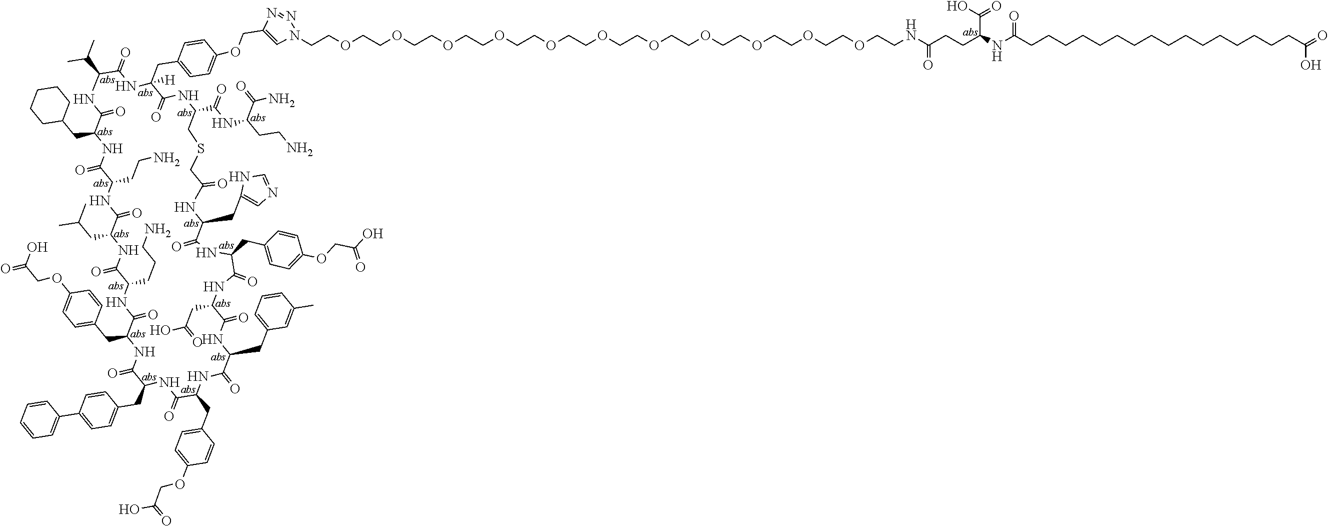 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cnc[nH]3)NC(=O)CSC[C@@H](C(=O)N[C@@H](CCN)C(N)=O)NC(=O)[C@H](Cc3ccc(OCc4cn(CCOCCOCCOCCOCCOCCOCCOCCOCCOCCOCCOCCNC(=O)CC[C@H](NC(=O)CCCCCCCCCCCCCCCCC(=O)O)C(=O)O)nn4)cc3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1015 was prepared on a 50 [μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 20% B, 20-72% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 17% B, 17-57% B over 20 minutes, then a 2-minute hold at 100% B; Flow Rate: 40 mL/min; Column Temperature: 25° C. Fraction collection was triggered by MS signals. Fractions containing the desired product were combined and dried via centrifugal evaporation. The yield of the product was 5.3 mg, and its estimated purity by LCMS analysis was 95.1%. Analysis condition A: Retention time=1.78 min: ESI-MS(+1 m z [M+3H]³⁺: 1113.3.

### Preparation of Compound 1016

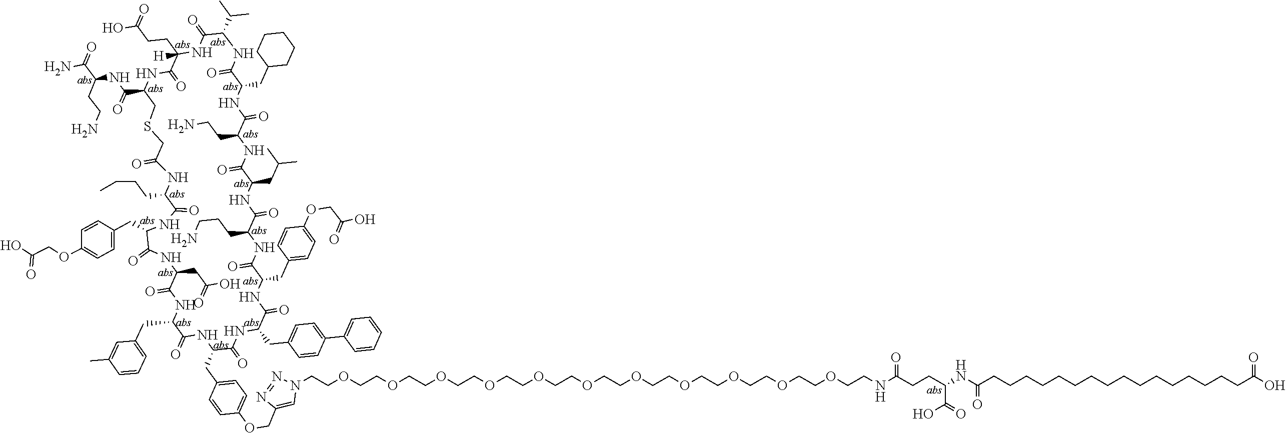 [SMILES: CCCC[C@@H]1NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCc3cn(CCOCCOCCOCCOCCOCCOCCOCCOCCOCCOCCOCCNC(=O)CC[C@H](NC(=O)CCCCCCCCCCCCCCCCC(=O)O)C(=O)O)nn3)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound was prepare on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 19% B, 19-74% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 20% B, 20-60% B over 20 minutes, then a 0-minute hold at 100% B; Flow Rate: 40 mL/min; Column Temperature: 25° C. Fraction collection was triggered by MS signals. Fractions containing the desired product were combined and dried via centrifugal evaporation. The yield of the product was 4.4 mg, and its estimated purity by LCMS analysis was 95.5%.

Analysis condition A: Retention time=1.71 min; ESI-MS(+) m/z [M+3H]³⁺: 1074.3.

### Preparation of Compound 1017

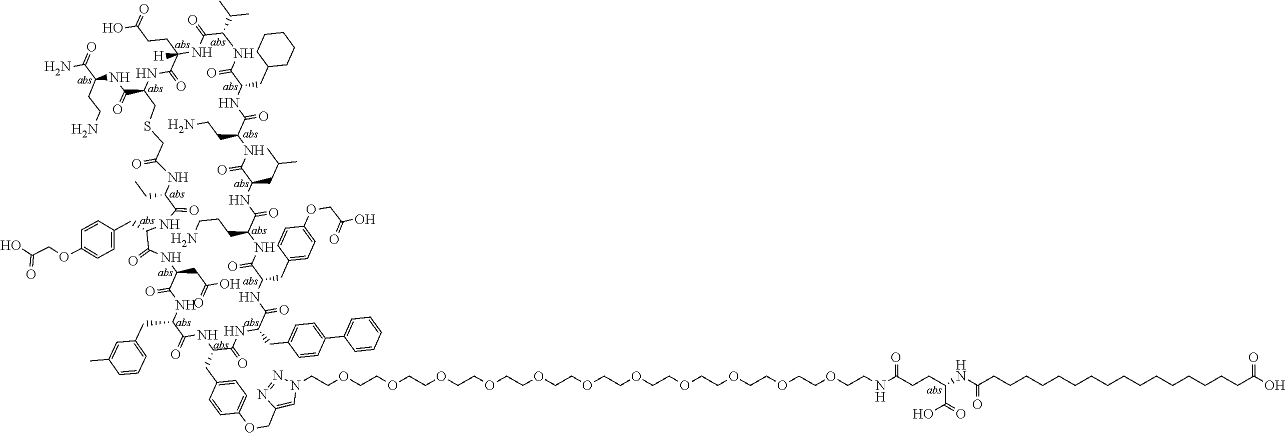 [SMILES: CC[C@@H]1NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCc3cn(CCOCCOCCOCCOCCOCCOCCOCCOCCOCCOCCOCCNC(=O)CC[C@H](NC(=O)CCCCCCCCCCCCCCCCC(=O)O)C(=O)O)nn3)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound was prepare on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 17% B, 17-72% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 18% B, 18-58% B over 20 minutes, then a 0-minute hold at 100% B; Flow Rate: 40 mL/min; Column Temperature: 25° C. Fraction collection was triggered by MS signals. Fractions containing the desired product were combined and dried via centrifugal evaporation. The yield of the product was 4.2 mg, and its estimated purity by LCMS analysis was 94.9%.

Analysis condition B: Retention time=2.08 min; ESI-MS(+) m/z [M+3H]³⁺: 1065.

### Preparation of Compound 1018

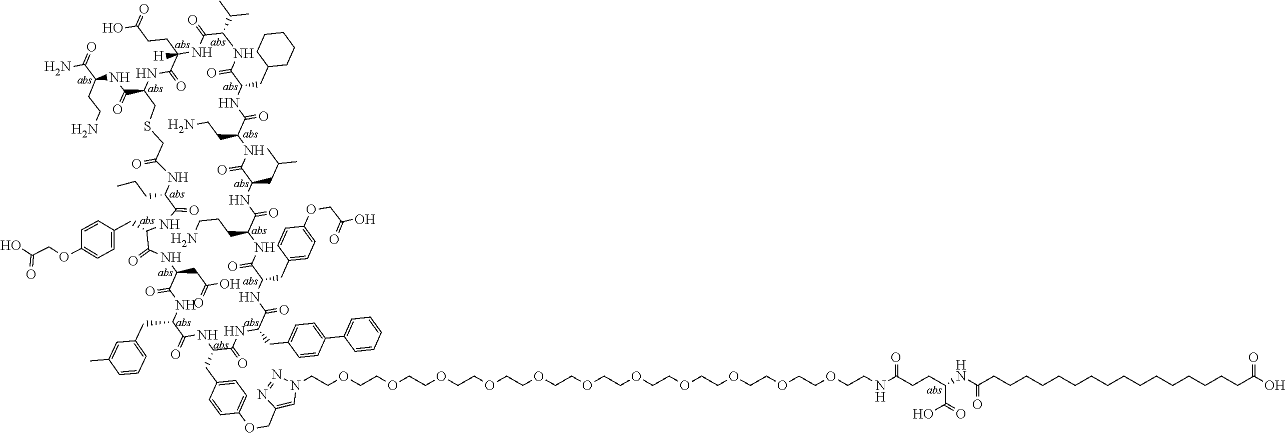 [SMILES: CCC[C@@H]1NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCc3cn(CCOCCOCCOCCOCCOCCOCCOCCOCCOCCOCCOCCNC(=O)CC[C@H](NC(=O)CCCCCCCCCCCCCCCCC(=O)O)C(=O)O)nn3)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 1018 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 18% B, 18-73% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 22% B, 22-62% B over 20 minutes, then a 0-minute hold at 100% B; Flow Rate: 40 mL/min; Column Temperature: 25° C. Fraction collection was triggered by MS signals. Fractions containing the desired product were combined and dried via centrifugal evaporation. The yield of the product was 5.3 mg, and its estimated purity by LCMS analysis was 99.2%.

Analysis condition B: Retention time=2.02 min; ESI-MS(+) m/z [M+2H]²⁺: 1604.

### Preparation of Compound 1019

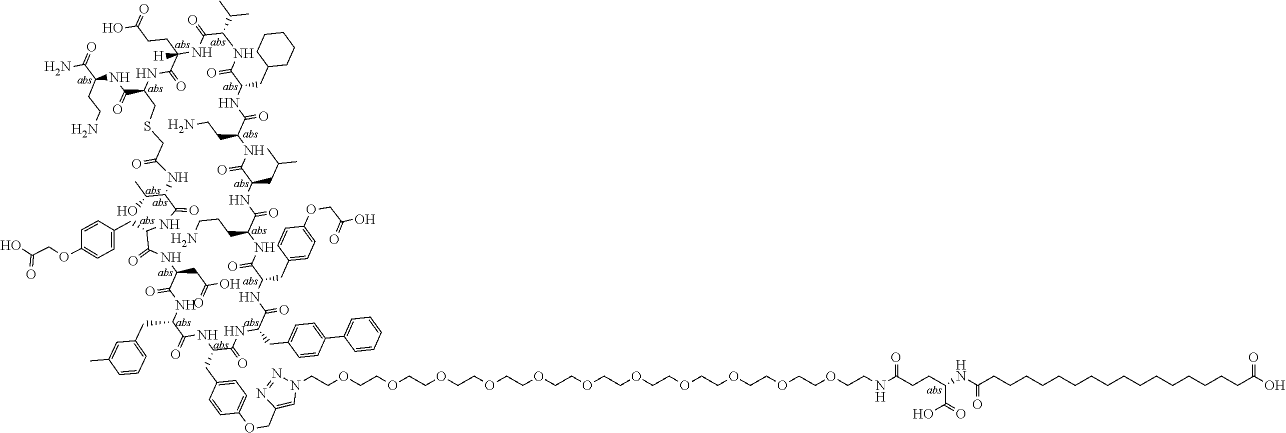 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCc4cn(CCOCCOCCOCCOCCOCCOCCOCCOCCOCCOCCOCCNC(=O)CC[C@H](NC(=O)CCCCCCCCCCCCCCCCC(=O)O)C(=O)O)nn4)cc3)NC2=O)c1]

Compound 1019 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 17% B, 17-72% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 20% B, 20-60% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 3.3 mg, and its estimated purity by LCMS analysis was 100%. Analysis condition A: Retention time=1.7 min; ESI-MS(+) m/z [M+3H]³⁺: 1071.1.

### Preparation of Compound 1020

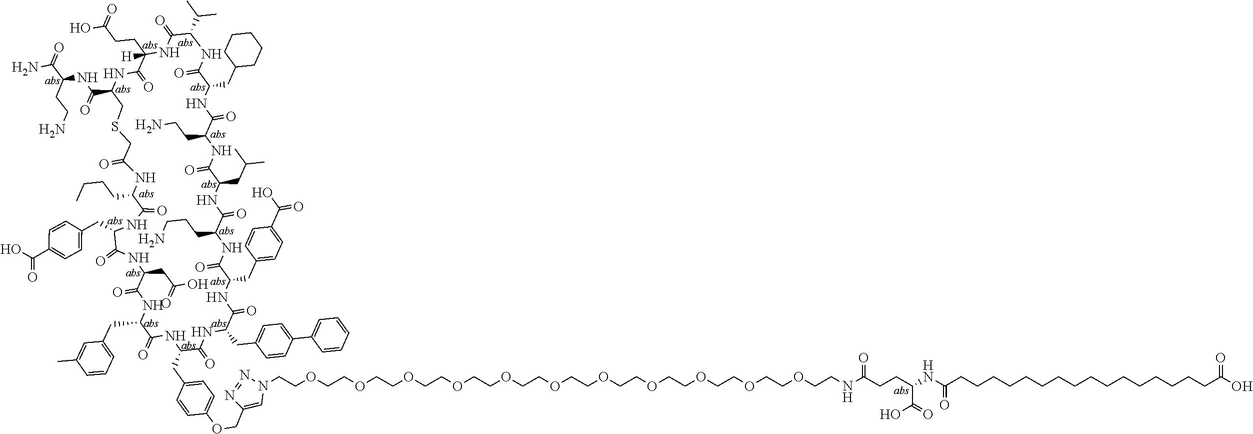 [SMILES: CCCC[C@@H]1NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(OCc3cn(CCOCCOCCOCCOCCOCCOCCOCCOCCOCCOCCOCCNC(=O)CC[C@H](NC(=O)CCCCCCCCCCCCCCCCC(=O)O)C(=O)O)nn3)cc2)NC(=O)[C@H](Cc2cccc(C)c2)NC(=O)[C@H](CC(=O)O)NC(=O)[C@H](Cc2ccc(C(=O)O)cc2)NC1=O]

Compound 1020 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 18% B, 18-73% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 20% B, 20-60% B over 20 minutes, then a 0-minute hold at 100% B; Flow Rate: 40 mL/min; Column Temperature: 25° C. Fraction collection was triggered by MS signals. Fractions containing the desired product were combined and dried via centrifugal evaporation. The yield of the product was 4.9 mg, and its estimated purity by LCMS analysis was 94.2%.

Analysis condition B: Retention time=2.03 min; ESI-MS(+) m/z [M+2H]²⁺: 1581.

### Preparation of Compound 1021

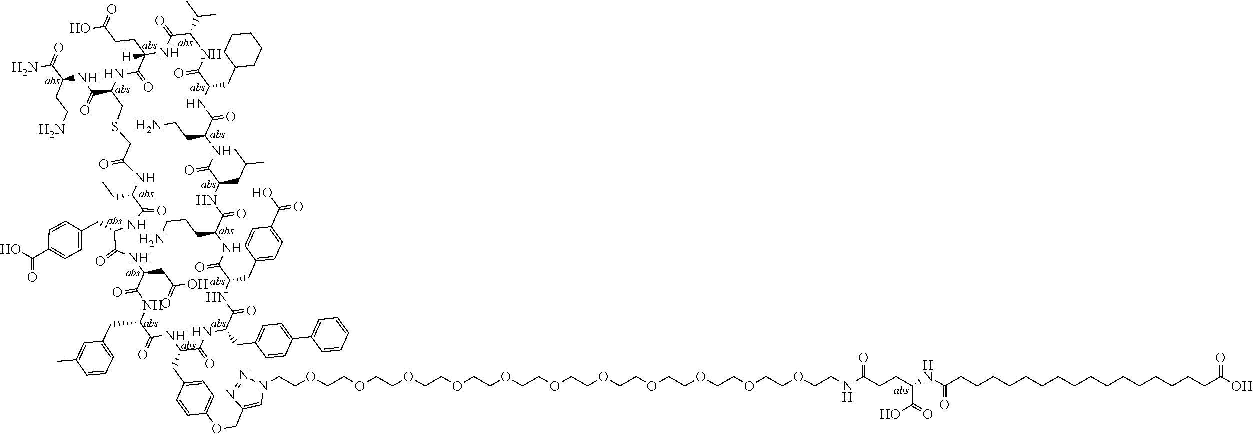 [SMILES: CC[C@@H]1NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(OCc3cn(CCOCCOCCOCCOCCOCCOCCOCCOCCOCCOCCOCCNC(=O)CC[C@H](NC(=O)CCCCCCCCCCCCCCCCC(=O)O)C(=O)O)nn3)cc2)NC(=O)[C@H](Cc2cccc(C)c2)NC(=O)[C@H](CC(=O)O)NC(=O)[C@H](Cc2ccc(C(=O)O)cc2)NC1=O]

Compound 1021 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 17% B, 17-72% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 11.1 mg, and its estimated purity by LCMS analysis was 76.9%.

Analysis condition A: Retention time=1.62 min; ESI-MS(+) m/z [M+3H]³⁺: 1045.

### Preparation of Compound 1022

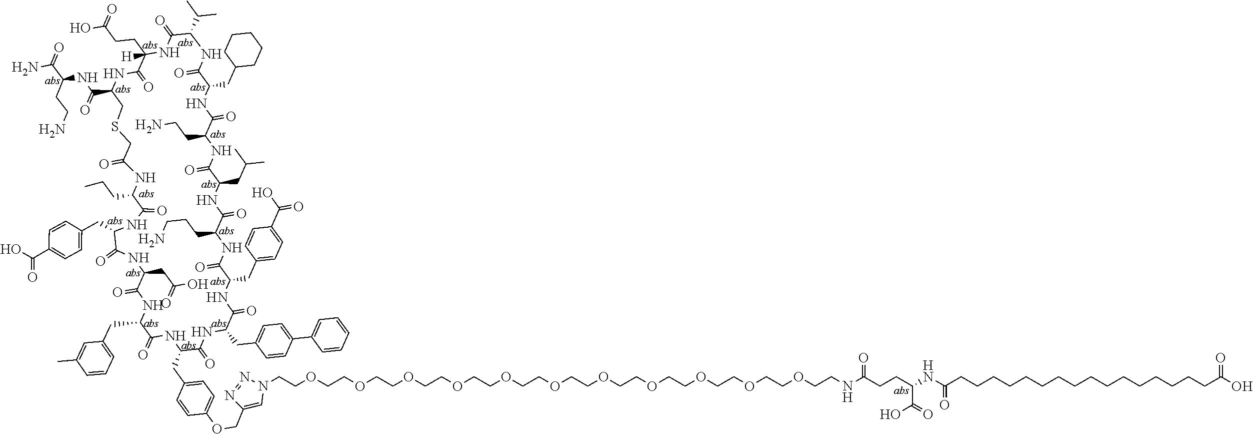 [SMILES: CCC[C@@H]1NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(OCc3cn(CCOCCOCCOCCOCCOCCOCCOCCOCCOCCOCCOCCNC(=O)CC[C@H](NC(=O)CCCCCCCCCCCCCCCCC(=O)O)C(=O)O)nn3)cc2)NC(=O)[C@H](Cc2cccc(C)c2)NC(=O)[C@H](CC(=O)O)NC(=O)[C@H](Cc2ccc(C(=O)O)cc2)NC1=O]

Compound 1022 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 18% B, 18-73% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 19% B, 19-59% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 5.3 mg, and its estimated purity by LCMS analysis was 96.5%.

Analysis condition B: Retention time=2.08 min; ESI-MS(+) m/z [M+3H]³⁺: 1050.

### Preparation of Compound 1023

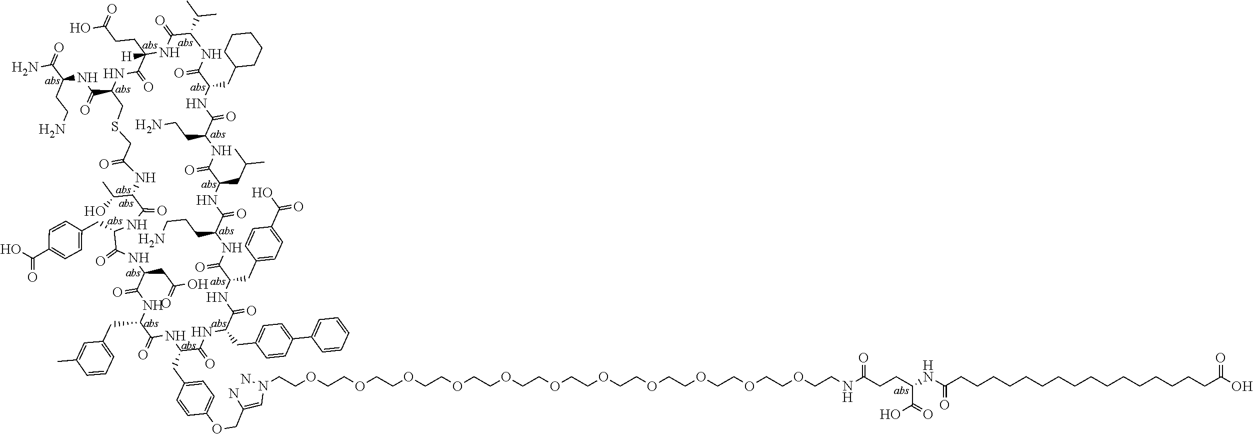 [SMILES: Cc1cccc(C[C@@H]2NC(=O)[C@H](CC(=O)O)NC(=O)[C@H](Cc3ccc(C(=O)O)cc3)NC(=O)[C@H]([C@@H](C)O)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(OCc4cn(CCOCCOCCOCCOCCOCCOCCOCCOCCOCCOCCOCCNC(=O)CC[C@H](NC(=O)CCCCCCCCCCCCCCCCC(=O)O)C(=O)O)nn4)cc3)NC2=O)c1]

Compound 1023 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 17% B, 17-72% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 18% B, 18-58% B over 20 minutes, then a 0-minute hold at 100% B; Flow Rate: 40 mL/min; Column Temperature: 25° C. Fraction collection was triggered by MS signals. Fractions containing the desired product were combined and dried via centrifugal evaporation. The yield of the product was 3.4 mg, and its estimated purity by LCMS analysis was 99.3%.

Analysis condition A: Retention time=1.7 min; ESI-MS(+) m/z [M+2H]²⁺: 1576.

### Preparation of Compound 1024

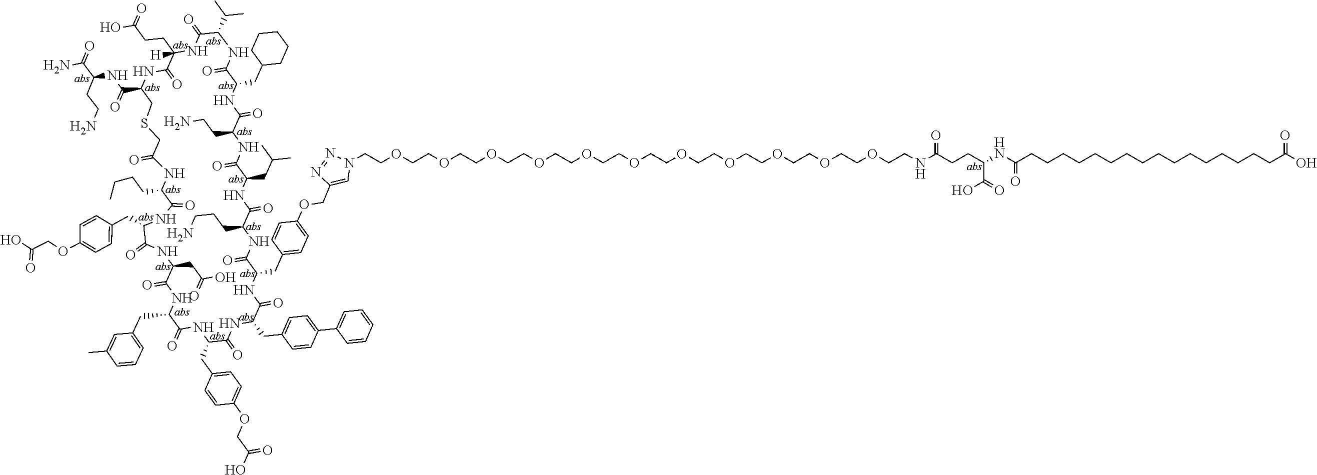

Compound 1024 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 20% B, 20-74% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 21% B, 21-61% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 2.6 mg, and its estimated purity by LCMS analysis was 100%. Analysis condition A: Retention time=1.74 min; ESI-MS(+) m z²⁺: 1074.8.

### Preparation of Compound 1025

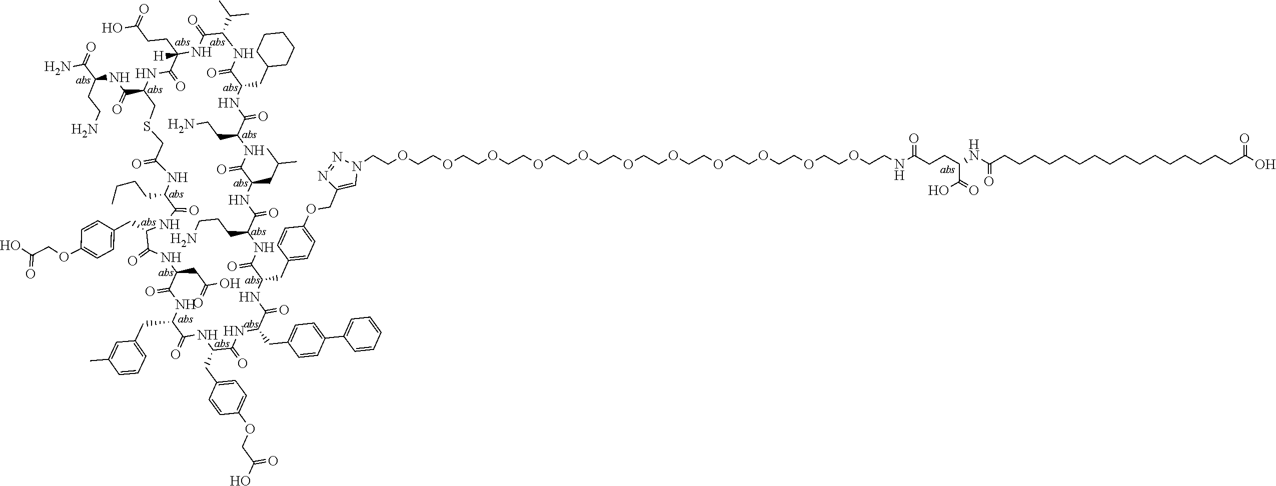

Compound 1025 was prepared on a 50 μmol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 18% B, 18-73% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 18% B, 18-58% B over 20 minutes, then a 0-minute hold at 100% B; Flow Rate: 40 mL/min; Column Temperature: 25° C. Fraction collection was triggered by MS signals. Fractions containing the desired product were combined and dried via centrifugal evaporation. The yield of the product was 2.9 mg, and its estimated purity by LCMS analysis was 99.2%.

Analysis condition A: Retention time=1.69 min; ESI-MS(+) m/z [M+3H]³⁺: 1065.1.

### Preparation of Compound 1026

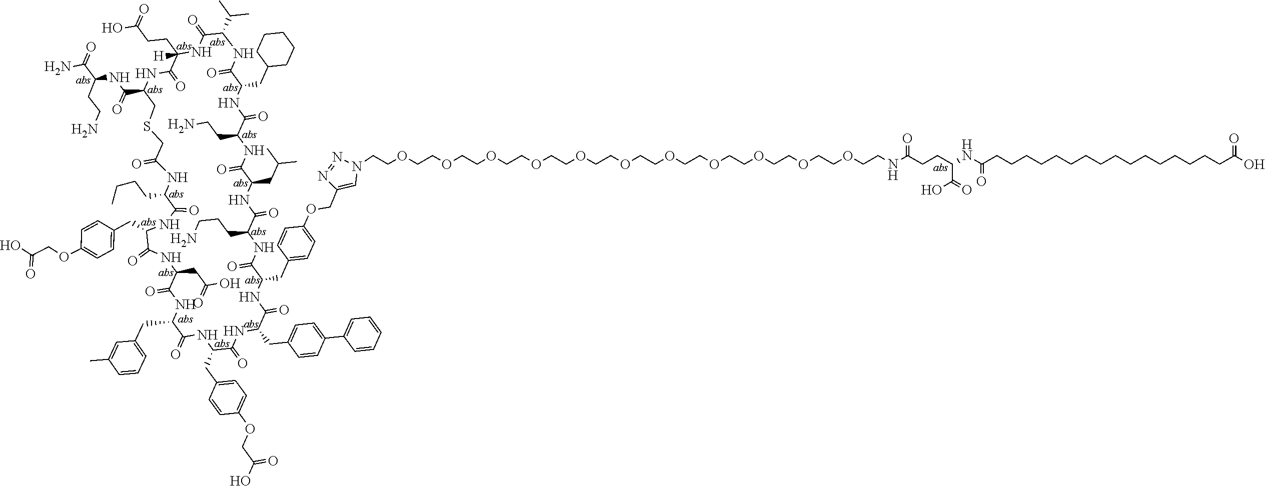

Compound 1026 was prepared on a 50 μmol scale, following the general synthetic sequence described for the preparation of composed of the following general procedures: The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 16% B, 16-71% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 21% B, 21-61% B over 20 minutes, then a 0-minute hold at 100% B; Flow Rate: 40 mL/min; Column Temperature: 25° C. Fraction collection was triggered by MS signals. Fractions containing the desired product were combined and dried via centrifugal evaporation. The yield of the product was 2.6 mg, and its estimated purity by LCMS analysis was 87.7%.

Analysis condition B: Retention time=2.08 min; ESI-MS(+) m/z [M+3H]³⁺: 1070.1.

### Preparation of Compound 1027

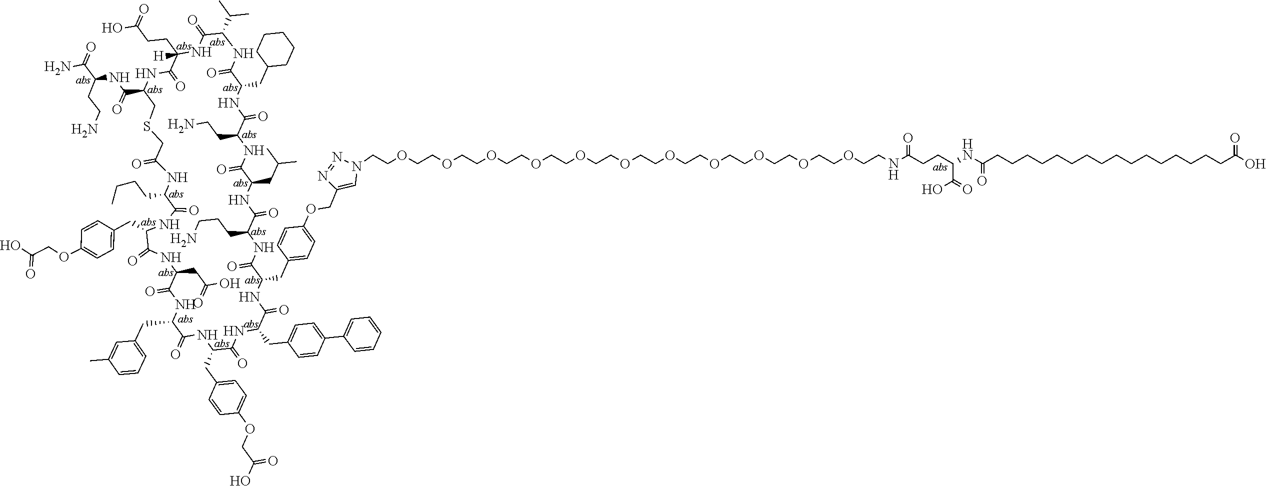

Compound 1027 was prepared on a 50 μmol scale, following the general synthetic sequence described for the preparation of composed of the following general procedures: The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 20% B, 20-72% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 15.1 mg, and its estimated purity by LCMS analysis was 86.3%.

Analysis condition A: Retention time=1.69 min; ESI-MS(+) m/z [M+3H]³⁺: 1070.2.

### Preparation of Compound 1028

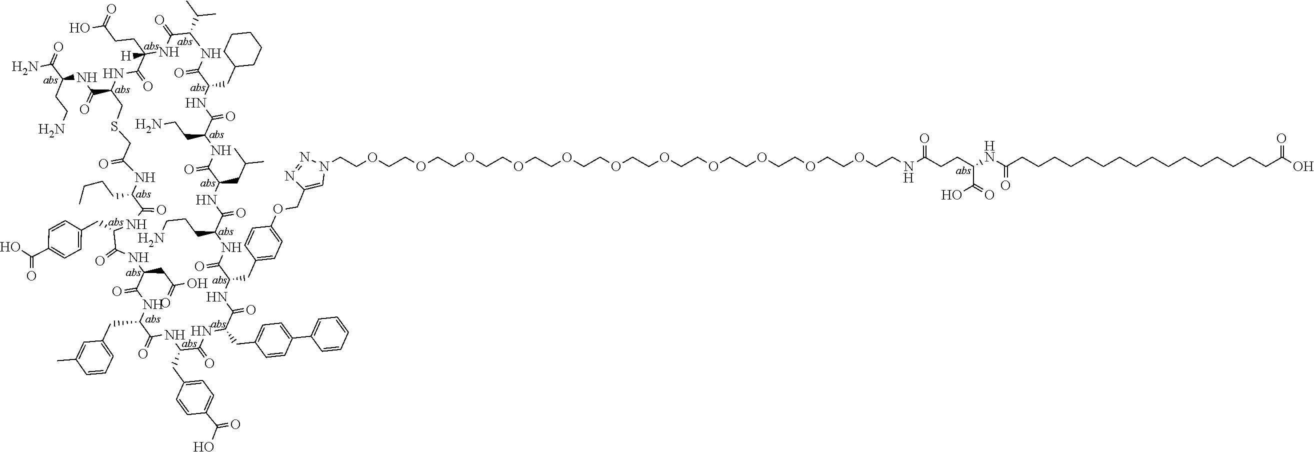

Compound 1028 was prepared on a 50 μmol scale, following the general synthetic sequence described for the preparation of composed of the following general procedures: The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 18% B, 18-73% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 13.8 mg, and its estimated purity by LCMS analysis was 91.3%.

Analysis condition A: Retention time=1.74 min; ESI-MS(+) m/z [M+3H]³⁺: 1054.9.

### Preparation of Compound 1029

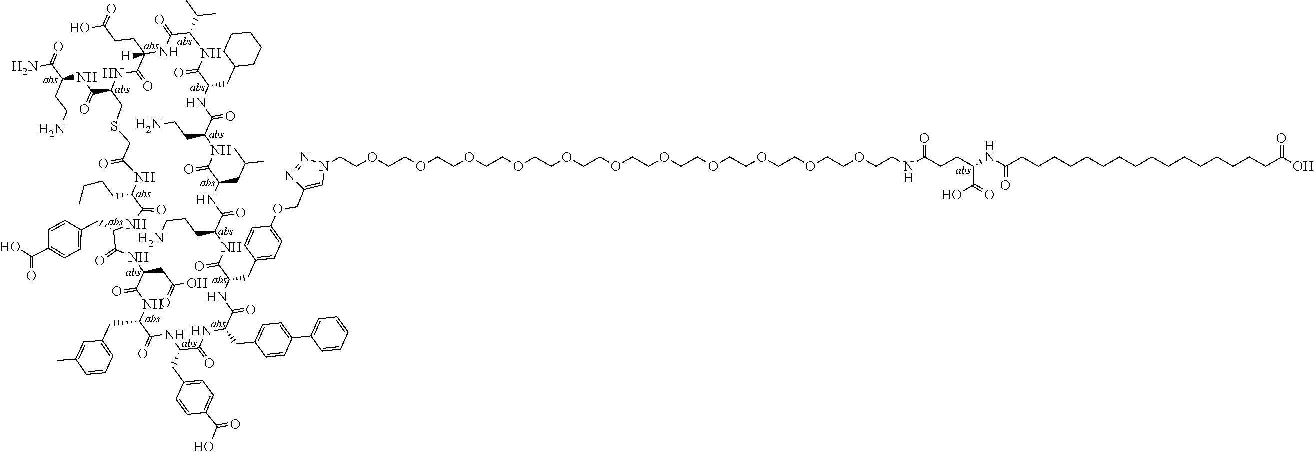

Compound 1029 was prepared on a 50 [mol scale. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 20% B, 20-72% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 21% B, 21-61% B over 20 minutes, then a 0-minute hold at 100% B; Flow Rate: 40 mL/min; Column Temperature: 25° C. Fraction collection was triggered by MS signals. Fractions containing the desired product were combined and dried via centrifugal evaporation. The yield of the product was 5 mg, and its estimated purity by LCMS analysis was 92%.

Analysis condition B: Retention time=2.01 min; ESI-MS(+) m/z [M+2H]²⁺: 1567.2.

### Preparation of Compound 1030

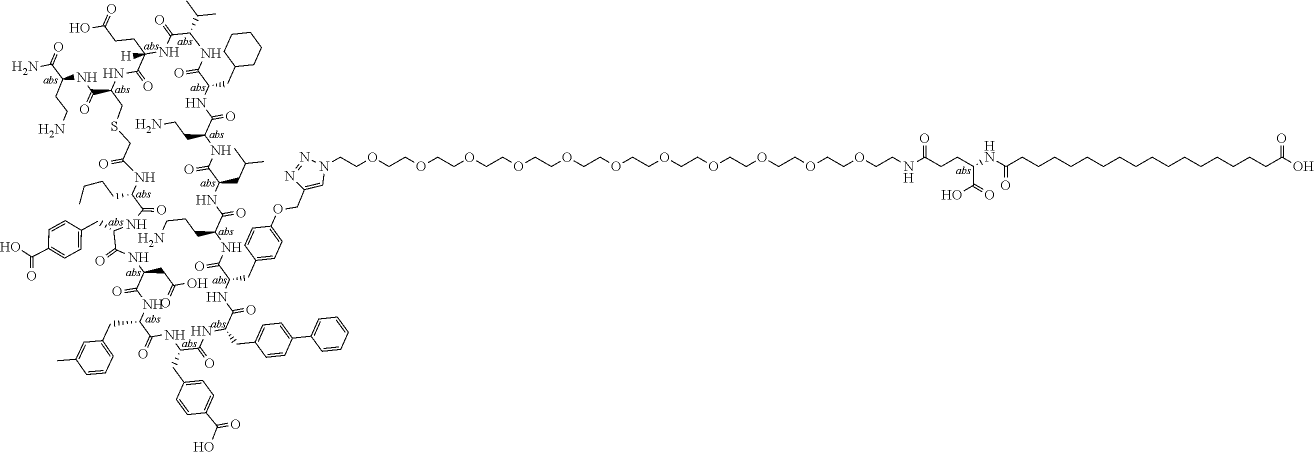

Compound 1030 was prepared on a 50 μmol scale, following the general synthetic sequence described for the preparation of composed of the following general procedures: The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 33% B, 33-73% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 13.3 mg, and its estimated purity by LCMS analysis was 75.5%.

Analysis condition B: Retention time=2.03 min; ESI-MS(+) m/z [M+3H]³⁺: 1050.1.

### Preparation of Compound 1031

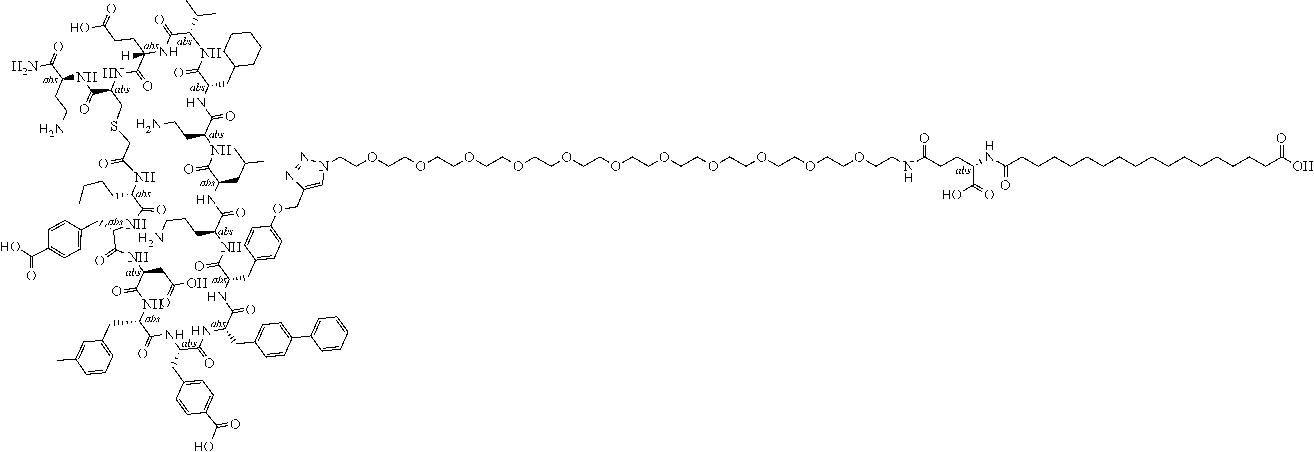

Compound 1031 was prepared on a 50 μmol scale, following the general synthetic sequence described for the preparation of composed of the following general procedures: The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 17% B, 17-72% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 18% B, 18-58% B over 20 minutes, then a 0-minute hold at 100% B; Flow Rate: 35 mL/min; Column Temperature: 25° C. Fraction collection was triggered by MS signals. Fractions containing the desired product were combined and dried via centrifugal evaporation. The yield of the product was 2.2 mg, and its estimated purity by LCMS analysis was 95.8%.

Analysis condition A: Retention time=1.72 min; ESI-MS(+) m/z [M+2H]²⁺: 1575.2.

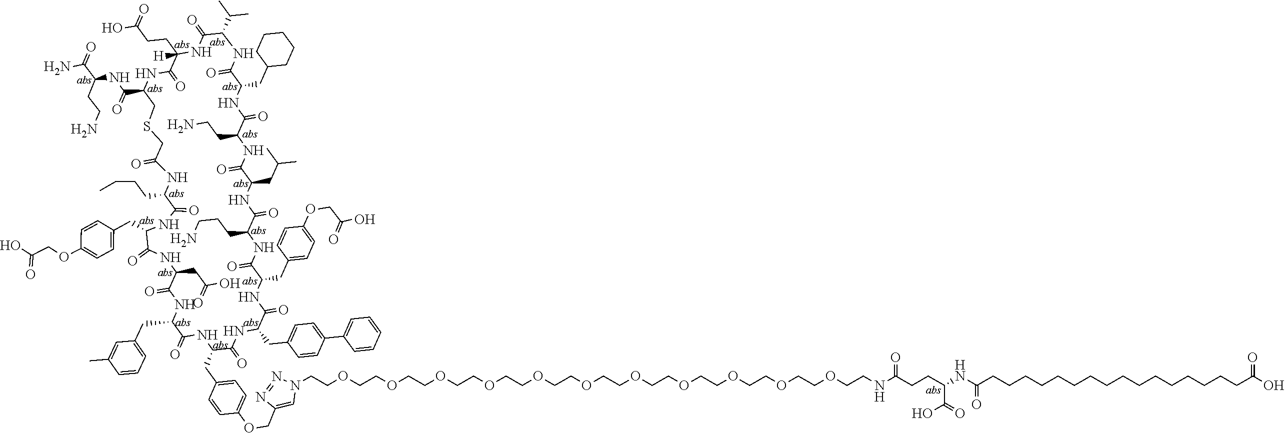 [SMILES: CCCC[C@@H]1NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCc3cn(CCOCCOCCOCCOCCOCCOCCOCCOCCOCCOCCOCCNC(=O)CC[C@H](NC(=O)CCCCCCCCCCCCCCCCC(=O)O)C(=O)O)nn3)cc2)NC(=O)[C@H](Cc2cccc(C)c2)NC(=O)[C@H](CC(=O)O)NC(=O)[C@H](Cc2ccc(OCC(=O)O)cc2)NC1=O]

### Preparation of Compound 1032

Compound 1032 was prepared on a 50 μmol scale, following the general synthetic sequence described for the preparation of composed of the following general procedures: The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 17% B, 17-72% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 1.4 mg, and its estimated purity by LCMS analysis was 81.5%.

Analysis condition A: Retention time=1.59 min; ESI-MS(+) m/z [M+2H]²⁺: 1664.9.

### Preparation of Compound 1033

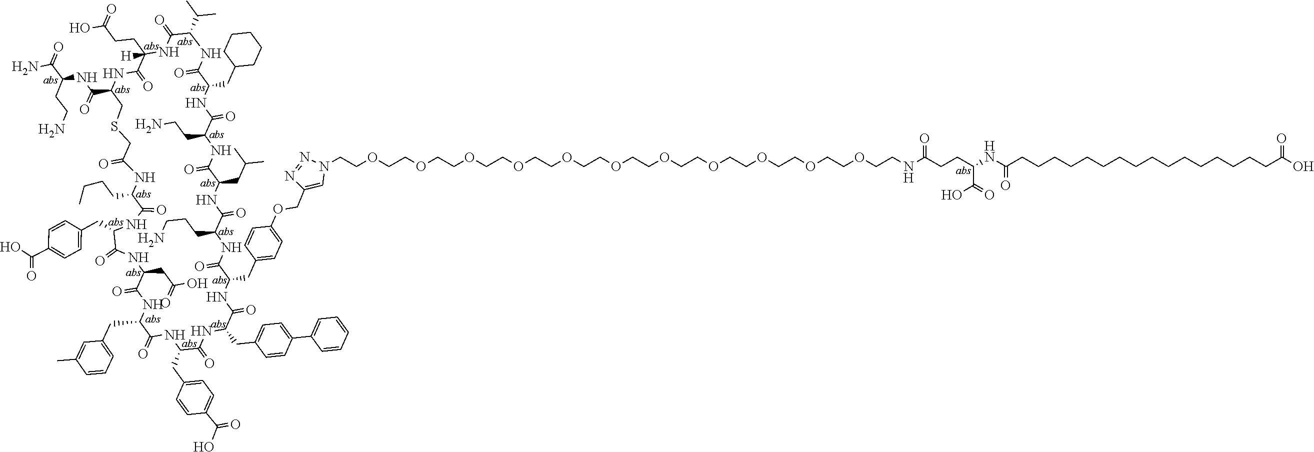

Compound 1033 was prepared on a 50 [μmol scale, following the general synthetic sequence described for the preparation of composed of the following general procedures: The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 17% B, 17-72% B over 30 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The yield of the product was 13.3 mg, and its estimated purity by LCMS analysis was 91.1%.

Analysis condition A: Retention time=1.64 min; ESI-MS(+) m/z [M+3H]³⁺: 1091.1.

### Preparation of Compound 1034

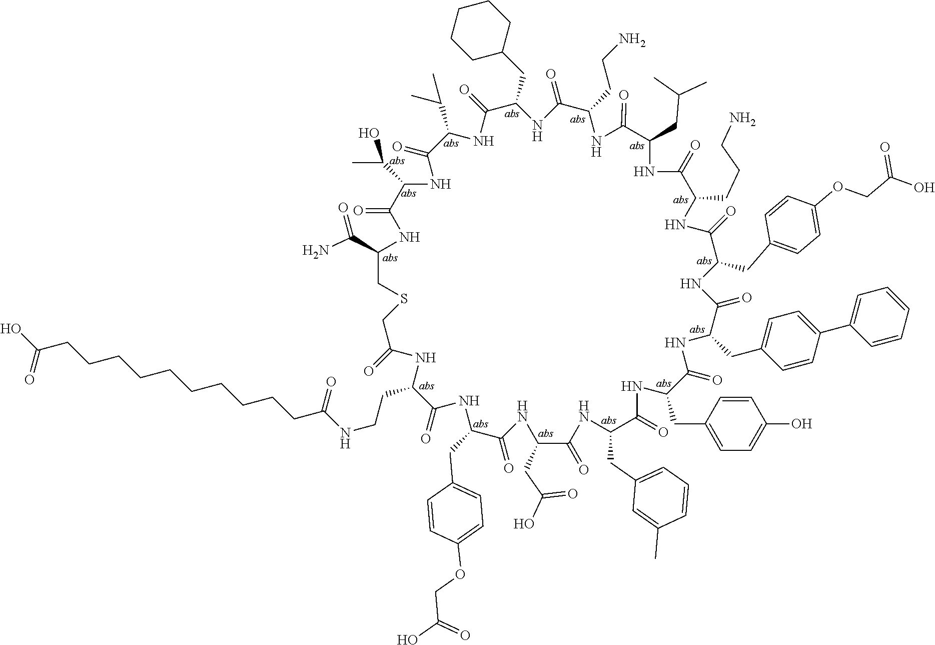 [SMILES: Cc1cccc(C[C@@H]2NC(=O)[C@H](CC(=O)O)NC(=O)[C@H](Cc3ccc(OCC(=O)O)cc3)NC(=O)[C@H](CCNC(=O)CCCCCCCCCCC(=O)O)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

To a small solid phase reactor was added the Rink resin o the following linear sequence: ClAc-Dab(Mtt)-Tyr(CH₂COOtBu)-Asp(OtBu)-Phe(3-Me)-Tyr(CH₂COOtBu)-Bip-Tyr(CH₂-propargyl)-Orn(Boc)-dLeu-Dab(Boc)-Cha-Val-Thr(tBu)-Cys(Trt)-Dab(Boc)-Sieber resin (50 μmol). The linear sequence was assembled using Symphony X peptide synthesizer following the general procedures composing of the following general procedures:

“Symphony X Resin-Swelling procedure”, “Symphony X Single-Coupling procedure “wherein 10 equiv. of amino acid, 1 h-2 h coupling time was used for all the amino acid coupling steps except for Fmoc-Dab(Mtt)—OH was installed using “Single-Coupling Manual Addition Procedure A “, “Symphony X Chloroacetic Anhydride coupling procedure “. The linear peptide containing Dap(Mtt) (total 100 μmol) on a Rink resin was transferred into a Bio-Rad tube with a frit. The resin was washed 3 times with CH₂Cl₂. About 5 mL of 1.5% TFA in CH₂Cl₂ was added and the vessel was shaken for 3-5 min. The solvents were drained. The deprotection was repeated two more times. The resin containing the Mtt-free Dap residue was then resined with CH₂Cl₂ (5×). The resin was divided into 4 vessels with the frit. To each vessel, DMF (5 mL) was added and the vessel was shaken for 10 min. DMF was drained. 3-5 mLof fresh DMF, DIEA (0.1 mL) was added followed by 50-100 mg of 1-tert-butyl 2,3,4,5,6-pentafluorophenyl dodecanedioate (or other activated esters or acyl chlorides in other reactions). The mixture was shaken for 2-16 h at rt. It was drained, rinsed with DMF (5×), then CH₂Cl₂ (3×), and dried. About 4-5 mL of TFA/TIS/DTT (96: 3:1) was added and the vessel was shaken for 1.5 h at rt. The TFA solution was drained through the frit and into a vial. Et20 (40 mL) was added. The cold vessel was centrifuged (2×) and the solids were collected and air dried. The solids were dissolved in DMF and 1.5 −2 mL of DIEA was added. The resulting solution was shaken overnight. It was concentrated and the residue was dissolved in 1.5-2 mL of DMF and submitted to purification.

Compound 1034 was prepared on a 25 μmol scale. The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 18% B, 18-58% B over 20 minutes, then a 4-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-minute hold at 25% B, 25-65% B over 20 minutes, then a 2-minute hold at 100% B; Flow Rate: 40 mL/min; Column Temperature: 25° C. Fraction collection was triggered by MS and UV signals. Fractions containing the desired product were combined and dried via centrifugal evaporation. The yield of the product was 1.2 mg, and its estimated purity by LCMS analysis was 96.4%. Analysis condition A: Retention time=1.62 min; ESI-MS(+) m z [M+2H]²⁺: 1130.

### Preparation of Compound 1035

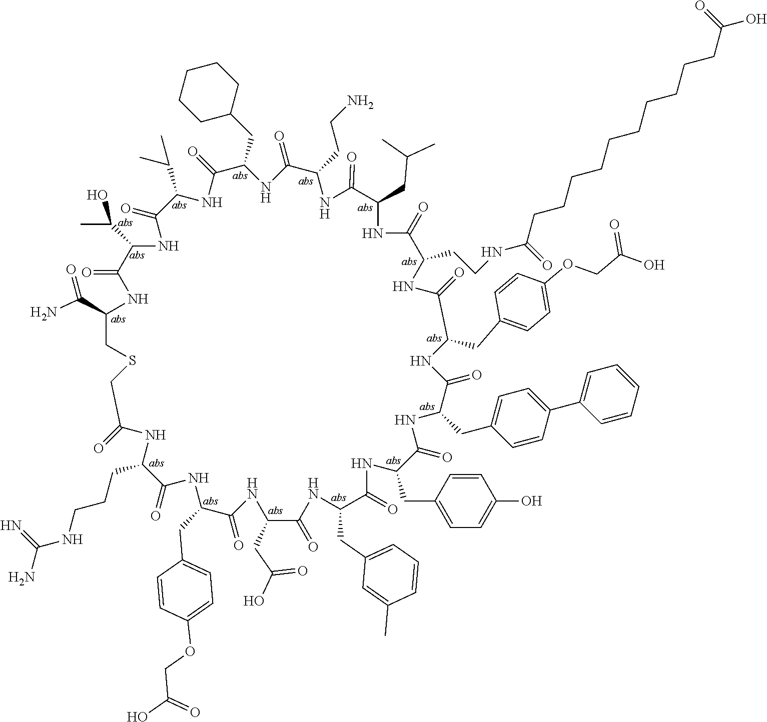 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCNC(=O)CCCCCCCCCCC(=O)O)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 1035 was prepared on a 25 μmol scale following the general synthetic procedures described for the preparation of Compound 1034. The crude material was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 18% B, 18-61% B over 20 minutes, then a 4-minute hold at 100% B; Flow Rate: 20 mL/min; Column Temperature: 25° C. Fraction collection was triggered by MS signals. Fractions containing the desired product were combined and dried via centrifugal evaporation. The yield of the product was 2.9 mg, and its estimated purity by LCMS analysis was 92.1%.

Analysis condition A: Retention time=1.56 min; ESI-MS(+) m/z [M+2H]²⁺: 1157.7.

### Preparation of Compound 1036

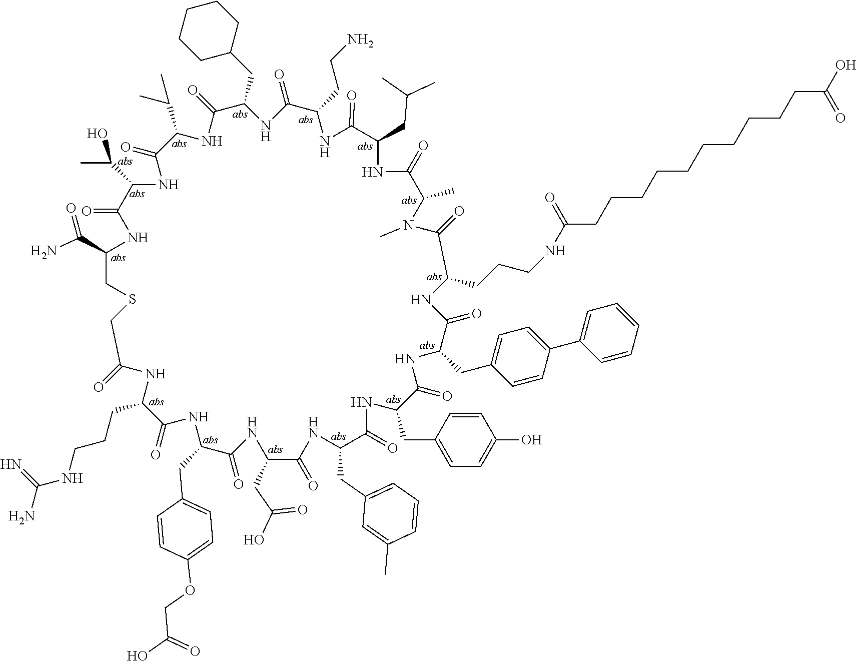 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](C)N(C)C(=O)[C@H](CCCNC(=O)CCCCCCCCCCC(=O)O)NC(=O)[C@H](Cc3ccc(-c4ccccc4)cc3)NC(=O)[C@H](Cc3ccc(O)cc3)NC2=O)c1]

Compound 1036 was prepared on a 25 μmol scale, following the general synthetic procedures described for the preparation of Compound 1034. The crude material was purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-minute hold at 49% B, 49-100% B over 20 minutes, then a 4-minute hold at 100% B; Flow Rate: 20 mL/min; Column Temperature: 25° C. Fraction collection was triggered by MS signals. Fractions containing the desired product were combined and dried via centrifugal evaporation. The yield of the product was 9.4 mg, and its estimated purity by LCMS analysis was 95.6%.

Analysis condition A: Retention time=1.79 min; ESI-MS(+) m/z [M+2H]²⁺: 1090.1.

### Preparation of Compound 1037

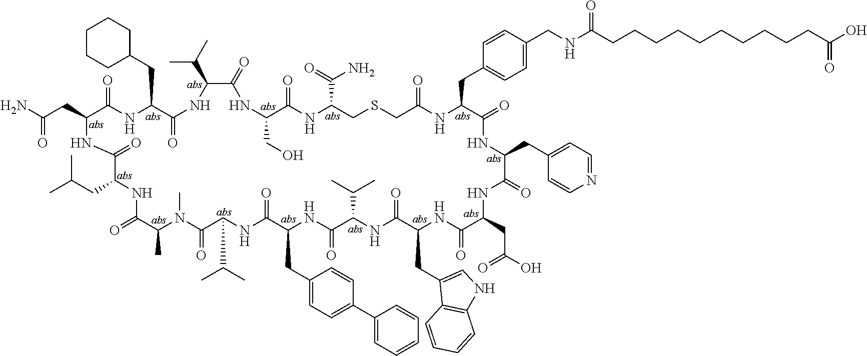 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(CNC(=O)CCCCCCCCCCC(=O)O)cc2)NC(=O)CSC[C@@H](C(N)=O)NC(=O)[C@H](CO)NC(=O)[C@H](C(C)C)NC(=O)[C@H](CC2CCCCC2)NC(=O)[C@H](CC(N)=O)NC1=O]

### Compound 1037A

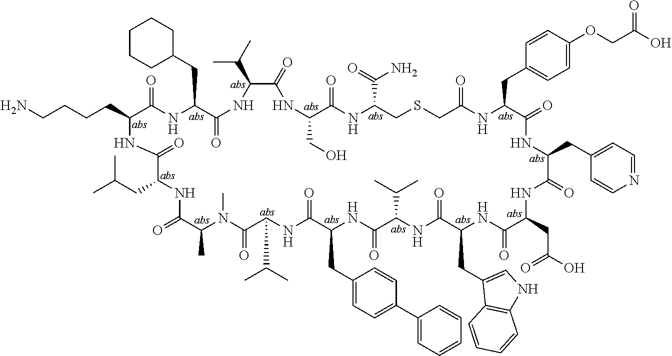 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(N)=O)NC(=O)[C@H](CO)NC(=O)[C@H](C(C)C)NC(=O)[C@H](CC2CCCCC2)NC(=O)[C@H](CCCCN)NC1=O]

Compound 1037A was prepared following the procedures for Compound 1038 on a 50 μmol scale. The yield of the product was 24.5 mg, and its estimated purity by LCMS analysis was 99.3%. Analysis condition B: Retention time=1.64 min; ESI-MS(+) m/z [M+H]⁺: 1919.

Compound 1037 was prepared from Compound 1037A following the procedure for the preparation of Compound 1038 on a 10.4 μmol scale. The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 30% B, 30-70% B over 20 minutes, then a 4-minute hold at 100% B; Flow Rate: 20 mL/min; Column Temperature: 25° C. Fraction collection was triggered by MS signals. Fractions containing the desired product were combined and dried via centrifugal evaporation. The yield of the product was 7 mg, and its estimated purity by LCMS analysis was 100%. Analysis condition B: Retention time=1.85 min; ESI-MS(+) m/z [M+2H]²⁺: 1036.2.

### Preparation of Compound 1038

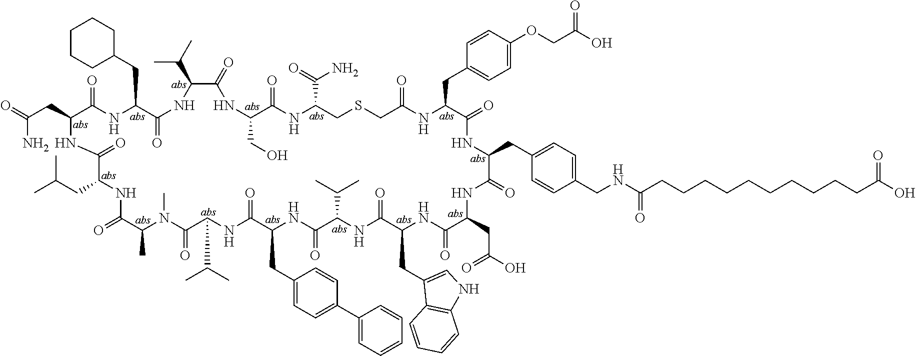 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(CNC(=O)CCCCCCCCCCC(=O)O)cc2)NC(=O)[C@H](Cc2ccc(OCC(=O)O)cc2)NC(=O)CSC[C@@H](C(N)=O)NC(=O)[C@H](CO)NC(=O)[C@H](C(C)C)NC(=O)[C@H](CC2CCCCC2)NC(=O)[C@H](CC(N)=O)NC1=O]

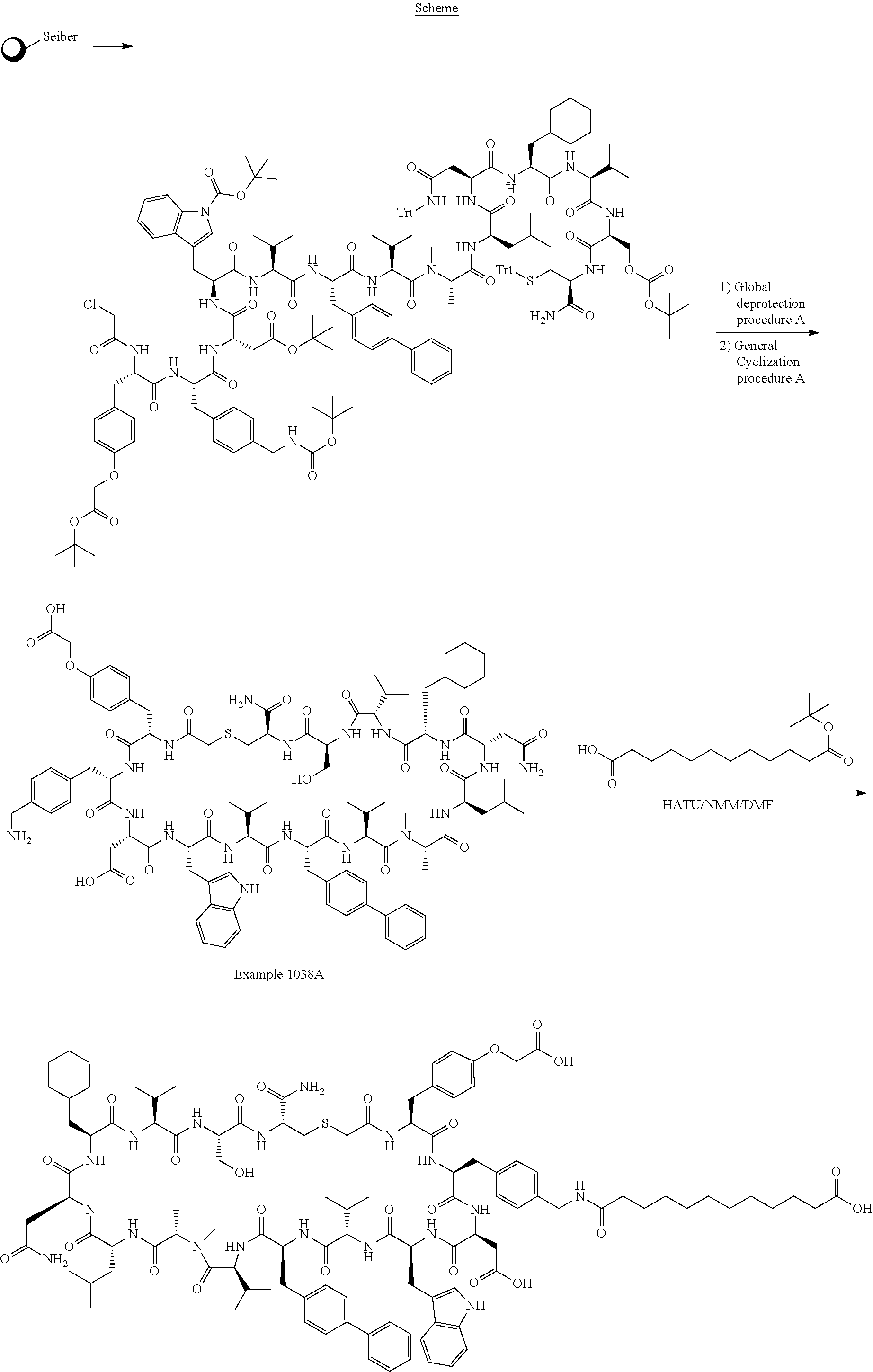

Compound 1038A: The amine-containing macrocycle was prepared following the general procedures for solid-phase peptide synthesis (SPPS) and macrocyclization, composing of the following general procedures. To a 25-mL polypropylene solid-phase reaction vessel was added Sieber amide resin with Fmoc-Cys(Trt)—OH on a 50 mol scale (80 mg), and the reaction vessel was placed on the Symphony peptide synthesizer. The following procedures were then performed sequentially: “Symphony Resin-Swelling procedure” was followed; “Symphony Single-Coupling procedure “was followed with Fmoc-Ser(tBu)—OH; “Symphony Single-Coupling procedure” was followed with Fmoc-Val-OH; “Symphony Single-Coupling procedure “was followed with Fmoc-Cha-OH; “Symphony Single-Coupling procedure “was followed with Fmoc-Asn(trt)—OH; “Symphony Single-Coupling procedure “was followed with Fmoc-D-Leu-OH; “Symphony Single-Coupling procedure “was followed with Fmoc-N-methyl-Ala-OH; “Symphony Single-Coupling procedure “was followed with Fmoc-Val-OH; “Symphony Single-Coupling procedure” was followed with Fmoc-Bip-OH; “Symphony Single-Coupling procedure” was followed with Fmoc-Val-OH; “Symphony Single-Coupling procedure “was followed with Fmoc-Tyr(tBu)—OH; “Symphony Single-Coupling procedure “was followed with Fmoc-Asp(OtBu)—OH; “Symphony Single-Coupling procedure “was followed with Fmoc-Phe(4-Boc-aminomethyl)—OH; “Symphony Single-Coupling procedure “was followed with Fmoc-Phe(4—COOtBu)—OH; “Symphony Chloroacetic Anhydride coupling procedure “was followed;

“Global Deprotection MethodA “was followed; “Cyclization Method A” was followed. To the residue was added DMF (40 mL containing 1 mL of DIEA). The reaction was shaken for 16 h. LCMS indicates desired product. It was concentrated to dryness on genevac, and then diluted with DMF (2 mL), filtered and submitted to single compound purification. Analysis condition B: Retention time=1.88 min; ESI-MS(+) m/z [M+H]⁺: 1932.2.

Compound 1038: To a solution of 12-(tert-butoxy)-12-oxododecanoic acid (3.08 mg, 10.76 μmol) in 3 mL of DMF was added HATU (6.14 mg, 0.016 mmol) and half portion of N-methylmorpholine (3.55 μl, 0.032 mmol). The reaction mixture was stirred at rt for 30 minutes, followed by Compound 1038A (20.79 mg, 10.76 μmol) and the rest of base. After 16 h stirring, The solvent was evaporated under pressure and treated with 10% TFA in DCM (2 mL) for 30 minutes. Upon completion, the reaction was quenched with ether (1 mL) and the desired was filtered, diluted with CH₃CN and submitted to single compound purification. The crude material was purified via preparative LC/MS with the following conditions: Column: XBridge C18, 150 mm×30 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 23% B, 23-63% B over 20 minutes, then a 2-minute hold at 100% B; Flow Rate: 40 mL/min; Column Temperature: 25° C. Fraction collection was triggered by MS and UV signals. Fractions containing the desired product were combined and dried via centrifugal evaporation. The material was further purified via preparative LC/MS with the following conditions: Column: XBridge C18, 200 mm×30 mm, 5-μm particles; Mobile Phase A: 5:95 acetonitrile: water with ammonium acetate; Mobile Phase B: 95:5 acetonitrile: water with ammonium acetate; Gradient: a 0-minute hold at 22% B, 22-62% B over 20 minutes, then a 2-minute hold at 100% B; Flow Rate: 45 mL/min; Column Temperature: 25° C. Fraction collection was triggered by MS and UV signals. Fractions containing the desired product were combined and dried via centrifugal evaporation. The yield of the product was 2.5 mg, and its estimated purity by LCMS analysis was 99%. Analysis condition A: Retention time=1.53 min; ESI-MS(+) m/z [M+2H]²⁺: 1073.04. Analysis condition B: Retention time=1.91 min; ESI-MS(+) m/z [M+2H]²⁺: 1072.98.

### Preparation of Compound 1039

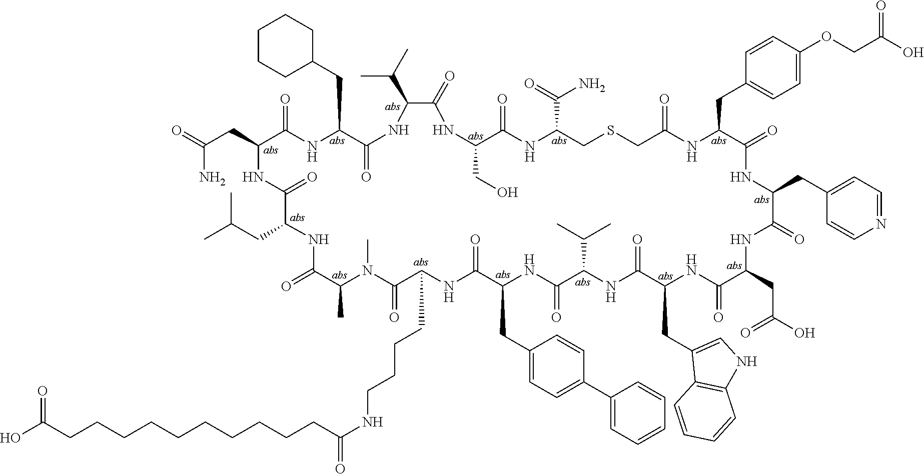 [SMILES: CC(C)C[C@H]1NC(=O)[C@H](C)N(C)C(=O)[C@H](CCCCNC(=O)CCCCCCCCCCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(N)=O)NC(=O)[C@H](CO)NC(=O)[C@H](C(C)C)NC(=O)[C@H](CC2CCCCC2)NC(=O)[C@H](CC(N)=O)NC1=O]

### Preparation of Compound 1039A

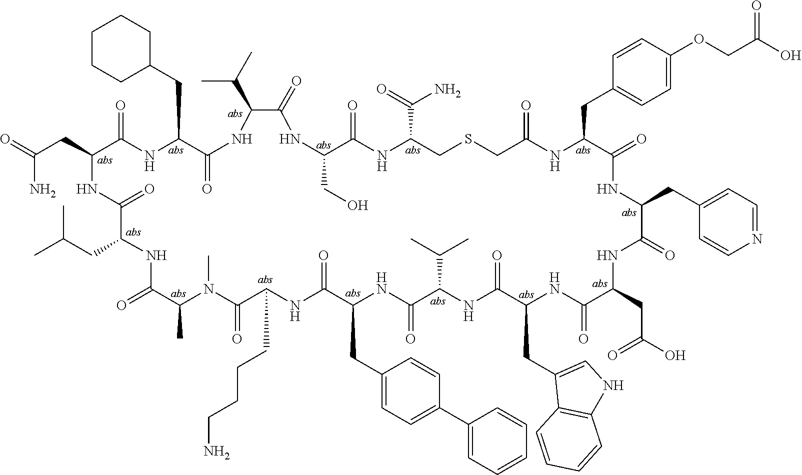 [SMILES: CC(C)C[C@H]1NC(=O)[C@H](C)N(C)C(=O)[C@H](CCCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(N)=O)NC(=O)[C@H](CO)NC(=O)[C@H](C(C)C)NC(=O)[C@H](CC2CCCCC2)NC(=O)[C@H](CC(N)=O)NC1=O]

Compound 1039A was prepared following the procedures for Compound 1038 on a 50 μmol scale. The yield of the product was 30.9 mg, and its estimated purity by LCMS analysis was 99.1%. Analysis condition A: Retention time=1.7 min; ESI-MS(+) m/z [M+2H]²+: 967.2.

Compound 1039 was prepared from Compound 1039A following the procedure for the preparation of Compound 1038 on a 10.4 μmol scale. The crude material was purified via preparative LC/MS with the following conditions: Column: XBridge C18, 150 mm×30 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 22% B, 22-62% B over 20 minutes, then a 2-minute hold at 100% B; Flow Rate: 40 mL/min; Column Temperature: 25° C. Fraction collection was triggered by MS and UV signals. Fractions containing the desired product were combined and dried via centrifugal evaporation. The yield of the product was 2.4 mg, and its estimated purity by LCMS analysis was 94.6%.

Analysis condition B: Retention time=1.75, 1.82 min; ESI-MS(+) m/z [M+2H]²⁺: 1074.16, 1074.12.

### Preparation of Compound 1040

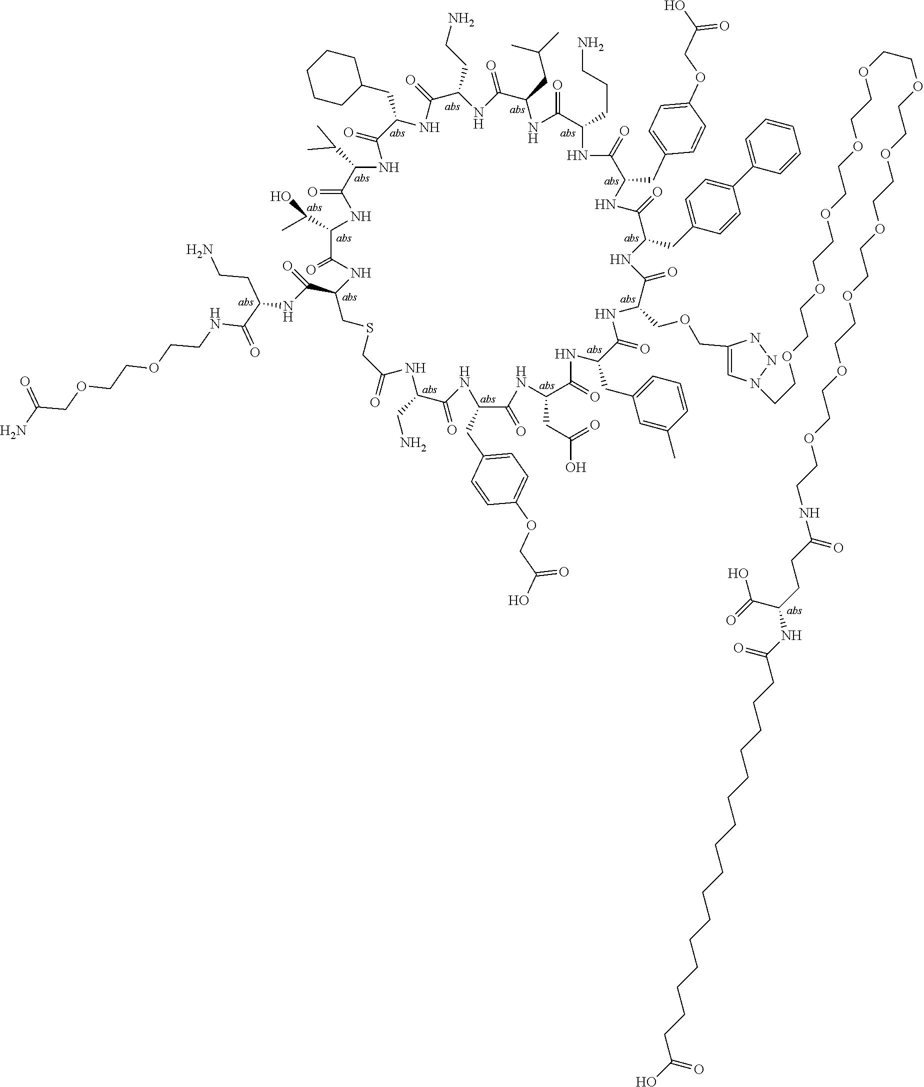 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](COCC3=CN(CCOCCOCCOCCOCCOCCOCCOCCOCCOCCOCCOCCNC(=O)CC[C@H](NC(=O)CCCCCCCCCCCCCCCCC(=O)O)C(=O)O)NN3)NC2=O)c1]

### Compound 1040A

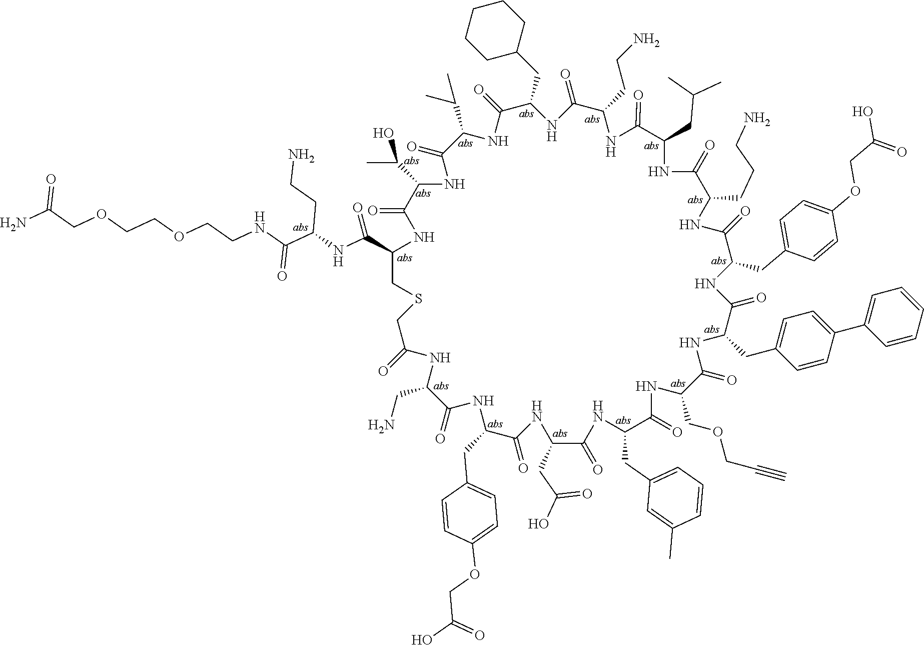 [SMILES: C#CCOC[C@@H]1NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC1=O]

Compound 1040A: To a 45-mL polypropylene solid-phase reaction vessel was added Sieber resin on a 100 μmol scale, and the reaction vessel was placed on the Symphony X peptide synthesizer. The following procedures were then performed sequentially: “Symphony X Resin-Swelling procedure” was followed; “Symphony X Single-Coupling procedure “was followed with FmocNHCH₂CH₂OCH₂CH₂OCH₂COOH; “Symphony XSingle-Coupling procedure “was followed with Fmoc-Dab(Boc)—OH; “Symphony XSingle-Couplingprocedure “was followed with Fmoc-Cys(Trt)—OH; “Symphony XSingle-Couplingprocedure “was followed with Fmoc-Thr(tBu)—OH; “Symphony XSingle-Couplingprocedure” was followed with Fmoc-Val-OH; “Symphony XSingle-Couplingprocedure “was followed with Fmoc-Cha-OH; “Symphony XSingle-Couplingprocedure “was followed with Fmoc-Dab(OtBu)—OH; “Symphony X Single-Coupling procedure” or “Symphony Double-Coupling procedure” was followed with Fmoc-D-Leu-OH; “Symphony X Single-Coupling procedure” or “Symphony Double-Coupling procedure” was followed with Fmoc-N-Me-Ala-OH; “Symphony X Single-Coupling procedure “was followed with Fmoc-Gln(Trt)—OH; “Symphony XSingle-Coupling procedure “was followed with Fmoc-Bip-OH; “Symphony XSingle-Couplingprocedure “was followed with Fmoc-Ser(Propargyl)—OH; “Symphony XSingle-Couplingprocedure” was followed with Fmoc-Phe(3-Me)—OH; “Symphony XSingle-Couplingprocedure “was followed with Fmoc-Asp(OtBu)—OH; “Symphony XSingle-Coupling procedure “was followed with Fmoc-Tyr(CH₂COOtBu)—OH; “Symphony XSingle-Couplingprocedure “was followed with Fmoc-Dap(boc)—OH. The crude material was purified via preparative LC/MS with the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 19% B, 19-59% B over 25 minutes, then a 0-minute hold at 100% B; Flow Rate: 20 mL/min; Column Temperature: 25° C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 14% B, 14-54% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 26.2 mg, and its estimated purity by LCMS analysis was 96%. Analysis condition A: Retention time=1.52, 1.54 min; ESI-MS(+) m/z [M+3H]^{3+: 860.9, 860.9.}

Compound 1040: The compound was synthesized following the general procedure “Solution Phase Click Method A”. To a 20-mL scintillation vial is added 100-times of sodium (R)-2-((S)-1,2-dihydroxyethyl)-4-hydroxy-5-oxo-2,5-dihydrofuran-3-olate (0.572 mg, 2.89 μmol) and copper(II) sulfate pentahydrate (0.360 mg, 1.444 μmol). The reaction was diluted with Water (10 mL). This solution was shaken at RT for 1-10 min. The resulting yellowish slurry was added to the reaction.

To the vial containing Compound 1040A (44.9 mg, 20 μmol) was added tBuOH/Water (v/v 1:1, 1 mL) and (S)-1-azido-40-carboxy-37,42-dioxo-3,6,9,12,15,18,21,24,27,30,33-undecaoxa-36,41-diazanonapentacontan-59-oic acid, N₃-Peg11-γGLu-FDA18 (26.0 mg, 26 μmol), followed by 100 μL of the above copper solution. The mixture was shaken at rt for 1 h and the progress was monitored by LC/MS. After completion, the mixture was diluted with CH₃CN: aq. NH₄CO₃ solution (v/v 1:1), filtered, and submitted to single compound purification.

The crude material was purified via preparative LC/MS with the following conditions: Column: Waters CSH Fluoro Phenyl, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 18% B, 18-58% B over 20 minutes, then a 2-minute hold at 100% B; Flow Rate: 25 mL/min; Column Temperature: 25° C. Fraction collection was triggered by MS signals. Fractions containing the desired product were combined and dried via centrifugal evaporation. The yield of the product was 21.6 mg, and its estimated purity by LCMS analysis was 96.7%.

Analysis condition 2: Retention time=1.95 min; ESI-MS(+) m z²⁺: 1079.

### Preparation of Compound 1041

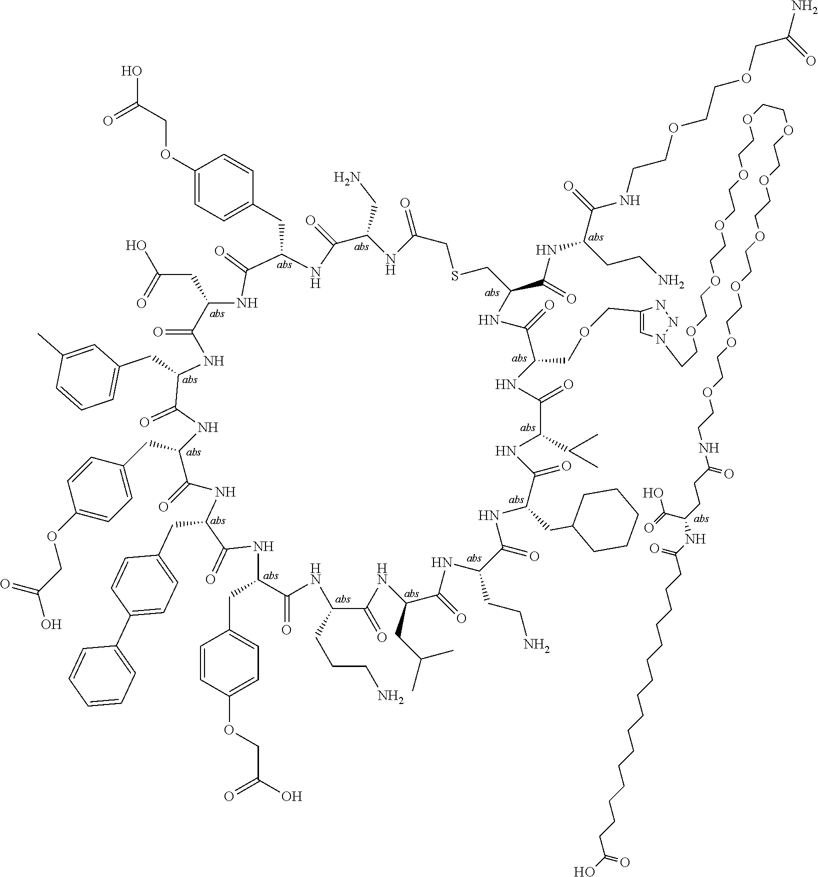 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(N)=O)NC(=O)[C@H](COCC3=CN(CCOCCOCCOCCOCCOCCOCCOCCOCCOCCOCCOCCNC(=O)CC[C@H](NC(=O)CCCCCCCCCCCCCCCCC(=O)O)C(=O)O)NN3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

### Compound 1041A

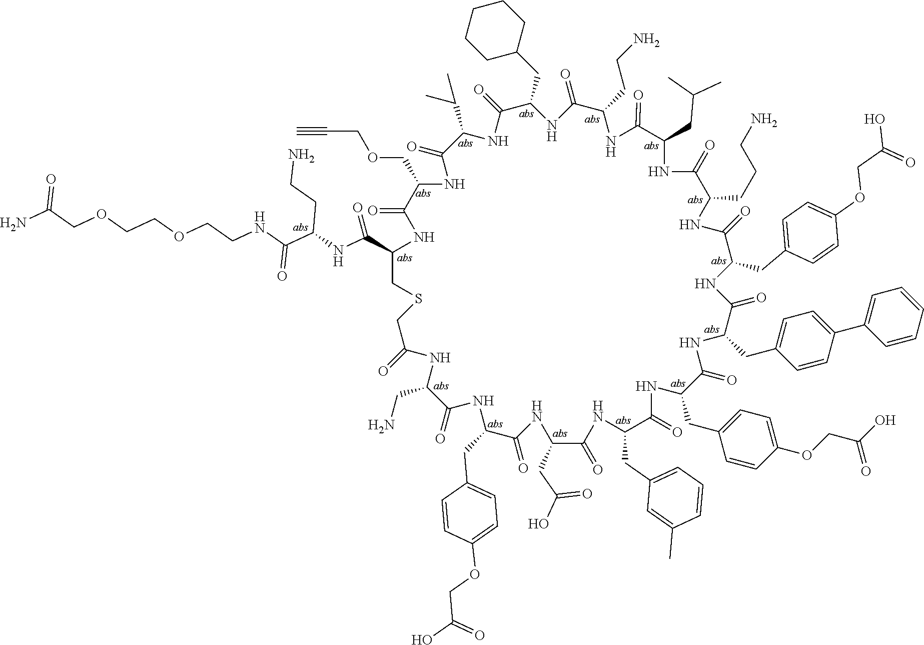 [SMILES: C#CCOC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(N)=O)NC1=O]

Compound 1041A was prepared following the procedures described for Compound 1040A on a 50 μmol scale. The yield of the product was 15.4 mg, and its estimated purity by LCMS analysis was 91.8%. Analysis condition A: Retention time=1.5, 1.54 min; ESI-MS(+) m/z [M+3H]^{3+: 787.01, 787.21.}

Compound 1041: The compound was prepared on a 4.5 μmol scale from Compound 1041A following the procedures described for the preparation Compound 1040. The crude material was purified via preparative LC/MS with the following conditions: Column: XBridge C18, 200 mm×30 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 33% B, 33-73% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 7 mg, and its estimated purity by LCMS analysis was 95.3%.

Analysis condition A: Retention time=1.7 min; ESI-MS(+) m/z [M+2H]²⁺: 1679.2.

### Preparation of Compound 1042

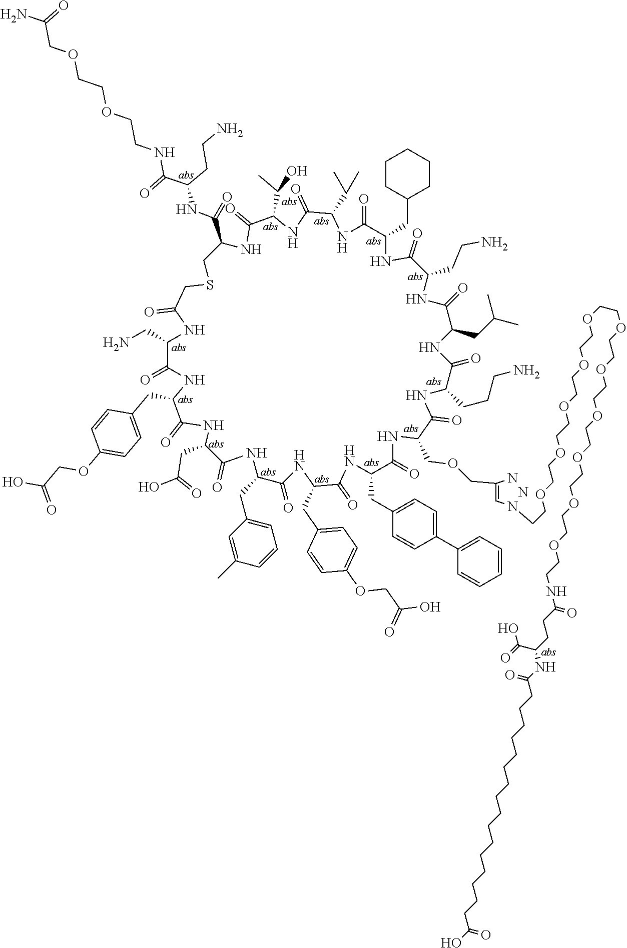 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](COCC3=CN(CCOCCOCCOCCOCCOCCOCCOCCOCCOCCOCCOCCNC(=O)CC[C@H](NC(=O)CCCCCCCCCCCCCCCCC(=O)O)C(=O)O)NN3)NC(=O)[C@H](Cc3ccc(-c4ccccc4)cc3)NC(=O)[C@H](Cc3ccc(OCC(=O)O)cc3)NC2=O)c1]

### Compound 1042A

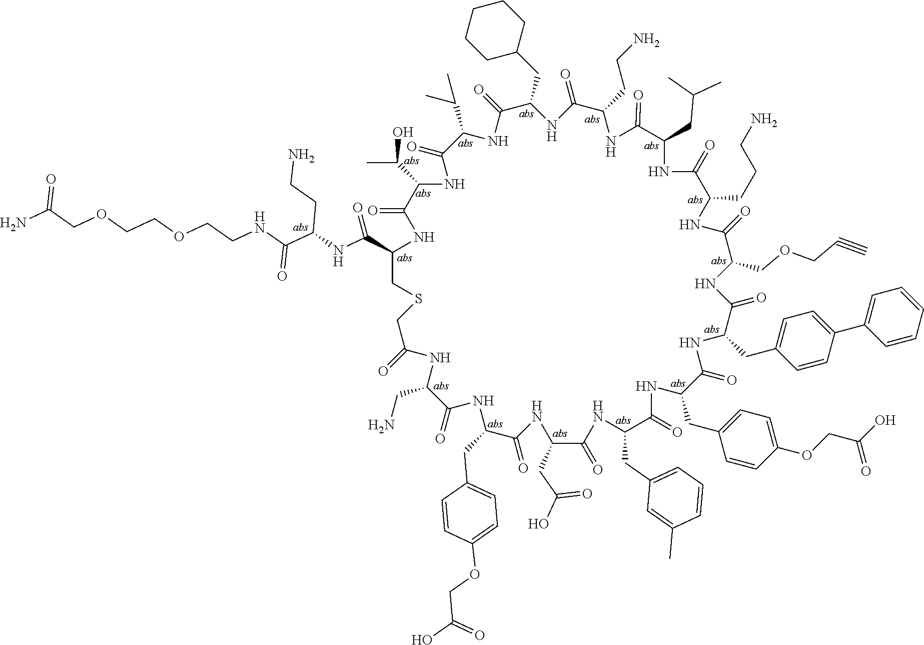 [SMILES: C#CCOC[C@@H]1NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1042A was prepared following the procedures described for Compound 1040A on a 50 μmol scale. The yield of the product was 26.7 mg, and its estimated purity by LCMS analysis was 97.5%. Analysis condition A: Retention time=1.45 min; ESI-MS(+) m/z [M+2H]²⁺: 1120.2.

Compound 1042: The compound was prepared on a 8.7 μmol scale from Compound 1042A following the procedures described for the preparation Compound 1040. The crude material was purified via preparative LC/MS with the following conditions: Column: XBridge C18, 200 mm×30 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 30% B, 30-70% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 9.9 mg, and its estimated purity by LCMS analysis was 97.2%.

Analysis condition A: Retention time=1.83 min; ESI-MS(+) m/z [M+3H]³⁺: 1079.

### Preparation of Compound 1043

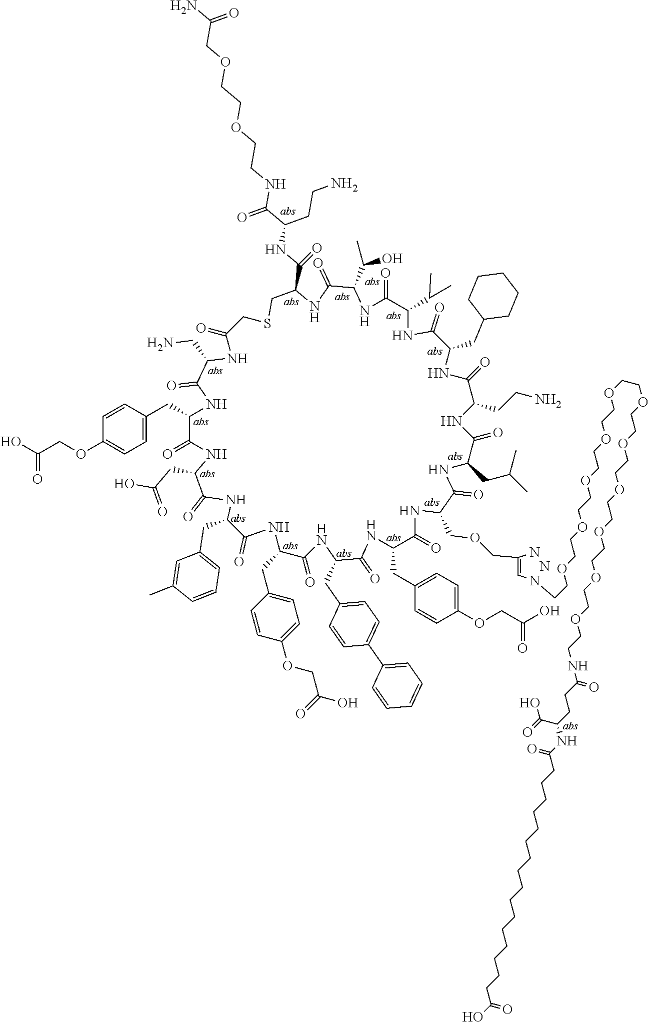 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(=O)NCCOCCOCC(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](COCC3=CN(CCOCCOCCOCCOCCOCCOCCOCCOCCOCCOCCOCCNC(=O)CC[C@H](NC(=O)CCCCCCCCCCCCCCCCC(=O)O)C(=O)O)NN3)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

### Compound 1043A

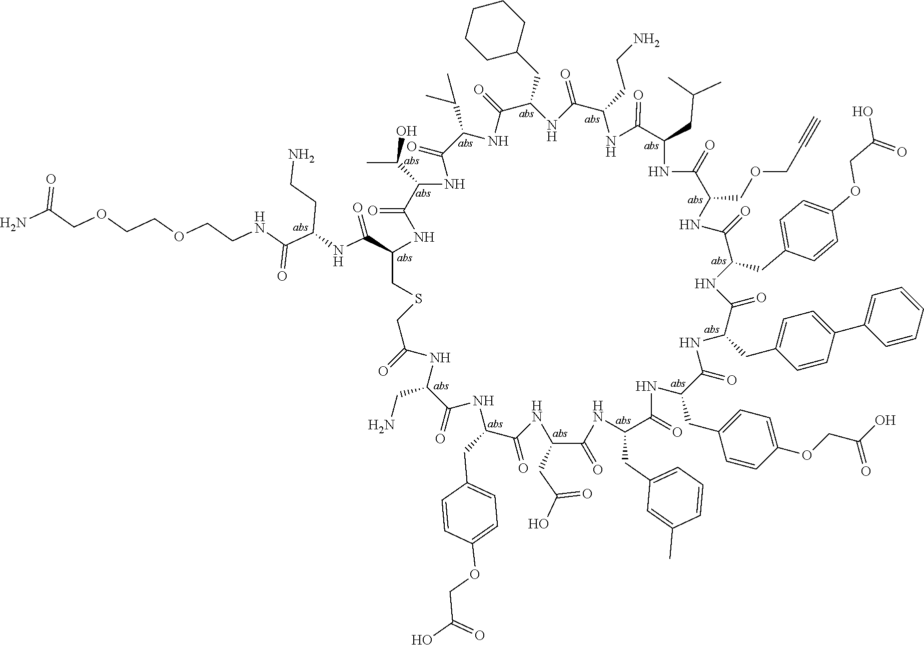 [SMILES: C#CCOC[C@@H]1NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(=O)NCCOCCOCC(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC1=O]

Compound 1043A was prepared following the procedures described for Compound 1040A on a 50 μmol scale. The yield of the product was 31.6 mg, and its estimated purity by LCMS analysis was 93.1%. Analysis condition B: Retention time=1.54 min; ESI-MS(+) m/z [M+2H]²⁺: 1173.9.

Compound 1043: The compound was prepared on a 9.4 μmol scale from Compound 1043A following the procedures described for the preparation Compound 1040. The crude material was purified via preparative LC/MS with the following conditions: Column: XBridge C18, 200 mm×30 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-minute hold at 33% B, 33-73% B over 20 minutes, then a 0-minute hold at 100% B; Flow Rate: 45 mL/min; Column Temperature: 25° C. Fraction collection was triggered by MS signals. Fractions containing the desired product were combined and dried via centrifugal evaporation. The yield of the product was 10.2 mg, and its estimated purity by LCMS analysis was 93.8%.

Analysis condition B: Retention time=2.06 min; ESI-MS(+) m/z [M+3H]³⁺: 1115.2.

### Preparation of Compound 1044

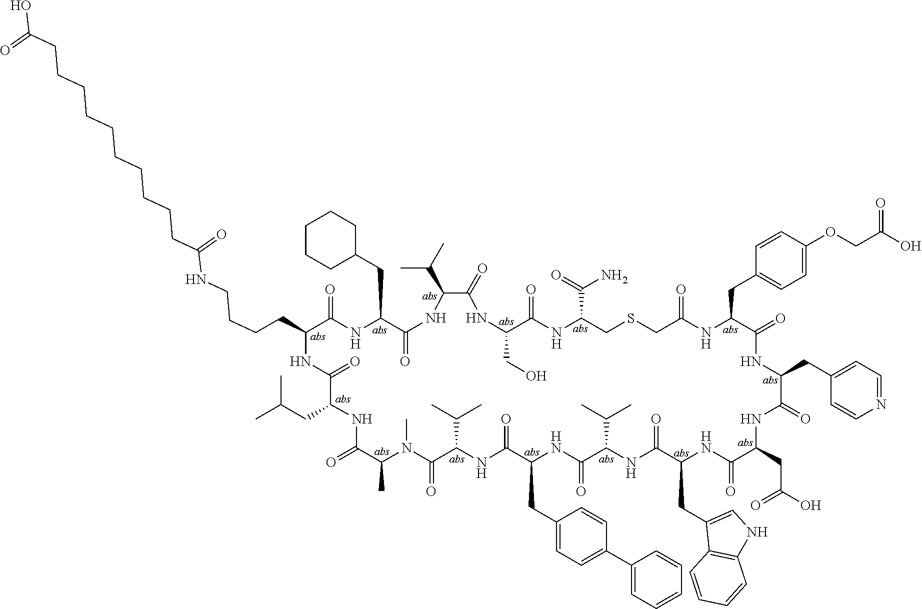 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(N)=O)NC(=O)[C@H](CO)NC(=O)[C@H](C(C)C)NC(=O)[C@H](CC2CCCCC2)NC(=O)[C@H](CCCCNC(=O)CCCCCCCCCCC(=O)O)NC1=O]

### Compound 1044A

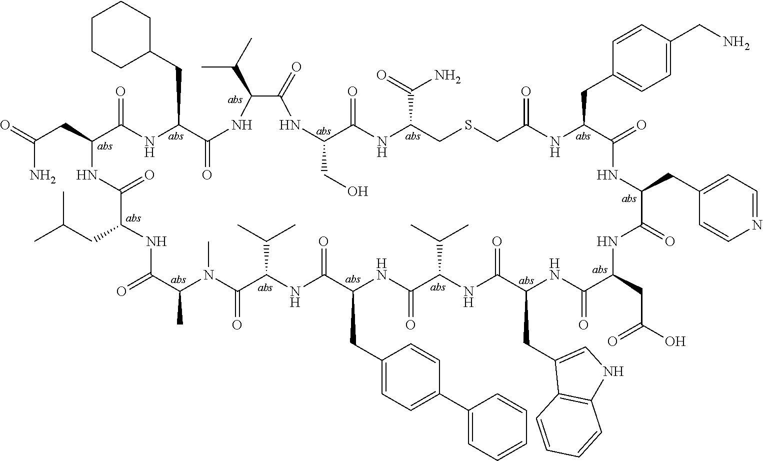 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(CN)cc2)NC(=O)CSC[C@@H](C(N)=O)NC(=O)[C@H](CO)NC(=O)[C@H](C(C)C)NC(=O)[C@H](CC2CCCCC2)NC(=O)[C@H](CC(N)=O)NC1=O]

Compound 1044A was prepared following the procedures for Compound 1038A on a 50 μmol scale. The yield of the product was 27.3 mg, and its estimated purity by LCMS analysis was 100%. Analysis condition A: Retention time=1.84 min; ESI-MS(+) m/z [M+H]⁺: 1858.5.

Compound 1044 was prepared from Compound 1044A following the procedure for the preparation of Compound 1038 on a 10.8 μmol scale. The crude material was purified via preparative LC/MS with the following conditions: Column: XBridge C18, 200 mm×30 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 21% B, 21-61% B over 20 minutes, then a 2-minute hold at 100% B; Flow Rate: 45 mL/min; Column Temperature: 25° C. Fraction collection was triggered by MS and UV signals. Fractions containing the desired product were combined and dried via centrifugal evaporation. The yield of the product was 1.4 mg, and its estimated purity by LCMS analysis was 91.1%.

Analysis condition B: Retention time=1.7 min; ESI-MS(+) m/z [M+2H]²⁺: 1066.

### Preparation of Compound 1045

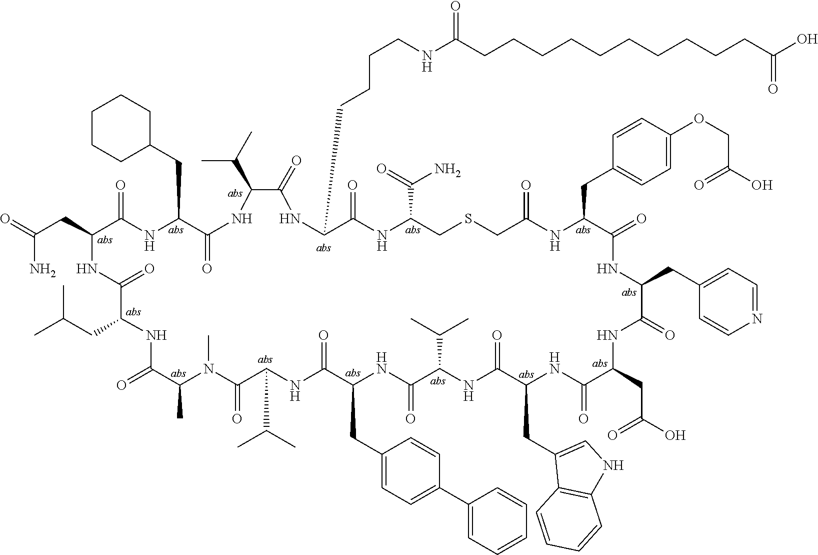 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(N)=O)NC(=O)[C@H](CCCCNC(=O)CCCCCCCCCCC(=O)O)NC(=O)[C@H](C(C)C)NC(=O)[C@H](CC2CCCCC2)NC(=O)[C@H](CC(N)=O)NC1=O]

### Compound 1045A

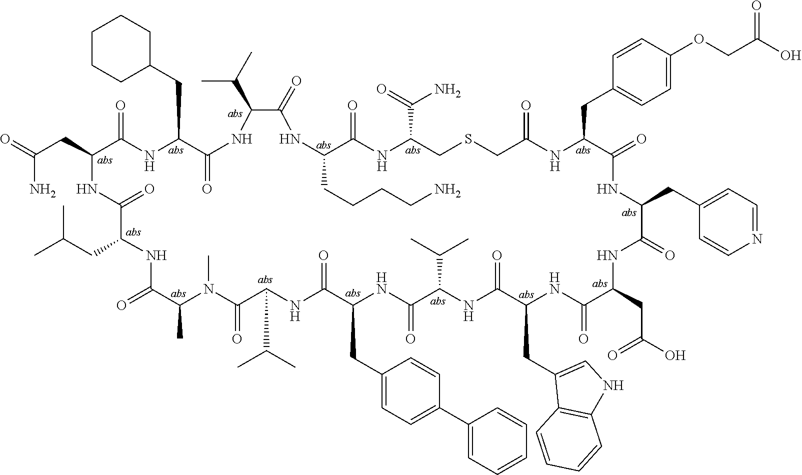 [SMILES: CC(C)C[C@H]1NC(=O)[C@H](C)N(C)C(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(N)=O)NC(=O)[C@H](CCCCN)NC(=O)[C@H](C(C)C)NC(=O)[C@H](CC2CCCCC2)NC(=O)[C@H](CC(N)=O)NC1=O]

Compound 1045A was prepared following the procedures for Compound 1038A on a 50 μmol scale. The yield of the product was 29.2 mg, and its estimated purity by LCMS analysis was 97.9%. Analysis condition B: Retention time=1.78 min; ESI-MS(+) m/z [M+H]⁺: 1944.8.

Compound 1045 was prepared from Compound 1045A following the procedure for the preparation of Compound 1038 on a 10.4 μmol scale. The crude material was purified via preparative LC/MS with the following conditions: Column: XBridge C18, 150 mm×30 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 22% B, 22-62% B over 20 minutes, then a 2-minute hold at 100% B; Flow Rate: 40 mL/min; Column Temperature: 25° C. Fraction collection was triggered by MS and UV signals. Fractions containing the desired product were combined and dried via centrifugal evaporation. The yield of the product was 2.5 mg, and its estimated purity by LCMS analysis was 91.6%. Analysis condition A: Retention time=1.72 min; ESI-MS(+) m/z [M+2H]²⁺: 1078.5.

### Biological Activity

The ability of the compounds of formula (I) to bind to PD-1 was investigated using a Jurkat-PD-1 Cell Binding High-Content Screening Assay.

### Jurkat-PD-1 Cell Binding

Phycoerythrin (PE) was covalently linked to the Ig epitope tag of human PD-L1-Ig and fluorescently-labeled PD-L1-Ig was used for binding studies with a Jurkat cell line over-expressing human PD-1 (Jurkat-PD-1). Briefly, 8×10³ Jurkat-hPD-1 cells were seeded into 384 well plates in 20 μl of DIVEM supplemented with 10% fetal calf serum. 100 nl of compound was added to cells followed by incubation at 37° C. for 2 h. Then, 5 μl of PE-labeled PD-L1-Ig (20 nM final), diluted in DiVEM supplemented with 10 fetal calf serum. After 1 hour incubation, cells were fixed with 400 paraformaldehyde in dPBS containing 10 g/mL Hoechst 33342 and then washed 3× in 100 μl dPB S. Data was collected and processed using a Cell Insight NXT High Content Imager and associated software. IC50 values are shown in Table 3.

TABLE 3

| Example Number | Jurkat hPD1 PDL1 IC50 (uM) |
|---|---|
| 1001 | 0.16 |
| 1002 | 0.14 |
| 1003 | 0.21 |
| 1004 | 0.11 |
| 1005 | 0.039 |
| 1006 | 0.055 |
| 1007 | 0.51 |
| 1008 | 0.71 |
| 1009 | 0.72 |
| 1010 | 0.63 |
| 1011 | 0.34 |
| 1012 | 0.38 |
| 1013 | 0.24 |
| 1014 | 0.073 |
| 1015 | 0.10 |
| 1016 | 0.10 |
| 1017 | 0.090 |
| 1018 | 0.11 |
| 1019 | 0.14 |
| 1020 | 0.096 |
| 1021 | 0.095 |
| 1022 | 0.054 |
| 1023 | 0.099 |
| 1024 | 0.40 |
| 1025 | 0.16 |
| 1026 | 0.18 |
| 1027 | 0.47 |
| 1028 | 0.43 |
| 1029 | 0.47 |
| 1030 | 0.27 |
| 1031 | 0.29 |
| 1032 | 0.12 |
| 1033 | 0.63 |
| 1034 | 0.17 |
| 1035 | 0.13 |
| 1036 | 0.13 |
| 1037 | 0.12 |
| 1038 | 0.18 |
| 1039 | 0.87 |
| 1040 | 0.25 |
| 1041 | 0.14 |
| 1042 | 0.22 |
| 1043 | 0.61 |
| 1044 | 0.36 |
| 1045 | 0.18 |

The compounds of formula (I) possess activity as inhibitors of the PD-1/PD-L1 interaction, and therefore, can be used in the treatment of diseases or deficiencies associated with the PD-1/PD-L1 interaction. Via inhibition of the PD-1/PD-L1 interaction, the compounds of the present disclosure can be employed to treat infectious diseases such as HIV, septic shock, Hepatitis A, B, C, or D and cancer.

It is to be appreciated that the Detailed Description section, and not the Summary and Abstract sections, is intended to be used to interpret the claims. The Summary and Abstract sections can set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

What is claimed is:

1. A compound of formula (I) 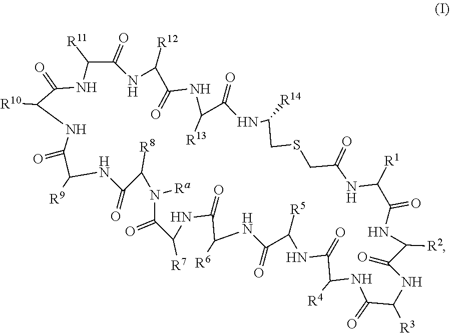
or a pharmaceutically acceptable salt thereof, wherein:
R¹ is selected from C₁-C₆alkyl, aminoC₁-C₆alkyl, arylC₁-C₆alkyl, heteroarylC₁-C₆alkyl, hydroxyC₁-C₆alkyl, —X—R³¹, —(CH₂)_{z}—O—(CH₂)_{z}-triazolyl-X—R³⁵, and NH₂C(X″)NHC₁-C₆alkyl, wherein X″ is O or NH, and wherein the aryl part of the arylC₁-C₆alkyl and the heteroaryl part of the heteroarylC₁-C₆alkyl are optionally substituted with one or two groups independently selected from carboxy, carboxyC₁-C₆alkoxy, —X—R³¹, and —O—(CH₂)_{z}-triazolyl-X—R³⁵
R² is selected from arylC₁-C₆alkyl, and heteroarylC₁-C₆alkyl, wherein the aryl part of the arylC₁-C₆alkyl and the heteroaryl part of the heteroarylC₁-C₆alkyl are optionally substituted with one or two groups independently selected from carboxy, carboxyC₁-C₆alkoxy, —X—R³¹, and —O—(CH₂)_{z}-triazolyl-X—R³⁵
R³ is carboxyC₁-C₃alkyl;
R⁴ is selected from arylC₁-C₆alkyl and heteroarylC₁-C₆alkyl, wherein the aryl part of the arylC₁-C₆alkyl and the heteroaryl part of the heteroarylC₁-C₆alkyl are optionally substituted with one or two C₁-C₆alkyl groups;
R⁵ is selected from C₁-C₆alkyl, arylC₁-C₆alkyl, —X—R³¹, and —(CH₂)_{z}—O—(CH₂)_{z}-triazolyl-X—R³⁵, wherein the aryl part of the arylC₁-C₆alkyl is optionally substituted with one or two groups independently selected from carboxy, carboxyC₁-C₆alkoxy, hydroxy, —X—R³¹, and —O—(CH₂)_{z}-triazolyl-X—R³⁵;
R⁶ is aryl-arylC₁-C₃alkyl;
R⁷ is selected from C₁-C₆alkyl, arylC₁-C₆alkyl, —X—R³¹, and —(CH₂)_{z}—O—(CH₂)_{z}-triazolyl-X—R³⁵, wherein the aryl part of the arylC₁-C₆alkyl is optionally substituted with one or two groups independently selected carboxy, carboxyC₁-C₆alkoxy, —X—R³¹, and —O—(CH₂)_{z}-triazolyl-X-R³⁵
R⁸ is selected from C₁-C₆alkyl, aminoC₁-C₆alkyl, —X—R³¹, and —O—(CH₂)_{z}-triazolyl-X—R³⁵;
R⁹ is C₁-C₆alkyl;
R¹⁰ is selected from amidoC₁-C₆alkyl, aminoC₁-C₆alkyl, —X—R³¹, and —(CH₂)_{z}—O—(CH₂)_{z}—triazolyl-X—R³⁵;
R¹¹ is (C₃-C₈cycloalkyl)C₁-C₆alkyl;
R¹² is selected from C₁-C₆alkyl;
R¹³ is selected from arylC₁-C₆alkyl, carboxyC₁-C₆alkyl, hydroxyC₁-C₆alkyl, —X—R³¹, and —(CH₂)_{z}—O—(CH₂)_{z}-triazolyl-X—R³⁵, wherein the aryl part of the arylC₁-C₆alkyl is optionally substituted with one or two groups independently selected carboxy, carboxyC₁-C₆alkoxy, —X—R³¹, and —O—(CH₂)_{z}-triazolyl-X—R³⁵;
R¹⁴ is —C(O)NH₂ or —C(O)NHCHR¹⁵C(O)NHR⁵⁰, wherein:
R¹⁵ is selected from hydrogen, C₁-C₆alkyl, and aminoC₁-C₆alkyl; and
R⁵⁰ is selected from hydrogen and NH₂C(O)CH₂(OCH₂CH₂)₂—; and
R³¹ is —CO₂H, —C(O)NR^{w}R^{x}, —CH₃, alexa-5-SDP, and biotin;
each z is independently 1, 2, 3, 4, 5, or 6; and
R³⁵ is selected from —CO₂H, —C(O)NR^{w}R^{x}, CH₃, biotin, 2-fluoropyridine, —C(O)—(CH₂)₂-C(O)-vitamin E, and —C(O)-vitamin E; wherein R^{x} and R^{W} are independently selected from hydrogen and C₁-C₆alkyl;
X is a chain of between 1 and 172 atoms wherein the atoms are selected from carbon and oxygen and wherein the chain may contain one, two, three, or four groups selected from —NHC(O)NH—, and —C(O)NH— embedded therein; and wherein the chain is optionally substituted with one to six groups independently selected from —CO₂H, —C(O)NH₂, —CH₂C(O)NH₂, and —(CH₂)CO₂H;
provided that at least one of R¹, R², R⁵, R⁷, R⁸, R¹⁰, and R¹³ is, or is substituted with, a group selected from —X—R³¹, —(CH₂)_{z}—O—(CH₂)_{z}-triazolyl-X—R^{35′} and —O—(CH₂)_{z}-triazolyl-X—R³⁵.

2. The compound of claim 1, wherein:
R¹ is selected from C₁-C₆alkyl, aminoC₁-C₆alkyl, arylC₁-C₆alkyl, heteroarylC₁-C₆alkyl, hydroxyC₁-C₆alkyl, HO₂C(CH₂)₁₀C(O)NH(CH₂)_{z}— and NH₂C(X″)NHC₁-C₆alkyl, wherein z is 1, 2, 3, or 4 and X″ is O or NH, and wherein the aryl part of the arylC₁-C₆alkyl and the heteroaryl part of the heteroarylC₁-C₆alkyl are optionally substituted with one or two groups independently selected from carboxy, carboxyC₁-C₆alkoxy, and HO₂C(CH₂)₁₆C(O)NHCH(CO₂H)(CH₂)₂C(O)NH(CH₂CH₂O)₁₁(CH₂)₂triazolylCH₂O—;
R² is selected from arylC₁-C₆alkyl, and heteroarylC₁-C₆alkyl, wherein the aryl part of the arylC₁-C₆alkyl and the heteroaryl part of the heteroarylC₁-C₆alkyl are optionally substituted with one or two groups independently selected from carboxy, carboxyC₁-C₆alkoxy, HO₂C(CH₂)₁₀C(O)NH(CH₂)_{z}—, and HO₂C(CH₂)₁₆C(O)NHCH(CO₂H)(CH₂)₂C(O)NH(CH₂CH₂O)₁₁(CH₂)₂triazolylCH₂O—, wherein z is 1, 2, 3, or 4;
R⁵ is selected from C₁-C₆alkyl, arylC₁-C₆alkyl, and HO₂C(CH₂)₁₆C(O)NHCH(CO₂H)(CH₂)₂C(O)NH(CH₂CH₂O)₁₁(CH₂)₂triazolylCH₂OCH₂—, wherein the aryl part of the arylC₁-C₆alkyl is optionally substituted with one or two groups independently selected from carboxy, carboxyC₁-C₆alkoxy, hydroxy, and HO₂C(CH₂)₁₆C(O)NHCH(CO₂H)(CH₂)₂C(O)NH(CH₂CH₂O)₁₁(CH₂)₂triazolylCH₂O—;
R⁷ is selected from C₁-C₆alkyl, arylC₁-C₆alkyl, HO₂C(CH₂)₁₀C(O)NH(CH₂)_{z}—, HO₂C(CH₂)₁₆C(O)NHCH(CO₂H)(CH₂)₂C(O)NH(CH₂CH₂O)₁₁(CH₂)₂triazolylCH₂OCH₂—, wherein z is 1, 2, 3, or 4, and wherein the aryl part of the arylC₁-C₆alkyl is optionally substituted with one or two groups independently selected carboxy, carboxyC₁-C₆alkoxy, and HO₂C(CH₂)₁₆C(O)NHCH(CO₂H)(CH₂)₂C(O)NH(CH₂CH₂O)₁₁(CH₂)₂triazolylCH₂O—;
R⁸ is selected from C₁-C₆alkyl, aminoC₁-C₆alkyl, HO₂C(CH₂)₁₀C(O)NH(CH₂)_{z}—, HO₂C(CH₂)₁₆C(O)NHCH(CO₂H)(CH₂)₂C(O)NH(CH₂CH₂O)₁₁(CH₂)₂triazolylCH₂OCH₂—, wherein z is 1, 2, 3, or 4;
R¹⁰ is selected from amidoC₁-C₆alkyl, aminoC₁-C₆alkyl, and HO₂C(CH₂)₁₀C(O)NH(CH₂)_{z}—, wherein z is 1, 2, 3, or 4;
R¹³ is selected from arylC₁-C₆alkyl, carboxyC₁-C₆alkyl, hydroxyC₁-C₆alkyl, HO₂C(CH₂)₁₀C(O)NH(CH₂)_{z}—, wherein z is 1, 2,3 or 4, and
HO₂C(CH₂)₁₆C(O)NHCH(CO₂H)(CH₂)₂C(O)NH(CH₂CH₂O)₁₁(CH₂)₂triazolylCH₂OCH₂—;
provided that at least one of R¹, R², R⁵, R⁷, R⁸, R¹⁰, and R¹³ is, or is substituted with, a group selected from
HO₂C(CH₂)₁₆C(O)NHCH(CO₂H)(CH₂)₂C(O)NH(CH₂CH₂O)₁₁(CH₂)₂triazolylCH₂O—,
HO₂C(CH₂)₁₀C(O)NH(CH₂)_{z}, and
HO₂C(CH₂)₁₆C(O)NHCH(CO₂H)(CH₂)₂C(O)NH(CH₂CH₂O)ln(CH₂)₂triazolylCH₂OCH₂—.

3. The compound of claim 1 or 2, or a pharmaceutically acceptable salt thereof, wherein at least one of R¹, R⁷, R⁸, R¹⁰, and R¹³ is HO₂C(CH₂)₁₀C(O)NH(CH₂)_{z}—, wherein z is 1, 2, 3, or 4.

4. The compound of claim 1 or 2, or a pharmaceutically acceptable salt thereof, wherein at least one of R¹, R², R⁵, and R⁷ is arylC₁-C₆alkyl or heteroarylC₁-C₆alkyl wherein the aryl part of the arylC₁-C₆alkyl and the heteroaryl part of the heteroarylC₁-C₆alkyl are substituted with HO₂C(CH₂)₁₆C(O)NHCH(CO₂H)(CH₂)₂C(O)NH(CH₂CH₂O)₁₁(CH₂)₂triazolylCH₂O— or HO₂C(CH₂)₁₀C(O)NH(CH₂)_{z}—.

5. The compound of claim 1 or 2, or a pharmaceutically acceptable salt thereof, wherein at least one of R⁵, R⁷, R⁸, and R¹³ is HO₂C(CH₂)₁₆C(O)NHCH(CO₂H)(CH₂)₂C(O)NH(CH₂CH₂O)₁₁(CH₂)₂triazolylCH₂OCH₂—.

6. The compound of any one of claims 1 to 5, or a pharmaceutically acceptable salt thereof, wherein R² is arylC₁-C₆alkyl, wherein the aryl part of the arylC₁-C₆alkyl is optionally substituted with one or two groups independently selected from carboxy, carboxyC₁-C₆alkoxy, HO₂C(CH₂)₁₀C(O)NHCH₂—, and HO₂C(CH₂)₁₆C(O)NHCH(CO₂H)(CH₂)₂C(O)NH(CH₂CH₂O)₁₁(CH₂)₂triazolylCH₂O—.

7. The compound of claim 1 or 6, or a pharmaceutically acceptable salt thereof, wherein R⁴ is arylC₁-C₆alkyl, wherein the aryl part of the arylC₁-C₆alkyl is optionally substituted with one or two C₁-C₆alkyl groups.

8. The compound of any one of claims 1 to 7, or a pharmaceutically acceptable salt thereof, wherein R⁵ is arylC₁-C₆alkyl, wherein the aryl part of the arylC₁-C₆alkyl is optionally substituted with one or two groups independently selected from carboxy, carboxyC₁-C₆alkoxy, hydroxy, and HO₂C(CH₂)₁₆C(O)NHCH(CO₂H)(CH₂)₂C(O)NH(CH₂CH₂O)₁₁(CH₂)₂triazolylCH₂O—.

9. The compound of any one of claims 1 to 8, or a pharmaceutically acceptable salt thereof, wherein R⁷ is arylC₁-C₆alkyl, wherein the aryl part of the arylC₁-C₆alkyl is optionally substituted with one or two groups independently selected carboxy, carboxyC₁-C₆alkoxy, and HO₂C(CH₂)₁₆C(O)NHCH(CO₂H)(CH₂)₂C(O)NH(CH₂CH₂O)ln(CH₂)₂triazolylCH₂O—.

10. The compound of any one of claims 1 to 9, or a pharmaceutically acceptable salt thereof, wherein R⁸ is aminoC₁-C₆alkyl.

11. The compound of any one of claims 1 to 10, or a pharmaceutically acceptable salt thereof, wherein R¹⁰ is aminoC₁-C₆alkyl.

12. A pharmaceutical composition comprising a compound of any one of claims 1 to 11, or a pharmaceutically acceptable salt thereof.

13. A method of enhancing, stimulating, and/or increasing an immune response in a subject in need thereof, wherein the method comprises administering to the subject a therapeutically effective amount of a compound of any one of claims 1 to 11, or a pharmaceutically acceptable salt thereof.

14. A method of blocking the interaction of PD-1 with PD-L1 in a subject, wherein the method comprises administering to the subject a therapeutically effective amount of a compound of any one of claims 1 to 11, or a pharmaceutically acceptable salt thereof.
